(19) United States
(12) Patent Application Publication
(10) Pub. No.: US 2013/0281399 A1
(43) Pub. Date: Oct. 24, 2013

(54) TREATMENT OF DISEASES BY EPIGENETIC REGULATION

(71) Applicants: Kevin G. McLure, Calgary (CA); Peter R. Young, San Francisco, CA (US)

(72) Inventors: Kevin G. McLure, Calgary (CA); Peter R. Young, San Francisco, CA (US)

(73) Assignee: RVX Therapeutics Inc., Calgary (CA)

(21) Appl. No.: 13/832,970

(22) Filed: Mar. 15, 2013

Related U.S. Application Data
(60) Provisional application No. 61/635,703, filed on Apr. 19, 2012.

(51) Int. Cl. C07D 239/91; C07D 401/10; C07D 403/10; C07D 401/04; A61K 31/519; C07D 471/04; C07D 401/12; C07D 409/12; C07D 403/12; A61K 31/551; C07D 401/14; A61K 31/55; A61K 45/06; C07D 403/04; A61K 31/517

(52) U.S. Cl. CPC C07D 239/91; A61K 31/517; C07D 401/10; C07D 403/10; C07D 401/04; A61K 31/519; C07D 471/04; C07D 401/12; C07D 409/12; C07D 403/12; A61K 31/551; C07D 401/14; A61K 31/55; A61K 45/06; C07D 403/04
USPC 514/43; 514/252.17; 514/266.22; 514/266.2; 514/210.18; 514/266.21; 514/264.1; 514/252.16; 514/218; 514/217.06; 514/210.21; 514/171; 514/110; 514/64; 514/266.23; 435/375

(57) ABSTRACT

The present disclosure provides non-naturally occurring polyphenol compounds that inhibit the bromodomain and extra terminal domain (BET) proteins. The disclosed compositions and methods can be used for treatment and prevention of diseases or disorders that are susceptible to administration of a BET inhibitor.

### TECHNICAL FIELD

The present disclosure relates to a method for inhibiting BET (bromodomain and extra terminal domain) proteins.

### BACKGROUND

Cancer is a group of diseases caused by dysregulated cell proliferation. Therapeutic approaches aim to decrease the numbers of cancer cells by inhibiting cell replication or by inducing cancer cell differentiation or death, but there is still significant unmet medical need for more efficacious therapeutic agents. Cancer cells accumulate genetic and epigenetic changes that alter cell growth and metabolism in order to promote cell proliferation and increased resistance to programmed cell death, or apoptosis. Some of these changes include inactivation of tumor suppressor genes, activation of oncogenes, as well as modifications of the regulation of chromatin structure. Watson, Cancer Discovery 1:477-480 (2011); Morin et al., Nature 476:298-303 (2011).

Many modifications of histones in chromatin have been characterized, including acetylation at multiple lysines in histones H3 and H4. Peserico and Simone, J. Biomed. Biotechnol. 2011:371832 (2011). Histone acetylation is controlled by acetylases (HATs) as well as deacetylases (HDACs), and small molecule HDAC inhibitors have been developed with cancer as an indication. Hoshino and Matsubara, Surg. Today 40:809-815 (2010). Histone acetylation controls gene expression by recruiting protein complexes that bind directly to acetylated lysine via bromodomains. Sanchez and Zhou, Curr. Opin. Drug Discov. Devel. 12(5):659-665 (2009). One such family, the bromodomain and extra terminal domain (BET) proteins, comprises Brd2, Brd3, Brd4, and BrdT each of which contains two bromodomains in tandem that can independently bind to acetylated lysines. Wu and Chiang, J. Biol. Chem. 282(18):13141-13145 (2007). BET proteins exert some of their effects on transcription by recruiting the positive transcription elongation factor b (p-TEFb), which stimulates transcription elongation by phosphorylating the C-terminal domain of RNA polymerase II and results in increased expression of growth promoting genes, such as, for example, c-Myc and the well established cancer target Aurora B. Filippakopoulos et al., Nature 468:1067-1073 (2010).

Molecules that bind to BET proteins and prevent them from binding to chromatin, inhibit transcription and prevent cell replication, which is useful in cancer therapy and other settings. For example, it has been shown that BET proteins can be displaced from the chromatin by small molecule inhibitors, such as, for example, JQ1, I-BET, and I-BET151, which specifically compete with the acetyl-lysine binding pocket of the BET protein bromodomains thereby preventing transcription elongation of their target genes. Filippakopoulos et al. (2010); Nicodeme et al., Nature 468:1119-1123 (2010); Dawson et al., Nature 478:529-533 (2011).

Inhibition of BET bromodomain-promoter interactions results in a subsequent reduction of myc transcription and protein levels. This results in G₁ arrest and extensive apoptosis in a variety of leukemia and lymphoma cell lines. Mertz et al., PNAS 108(40):16669-16674 (2011). The Myc family of proto-oncogenes (c-myc, I-myc, n-myc) is activated in 25-35% of all human cancers. Vita and Henrickson, Seminars in Cancer Biol. 16:318-330 (2006). Mouse models of cancer driven by overexpression of c-myc demonstrate that transiently inhibiting c-myc expression can cause tumor regression, cell death, and in some cancers such as leukemia, complete disease remission. Soucek et al., Nature 455:679-683 (2008). The absence of a clear ligand-binding domain of c-myc has made the development of an inhibitor a formidable challenge, thus alternative strategies to targeting c-myc transcription must be developed. Delmore et al., Cell 146:904-917 (2011). A mouse model of aggressive human medulloblastoma, in which c-myc is overexpressed, suggests that BET inhibitors may be useful for treating myc-amplified medulloblastoma. Kawauchi et al., Cancer Cell 21:168-180 (2012); Pei et al., Cancer Cell 21:155-167 (2012). Similarly, inhibition of n-myc through RNA interference significantly reduced tumor growth in neuroblastoma mouse models. Jiang et al., Biochem. Biophs. Res. Commun. 410:364-370 (2011). A similar role for l-myc was suggested in small cell lung carcinoma cell lines using antisense oligonucleotides to inhibit l-myc amplification. Dosaka-Akita et al., Cancer Res. 55:1559-1564 (1995). Therefore BET inhibitors have potential to be efficacious in treating multiple types of cancer.

In fact, small molecules that target the bromodomains of BET family members have demonstrated potential therapeutic use in treating cancer. See, for example, Dawson et al. (2011), showing that a small molecule inhibitor of the BET family has a profound efficacy against human and murine mixed lineage leukemia (MLL)-fusion cell lines by early cell cycle arrest and apoptosis. Its mechanism of efficacy is the selective abrogation of Brd3/4 recruitment to chromatin. BET inhibitor JQ1 has demonstrated potent antitumor activity in murine xenograoft models of NUT (nuclear protein in testis) midline carcinoma (NMC), a rare but lethal form of cancer. NMC tumor cell growth is driven by a translocation of the Brd4 gene to the nutlin 1 gene. Filippakopoulos et al., (2010). JQ1 was also shown to be a potent antiproliferator in multiple myeloma, associated with cell cycle arrest and cellular senescence. Delmore et al. (2011).

BET inhibitors are also expected to be potential therapeutics for other types of cancer. For example, in acute myeloid leukemia (AML), Brd4 is required to sustain myc expression and continued disease progression. Zuber et al., Nature 478:524-8 (2011). Moreover, inactivation of Brd4 results in a rapid and drastic down-regulation of the transcription of the proto-oncogenes c-myc and n-myc in cell lines they are amplified. Dawson et al. (2011); Delmore et al. (2011); Zuber et al. (2011); Mertz et al. (2011). Consequently, treatment of tumors that are characterized by activation or overexpression of c-myc with a BET inhibitor resulted in tumor regression through inactivation of c-myc transcription. BET inhibitors are also expected to have application in multiple myeloma, as the multiple myeloma SET domain (MMSET) which is implicated in this disease also binds to BET proteins. Dawson et al. (2011).

In addition to cancer, BET inhibitors are also expected to have anti-inflammatory and immunomodulatory properties. Lamotte et al., Bioorganic & Med. Chem. Letters (Feb. 24, 2012); Prinjha et al., Trends Pharmacol. Sci. 33(3):146-153 (2012). BET inhibitors I-BET and I-BET151 decrease IL-6 expression in vivo. I-BET was shown to confer protection against lipopolysaccharide-induced endotoxic shock and bacteria-induced sepsis and I-BET151 was shown to suppress bacterial-induced inflammation and sepsis in a murine model. Nicodeme et al. (2010); Lamotte et al. (2012). In addition, BET inhibitors may modulate responses to viral and bacterial infections, including HIV, herpes, and papilloma viruses.

### DESCRIPTION OF THE INVENTION

The present invention provides a method for inhibiting BET proteins by administering a compound of any one of Formulas I-V. The methods of the invention may be used to treat diseases that are sensitive to a compound that binds to bromodomains of BET family proteins, including NUT midline carcinoma, as well as cancers that exhibit c-myc overexpression, including, but not limited to, Burkitt's lymphoma, acute myelogenous leukemia, multiple myeloma, aggressive human medulloblastoma; cancers overexpressing n-myc, cancers that rely on the recruitment of p-TEFb to regulate activated oncogenes such as, for example, NOTCH1. In some embodiments, BET inhibitors may induce apoptosis in cancer cells by decreasing expression of the anti-apoptosis gene Bcl2. In certain embodiments, the methods of the invention are used to treat or prevent cancers, including hematological, epithelial including lung, breast and colon carcinomas, midline carcinomas, mesenchymal, hepatic, renal and neurological tumours.

The methods of invention include administering to a mammal, such as a human, for the purpose of inhibiting a BET protein, a therapeutically effective amount of at least one compound of Formula I:

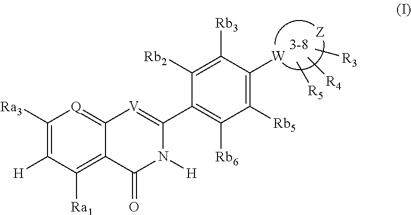

or stereoisomer, tautomer, pharmaceutically acceptable salt, or hydrate thereof, wherein:

Q and V are independently selected from CH and nitrogen;

Ra₁ and Ra₃ are independently selected from hydrogen, C₁-C₆ alkyl, C₁-C₆ alkoxy, halogen, amino, amide, hydroxyl, heterocycle, and C₃-C₆ cycloalkyl;

Rb₂ and Rb₆ are each hydrogen;

Rb₃ and Rb₅ are independently selected from hydrogen, halogen, C₁-C₆ alkyl, C₁-C₆ alkoxy, C₃-C₆ cycloalkyl, hydroxyl, and amino;

wherein Rb₂ and Rb₃ and/or Rb₅ and Rb₆ may be connected to form a cycloalkyl or a heterocycle;

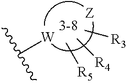

represents a 3-8 membered ring system wherein:

W is selected from carbon and nitrogen;

Z is selected from CR₆R₇, NR₈, oxygen, sulfur, —S(O)—, and —SO₂—; said ring system being optionally fused to another ring selected from cycloakyl, heterocycle, and phenyl, and wherein said ring system is selected from, for example, rings having the structures

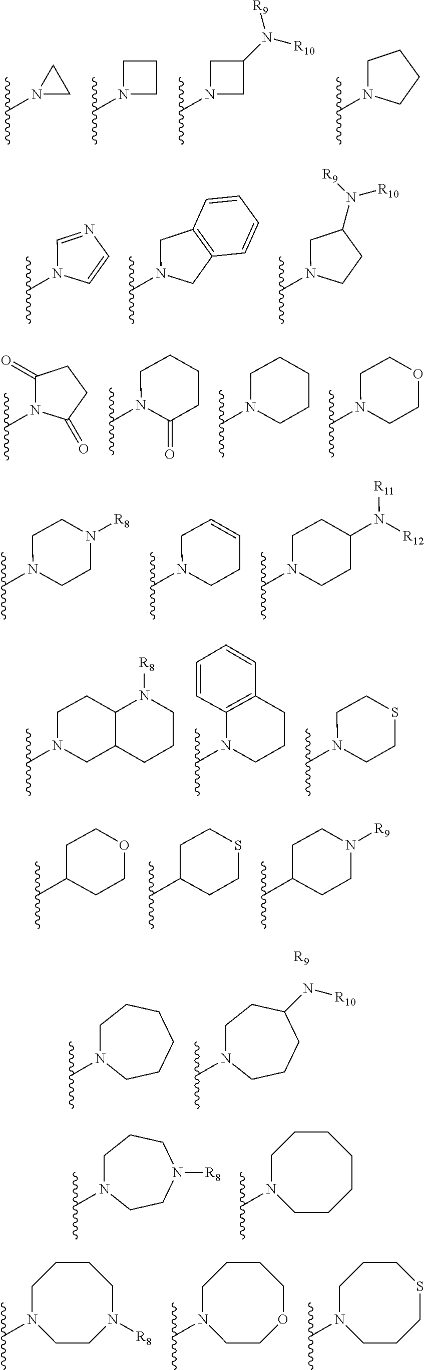

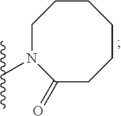 [SMILES: *N1CCCCCCC1=O]

R₃, R₄, and R₅ are independently selected from hydrogen, C₁-C₆ alkyl, C₁-C₆ alkenyl, C₁-C₆ alkynyl, C₁-C₆ alkoxy, C₃-C₆ cycloalkyl, aryl, aryloxy, hydroxyl, amino, amide, oxo, —CN, and sulfonamide;

R₆ and R₇ are independently selected from hydrogen, C₁-C₆ alkyl, C₁-C₆ alkenyl, C₁-C₆ alkynyl, C₃-C₆ cycloalkyl, aryl, halogen, hydroxyl, —CN, amino, and amido;

R₈ is selected from hydrogen, C₁-C₆ alkyl, C₁-C₆ alkenyl, C₁-C₆ alkynyl, acyl, and C₃-C₆ cycloalkyl; and

R₉, R₁₀, R₁₁, and R₁₂ are independently selected from hydrogen, C₁-C₆ alkyl, C₁-C₆ alkenyl, C₁-C₆ alkynyl, C₃-C₆ cycloalkyl, aryl, heterocycle, hydroxyl, sulfonyl, and acyl,

provided that:

if Q is CH, then at least one of Ra₁ and Ra₃ is not hydrogen;

if Z os NAc, then Ra₁ and Ra₃ are not hydrogen, and Ra₁ is not —OCH₂CH₂OMe; and

if Ra₁ and Ra₃ are both OMe, then R₈ is not —C(O)CH₂OH.

In certain embodiments, the method for inhibiting a BET protein in a subject comprises administering a therapeutically effective amount of at least one compound of Formula II:

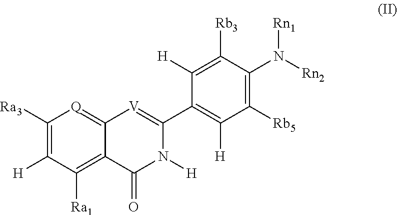

or a stereoisomer, tautomer, pharmaceutically acceptable salt, or hydrate thereof, wherein:

Q and V are independently selected from CH and nitrogen;

Ra₁ and Ra₃ are independently selected from hydrogen, C₁-C₆ alkyl, C₁-C₆ alkoxy, C₃-C₆ cycloalkyl, halogen, amino, amide, hydroxyl, cycloalkyl, and heterocycle;

Rb₃ and Rb₅ are independently selected from hydrogen, halogen, C₁-C₆ alkyl, C₁-C₆ alkoxy, C₃-C₆ cycloalkyl, hydroxyl, and amino;

Rn₁ is selected from hydrogen, C₁-C₆ alkyl, and C₃-C₆ cycloalkyl; and

Rn₂ is selected from C₁-C₆ alkyl, C₃-C₆ cycloalkyl, heterocycle, aryl, alkenyl, sulfonyl and acyl;

wherein Rn₁ and/or Rn₂ may be connected with Rb₃ and/or Rb₅ to form a 5- or 6-membered heterocyclic ring;

provided that:

at least one of Ra₁ and Ra₃ are not hydrogen; and

Rn₁ and Rn₂ are not both methyl or ethyl.

In other embodiments, the method inhibiting a BET protein in a subject comprises administering a therapeutically effective amount of at least one compound of Formula III:

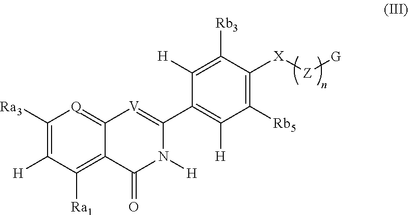

or a stereoisomer, tautomer, pharmaceutically acceptable salt, or hydrate thereof, wherein:

Q is selected from CH and nitrogen;

V is selected from CH and nitrogen;

X is selected from oxygen, sulfur, SR₁, nitrogen, NR₆R₇, and CR₆R₇;

Z is selected from unsubstituted C₁-C₆ alkyl and C₁-C₆ alkyl substituted with one or more groups selected from C₁-C₃ alkyl, C₁-C₃ alkoxy, cyclopropyl, hydroxyl, amino, and halogen;

n is selected from 0, 1, 2, or 3;

G is selected from heterocycle, cycloalkyl, and aryl;

R₁ is selected from hydrogen, and C₁-C₆ alkyl;

R₆ and R₇ are independently selected from hydrogen, C₁-C₆ alkyl, C₃-C₆ cycloalkyl, heterocycle, C₁-C₆ alkoxy, and halogen;

Ra₁ and Ra₃ are independently selected from hydrogen, C₁-C₆ alkyl, C₁-C₆ alkoxy, C₃-C₆ cycloalkyl, halogen, amino, amide, hydroxyl, and heterocycle;

Rb₃ and Rb₅ are independently selected from hydrogen, halogen, C₁-C₆ alkyl, C₃-C₆ cycloalkyl, C₁-C₆ alkoxy, hydroxyl, and amino;

provided that:

if Ra₁ and Ra₃ are OMe, and Q is CH, then

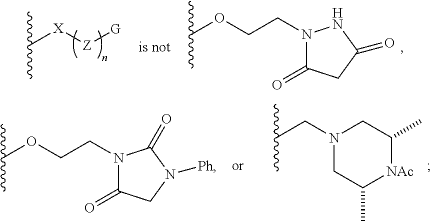

at least one of Ra₁ and Ra₃ is not hydrogen; and

if Ra₃ is chloro, then Ra₁ is not hydrogen.

In some embodiments the method for inhibiting a BET protein in a subject comprises administering a therapeutically effective amount of at least one compound of Formula IV:

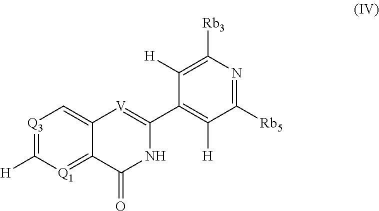

or a stereoisomer, tautomer, pharmaceutically acceptable salt, or hydrate thereof, wherein:

Q₁ is selected from nitrogen and C—Ra₁;

Q₃ is selected from nitrogen and C—Ra₃;

V is selected from CH and nitrogen;

Ra₁ and Ra₃ are independently selected from hydrogen, C₁-C₆ alkyl, C₁-C₆ alkenyl, C₁-C₆ alkynyl, C₁-C₆ alkoxy, C₃-C₆ cycloalkyl, amino, amide, and heterocycle, wherein Ra₁ and Ra₂ and/or Ra₂ and Ra₃ may be connected to form a cycloalkyl or a heterocycle;

Rb₃ and Rb₅ are independently selected from hydrogen, methyl, ethyl, C₃-C₆ cycloalkyl, C₁-C₃ alkoxy, and amino;

provided that:

if Ra₃ is alkoxy, then Ra₁ is not hydrogen; and

if Rb₅ is hydrogen, then Rb₃ is not —CH₂OH.

In a further embodiment, the method for inhibiting BET proteins in a subject comprises administering a therapeutically effective amount of at least one compound of Formula V:

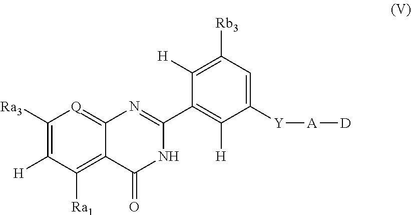

or a stereoisomer, tautomer, pharmaceutically acceptable salt, or hydrate thereof, wherein:

Q is selected from CH and nitrogen;

Y is selected from oxygen, nitrogen, sulfur, NR₆, CR₆R₇;

A is C₁-C₄ alkyl, wherein the alkyl chain may be connected to Y, D, and/or Rb₃ to form a cycloalkyl or heterocycle;

D may be absent or present, and if present, is selected from —OR₁, —NR₁R₂;

R₁ and R₂ are independently selected from hydrogen, C₁-C₆ alkyl, C₃-C₆ cycloalkyl, sulfonamide, carboxamide, acyl, and nitrile, wherein R₁ and R₂ may be connected to form a cycloalkyl or a heterocycle;

R₆ and R₇ are independently selected from hydrogen, C₁-C₆ alkyl, C₃-C₆ cycloalkyl, C₁-C₆ alkoxy, hydroxyl, and halogen;

Ra₁ and Ra₃ are independently selected from hydrogen, C₁-C₆ alkyl, C₁-C₆ alkoxy, C₃-C₆ cycloalkyl, halogen, amino, amide, hydroxyl, and heterocycle;

Rb₃ is selected from hydrogen, halogen, C₁-C₆ alkyl, C₃-C₆ cycloalkyl, C₁-C₆ alkoxy, hydroxyl, and amino;

provided that:

at least one of Ra₁ and Ra₃ is not hydrogen.

The invention also provides methods of using a pharmaceutical composition comprising one or more compounds of Formula I, Formula II, Formula III, Formula IV, and Formula V, or a stereoisomer, tautomer, pharmaceutically acceptable salt, or hydrate of compounds of Formula I, II, III, IV, and V, together with at least one pharmaceutically acceptable carrier, adjuvant, and/or excipient to inhibit BET proteins.

In certain embodiments, the methods of the invention are useful for the prevention or treatment of diseases that benefit from increased cell death or differentiation, or decreased cell proliferation. This may occur by, for example, decreased expression of a Myc family member or an oncogene required for tumor growth, or increase of a tumor suppressor gene, the latter antagonized by BET proteins. The method of the invention can be used to increase cancer cell death or decrease cancer cell proliferation, including, for example, by decreasing expression of Myc family member. Decreasing expression of the Myc family member may refer to, but is not limited to, transcriptionally modulating the expression of its gene or genes that have been either amplified in the genome or translocated to another chromosomal location, or transcriptionally altered in order to increase its expression (i.e. overexpression) thereby affecting the level of the c-myc protein produced. A decrease in the Myc family member mRNA levels may decrease proliferation of cancer cells and/or increase cancer cell death, including but not limited to apoptosis.

In other embodiments, the methods of the invention are useful for the prevention or treatment of diseases such as cancer in combination with other drugs. In some embodiments, a therapeutically effective amount of one or more compounds of Formula I, Formula II, Formula III, Formula IV, Formula V, or a tautomer, stereoisomer, pharmaceutically acceptable salt or hydrate of compounds of Formula I, Formula II, Formula III, Formula IV, and Formula V may be administered in combination with a standard of care drug(s) for any given tumor type, including, but not limited to, bortezomib, thalidomide, dexamethasone, 5-azacitidine, decitabine, vorinostat, or cyclophosphamide in multiple myeloma. In another embodiment, a compound of Formula I may be administered in combination with a PI3K or mTOR inhibitor such as rapamycin. Similarly, a compound of Formula I could be administered in combination with gamma secretase inhibitors which inhibit NOTCH1 (given the relationship between c-myc and NOTCH1) or AMPK inducers such as metformin or phenformin for leukemia. Another example of a potentially useful combination is combining a BET inhibitor which decreases myc expression, with an ornithine decarboxylase inhibitor such as difluoromethylornithine, which inhibits a myc target.

In certain embodiments, the methods of the invention provide treatment of auto-immune and inflammatory diseases or conditions by administering one or more compounds of Formula I, Formula II, Formula III, Formula IV, Formula V, or a tautomer, stereoisomer, pharmaceutically acceptable salt or hydrate of compounds of Formula I, Formula II, Formula III, Formula IV, and Formula V. In other embodiments, one or more compounds of Formula I, Formula II, Formula III, Formula IV, Formula V, or a tautomer, stereoisomer, pharmaceutically acceptable salt or hydrate of compounds of Formula I, Formula II, Formula III, Formula IV, and Formula V may be employed to treat diseases or disorders caused by bacterial or viral infection, such as, for example, HIV, HPV, and herpes virus. Certain embodiments of the invention provide, for use of a one or more compounds of Formula I, Formula II, Formula III, Formula IV, Formula V, or a tautomer, stereoisomer, pharmaceutically acceptable salt or hydrate of compounds of Formula I, Formula II, Formula III, Formula IV, and Formula V in the manufacture of a medicament for the treatment of cancer, immune related disorders, inflammatory disease, AIDS, or sepsis.

### DEFINITIONS

As used in the present specification, the following words, phrases and symbols are generally intended to have the meanings as set forth below, except to the extent that the context in which they are used indicates otherwise. The following abbreviations and terms have the indicated meanings throughout.

“Subject” refers to an animal, such as a mammal, that has been or will be the object of treatment, observation, or experiment. The methods described herein may be useful for both human therapy and veterinary applications. In one embodiment, the subject is a human.

As used herein, “treatment” or “treating” refers to an amelioration of a disease or disorder, or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, for example, stabilization of a discernible symptom, physiologically, for example, stabilization of a physical parameter, or both. In yet another embodiment, “treatment” or “treating” refers to delaying the onset of a disease or disorder.

As used herein, “prevention” or “preventing” refers to a reduction of the risk of acquiring a given disease or disorder.

A dash (“-”) that is not between two letters or symbols is used to indicate a point of attachment for a substituent. For example, —CONH₂ is attached through the carbon atom.

By “optional” or “optionally” is meant that the subsequently described event or circumstance may or may not occur, and that the description includes instances where the event or circumstance occurs and instances in which is does not. For example, “optionally substituted aryl” encompasses both “aryl” and “substituted aryl” as defined below. It will be understood by those skilled in the art, with respect to any group containing one or more substituents, that such groups are not intended to introduce any substitution or substitution patterns that are sterically impractical, synthetically non-feasible and/or inherently unstable.

As used herein, the term “hydrate” refers to a crystal form with either a stoichiometric or non-stoichiometric amount of water is incorporated into the crystal structure.

The term “acyl” term as used herein refers to a carbonyl radical attached to an alkyl, alkenyl, alkynyl, cycloalkyl, heterocycyl, aryl, or heteroaryl. Exemplary acyl groups include, but are not limited to, acetyl, formyl, propionyl, benzoyl, and the like.

The term “aldehyde” or “formyl” as used herein refers to —CHO.

The term “alkenyl” as used herein refers to an unsaturated straight or branched hydrocarbon having at least one carbon-carbon double bond, such as a straight or branched group of 2-22, 2-8, or 2-6 carbon atoms, referred to herein as (C₂-C₂₂)alkenyl, (C₂-C₈)alkenyl, and (C₂-C₆)alkenyl, respectively. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, and 4-(2-methyl-3-butene)-pentenyl.

The term “alkoxy” as used herein refers to an alkyl group attached to an oxygen (—O-alkyl-). “Alkoxy” groups also include an alkenyl group attached to an oxygen (“alkenyloxy”) or an alkynyl group attached to an oxygen (“alkynyloxy”) groups. Exemplary alkoxy groups include, but are not limited to, groups with an alkyl, alkenyl or alkynyl group of 1-22, 1-8, or 1-6 carbon atoms, referred to herein as (C₁-C₂₂)alkoxy, (C₁-C₈)alkoxy, and (C₁-C₆)alkoxy, respectively. Exemplary alkoxy groups include, but are not limited to methoxy and ethoxy.

The term “alkyl” as used herein refers to a saturated straight or branched hydrocarbon, such as a straight or branched group of 1-22, 1-8, or 1-6 carbon atoms, referred to herein as (C₁-C₂₂)alkyl, (C₁-C₈)alkyl, and (C₁-C₆)alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl.

The term “alkynyl” as used herein refers to an unsaturated straight or branched hydrocarbon having at least one carbon-carbon triple bond, such as a straight or branched group of 2-22, 2-8, or 2-6 carbon atoms, referred to herein as (C₂-C₂₂)alkynyl, (C₂-C₈)alkynyl, and (C₂-C₆)alkynyl, respectively. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “amide” as used herein refers to the form —NRₐC(O)(R_{b})— or —C(O)NR_{b}R_{c}, wherein Rₐ, R_{b} and R_{c} are each independently selected from alkyl, alkenyl, alkynyl, aryl, arylalkyl, cycloalkyl, haloalkyl, heteroaryl, heterocyclyl, and hydrogen. The amide can be attached to another group through the carbon, the nitrogen, R_{b}, or R_{c}. The amide also may be cyclic, for example R_{b} and R_{c}, may be joined to form a 3- to 12-membered ring, such as a 3- to 10-membered ring or a 5- or 6-membered ring. The term “amide” encompasses groups such as sulfonamide, urea, ureido, carbamate, carbamic acid, and cyclic versions thereof. The term “amide” also encompasses an amide group attached to a carboxy group, for example, -amide-COON or salts such as -amide-COONa, an amino group attached to a carboxy group (for example, -amino-COON or salts such as -amino-COONa).

The term “amine” or “amino” as used herein refers to the form —NR_{d}Rₑ or —N(R_{d})Rₑ—, where R_{d} and Rₑ are independently selected from alkyl, alkenyl, alkynyl, aryl, arylalkyl, carbamate, cycloalkyl, haloalkyl, heteroaryl, heterocyclyl, and hydrogen. The amino can be attached to the parent molecular group through the nitrogen. The amino also may be cyclic, for example any two of R_{d} and Rₑ may be joined together or with the N to form a 3- to 12-membered ring (for example, morpholino or piperidinyl). The term amino also includes the corresponding quaternary ammonium salt of any amino group. Exemplary amino groups include alkylamino groups, wherein at least one of R_{d} or Rₑ is an alkyl group.

The term “aryl” as used herein refers to a mono-, bi-, or other multi-carbocyclic, aromatic ring system. The aryl group can optionally be fused to one or more rings selected from aryls, cycloalkyls, and heterocyclyls. The aryl groups of this invention can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include, but are not limited to a monocyclic aromatic ring system, wherein the ring comprises 6 carbon atoms, referred to herein as “(C₆)aryl.”

The term “arylalkyl” as used herein refers to an alkyl group having at least one aryl substituent (for example, -aryl-alkyl-). Exemplary arylalkyl groups include, but are not limited to, arylalkyls having a monocyclic aromatic ring system, wherein the ring comprises 6 carbon atoms, referred to herein as “(C₆)arylalkyl.”

The term “aryloxy” as used herein refers to an aryl group attached to an oxygen atom. Exemplary aryloxy groups include, but are not limited to, aryloxys having a monocyclic aromatic ring system, wherein the ring comprises 6 carbon atoms, referred to herein as “(C₆)aryloxy.”

The term “arylthio” as used herein refers to an aryl group attached to an sulfur atom. Exemplary arylthio groups include, but are not limited to, arylthios having a monocyclic aromatic ring system, wherein the ring comprises 6 carbon atoms, referred to herein as “(C₆)arylthio.”

The term “arylsulfonyl” as used herein refers to an aryl group attached to a sulfonyl group, for example, —S(O)₂-aryl-. Exemplary arylsulfonyl groups include, but are not limited to, arylsulfonyls having a monocyclic aromatic ring system, wherein the ring comprises 6 carbon atoms, referred to herein as “(C₆)arylsulfonyl.”

The term “benzyl” as used herein refers to the group —CH₂-phenyl.

The term “bicyclic aryl” as used herein refers to an aryl group fused to another aromatic or non-aromatic carbocylic or heterocyclic ring. Exemplary bicyclic aryl groups include, but are not limited to, naphthyl or partly reduced forms thereof, such as di-, tetra-, or hexahydronaphthyl.

The term “bicyclic heteroaryl” as used herein refers to a heteroaryl group fused to another aromatic or non-aromatic carbocylic or heterocyclic ring. Exemplary bicyclic heteroaryls include, but are not limited to 5,6- or 6,6-fused systems, wherein one or both rings contain heteroatoms. The term “bicyclic heteroaryl” also encompasses reduced or partly reduced forms of fused aromatic system wherein one or both rings contain ring heteroatoms. The ring system may contain up to three heteroatoms, independently selected from oxygen, nitrogen, and sulfur. The bicyclic system may be optionally substituted with one or more groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary bicyclic heteroaryl's include, but are not limited to, quinazolinyl, benzothiophenyl, benzoxazolyl, benzimidazolyl, benzothiazolyl, benzofuranyl, indolyl, quinolinyl, isoquinolinyl, phthalazinyl, benzotriazolyl, benzopyridinyl, and benzofuranyl.

The term “carbamate” as used herein refers to the form —R_{g}OC(O)N(Rₕ)—, —R_{g}OC(O)N(Rₕ)Rᵢ—, or —OC(O)NRₕRᵢ, wherein R_{g}, Rₕ and Rᵢ are each independently selected from alkyl, alkenyl, alkynyl, aryl, arylalkyl, cycloalkyl, haloalkyl, heteroaryl, heterocyclyl, and hydrogen. Exemplary carbamates include, but are not limited to, arylcarbamates or heteroaryl carbamates (for example, wherein at least one of R_{g}, Rₕ and Rᵢ are independently selected from aryl or heteroaryl, such as pyridine, pyridazine, pyrimidine, and pyrazine).

The term “carbonyl” as used herein refers to —C(O)—.

The term “carboxy” as used herein refers to —COON or its corresponding carboxylate salts (for example, —COONa). The term carboxy also includes “carboxycarbonyl,” for example a carboxy group attached to a carbonyl group, for example, —C(O)—COON or salts, such as —C(O)—COONa.

The term “cyano” as used herein refers to —CN.

The term “cycloalkoxy” as used herein refers to a cycloalkyl group attached to an oxygen.

The term “cycloalkyl” as used herein refers to a saturated or unsaturated cyclic, bicyclic, or bridged bicyclic hydrocarbon group of 3-12 carbons, or 3-8 carbons, referred to herein as “(C₃-C₈)cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl saturated or unsaturated, aryl, or heterocyclyl groups.

The term “dicarboxylic acid” as used herein refers to a group containing at least two carboxylic acid groups such as saturated and unsaturated hydrocarbon dicarboxylic acids and salts thereof. Exemplary dicarboxylic acids include alkyl dicarboxylic acids. Dicarboxylic acids may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, nitro, phosphate, sulfide, sulfinyl, sulfonyl, sulfonic acid, sulfonamide and thioketone. Dicarboxylic acids include, but are not limited to succinic acid, glutaric acid, adipic acid, suberic acid, sebacic acid, azelaic acid, maleic acid, phthalic acid, aspartic acid, glutamic acid, malonic acid, fumaric acid, (+)/(−)-malic acid, (+)/(−) tartaric acid, isophthalic acid, and terephthalic acid. Dicarboxylic acids further include carboxylic acid derivatives thereof, such as anhydrides, imides, hydrazides (for example, succinic anhydride and succinimide).

The term “ester” refers to the structure —C(O)O—, —C(O)O—Rⱼ—, —RₖC(O)O—Rⱼ—, or —RₖC(O)O—, where O is not bound to hydrogen, and Rⱼ and Rₖ can independently be selected from alkoxy, aryloxy, alkyl, alkenyl, alkynyl, amide, amino, aryl, arylalkyl, cycloalkyl, ether, haloalkyl, heteroaryl, and heterocyclyl. Rₖ can be a hydrogen, but Rⱼ cannot be hydrogen. The ester may be cyclic, for example the carbon atom and Rⱼ, the oxygen atom and Rₖ, or Rⱼ and Rₖ may be joined to form a 3- to 12-membered ring. Exemplary esters include, but are not limited to, alkyl esters wherein at least one of Rⱼ or Rₖ is alkyl, such as —O—C(O)-alkyl, —C(O)—O-alkyl-, and -alkyl-C(O)—O-alkyl-. Exemplary esters also include aryl or heteroaryl esters, for example wherein at least one of Rⱼ or Rₖ is a heteroaryl group such as pyridine, pyridazine, pyrmidine and pyrazine, such as a nicotinate ester. Exemplary esters also include reverse esters having the structure —RₖC(O)O—, where the oxygen is bound to the parent molecule. Exemplary reverse esters include succinate, D-argininate, L-argininate, L-lysinate and D-lysinate. Esters also include carboxylic acid anhydrides and acid halides.

The term “ether” refers to the structure —RₗO—Rₘ—, where Rₗ and Rₘ can independently be alkyl, alkenyl, alkynyl, aryl, cycloalkyl, heterocyclyl, and ether. The ether can be attached to the parent molecular group through Rₗ or Rₘ. Exemplary ethers include, but are not limited to, alkoxyalkyl and alkoxyaryl groups. Ethers also includes polyethers, for example, where one or both of Rₗ and Rₘ are ethers.

The terms “halo” or “halogen” or “Hal” as used herein refer to F, Cl, Br, or I.

The term “haloalkyl” as used herein refers to an alkyl group substituted with one or more halogen atoms. “Haloalkyls” also encompass alkenyl or alkynyl groups substituted with one or more halogen atoms.

The term “heteroaryl” as used herein refers to a mono-, bi-, or multi-cyclic, aromatic ring system containing one or more heteroatoms, for example 1-3 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2-5 carbon atoms and 1-3 heteroatoms, referred to herein as “(C₂-C₅)heteroaryl.”

The terms “heterocycle,” “heterocyclyl,” or “heterocyclic” as used herein refer to a saturated or unsaturated 3-, 4-, 5-, 6-, or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Heterocycles also include bicyclic, tricyclic, and tetracyclic groups in which any of the above heterocyclic rings is fused to one or two rings independently selected from aryl, cycloalkyl, and heterocycle. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin-2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refers to —OH.

The term “hydroxyalkyl” as used herein refers to a hydroxy attached to an alkyl group.

The term “hydroxyaryl” as used herein refers to a hydroxy attached to an aryl group.

The term “ketone” as used herein refers to the structure —C(O)—Rₙ (such as acetyl, —C(O)CH₃ or —Rₙ—C(O)—Rₒ—. The ketone can be attached to another group through Rₙ or Rₒ. Rₙ or Rₒ can be alkyl, alkenyl, alkynyl, cycloalkyl, heterocyclyl, or aryl, or Rₙ and Rₒ can be joined to form a 3- to 12-membered ring.

The term “monoester” as used herein refers to an analogue of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic, and maleic acid.

The term “nitro” as used herein refers to —NO₂.

The term “perfluoroalkoxy” as used herein refers to an alkoxy group in which all of the hydrogen atoms have been replaced by fluorine atoms.

The term “perfluoroalkyl” as used herein refers to an alkyl group in which all of the hydrogen atoms have been replaced by fluorine atoms. Exemplary perfluoroalkyl groups include, but are not limited to, C₁-C₅ perfluoroalkyl, such as trifluoromethyl.

The term “perfluorocycloalkyl” as used herein refers to a cycloalkyl group in which all of the hydrogen atoms have been replaced by fluorine atoms.

The term “phenyl” as used herein refers to a 6-membered carbocyclic aromatic ring. The phenyl group can also be fused to a cyclohexane or cyclopentane ring. Phenyl can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone.

The term “phosphate” as used herein refers to the structure —OP(O)O₂—, —RₓOP(O)O₂—, —OP(O)O₂R_{y}—, or —RₓOP(O)O₂R_{y}—, wherein Rₓ and R_{y} can be alkyl, alkenyl, alkynyl, aryl, cycloalkyl, heterocyclyl, and hydrogen.

The term “sulfide” as used herein refers to the structure —R_{z}S—, where R_{z} can be alkyl, alkenyl, alkynyl, aryl, arylalkyl, cycloalkyl, haloalkyl, heteroaryl, heterocyclyl. The sulfide may be cyclic, forming a 3 to 12-membered ring. The term “alkylsulfide” as used herein refers to an alkyl group attached to a sulfur atom.

The term “sulfinyl” as used herein refers to the structure —S(O)O—, —RₚS(O)O—, —RₚS(O)OR_{q}—, or —S(O)OR_{q}—, wherein Rₚ and R_{q} can be alkyl, alkenyl, aryl, arylalkyl, cycloalkyl, haloalkyl, heteroaryl, heterocyclyl, and hydroxyl. Exemplary sulfinyl groups include, but are not limited to, alkylsulfinyls wherein at least one of Rₚ or R_{q} is alkyl, alkenyl, or alkynyl.

The term “sulfonamide” as used herein refers to the structure —(Rᵣ)—N—S(O)₂—Rₛ— or —Rₜ(Rᵣ)—N—S(O)₂—Rₛ, where Rₜ, Rᵣ, and Rₛ can be, for example, hydrogen, alkyl, alkenyl, alkynyl, aryl, cycloalkyl, and heterocyclyl. Exemplary sulfonamides include alkylsulfonamides (for example, where Rₛ is alkyl), arylsulfonamides (for example, where Rₛ is aryl), cycloalkyl sulfonamides (for example, where Rₛ is cycloalkyl), and heterocyclyl sulfonamides (for example, where Rₛ is heterocyclyl).

The term “sulfonate” as used herein refers to −OSO₃—. Sulfonate includes salts such as —OSO₃Na, —OSO₃K and the acid —OSO₃H.

The term “sulfonic acid” refers to —SO₃H— and its corresponding salts (for example, —SO₃K— and —SO₃Na—).

The term “sulfonyl” as used herein refers to the structure RᵤSO₂—, where Rᵤ can be alkyl, alkenyl, alkynyl, aryl, cycloalkyl, and heterocyclyl (for example, alkylsulfonyl). The term “alkylsulfonyl” as used herein refers to an alkyl group attached to a sulfonyl group. “Alkylsulfonyl” groups can optionally contain alkenyl or alkynyl groups.

The term “thioketone” refers to the structure —Rᵥ—C(S)—R_{w}—. The ketone can be attached to another group through Rᵥ or R_{w}. Rᵥ or R_{w} can be alkyl, alkenyl, alkynyl, cycloalkyl, heterocyclyl, or aryl, or Rᵥ and R_{w} can be joined to form a 3- to 12-membered ring.

“Alkyl” groups can be substituted with or interrupted by or branched with at least one group selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, ketone, heteroaryl, heterocyclyl, hydroxyl, nitro, phosphate, sulfide, sulfinyl, sulfonyl, sulfonic acid, sulfonamide, thioketone, ureido, and N. The substituents may be branched to form a substituted or unsubstituted heterocycle or cycloalkyl.

“Alkenyl,” “alkynyl”, “alkoxy”, “amino” and “amide” groups can be substituted with or interrupted by or branched with at least one group selected from alkoxy, aryloxy, alkyl, alkenyl, alkynyl, amide, amino, aryl, arylalkyl, carbamate, carbonyl, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, thioketone, ureido, and N. The substituents may be branched to form a substituted or unsubstituted heterocycle or cycloalkyl.

As used herein, a “suitable substituent” refers to a group that does not nullify the synthetic or pharmaceutical utility of the compounds of the invention or the intermediates useful for preparing them. Examples of suitable substituents include, but are not limited to: C₁₋₂₂, C₁₋₈, and C₁₋₆ alkyl, alkenyl or alkynyl; C₁₋₆ aryl, C₂₋₅ heteroaryl; C₃₋₇ cycloalkyl; C₁₋₂₂, C₁₋₈, and C₁₋₆ alkoxy; C₆ aryloxy; —CN; —OH; oxo; halo, carboxy; amino, such as —NH(C₁₋₂₂, C₁₋₈, or C₁₋₆ alkyl), —N(C₁₋₂₂, C₁₋₈, and C₁₋₆ alkyl)₂, —NH((C₆)aryl), or —N((C₆)aryl)₂; formyl; ketones, such as —CO(C₁₋₂₂, C₁₋₈, and C₁₋₆ alkyl), —CO((C₆ aryl) esters, such as —CO₂(C₁₋₂₂, C₁₋₈, and C₁₋₆ alkyl) and —CO₂ (C₆ aryl). One of skill in art can readily choose a suitable substituent based on the stability and pharmacological and synthetic activity of the compound of the invention.

As used herein, “inhibiting” refers to blocking, suppressing, or in any other way, reducing, the biological function of a BETprotein in a subject.

As used herein, “reducing” refers to reducing the overall levels of BET biological activity, for example, by inhibiting the availability of the level of BET protein in the body for other biological interactions.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

The term “pharmaceutically acceptable prodrugs” as used herein represents those prodrugs of the compounds of the present invention that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of the invention. A discussion is provided in Higuchi et al., “Prodrugs as Novel Delivery Systems,” ACS Symposium Series, Vol. 14, and in Roche, E. B., ed. Bioreversible Carriers in Drug Design, American Pharmaceutical Association and Pergamon Press, 1987, both of which are incorporated herein by reference.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present invention encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may contain an implicit chiral center.

Individual stereoisomers of compounds of the present invention can be prepared synthetically from commercially available starting materials that contain asymmetric or stereogenic centers, or by preparation of racemic mixtures followed by resolution methods well known to those of ordinary skill in the art. These methods of resolution include, but are not limited to (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well known methods, including, but not limited to chiral-phase gas chromatography, chiral-phase high performance liquid chromatography, crystallizing the compound as a chiral salt complex, and/or crystallizing the compound in a chiral solvent. Stereoisomers can also be obtained from stereomerically-pure intermediates, reagents, and catalysts by well known asymmetric synthetic methods.

Geometric isomers can also exist in the compounds of the present invention. The present invention encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the E and Z isomers.

Substituents around a carbon-carbon double bond alternatively can be referred to as “cis” or “trans,” where “cis” represents substituents on the same side of the double bond and “trans” represents substituents on opposite sides of the double bond. The arrangements of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The compounds disclosed herein may exist as tautomers and both tautomeric forms are intended to be encompassed by the scope of the invention, even though only one tautomeric structure is depicted. For example, any claim to compound A below is understood to include tautomeric structure B, and vice versa, as well as mixtures thereof.

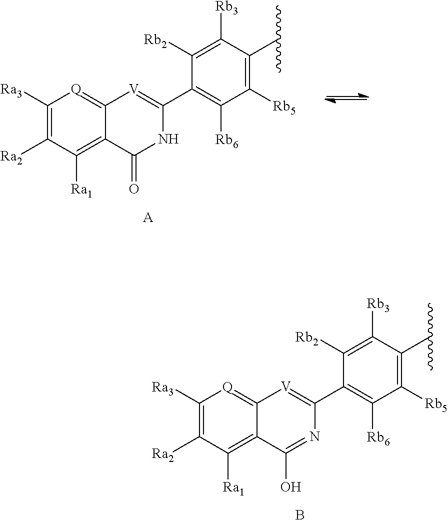

### Exemplary Embodiments

### Embodiments Employing Compounds of Formula I

In certain embodiments, the method inhibiting BET proteins in a subject comprises administering a therapeutically effective amount of at least one compound of Formula I:

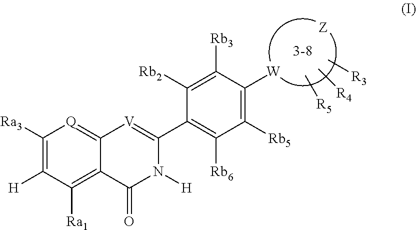

or stereoisomer, tautomer, pharmaceutically acceptable salt, or hydrate thereof, wherein:

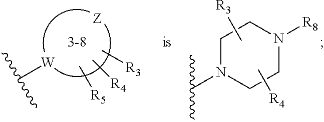

Ra₁ is selected from methyl, ethyl, methoxy, ethoxy, and propoxy;

R₃ and R₄ are independently selected from hydrogen, C₁-C₆ alkyl, C₁-C₆ alkenyl, C₁-C₆ alkynyl, C₁-C₆ alkoxy, C₃-C₆ cycloalkyl, aryloxy, aryl, hydroxyl, amino, amide, oxo, —CN, and sulfonamide; and

R₈ is selected from hydrogen, C₁-C₆ alkyl, C₁-C₆ alkenyl, acyl, and C₁-C₆ alkynyl.

In some embodiments, the method for inhibiting BET proteins in a subject comprises administering a therapeutically effective amount of at least one compound of Formula I, wherein:

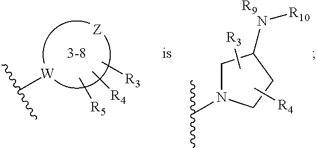

Ra₁ is selected from methyl, ethyl, methoxy, ethoxy, and propoxy;

R₃ and R₄ are independently selected from hydrogen, C₁-C₆ alkyl, C₁-C₆ alkenyl, C₁-C₆ alkynyl, C₁-C₆ alkoxy, C₃-C₆ cycloalkyl, aryloxy, aryl, hydroxyl, amino, amide, oxo, —CN, and sulfonamide; and

R₉ and R₁₀ are independently selected from hydrogen, C₁-C₆ alkyl, C₁-C₆ alkenyl, C₁-C₆ alkynyl, C₃-C₆ cycloalkyl, aryl, heterocycle, sulfonyl, and acyl.

In some embodiments, the method for inhibiting BET proteins in a subject comprises administering a therapeutically effective amount of at least one compound of Formula I, wherein:

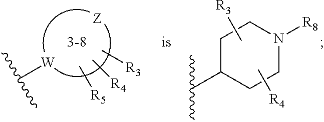

Ra₁ is selected from methyl, ethyl, methoxy, ethoxy, and propoxy;

R₃ and R₄ are independently selected from hydrogen, C₁-C₆ alkyl, C₁-C₆ alkenyl, C₁-C₆ alkynyl, C₁-C₆ alkoxy, C₃-C₆ cycloalkyl, aryloxy, aryl, hydroxyl, amino, amido, oxo, —CN, and sulfonamide; and

R₈ is selected from hydrogen, C₁-C₆ alkyl, C₁-C₆ alkenyl, C₁-C₆ alkynyl, acyl, and C₃-C₆ cycloalkyl.

In some embodiments, the method for inhibiting BET proteins in a subject comprises administering a therapeutically effective amount of at least one compound of Formula I, wherein:

Ra₁ is selected from methyl, ethyl, methoxy, ethoxy, and propoxy;

Ra₃ is selected from C₁-C₆ alkoxy, hydrogen, and halogen;

Rb₂, Rb₃, Rb₅, and Rb₆ are each hydrogen;

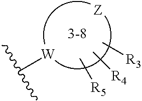

is selected from

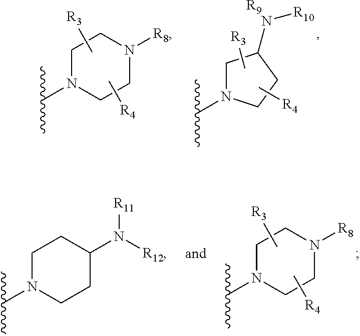

R₃ and R₄ are independently selected from hydrogen and C₁-C₆ alkyl;

R₈ is selected from C₁-C₆ alkyl and hydrogen; and

R₉, R₁₀, R₁₁, and R₁₂ are independently selected from C₁-C₆ alkyl, hydrogen, acyl, and sulfonyl.

In some embodiments, the method for inhibiting BET proteins in a subject comprises administering a therapeutically effective amount of at least one compound of Formula I, wherein:

Ra₁ is selected from methyl, ethyl, methoxy, ethoxy, and propoxy;

Ra₃ is selected from methoxy, hydrogen, and halogen;

Rb₃ and Rb₅ are each hydrogen;

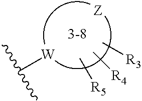

is selected from

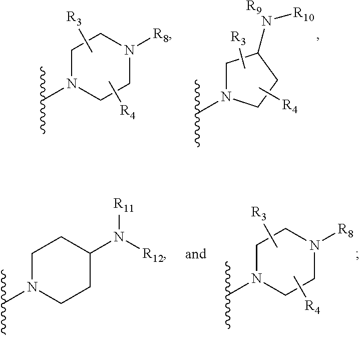

R₃ and R₄ are independently selected from hydrogen and methyl;

R₈ is selected from hydrogen, hydroxyethyl, butyl, acetyl, isopropyl, 4-hexanoyl, 4-isobutyryl, benzoyl, 4-fluorobenzoyl, 4-picolinoyl, 4-nicotinoyl, 4-isonicotinoyl, thiophene-2-carbonyl, 5-chloro-1-methyl-1H-pyrazole-4-carbonyl, 3,3,3-trifluoropropanoyl, 2,5-dichlorothiopene-3-carbonyl, cyclopropanecarbonyl, 4-fluorobenzyl, benzyl, 2,2,2-trifluoroethyl, tertbutoxycarbonyl, and formyl;

R₉ and R₁₀ are independently selected from hydrogen, methyl, cyclopropylmethyl, and acetyl; and

R₁₁ and R₁₂ are independently selected from hydrogen, acetyl, methanesulfonyl, benzoyl, benzyl, ethyl, and isopropyl.

In certain embodiments, the method for inhibiting BET proteins in a subject comprises administering a therapeutically effective amount of at least one compound of Formula I selected from:
5,7-dimethoxy-2-(4-morpholinophenyl)quinazolin-4(3H)-one;
2-(4-((3R,5S)-4-acetyl-3,5-dimethylpiperazin-1-yl)phenyl)-5,7-dimethoxypyrido[2,3-d]pyrimidin-4(3H)-one;
2-(4-(4-hydroxypiperidin-1-yl)phenyl)-5,7-dimethoxypyrido[2,3-d]pyrimidin-4(3H)-one;
2-(4-((3R,5S)-4-acetyl-3,5-dimethylpiperazin-1-yl)phenyl)-5-methoxy-7-(2-methoxyethoxy)quinazolin-4(3H)-one;
2-(4-(4-isopropylpiperazin-1-yl)phenyl)-5,7-dimethoxyquinazolin-4(3H)-one;
2-(4-(4-acetylpiperazin-1-yl)phenyl)-5,7-dimethoxyquinazolin-4(3H)-one;
5,7-dimethoxy-2-(4-(piperazin-1-yl)phenyl)quinazolin-4(3H)-one;
N-(1-(4-(5,7-dimethoxy-4-oxo-3,4-dihydroquinazolin-2-yl)phenyl)piperidin-4-yl)acetamide;
N-(1-(4-(5,7-dimethoxy-4-oxo-3,4-dihydroquinazolin-2-yl)phenyl)piperidin-4-yl)methanesulfonamide;
3-(1-(4-(5,7-dimethoxy-4-oxo-3,4-dihydroquinazolin-2-yl)phenyl)piperidin-4-yl)-1,1-dimethylurea;
2-(4-(4-hexanoylpiperazin-1-yl)phenyl)-5,7-dimethoxyquinazolin-4(3H)-one;
2-(4-(4-isobutyrylpiperazin-1-yl)phenyl)-5,7-dimethoxyquinazolin-4(3H)-one;
2-(4-(4-benzoylpiperazin-1-yl)phenyl)-5,7-dimethoxyquinazolin-4(3H)-one;
2-(4-(4-(4-fluorobenzoyl)piperazin-1-yl)phenyl)-5,7-dimethoxyquinazolin-4(3H)-one;
N-(1-(4-(5,7-dimethoxy-4-oxo-3,4-dihydroquinazolin-2-yl)phenyl)piperidin-4-yl)benzamide;
5,7-dimethoxy-2-(4-(4-picolinoylpiperazin-1-yl)phenyl)quinazolin-4(3H)-one;
5,7-dimethoxy-2-(4-(4-nicotinoylpiperazin-1-yl)phenyl)quinazolin-4(3H)-one;
2-(4-(4-isonicotinoylpiperazin-1-yl)phenyl)-5,7-dimethoxyquinazolin-4(3H)-one;
5,7-dimethoxy-2-(4-(4-(thiophene-2-carbonyl)piperazin-1-yl)phenyl)quinazolin-4(3H)-one;
2-(4-(4-(5-chloro-1-methyl-1H-pyrazole-4-carbonyl)piperazin-1-yl)phenyl)-5,7-dimethoxyquinazolin-4(3H)-one;
5,7-dimethoxy-2-(4-(4-(3,3,3-trifluoropropanoyl)piperazin-1-yl)phenyl)quinazolin-4(3H)-one;
2-(4-(4-(2,5-dichlorothiophene-3-carbonyl)piperazin-1-yl)phenyl)-5,7-dimethoxyquinazolin-4(3H)-one;
2-(4-(4-(cyclopropanecarbonyl)piperazin-1-yl)phenyl)-5,7-dimethoxyquinazolin-4(3H)-one;
2-(4-(4-(4-fluorobenzyl)piperazin-1-yl)phenyl)-5,7-dimethoxyquinazolin-4(3H)-one;
2-(4-(4-benzylpiperazin-1-yl)phenyl)-5,7-dimethoxyquinazolin-4(3H)-one;
2-(4-(4-(2,2,2-trifluoroethyl)piperazin-1-yl)phenyl)quinazolin-4(3H)-one;
2-(4-(4-butylpiperazin-1-yl)phenyl)-5,7-dimethoxyquinazolin-4(3H)-one;
2-(4-(4-acetyl-1,4-diazepan-1-yl)phenyl)-5,7-dimethoxyquinazolin-4(3H)-one;
2-(4-(1,4-diazepan-1-yl)phenyl)-5,7-dimethoxyquinazolin-4(3H)-one;
5,7-dimethoxy-2-(4-(4-methyl-1,4-diazepan-1-yl)phenyl)quinazolin-4(3H)-one;
N-(1-(4-(5,7-dimethoxy-4-oxo-3,4-dihydroquinazolin-2-yl)phenyl)piperidin-4-yl)-N-ethylacetamide;
2-(4-((3R,5S)-4-acetyl-3,5-dimethylpiperazin-1-yl)phenyl)-5,7-dimethoxyquinazolin-4(3H)-one;
2-(4-((3R,5S)-3,5-dimethylpiperazin-1-yl)phenyl)-5,7-dimethoxyquinazolin-4(3H)-one;
2-(4-(4-acetyl-3-methylpiperazin-1-yl)phenyl)-5,7-dimethoxyquinazolin-4(3H)-one;
N-(1-(4-(5,7-dimethoxy-4-oxo-3,4-dihydroquinazolin-2-yl)phenyl)pyrrolidin-3-yl)acetamide;
2-(4-(4-(2-hydroxyethyl)piperazin-1-yl)phenyl)-5,7-dimethoxyquinazolin-4(3H)-one;
N-(1-(4-(5,7-dimethoxy-4-oxo-3,4-dihydroquinazolin-2-yl)phenyl)piperidin-4-yl)-N-isopropylacetamide;
5-chloro-2-(4-(4-isopropylpiperazin-1-yl)phenyl)quinazolin-4(3H)-one;
2-(4-((3R,5S)-4-isopropyl-3,5-dimethylpiperazin-1-yl)phenyl)-5,7-dimethoxyquinazolin-4(3H)-one;
5,7-dimethoxy-2-(4-(piperidin-4-yl)phenyl)quinazolin-4(3H)-one;
5,7-dimethoxy-2-(4-(3-(methylamino)pyrrolidin-1-yl)phenyl)quinazolin-4(3H)-one;
tert-butyl 4-(4-(5,7-dimethoxy-4-oxo-3,4-dihydroquinazolin-2-yl)phenyl)piperidine-1-carboxylate;
N-(1-(4-(5,7-dimethoxy-4-oxo-3,4-dihydroquinazolin-2-yl)phenyl)pyrrolidin-3-yl)-N-methylacetamide;
2-(4-(4-(isopropylamino)piperidin-1-yl)phenyl)-5,7-dimethoxyquinazolin-4(3H)-one;
2-(4-(1-acetylpiperidin-4-yl)phenyl)-5,7-dimethoxyquinazolin-4(3H)-one;
5,7-dimethoxy-2-(4-(3-methylpiperazin-1-yl)phenyl)quinazolin-4(3H)-one;
N-benzyl-N-(1-(5-(5,7-dimethoxy-4-oxo-3,4-dihydroquinazolin-2-yl)pyridin-2-yl)piperidin-4-yl)acetamide;
2-(6-(4-(benzylamino)piperidin-1-yl)pyridin-3-yl)-5,7-dimethoxyquinazolin-4(3H)-one;
4-(4-(5,7-dimethoxy-4-oxo-3,4-dihydroquinazolin-2-yl)phenyl)piperazine-1-carbaldehyde;
2-(4-(3-(cyclopropylmethylamino)pyrrolidin-1-yl)phenyl)-5,7-dimethoxyquinazolin-4(3H)-one;
5,7-dimethoxy-2-(4-(4-oxopiperidin-1-yl)phenyl)pyrido[2,3-d]pyrimidin-4(3H)-one; and stereoisomers, tautomers, pharmaceutically acceptable salts, and hydrates thereof.

### Embodiments Employing Compounds of Formula II

In certain embodiments, the method for inhibiting BET proteins in a subject comprises administering a therapeutically effective amount of at least one compound of Formula II:

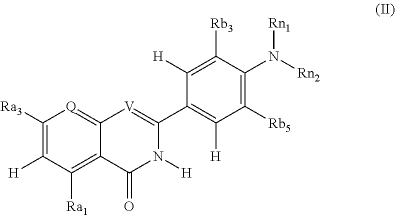

or stereoisomer, tautomer, pharmaceutically acceptable salt, or hydrate thereof, wherein:

Q is CH;

V is nitrogen;

Ra₁ and Ra₃ are each C₁-C₆ alkoxy;

Rb₃ is hydrogen;

Rn₁ is hydrogen;

Rn₂ is selected from sulfonyl, heterocycle, and aryl; and

Rb₅ is hydrogen or Rb₅ may be connected with Rn₂ to form a 5- or 6-membered heterocycle.

In some embodiments, the method for inhibiting BET proteins in a subject comprises administering a therapeutically effective amount of at least one compound of Formula II, wherein:

Q is CH;

V is nitrogen;

Ra₁ and Ra₃ are each methoxy;

Rb₃ is hydrogen;

Rn₁ is hydrogen;

Rn₂ is selected from methanesulfonyl, pyridin-4-yl, 4-methylphenyl, and pyridin-3-yl; and

Rb₅ is hydrogen or Rb₅ may be connected with Rn₂ to form a heterocycle selected from (2-hydroxymethyl)-1H-pyrrol-5-yl, (2-hydroxyethyl)-1H-pyrrol-5-yl, 2-(pyrrolidin-1-yl-ylmethyl)-1H-pyrrol-5-yl, 3-(hydroxymethyl)-1H-pyrazol-5-yl, 2-(pyrrolidin-1-yl-ylethyl)-1H-pyrrol-5-yl, and 2-((dimethylamino)methyl)-1H-pyrrol-5-yl.

In certain embodiments, the method for inhibiting BET proteins in a subject comprises administering a therapeutically effective amount of at least one compound of Formula II selected from:
2-(4-(bis(2-hydroxyethyl)amino)phenyl)-5,7-dimethoxypyrido[2,3-d]pyrimidin-4(3H)-one;
2-(2-(hydroxymethyl)-1H-indol-5-yl)-5,7-dimethoxyquinazolin-4(3H)-one;
2-(2-(2-hydroxyethyl)-1H-indol-5-yl)-5,7-dimethoxyquinazolin-4(3H)-one;
5,7-dimethoxy-2-(2-(pyrrolidin-1-ylmethyl)-1H-indol-5-yl)quinazolin-4(3H)-one;
2-(3-(hydroxymethyl)-1H-indazol-5-yl)-5,7-dimethoxyquinazolin-4(3H)-one;
5,7-dimethoxy-2-(2-(2-(pyrrolidin-1-yl)ethyl)-1H-indol-5-yl)quinazolin-4(3H)-one;
2-(2-((dimethylamino)methyl)-1H-indol-5-yl)-5,7-dimethoxyquinazolin-4(3H)-one;
N-(4-(5,7-dimethoxy-4-oxo-3,4-dihydroquinazolin-2-yl)phenyl)methanesulfonamide;
5,7-dimethoxy-2-(4-(pyridin-4-ylamino)phenyl)quinazolin-4(3H)-one;
5,7-dimethoxy-2-(4-(p-tolylamino)phenyl)quinazolin-4(3H)-one;
5,7-dimethoxy-2-(4-(pyridin-3-ylamino)phenyl)quinazolin-4(3H)-one; and stereoisomers, tautomers, pharmaceutically acceptable salts, and hydrates thereof.

### Embodiments Employing Compounds of Formula III

In certain embodiments, the method for inhibiting BET proteins in a subject comprises administering a therapeutically effective amount of at least one compound of Formula III:

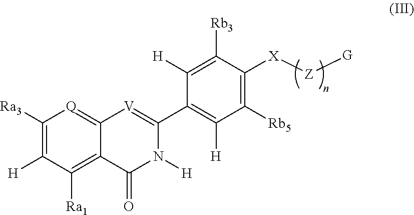

or stereoisomer, tautomer, pharmaceutically acceptable salt, or hydrate thereof, wherein:

V is nitrogen;

Z is selected from unsubstituted C₁-C₆ alkyl;

Ra1 is selected from methyl, ethyl, methoxy, ethoxy, and propoxy;

Ra₃ selected from hydrogen, C₁-C₆ alkyl, C₁-C₆ alkoxy, halogen, and heterocycle;

Rb₃ and Rb₅ are independently selected from hydrogen and C₁-C₆ alkyl; and

X is selected from oxygen and CH₂.

In other embodiments, compounds of Formula III that may be used to treat or prevent a disease or disorder that responds to a BET inhibitor in subject, include those in which:

V is nitrogen;

Ra₁ is selected from methyl, ethyl, methoxy, ethoxy, and propoxy;

Ra₃ is selected from hydrogen, methyl, chlorine, fluorine, methoxy, isopropoxy, and pyrrolidin-1-yl;

Rb₃ and Rb₅ are independently selected from hydrogen and methyl; and

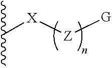

is selected from (N,N-dimethylpiperidine-1-carboxamide)-4-oxy, 1-acetylpiperidin-4-yloxy, 2-(isoindolin-2-yl)ethoxy, 2-(pyrrolidin-1-yl)ethoxy, 3-(pyrrolidin-1-yl)propoxy, 4-(pyrrolidin-1-yl)butoxy, (4-acetylpiperazin-1-yl)ethoxy, (1H-imidazol-1-yl)ethoxy, (4-methylpiperazin-1-yl)ethoxy, (piperidin-1-yl)ethoxy, (1-isopropylimidazolidine-2,4-dione)-3-ethoxy, (5-phenylimidazolidine-2,4-dione)-3-ethoxy, (imidazolidine-2,4-dione)-3-methyl, (2-azepan-1-yl)ethoxy, (2-azetidin-1-yl)ethoxy, N-(azetidin-3-yl)acetamide-1-ethoxy, (isoindoline-1,3-dione)-2-ethoxy, (5-oxopyrrolidin-2-yl)methoxy, (4-isopropylpiperazin-1-yl)methyl, N-isopropyl-N-(piperidin-4-methyl)acetamide-1-methyl, (4-(isopropylamino)piperidin-1-yl)methyl, (pyrrolidine-2,5-dione)ethoxy, and (1H-tetrazol-5-yl)methyl.

In certain embodiments, the method for inhibiting BET proteins in a subject comprises administering a therapeutically effective amount of at least one compound of Formula III selected from:
3-(3,5-dimethyl-4-(2-morpholinoethoxy)phenyl)-6,8-dimethoxyisoquinolin-1(2H)-one;
2-(3,5-dimethyl-4-(2-morpholinoethoxy)phenyl)-5,7-dimethoxyquinazolin-4(3H)-one;
3-(3,5-dimethyl-4-(2-(4-methylpiperazin-1-yl)ethoxy)phenyl)-6,8-dimethoxyisoquinolin-1(2H)-one;
2-(3,5-dimethyl-4-(2-morpholinoethoxy)phenyl)quinazolin-4(3H)-one;
7-(3,5-dimethyl-4-(2-morpholinoethoxy)phenyl)-2,4-dimethoxy-1,6-naphthyridin-5(6H)-one;
5,7-dimethoxy-2-(4-((4-methylpiperazin-1-yl)methyl)phenyl)quinazolin-4(3H)-one;
5,7-dimethoxy-2-(4-(morpholinomethyl)phenyl)quinazolin-4(3H)-one;
2-(4-((4-ethylpiperazin-1-yl)methyl)phenyl)-5,7-dimethoxyquinazolin-4(3H)-one;
2-(3,5-dimethyl-4-(2-(pyrrolidin-1-yl)ethoxy)phenyl)-5,7-dimethoxyquinazolin-4(3H)-one;
4-(4-(5,7-dimethoxy-4-oxo-3,4-dihydroquinazolin-2-yl)phenoxy)-N,N-dimethylpiperidine-1-carboxamide;
2-(4-(1-acetylpiperidin-4-yloxy)phenyl)-5,7-dimethoxyquinazolin-4(3H)-one;
2-(4-(2-(isoindolin-2-yl)ethoxy)-3,5-dimethylphenyl)-5,7-dimethoxyquinazolin-4(3H)-one;
2-(3,5-dimethyl-4-(2-(pyrrolidin-1-yl)ethoxy)phenyl)-5-methoxyquinazolin-4(3H)-one;
5,7-dichloro-2-(3,5-dimethyl-4-(2-(pyrrolidin-1-yl)ethoxy)phenyl)quinazolin-4(3H)-one;
2-(4-(2-(4-acetylpiperazin-1-yl)ethoxy)-3,5-dimethylphenyl)-5,7-dimethoxyquinazolin-4(3H)-one;
2-(4-(2-(1H-imidazol-1-yl)ethoxy)-3,5-dimethylphenyl)-5,7-dimethoxyquinazolin-4(3H)-one;
2-(3,5-dimethyl-4-(2-(pyrrolidin-1-yl)ethoxy)phenyl)-7-methoxyquinazolin-4(3H)-one;
2-(3,5-dimethyl-4-(2-(4-methylpiperazin-1-yl)ethoxy)phenyl)-5,7-dimethoxyquinazolin-4(3H)-one;
2-(3,5-dimethyl-4-(2-(piperidin-1-yl)ethoxy)phenyl)-5,7-dimethoxyquinazolin-4(3H)-one;
5,7-dimethoxy-2-(3-methyl-4-(2-(pyrrolidin-1-yl)ethoxy)phenyl)quinazolin-4(3H)-one;
3-(2-(4-(5,7-dimethoxy-4-oxo-3,4-dihydroquinazolin-2-yl)-2,6-dimethylphenoxy)ethyl)-1-isopropylimidazolidine-2,4-dione;
2-(3,5-dimethyl-4-(3-(pyrrolidin-1-yl)propoxy)phenyl)-5,7-dimethoxyquinazolin-4(3H)-one;
5,7-dimethoxy-2-(4-(2-(pyrrolidin-1-yl)ethoxy)phenyl)quinazolin-4(3H)-one;
2-(3,5-dimethyl-4-(3-(pyrrolidin-1-yl)propyl)phenyl)-5,7-dimethoxyquinazolin-4(3H)-one;
2-(3,5-dimethyl-4-(4-(pyrrolidin-1-yl)butoxy)phenyl)-5,7-dimethoxyquinazolin-4(3H)-one;
3-(2-(4-(5,7-dimethoxy-4-oxo-3,4-dihydroquinazolin-2-yl)-2,6-dimethylphenoxy)ethyl)-5-phenylimidazolidine-2,4-dione;
3-(4-(5,7-dimethoxy-4-oxo-3,4-dihydroquinazolin-2-yl)benzyl)imidazolidine-2,4-dione;
2-(3,5-dimethyl-4-(2-(pyrrolidin-1-yl)ethoxy)phenyl)-5,7-dimethoxypyrido[2,3-d]pyrimidin-4(3H)-one;
2-(3,5-dimethyl-4-(2-(pyrrolidin-1-yl)ethoxy)phenyl)-7-fluoro-5-(pyrrolidin-1-yl)quinazolin-4(3H)-one;
5-chloro-2-(3,5-dimethyl-4-(2-(pyrrolidin-1-yl)ethoxy)phenyl)quinazolin-4(3H)-one;
2-(4-(2-(azepan-1-yl)ethoxy)-3,5-dimethylphenyl)-5,7-dimethoxyquinazolin-4(3H)-one;
2-(3,5-dimethyl-4-(2-(pyrrolidin-1-yl)ethoxy)phenyl)-5,7-difluoroquinazolin-4(3H)-one;
2-(4-(2-(azetidin-1-yl)ethoxy)-3,5-dimethylphenyl)-5,7-dimethoxyquinazolin-4(3H)-one;
N-(1-(2-(4-(5,7-dimethoxy-4-oxo-3,4-dihydroquinazolin-2-yl)-2,6-dimethylphenoxy)ethyl)azetidin-3-yl)acetamide;
2-(3,5-dimethyl-4-(2-(pyrrolidin-1-yl)ethoxy)phenyl)-5,7-diisopropoxyquinazolin-4(3H)-one;
2-(3,5-dimethyl-4-(2-(pyrrolidin-1-yl)ethoxy)phenyl)-5,7-dimethylquinazolin-4(3H)-one;
2-(2-(4-(6,8-dimethoxy-1-oxo-1,2-dihydroisoquinolin-3-yl)-2,6-dimethylphenoxy)ethyl)isoindoline-1,3-dione;
2-(3,5-dimethyl-4-(2-(pyrrolidin-1-yl)ethoxy)phenyl)-5,7-diisopropoxypyrido[2,3-d]pyrimidin-4(3H)-one;
2-(2-(4-(5,7-dimethoxy-4-oxo-3,4-dihydroquinazolin-2-yl)-2,6-dimethylphenoxy)ethyl)isoindoline-1,3-dione;
(S)-2-(3,5-dimethyl-4-((5-oxopyrrolidin-2-yl)methoxy)phenyl)-5,7-dimethoxyquinazolin-4(3H)-one;
2-(4-((4-isopropylpiperazin-1-yl)methyl)phenyl)-5,7-dimethoxyquinazolin-4(3H)-one;
N-(1-(4-(5,7-dimethoxy-4-oxo-3,4-dihydroquinazolin-2-yl)benzyl)piperidin-4-yl)-N-isopropylacetamide;
2-(4-((4-(isopropylamino)piperidin-1-yl)methyl)phenyl)-5,7-dimethoxyquinazolin-4(3H)-one;
2-(4-(2-(1-acetylazetidin-3-yl)ethoxy)-3,5-dimethylphenyl)-5,7-dimethoxyquinazolin-4(3H)-one;
2-(4-((1H-tetrazol-5-yl)methyl)phenyl)-5,7-dimethoxyquinazolin-4(3H)-one;
1-(2-(4-(5,7-dimethoxy-4-oxo-3,4-dihydroquinazolin-2-yl)-2,6-dimethylphenoxy)ethyl)pyrrolidine-2,5-dione;
   and stereoisomers, tautomers, pharmaceutically acceptable salts, and hydrates thereof.

### Embodiments Employing Compounds of Formula IV

In certain embodiments, the method for inhibiting BET proteins in a subject comprises administering a therapeutically effective amount of at least one compound of Formula IV:

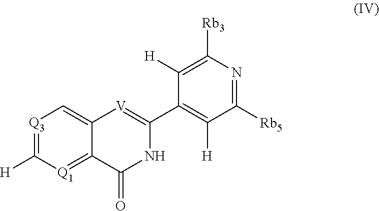

or stereoisomer, tautomer, pharmaceutically acceptable salt, or hydrate thereof, wherein:

V is nitrogen;

Rb₃ and Rb₅ are independently selected from C₁-C₆ alkyl and hydrogen;

Ra₃ is selected from hydrogen and C₁-C₆ alkoxy; and

Ra₁ is selected from methyl, ethyl, methoxy, ethoxy, and propoxy.

In some embodiments, compounds of Formula IV that may be used to treat or prevent cancer or other diseases or disorders that respond to BET inhibitors, are those in which:

V is nitrogen;

Rb₃ and Rb₅ are independently selected from methyl and hydrogen;

Ra₁ is selected from methyl, ethyl, methoxy, ethoxy, and propoxy;

Ra₃ is selected from hydrogen, benzyloxyethoxy, methoxy, methoxyethoxy, (pyrrolidin-1-yl)ethoxy, phenoxyethoxy, and isopropoxyethoxy.

In one embodiment, the method for inhibiting BET proteins in a subject comprises administering a therapeutically effective amount of at least one compound of Formula IV selected from:
7-(2-(benzyloxy)ethoxy)-5-methoxy-2-(pyridin-4-yl)quinazolin-4(3H)-one;
2-(2,6-dimethylpyridin-4-yl)-5,7-dimethoxyquinazolin-4(3H)-one;
2-(2,6-dimethylpyridin-4-yl)-5-methoxy-7-(2-methoxyethoxy)quinazolin-4(3H)-one;
2-(2,6-dimethylpyridin-4-yl)-5,7-bis(2-methoxyethoxy)quinazolin-4(3H)-one;
2-(2,6-dimethylpyridin-4-yl)-7-methoxy-5-(2-(pyrrolidin-1-yl)ethoxy)quinazolin-4(3H)-one;
2-(2,6-dimethylpyridin-4-yl)-5-methoxy-7-(2-phenoxyethoxy)quinazolin-4(3H)-one;
2-(2,6-dimethylpyridin-4-yl)-7-methoxy-5-(2-phenoxyethoxy)quinazolin-4(3H)-one;
2-(2,6-dimethylpyridin-4-yl)-7-methoxy-5-(2-methoxyethoxy)quinazolin-4(3H)-one;
2-(2,6-dimethylpyridin-4-yl)-5-methoxy-7-(2-(pyrrolidin-1-yl)ethoxy)quinazolin-4(3H)-one;
2-(2,6-dimethylpyridin-4-yl)-7-(2-isopropoxyethoxy)-5-methoxyquinazolin-4(3H)-one;
2-(2,6-dimethylpyridin-4-yl)-5,7-bis(2-isopropoxyethoxy)quinazolin-4(3H)-one;
7-(2-(benzyloxy)ethoxy)-2-(2,6-dimethylpyridin-4-yl)-5-methoxyquinazolin-4(3H)-one;
5-methoxy-7-(2-methoxyethoxy)-2-(2-methylpyridin-4-yl)quinazolin-4(3H)-one;
2-(2,6-dimethylpyridin-4-yl)-5-(2-isopropoxyethoxy)-7-methoxyquinazolin-4(3H)-one;
2-(2,6-dimethylpyridin-4-yl)-7-(2-methoxyethoxy)-5-(2-(pyrrolidin-1-yl)ethoxy)quinazolin-4(3H)-one;
   and stereoisomers, tautomers, pharmaceutically acceptable salts, and hydrates thereof.

### Embodiments Employing Compounds of Formula V

In certain embodiments, the method for inhibiting BET proteins in a subject comprises administering a therapeutically effective amount of at least one compound of Formula V:

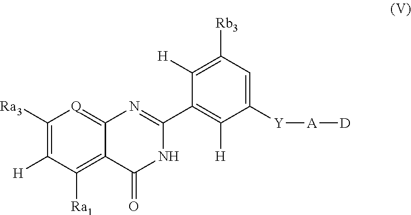

or stereoisomer, tautomer, pharmaceutically acceptable salt, or hydrate thereof, wherein:

Ra₁ is selected from methyl, ethyl, methoxy, ethoxy, and propoxy

Ra₃ are independently selected from hydrogen and C₁-C₆ alkoxy;

Q is CH;

Rb₃ is selected from hydrogen, C₁-C₆ alkyl, and C₁-C₆ alkoxy;

Y is oxygen;

A is C₁-C₄ alkyl;

D may be absent or present, and if present, is selected from hydroxy, heterocycle, and NR₁R₂; and

R₁ and R₂ are independently selected from hydrogen and C₁-C₆ alkyl, or alternatively R₁ and R₂ are joined to form a cycloalkyl or a heterocycle.

In some embodiments, the method for inhibiting BET proteins in a subject comprises administering a therapeutically effective amount of at least one compound of Formula V, wherein:

Ra₁ is selected from methyl, ethyl, methoxy, ethoxy, and propoxy;

Ra₃ is selected from hydrogen and C₁-C₆ alkoxy;

Q is CH;

Rb₃ is selected from hydrogen, methyl, and methoxy;

Y is oxygen;

A is selected from methyl and ethyl;

D may be absent or present, and if present, is selected from hydroxy, pyrrolidin-1-yl, and NR₁R₂; and

R₁ and R₂ are independently selected from hydrogen and acetyl, or alternatively

R₁ and R₂ are joined to form a cycloalkyl or a heterocycle.

In one embodiment, the method for inhibiting BET proteins in a subject comprises administering a therapeutically effective amount of at least one compound of Formula V selected from:
2-(3,5-dimethoxyphenyl)-5,7-dimethoxyquinazolin-4(3H)-one;
2-(3-(2-hydroxyethoxy)phenyl)-5,7-dimethoxyquinazolin-4(3H)-one;
2-(3-(2-hydroxyethoxy)-5-methylphenyl)-5,7-dimethoxyquinazolin-4(3H)-one;
5,7-dimethoxy-2-(3-methoxy-5-(2-(pyrrolidin-1-yl)ethoxy)phenyl)quinazolin-4(3H)-one;
N-(2-(3-(5,7-dimethoxy-4-oxo-3,4-dihydroquinazolin-2-yl)-5-methoxyphenoxy)ethyl)acetamide;
5,7-dimethoxy-2-(3-methoxyphenyl)quinazolin-4(3H)-one; and stereoisomers, tautomers, pharmaceutically acceptable salts, and hydrates thereof.

### Pharmaceutical Compositions

Pharmaceutical compositions employed in the methods of the invention comprise at least one compound of Formula I, II, III, IV, V, or tautomer, stereoisomer, pharmaceutically acceptable salt or hydrate thereof formulated together with one or more pharmaceutically acceptable carriers. These formulations include those suitable for oral, rectal, topical, intraocular, buccal and parenteral (for example, subcutaneous, intramuscular, intradermal, intravenous, or via implants) administration. The most suitable form of administration in any given case will depend on the degree and severity of the condition being treated and on the nature of the particular compound being used.

Formulations suitable for oral administration may be presented in discrete units, such as capsules, cachets, lozenges, or tablets, each containing a predetermined amount of a compound of the invention as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association at least one compound of the invention as the active compound and a carrier or excipient (which may constitute one or more accessory ingredients). The carrier must be acceptable in the sense of being compatible with the other ingredients of the formulation and must not be deleterious to the recipient. The carrier may be a solid or a liquid, or both, and may be formulated with at least one compound described herein as the active compound in a unit-dose formulation, for example, a tablet, which may contain from about 0.05% to about 95% by weight of the at least one active compound. Other pharmacologically active substances may also be present including other compounds. The formulations of the invention may be prepared by any of the well known techniques of pharmacy consisting essentially of admixing the components.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by, for example, dissolving or dispersing, at least one active compound of the invention as described herein and optional pharmaceutical adjuvants in an excipient, such as, for example, water, saline, aqueous dextrose, glycerol, ethanol, and the like, to thereby form a solution or suspension. In general, suitable formulations may be prepared by uniformly and intimately admixing the at least one active compound of the invention with a liquid or finely divided solid carrier, or both, and then, if necessary, shaping the product. For example, a tablet may be prepared by compressing or molding a powder or granules of at least one compound of the invention, which may be optionally combined with one or more accessory ingredients. Compressed tablets may be prepared by compressing, in a suitable machine, at least one compound of the invention in a free-flowing form, such as a powder or granules, which may be optionally mixed with a binder, lubricant, inert diluent and/or surface active/dispersing agent(s). Molded tablets may be made by molding, in a suitable machine, where the powdered form of at least one compound of the invention is moistened with an inert liquid diluent.

Formulations suitable for buccal (sub-lingual) administration include lozenges comprising at least one compound of the invention in a flavored base, usually sucrose and acacia or tragacanth, and pastilles comprising the at least one compound in an inert base such as gelatin and glycerin or sucrose and acacia.

Formulations of the invention suitable for parenteral administration comprise sterile aqueous preparations of at least one compound of Formula I, II, III, IV, V, or a tautomer, stereoisomer, pharmaceutically acceptable salt or hydrate thereof, which are approximately isotonic with the blood of the intended recipient. These preparations are administered intravenously, although administration may also be effected by means of subcutaneous, intramuscular, or intradermal injection. Such preparations may conveniently be prepared by admixing at least one compound described herein with water and rendering the resulting solution sterile and isotonic with the blood. Injectable compositions according to the invention may contain from about 0.1 to about 5% w/w of the active compound.

Formulations suitable for rectal administration are presented as unit-dose suppositories. These may be prepared by admixing at least one compound as described herein with one or more conventional solid carriers, for example, cocoa butter, and then shaping the resulting mixture.

Formulations suitable for topical application to the skin may take the form of an ointment, cream, lotion, paste, gel, spray, aerosol, or oil. Carriers and excipients which may be used include Vaseline, lanoline, polyethylene glycols, alcohols, and combinations of two or more thereof. The active compound (i.e., at least one compound of Formula I, II, III, IV, V, or a tautomer, stereoisomer, pharmaceutically acceptable salt or hydrate thereof) is generally present at a concentration of from about 0.1% to about 15% w/w of the composition, for example, from about 0.5 to about 2%.

The amount of active compound administered may be dependent on the subject being treated, the subject's weight, the manner of administration and the judgment of the prescribing physician. For example, a dosing schedule may involve the daily or semi-daily administration of the encapsulated compound at a perceived dosage of about 1 μg to about 1000 mg. In another embodiment, intermittent administration, such as on a monthly or yearly basis, of a dose of the encapsulated compound may be employed. Encapsulation facilitates access to the site of action and allows the administration of the active ingredients simultaneously, in theory producing a synergistic effect. In accordance with standard dosing regimens, physicians will readily determine optimum dosages and will be able to readily modify administration to achieve such dosages.

A therapeutically effective amount of a compound or composition disclosed for use in the methods of the invention can be measured by the therapeutic effectiveness of the compound. The dosages, however, may be varied depending upon the requirements of the patient, the severity of the condition being treated, and the compound being used. In one embodiment, the therapeutically effective amount of a disclosed compound is sufficient to establish a maximal plasma concentration. Preliminary doses as, for example, determined according to animal tests, and the scaling of dosages for human administration is performed according to art-accepted practices.

Toxicity and therapeutic efficacy can be determined by standard pharmaceutical procedures in cell cultures or experimental animals, for example, for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferable.

Data obtained from the cell culture assays or animal studies can be used in formulating a range of dosage for use in humans. Therapeutically effective dosages achieved in one animal model may be converted for use in another animal, including humans, using conversion factors known in the art (see, for example, Freireich et al., Cancer Chemother. Reports 50(4):219-244 (1966) and Table 1 for Equivalent Surface Area Dosage Factors).

TABLE 1
Equivalent Surface Area Dosage Factors

| | To: | | | | |
|---|---|---|---|---|---|
| | Mouse | Rat | Monkey | Dog | Human |
| From: | (20 g) | (150 g) | (3.5 kg) | (8 kg) | (60 kg) |
| Mouse | 1 | 1/2 | 1/4 | 1/6 | 1/12 |
| Rat | 2 | 1 | 1/2 | 1/4 | 1/7 |
| Monkey | 4 | 2 | 1 | 3/5 | 1/3 |
| Dog | 6 | 4 | 3/5 | 1 | 1/2 |
| Human | 12 | 7 | 3 | 2 | 1 |

The dosage of such compounds lies preferably within a range of circulating concentrations that include the ED₅₀ with little or no toxicity. The dosage may vary within this range depending upon the dosage form employed and the route of administration utilized. Generally, a therapeutically effective amount may vary with the subject's age, condition, and gender, as well as the severity of the medical condition in the subject. The dosage may be determined by a physician and adjusted, as necessary, to suit observed effects of the treatment.

In one embodiment, a compound of Formula I, II, III, IV, V or a tautomer, stereoisomer, pharmaceutically acceptable salt or hydrate thereof, is administered in combination with another therapeutic agent. The other therapeutic agent can provide additive or synergistic value relative to the administration of a compound of the invention alone. In certain embodiments, a compound of Formula I, II, III, IV, V or a tautomer, stereoisomer, pharmaceutically acceptable salt or hydrate thereof, is administered in combination with one or more anti-cancer agents.

### Therapeutic Methods

The invention provides methods of treating or preventing diseases or disorders that respond to BET inhibitors, such as, for example, cancer, immune disorders, inflammatory disorders, and diseases caused by bacterial or viral infection. These methods comprise administering to a subject (for example, a mammal, such as a human) a therapeutically effective amount of at least one compound of Formula I, II, III, IV, V, or a tautomer, stereoisomer, pharmaceutically acceptable salt or hydrate thereof.

In another embodiment, at least one compound of the invention may be administered as a pharmaceutically acceptable composition, comprising one or more compounds of Formula I or II and a pharmaceutically acceptable carrier.

In some embodiments, the disease or disorder is a cancer which may be treated or prevented by administering a therapeutically effective amount of at least one compound of the invention, i.e., a compound of Formula I, II, III, IV, V or a tautomer, stereoisomer, pharmaceutically acceptable salt or hydrate thereof.

In certain embodiments, the cancer to be treated is a midline carcinoma. In some embodiments, the cancer is characterized by c-myc overexpression. In other embodiments, the cancer is characterized by overexpression of n-myc. In certain embodiments, the cancer is Burkitt's lymphoma, acute myelogenous leukemia, multiple myeloma, or aggressive human medulloblastoma. In some embodiments, the cancer is relies on the recruitment of p-TEFb to regulate activated oncogenes such as, for example, NOTCH1. In some embodiments, the cancer to be treated or prevented by the methods of the invention is selected from the group consisting of hematological, epithelial including lung, breast and colon carcinomas, midline carcinomas, mesenchymal, hepatic, renal and neurological tumours.

The certain embodiments, administration of at least one compound of Formula I, II, III, IV, V, or a tautomer, stereoisomer, pharmaceutically acceptable salt or hydrate thereof to a mammal suffering from a cancer, induces apoptosis in cancer cells by decreasing expression of the anti-apoptosis gene Bcl2. Thus, some embodiments of the invention provide a method of treating or preventing a disease or disorder in a mammal that benefits from increased cell death or differentiation, or decreased cell proliferation, comprising administering at least one compound of Formula I, II, III, IV, V, or a tautomer, stereoisomer, pharmaceutically acceptable salt or hydrate thereof.

In some embodiments of the invention, the at least one compound of Formula I, II, III, IV, V, or tautomer, stereoisomer, pharmaceutically acceptable salt or hydrate thereof is administered in combination with another anti-cancer agent, such as, for example, bortezomib, thalidomide, dexamethasone, 5-azacitidine, decitabine, vorinostat, or cyclophosphamide. In some embodiments, the anti-cancer agent is a PI3K or mTOR inhibitor, such as rapamycin or a rapamycin analog. In some embodiments, the anti-cancer agent is a gamma secretase inhibitor or a AMPK inducer, such as, for example, metformin or phenformin. In certain embodiments, the anti-cancer agent is an ornithine decarboxylase inhibitor, such as, for example, difluoromethylornithine.

At least one compound of Formula I, II, III, IV, V, or a tautomer, stereoisomer, pharmaceutically acceptable salt or hydrate thereof may also be administered to treat or prevent a disease or disorder resulting from an infection by bacteria or virus, such as for example, HIV, HPV, or herpes. In some embodiments, the disease or disorder to be treated by the methods of the invention is AIDS. In other embodiments, the at least one compound of Formula I, II, III, IV, V, or a tautomer, stereoisomer, pharmaceutically acceptable salt or hydrate thereof is administered to treat or prevent sepsis in a mammal.

### EXAMPLES

The invention is further illustrated by the following non-limiting examples, wherein the following abbreviations have the following meanings. If an abbreviation is not defined, it has its generally accepted meaning.

AcOH=acetic acid

BINAP=2,2′-bis(diphenylphosphino)-1,1′-binaphthyl

Boc=N-tert-butoxycarbonyl

TBDMS=tert-butyldimethylsilyl

dba=dibenzylidene acetone

DCM=dichloromethane

DMAP=dimethylaminopyridine

DMF=dimethylformamide

DMSO=dimethylsulfoxide

EDCI=1-ethyl-3-(3′-dimethylaminopropyl)carbodiimide

EtOH=ethanol

EtOAc=ethyl acetate

IBX=1,2-benziodexol-3(1H)-one-1-hydroxy-1-oxide

MeOH=methanol

HOBt=N-hydroxybenzotriazole

THF=tetrahydrofuran

TEA=triethylamine

p-TSA=p-toluenesulfonic acid

TBAF=tetrabutylammonium fluoride

DMA=N,N-dimethylacetamide

DIBAL-H=diisobutylaluminum hydride

TPAP=tetrapropylammonium perruthenate

NMO=N-methylmorpholine N-oxide

DDQ=2,3-dicyano-5,6-dichloro-parabenzoquinone

DME=1,2-dimethoxyethane

TFA=trifluoroacetic acid

DPPF=1,1′-bis(diphenylphosphino)ferrocene

Pd(OAc)₂=palladium(II) acetate

Pd(PPh₃)₄=tetrakis(triphenylphosphine)palladium(0)

### Example 1

### Preparation of 2-(4-(4-(2-hydroxyethyl)piperazin-1-yl)phenyl)-5,7-dimethoxyquinazolin-4(3H)-one (2)

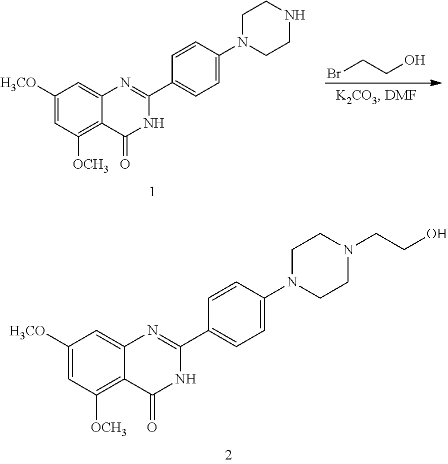

To a solution of 5,7-dimethoxy-2-(4-(piperazin-1-yl)phenyl)quinazolin-4(3H)-one (1) (0.68 mmol) in DMF (8 mL) was added potassium carbonate (0.68 mmol) and 2-bromoethanol (0.68 mmol). The resulting solution was stirred at room temperature overnight. Then, the mixture was diluted with water, extracted with EtOAc, washed with brine, dried over anhydrous Na₂SO₄, filtered, and concentrated in vacuo to afford 2. The material was purified by flash chromatography on silica gel, eluting with 50% to 100% of 92:7:1 CHCl₃/MeOH/concentrated NH₄OH in CH₂Cl₂. The product was further purified by reverse-phase chromatography, eluting with 10% to 90% CH₃CN in H₂O, to afford the title compound (0.025 g, 9%). ¹H NMR (300 MHz, DMSO-d₆): δ 11.45 (s, 1H), 8.08 (d, J=8.9 Hz, 2H), 7.00 (d, J=9.1 Hz, 2H), 6.68 (s, 1H), 6.46 (s, 1H), 4.30-4.55 (m, 1H), 3.88 (s, 3H), 3.83 (s, 3H), 3.43-3.67 (m, 2H), 3.10-3.43 (m, 7H), 2.77-3.04 (m, 1H), 2.31-2.64 (m, 2H). ESI MS m/z 411 [M+H]⁺.

### Example 2

### Preparation of 2-(4-(4-butylpiperazin-1-yl)phenyl)-5,7-dimethoxyquinazolin-4(3H)-one (7)

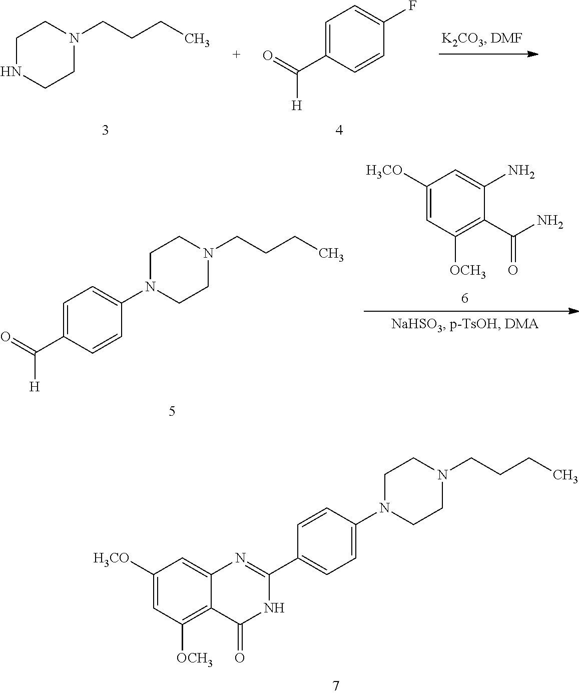

To a solution of 1-(N-butyl)-piperazine (3) (7.03 mmol) in DMF (8 mL) was added 4-fluorobenzaldehyde (4) (8.43 mmol) and potassium carbonate (8.43 mmol). The resulting solution was heated to 120° C. for 5 hours and diluted with water. The solution was extracted with EtOAc, washed with water, brine, dried over anhydrous Na₂SO₄, filtered, and concentrated in vacuo. The material was purified by flash chromatography on silica gel to afford 4-(4-butylpiperazin-1-yl)benzaldehyde (5).

To a solution of 2-amino-4,6-dimethoxybenzamide (6) (1.19 mmol) in DMA (10 mL) was added 4-(4-butylpiperazin-1-yl)benzaldehyde (5) (1.09 mmol), NaHSO₃ (1.30 mmol), and p-TsOH (0.10 mmol). The resulting solution was heated to 155° C. for 4 hours and cooled to room temperature. The solution was diluted with water, extracted with EtOAc, washed with brine, dried over anhydrous Na₂SO₄, filtered, and concentrated in vacuo. The material was purified by flash chromatography on silica gel eluting with 10% to 50% of 92:7:1 CHCl₃/MeOH/concentrated NH₄OH in CH₂Cl₂, to afford the compound 7 (0.06 g, 13%). ¹H NMR (300 MHz, DMSO-d₆): δ 11.76 (s, 1H), 8.09 (d, J=8.9 Hz, 2H), 7.00 (d, J=9.0 Hz, 2H), 6.68 (s, 1H), 6.47 (s, 1H), 3.88 (s, 3H), 3.83 (s, 3H), 3.17-3.42 (m, 4H), 2.39-2.58 (m, 4H), 2.23-2.37 (m, 2H), 1.37-1.56 (m, 2H), 1.26-1.37 (m, 2H), 0.84-0.94 (m, 3H). APCI MS m/z 423 [M+H]⁺.

### Example 3

### Preparation of 2-(4-(1-acetylpiperidin-4-yl)phenyl)-5,7-dimethoxyquinazolin-4(3H)-one (13)

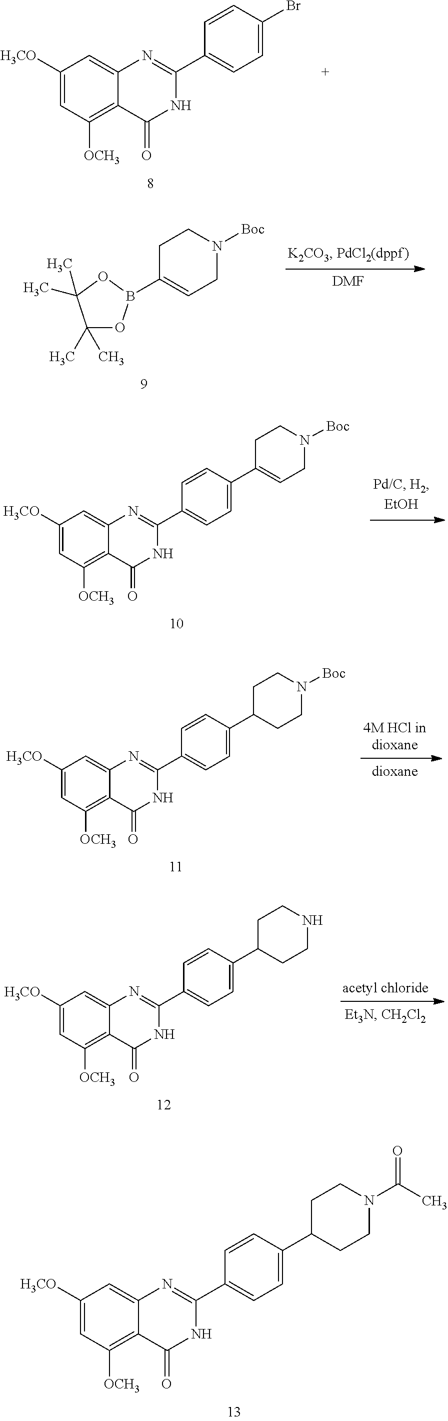

A solution of 2-(4-bromophenyl)-5,7-dimethoxyquinazolin-4(3H)-one (8) (3.23 mmol), K₂CO₃ (9.69 mmol), PdCl₂(dppf) (0.32 mmol) and tert-butyl 4-(4,4,5,5-tetramethyl-1,3,2-dioxaborolan-2-yl)-5,6-dihydropyridine-1(2H)-carboxylate (9) (3.23 mmol) in DMF (50 mL) was heated to 110° C. overnight. The resulting solution was concentrated in vacuo and the material was purified by flash chromatography on silica gel to give tert-butyl 4-(4-(5,7-dimethoxy-4-oxo-3,4-dihydroquinazolin-2-yl)phenyl)-5,6-dihydropyridine-1(2H)-carboxylate (10).

A solution of tert-butyl 4-(4-(5,7-dimethoxy-4-oxo-3,4-dihydroquinazolin-2-yl)phenyl)-5,6-dihydropyridine-1(2H)-carboxylate (10) (0.34 mmol) in EtOH (10 mL) and HOAc (5 mL) was purged with nitrogen and 10% Pd/C (0.016 g) was added. The mixture was stirred under 1 atmosphere of hydrogen overnight. Then, the solution was filtered through Celite, with MeOH washings, and the filtrate was concentrated in vacuo. The material was purified by flash chromatography on silica gel to afford tert-butyl 4-(4-(5,7-dimethoxy-4-oxo-3,4-dihydroquinazolin-2-yl)phenyl)piperidine-1-carboxylate (11).

To a solution of tert-butyl 4-(4-(5,7-dimethoxy-4-oxo-3,4-dihydroquinazolin-2-yl)phenyl)piperidine-1-carboxylate (11) (0.45 mmol) in 1,4-dioxane (2 mL) was added 4 M HCl in 1,4-dioxane (1 mL). The resulting solution was stirred at room temperature for 5 hours. Then, the mixture was concentrated in vacuo and the resulting material was purified by flash chromatography on silica gel to afford compound 5,7-dimethoxy-2-(4-(piperidin-4-yl)phenyl)quinazolin-4(3H)-one (12).

To a solution of 5,7-dimethoxy-2-(4-(piperidin-4-yl)phenyl)quinazolin-4(3H)-one (0.16 mmol) in CH₂Cl₂ (10 mL) was added Et₃N (0.32 mmol) and acetyl chloride (0.17 mmol). The resulting solution was stirred at 0° C. overnight. The solution was concentrated in vacuo, basified with NaHCO₃, extracted with CH₂Cl₂, and washed with water and brine. The material was dried (Na₂SO₄), filtered, and concentrated to afford the title compound 13 (0.020 g, 30%). ¹H NMR (300 MHz, DMSO-d₆): δ 11.93 (s, 1H), 8.11 (d, J=8.3 Hz, 2H), 7.40 (d, J=8.3 Hz, 2H), 6.73 (s, 1H), 6.53 (s, 1H), 4.42-4.64 (m, 1H), 3.89 (s, 3H), 3.85 (s, 3H), 3.06-3.21 (m, 1H), 2.77-2.94 (m, 1H), 2.54-2.68 (m, 1H), 2.03 (s, 3H), 1.73-1.91 (m, 2H), 1.56-1.73 (m, 1H), 1.36-1.56 (m, 1H), 1.06-1.36 (m, 1H). ESI MS m/z 408 [M+H]⁺.

### Example 4

### Preparation of 2-(4-(3-(cyclopropylmethylamino)pyrrolidin-1-yl)phenyl)-5,7-dimethoxyquinazolin-4(3H)-one (15)

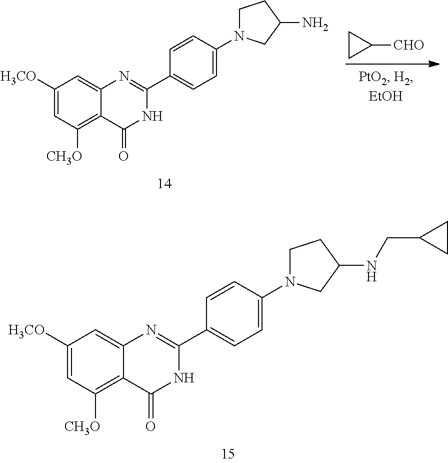

A suspension of 2-(4-(3-aminopyrrolidin-1-yl)phenyl)-5,7-dimethoxyquinazolin-4(3H)-one (14) (0.21 mmol) in ethanol (30 mL) was treated with PtO₂ (0.050 g) followed by cyclopropanecarbaldehyde (0.100 mL). The reaction was stirred under 1 atmosphere of hydrogen for 24 hours, filtered through Celite, with ethanol washes, concentrated, and purified by flash chromatography on silica gel, eluting to afford the title compound 15.

### Example 5

### Preparation of 2-(4-(2-(1-acetylazetidin-3-yl)ethoxy)-3,5-dimethylphenyl)-5,7-dimethoxyquinazolin-4(3H)-one (19)

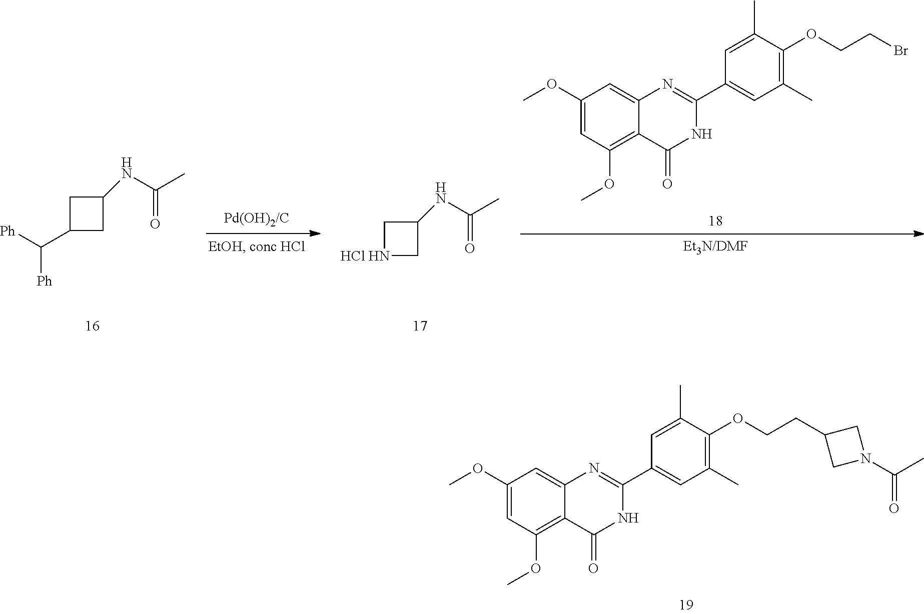

To a solution of N-(1-benzhydryl-azetidin-3-yl)-acetamide (16) (3.57 mmol) in ethanol (20 mL) were added palladium hydroxide on carbon (20 wt %, 0.20 g) and concentrated HCl (0.6 mL). The reaction mixture was hydrogenated at 50 psi at 40° C. for 2 hours, then filtered and washed with methanol (50 mL). The filtrate was collected and the solvent was evaporated, to give N-azetidin-3-yl-acetamide (17).

To a suspension of N-azetidin-3-yl-acetamide (17) (1.99 mmol) and 2-[4-(2-bromo-ethoxy)-3,5-dimethyl-phenyl]-5,7-dimethoxy-3H-quinazolin-4-one (18) (1.00 mmol) in anhydrous DMF (10 mL) was added triethylamine (3 mL). The reaction mixture was stirred at room temperature for 3 days under nitrogen. The solvent was evaporated under reduced pressure, water (50 mL) was added, and the precipitated solid was filtered off. The aqueous layer was extracted with ethyl acetate (2×100 mL). The organic phase was dried over anhydrous Na₂SO₄ and concentrated. The crude compound was purified by the Simpliflash system (0-5% 7 N ammonia in methanol and CH₂Cl₂ as eluent) to give the title compound 19 as a white solid.

### Example 6

### Preparation of 2-(2,6-dimethylpyridin-4-yl)-5-(2-isopropoxyethoxy)-7-methoxyquinazolin-4(3H)-one (23)

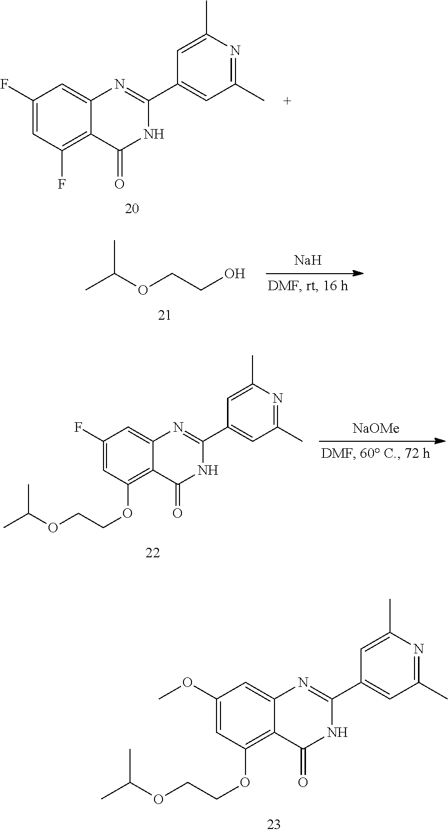

To a solution of 2-isopropoxy ethanol (21) (57.0 mmol) in anhydrous DMF (10 mL) was added a sodium hydride (60% suspension in mineral oil, 28.54 mmol) in small portions at room temperature under nitrogen. After the addition, the reaction mixture was stirred at room temperature for 30 minutes. Then, 2-(2,6-dimethyl-pyridin-4-yl)-5,7-difluoro-3H-quinazolin-4-one (20) (2.85 mmol) was added, and the reaction mixture was stirred at room temperature for 16 hours. The reaction mixture was cooled to room temperature and saturated NH₄Cl solution was added. The product was extracted with ethyl acetate (3×200 mL). The combined organic layer was washed with water, brine, dried over anhydrous Na₂SO₄, and evaporated to give crude product (22) as a white solid.

2-(2,6-Dimethyl-pyridin-4-yl)-7-fluoro-5-(2-isopropoxy-ethoxy)-3H-quinazolin-4-one (22) (960 mg, 2.58 mmol) was taken up in anhydrous DMF (10 mL). Sodium methoxide (25% solution in methanol, 12.9 mmol) was added. After the addition, the reaction mixture was stirred at 60° C. for 72 hours. The reaction mixture was cooled to room temperature, and quenched with saturated solution of NH₄Cl. The product was extracted with ethyl acetate (3×200 mL). The combined organic layer was washed with water, brine, dried over Na₂SO₄, and evaporated to give crude product. The crude compound was purified by preparative HPLC, to give the title compound 23 as a white solid.

### Example 7

### Preparation of 2-(4-((3R,5S)-4-Acetyl-3,5-dimethylpiperazin-1-yl)phenyl)-5,7-dimethoxypyrido[2,3-d]pyrimidin-4(3H)-one

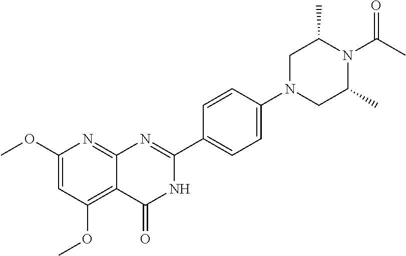 [SMILES: COc1cc(OC)c2c(=O)[nH]c(-c3ccc(N4C[C@@H](C)N(C(C)=O)[C@@H](C)C4)cc3)nc2n1]

To a solution of 4-fluoro-benzaldehyde (3.0 g, 0.024 mol) and 1-(2,6-dimethyl-piperazin-1-yl)-ethanone (3.0 g, 0.019 mol) in anhydrous DMF (15 mL) was added potassium carbonate (6.6 g, 0.048 mol). The reaction mixture was heated to 130° C. for 32 hours. The DMF was removed and the residue was purified by column chromatography (silica gel 230-400 mesh; eluting with 2:1 ethyl acetate and dichloromethane) to give 4-(4-acetyl-3,5-dimethyl-piperazin-1-yl)-benzaldehyde as light yellow solid (2.31 g, 46.2%).

A mixture of 2-amino-4,6-dimethoxy-nicotinamide (0.25 g, 1.26 mmol), 4-(4-acetyl-3,5-dimethyl-piperazin-1-yl)-benzaldehyde (0.43 g, 1.64 mmol), p-toluenesulfonic acid monohydrate (0.53 mg, 2.77 mmol) and sodium bisulfite (0.45 g, 2.52 mmol) in N,N-dimethylacetamide (5.0 mL) was stirred at 135° C. under N₂ for 16 hours and then cooled to room temperature. The mixture was concentrated to dryness under reduced pressure. Water (40 mL) was added to the residue and stirred for 0.5 hours. The precipitate was filtered and the solid was rinsed with water and dried over Na₂SO₄. The crude solid was purified by column chromatography (silica gel 230-400 mesh; eluting with 2.5% methanol in dichloromethane) to afford the title compound as yellow solid. Yield: 90 mg (16.3%). MP 279-279.8° C. ¹H NMR (400 MHz, CDCl₃): δ 10.18 (s, 1H), 8.14 (d, J=8.8 Hz, 2H), 6.99 (d, J=8.8 Hz, 2H), 6.20 (s, 1H), 4.78 (bs, 1H), 4.12 (s, 3H), 4.02 (s, 3H), 3.70 (d, J=12.0 Hz, 2H) 3.11 (d, J=10 Hz, 2H), 2.18 (s, 3H), 1.40 (bs, 6H).

### Example 8

### Preparation of 2-(4-(4-Hydroxypiperidin-1-yl)phenyl)-5,7-dimethoxypyrido[2,3-d]pyrimidin-4(3H)-one

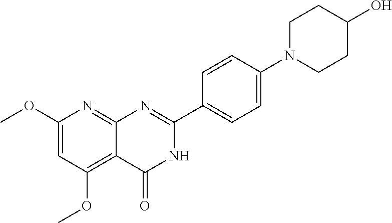 [SMILES: COc1cc(OC)c2c(=O)[nH]c(-c3ccc(N4CCC(O)CC4)cc3)nc2n1]

A mixture of 2-amino-4,6-dimethoxy-nicotinamide (0.60 g, 3.0 mmol), 4-(4-hydroxy-piperidin-1-yl)-benzaldehyde (0.81 g, 3.9 mmol), p-toluenesulfonic acid monohydrate (1.25 g, 6.6 mmol) and sodium bisulfite (1.06 g, 6.0 mmol) in N,N-dimethylacetamide (8.0 mL) was stirred at 135° C. under N₂ for 16 hours and then cooled to room temperature. The mixture was concentrated to dryness under reduced pressure. Water (40 mL) was added to the residue and stirred for 0.5 hours. The precipitate was filtered and the solid was rinsed with water and air-dried. The crude solid was purified by column chromatography (silica gel 230-400 mesh; eluting with 4% methanol in dichloromethane) to afford the title compound, as a yellow solid. Yield: 0.29 g (25.2%). MP 284-286° C. ¹H NMR (400 MHz, DMSO-d₆): δ 12.09 (s, 1H), 8.12 (d, J=8.8 Hz, 2H), 7.02 (d, J=8.8 Hz, 2H), 6.32 (s, 1H), 4.73 (d, J=4.4 Hz, 1H), 3.94 (s, 3H), 3.89 (s, 3H), 3.72 (m, 3H), 3.05 (m, 2H), 1.80 (m, 2H), 1.43 (m, 2H). MS (ES⁺) m/z: 383.06 (M+1).

### Example 9

### Preparation of 2-(4-((3R,5S)-4-Acetyl-3,5-dimethylpiperazin-1-yl)phenyl)-5-methoxy-7-(2-methoxyethoxy)quinazolin-4(3H)-one

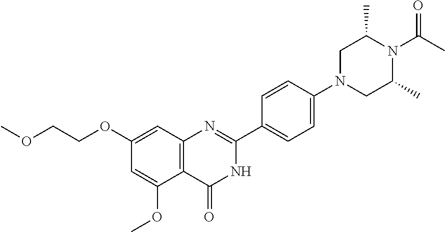 [SMILES: COCCOc1cc(OC)c2c(=O)[nH]c(-c3ccc(N4C[C@@H](C)N(C(C)=O)[C@@H](C)C4)cc3)nc2c1]

To a stirred solution of 2-amino-4,6-difluoro-benzamide (0.66 g, 3.84 mmol) and 4-(4-acetyl-3,5-dimethyl-piperazin-1-yl)-benzaldehyde (1.00 g, 3.84 mmol) in N,N-dimethyl acetamide (20 mL), was added sodium hydrogen sulfite (58.5 wt %, 1.04 g, 5.76 mmol) and p-toluenesulfonic acid monohydrate (0.88 g, 4.61 mmol) and the reaction mixture was stirred at 115° C. for 16 hours. The solvent was evaporated in vacuo, water was added, and the precipitated solid was filtered off, to give 2-[4-(4-acetyl-3,5-dimethyl-piperazin-1-yl)-phenyl]-5,7-difluoro-3H-quinazolin-4-one as a yellow solid, which was used in the next step without further purification.

To a solution of 2-[4-(4-acetyl-3,5-dimethyl-piperazin-1-yl)-phenyl]-5,7-difluoro-3H-quinazolin-4-one (0.66 g, 1.60 mmol) in DMF (10 mL), a solution of sodium methoxide in methanol (25 wt %, 3.5 mL, 16.0 mmol) was added and the reaction mixture was stirred at room temperature for 16 hours. Water was added, acidified to pH approximately 4-5 with acetic acid, and the precipitated solid was filtered and dried under vacuum to give crude compound, which was further purified by column chromatography (silica gel 230-400 mesh; eluting with 2% methanol solution in dichloromethane) to yield 2-[4-(4-acetyl-3,5-dimethyl-piperazin-1-yl)-phenyl]-7-fluoro-5-methoxy-3H-quinazolin-4-one as a light yellow solid.

To a solution of 2-methoxy-ethanol (1.00 g, 13.4 mmol) in dimethyl sulfoxide (4 mL), sodium hydride (60% suspension in mineral oil, 0.50 g, 12.5 mmol) was added in portions, and the reaction mixture was stirred at room temperature for 20 minutes. To this reaction mixture was added 2-[4-(4-acetyl-3,5-dimethyl-piperazin-1-yl)-phenyl]-7-fluoro-5-methoxy-3H-quinazolin-4-one (0.57 g, 1.34 mmol) and the reaction mixture was stirred at 85° C. for 24 hours. Water was added. The mixture was acidified to pH approximately 4-5 with acetic acid, and the precipitated solid was filtered to give crude product, which was purified by column chromatography (silica gel 230-400 mesh; eluting with 2% methanol in dichloromethane). The resulting mixture was purified by preparative HPLC to obtain the title compound as a white solid. Yield: 0.140 g (23.2%). MP 225-227° C. ¹H NMR (400 MHz, CDCl₃): δ 8.10 (d, J=8.8 Hz, 2H), 7.08 (d, J=8.8 Hz, 1H), 6.70 (d, J=2.4 Hz, 1H), 6.49 (d, J=2.4 Hz, 1H), 4.50 (bs, 1H), 4.23 (m, 2H), 4.14 (bs, 1H), 3.84 (s, 3H), 3.81 (m, 2H), 3.69 (m, 2H), 3.32 (s, 3H), 2.99 (bs, 2H), 2.07 (s, 3H), 1.25 (bs, 6H). MS (ES) m/z: 481.11 (M⁺+1).

### Example 10

### Preparation of 2-(4-(4-Isopropylpiperazin-1-yl)phenyl)-5,7-dimethoxyquinazolin-4(3H)-one

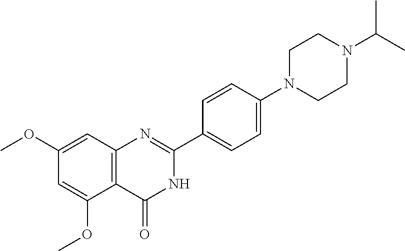 [SMILES: COc1cc(OC)c2c(=O)[nH]c(-c3ccc(N4CCN(C(C)C)CC4)cc3)nc2c1]

A mixture of 4-fluorobenzaldehyde (0.242 g, 1.95 mmol), 1-isopropylpiperazine (0.335 mL, 2.34 mmol), and K₂CO₃ (0.323 g, 2.34 mmol) in DMF (2.44 mL) was heated at 120° C. overnight. The mixture was diluted with EtOAc (200 mL), washed with 10% aqueous LiCl (3×75 mL) and brine (75 mL), dried over Na₂SO₄, and filtered. The volatiles were removed under vacuum to yield 4-(4-Isopropylpiperazin-1-yl)benzaldehyde (0.504 g) as an orange solid, which was used without further purification.

A mixture of 2-amino-4,6-dimethoxybenzamide (0.100 g, 0.510 mmol), aldehyde from above (0.118 g, 0.510 mmol), NaHSO₃ (94%, 0.0565 g, 0.510 mmol), and p-TsOH.H₂O (0.0097 g, 0.051 mmol) in DMA (3.40 mL) was heated at reflux for 1 hour. The mixture was diluted with EtOAc (250 mL), washed with 10% aqueous LiCl (3×75 mL) and brine (75 mL), dried over Na₂SO₄, filtered and concentrated under vacuum. The resulting residue was purified over silica gel (12 g, CH₂Cl₂/MeOH) and the product was freeze-dried from MeCN/H₂O to provide the title compound (0.0632 g, 30%) as a yellow solid. ¹H NMR (300 MHz, DMSO-d₆): δ 11.74 (s, 1H), 8.09 (d, J=9.05 Hz, 2H), 7.00 (d, J=9.05 Hz, 2H), 6.68 (d, J=2.31 Hz, 1H), 6.47 (d, J=2.31 Hz, 1H), 3.88 (s, 3H), 3.84 (s, 3H), 3.31-3.24 (m, 4H), 2.74-2.63 (m, 1H), 2.61-2.53 (m, 4H), 1.01 (d, J=6.52 Hz, 6H).

### Example 11

### Preparation of 2-(4-(4-Acetylpiperazin-1-yl)phenyl)-5,7-dimethoxyquinazolin-4(3H)-one

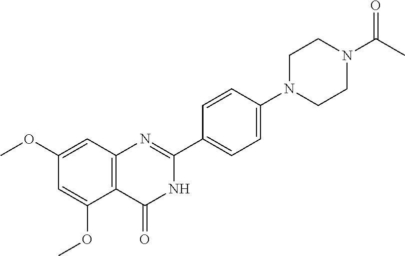 [SMILES: COc1cc(OC)c2c(=O)[nH]c(-c3ccc(N4CCN(C(C)=O)CC4)cc3)nc2c1]

Following the procedure described for Example 10, 4-(4-acetylpiperazin-1-yl)benzaldehyde was made from 1-acetylpiperazine and isolated as an orange oil in 67% yield. Following the procedure described for Example 10, the title compound was made from 4-(4-acetylpiperazin-1-yl)benzaldehyde and reluxing for 5 hours. The title compound was isolated as a yellow solid in 20% yield. ¹H NMR (300 MHz, DMSO-d₆): δ 11.76 (s, 1H), 8.11 (d, J=8.97 Hz, 2H), 7.03 (d, J=8.97 Hz, 2H), 6.69 (d, J=2.26 Hz, 1H), 6.47 (d, J=2.26 Hz, 1H), 3.88 (s, 3H), 3.84 (s, 3H), 3.62-3.53 (m, 4H), 3.41-3.25 (m, 4H), 2.05 (s, 3H); MS (ESI) m/z 409 [C₂₂H₂₄N₄O₄+H]⁺.

### Example 12

### Preparation of 5,7-Dimethoxy-2-(4-(piperazin-1-yl)phenyl)quinazolin-4(3H)-one

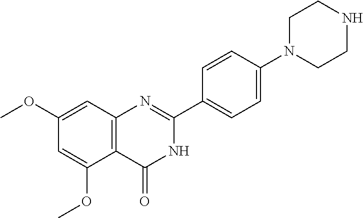 [SMILES: COc1cc(OC)c2c(=O)[nH]c(-c3ccc(N4CCNCC4)cc3)nc2c1]

A mixture of 4-(4-acetylpiperazin-1-yl)benzaldehyde (1.34 g, 5.77 mmol) and 2-amino-4,6-dimethoxybenzamide (1.03 g, 5.24 mmol) in DMA (30 mL) was treated with p-TsOH (0.100 g, 0.524 mmol) and NaHSO₃ (0.578 g, 5.55 mmol). The mixture was heated at 155° C. for 6 hours, cooled to room temperature, diluted with water (400 mL), and filtered to give brown solids. The filtrate was extracted with EtOAc (3×100 mL), concentrated, and combined with the brown solids from the filter cake. The combined solids were purified by silica gel chromatography, eluting with 92:7:1 CHCl₃/MeOH/concentrated NH₄OH to afford 2-(4-(4-acetylpiperazin-1-yl)phenyl)-5,7-dimethoxyquinazolin-4(3H)-one as a yellow solid (1.9 g, 90%).

A mixture of 2-(4-(4-acetylpiperazin-1-yl)phenyl)-5,7-dimethoxyquinazolin-4(3H)-one (1.93 g, 4.7 mmol) and 2 M HCl (200 mL) was heated at reflux for 9 hours. Then, the mixture was cooled to room temperature, basified to pH 8 with 2 N NaOH, extracted with CH₂Cl₂ (3×300 mL), dried over ahydrous MgSO₄, filtered, and concentrated. The residue was purified by silica gel chromatography, eluting with 92:7:1 to 6:3:1 CHCl₃/MeOH/concentrated NH₄OH, to afford the title compound (1.13 g, 66%). ¹H NMR (300 MHz, DMSO-d₆): δ 8.08 (d, J=8.9 Hz, 2H), 6.99 (d, J=8.9 Hz, 2H), 6.68 (d, J=2.3 Hz, 1H), 6.47 (d, J=2.3 Hz, 1H), 3.88 (s, 3H), 3.83 (s, 3H), 3.19-3.23 (m, 4H), 2.81-2.84 (m, 4H); APCI MS m/z 367 [M+H]⁺.

### Example 13

### Preparation of N-(1-(4-(5,7-Dimethoxy-4-oxo-3,4-dihydroquinazolin-2-yl)phenyl)piperidin-4-yl)acetamide

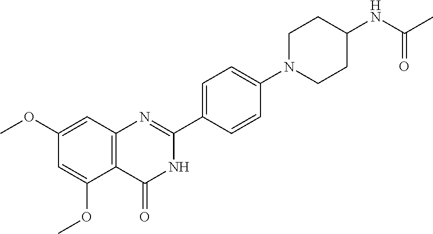 [SMILES: COc1cc(OC)c2c(=O)[nH]c(-c3ccc(N4CCC(NC(C)=O)CC4)cc3)nc2c1]

A solution of ethyl 4-fluorobenzoate (16.5 g, 98.1 mmol) and piperidin-4-ol (10.0 g, 98.8 mmol) in DMSO (20 mL) was heated at 120° C. under nitrogen for 48 hours. The mixture was cooled to room temperature, poured into water (400 mL), and the solids were filtered off, washed with water, followed by hexane, to afford ethyl 4-(4-hydroxypiperidin-1-yl)benzoate (20.0 g, 82%).

To a solution of ethyl 4-(4-hydroxypiperidin-1-yl)benzoate (8.0 g, 32.1 mmol) in CH₂Cl₂ (200 mL) was added Et₃N (23 mL, 165 mmol) under nitrogen, followed by MsCl (5.6 g, 48.9 mmol). The mixture was stirred for 5 minutes, washed with water (300 mL), dried over anhydrous MgSO₄, filtered, and concentrated to afford ethyl 4-(4-(methylsulfonyloxy)piperidin-1-yl)benzoate as a tan solid (10.5 g, 100%).

To a solution of ethyl 4-(4-(methylsulfonyloxy)piperidin-1-yl)benzoate (10.5 g, 32.1 mmol) in DMF (50 mL) was added sodium azide (4.17 g, 64.2 mmol). The mixture was heated at 80° C. for 5 hours, cooled to room temperature, diluted with brine (300 mL), and extracted with ethyl acetate (400 mL). The organic phase was washed with brine (2×300 mL), dried over anyhydrous MgSO₄, filtered, and concentrated, to afford ethyl 4-(4-azidopiperidin-1-yl)benzoate as a yellow solid (7.62 g, 87%).

To a solution of ethyl 4-(4-azidopiperidin-1-yl)benzoate (7.62 g, 27.8 mmol) in dioxane (190 mL) was added acetic acid (27 mL) and water (54 mL). Then, 10% Pd/C (0.750 g) was added and the mixture was hydrogenated under 1 atmosphere of hydrogen for 5 hours. The mixture was filtered through Celite, concentrated, and 0.5 M HCl (500 mL) was added. The solution was washed with ethyl acetate (2×300 mL) and the aqueous phase basified with ammonium hydroxide, to pH 12. The aqueous phase was saturated with sodium chloride, extracted with CH₂Cl₂ (2×300 mL), dried over anhydrous MgSO₄, filtered, and concentrated, to afford ethyl 4-(4-aminopiperidin-1-yl)benzoate.

To a solution of ethyl 4-(4-aminopiperidin-1-yl)benzoate (1.65 g, 6.65 mmol) in CH₂Cl₂ (200 mL) was added Et₃N (1.35 g, 13.3 mmol), followed by acetyl chloride (0.573 g, 7.3 mmol). The reaction mixture was stirred at room temperature for 5 minutes, washed with brine (300 mL), dried over anhydrous Na₂SO₄, filtered, and concentrated, to afford ethyl 4-(4-acetamidopiperidin-1-yl)benzoate as a white solid (1.9 g, 100%).

A solution of ethyl 4-(4-acetamidopiperidin-1-yl)benzoate (0.123 g, 0.42 mmol) in CH₂Cl₂ (10 mL) under nitrogen at −78° C. was treated with DIBAL-H (1.0M in hexanes, 0.950 mL, 0.95 mmol) dropwise, via a syringe. After 20 minutes, the mixture was warmed to room temperature, stirred for 1 hour, and quenched with 10% Rochelle's salt. After stirring for 10 minutes, CH₂Cl₂ (50 mL) was added, and the stirring was continued for 15 additional minutes. The layers were separated and the aqueous phase was extracted with CH₂Cl₂ (50 mL) and ethyl acetate (50 mL). The combined organic phases were dried (MgSO₄), filtered, concentrated, and purified by flash chromatography on silica gel, eluting with 100% ethyl acetate to 10% MeOH/ethyl acetate to afford N-(1-(4-(hydroxymethyl)phenyl)piperidin-4-yl)acetamide as a white solid (0.025 g, 24%).

A mixture of N-(1-(4-(hydroxymethyl)phenyl)piperidin-4-yl)acetamide

(0.380 g, 1.53 mmol), TPAP (0.026 g, 0.08 mmol), NMO (0.268 g, 2.30 mmol), and molecular sieves (3 Angstrom, 0.300 g) in CH₂Cl₂ was stirred at room temperature for 19 hours. The mixture was filtered through Celite, concentrated, and purified by flash chromatography on silica gel, eluting with 100% ethyl acetate to 10% MeOH/ethyl acetate, to afford N-(1-(4-formylphenyl)piperidin-4-yl)acetamide as a white solid (0.280 g, 74%).

A mixture of N-(1-(4-formylphenyl)piperidin-4-yl)acetamide (0.280 g, 1.14 mmol), 2-amino-4,6-dimethoxybenzamide (0.224 g, 1.14 mmol), p-TsOH (0.022 g, 0.114 mmol), and NaHSO₃ (0.125 g, 1.21 mmol) in DMA was heated at 155° C. for 6 hours. The reaction mixture was cooled, diluted with water (100 mL), basified with saturated NaHCO₃, and extracted with ethyl acetate (3×150 mL). The organic phase was concentrated and purified by flash chromatography on silica gel, eluting with 1:1 CH₂Cl₂/(92:7:1 CHCl₃/MeOH/concentrated NH₄OH) to 100% 92:7:1 CHCl₃/MeOH/concentrated NH₄OH. Further purification by reverse-phase HPLC, eluting with 10% to 90% CH₃CN in H₂O with 0.1% TFA, afforded the title compound as a yellow solid (0.140 g, 29%): ¹H NMR (300 MHz, DMSO-d₆): δ 11.74 (s, 1H), 8.08 (d, J=9.0 Hz, 2H), 7.83 (d, J=7.7 Hz, 1H), 7.01 (d, J=9.0 Hz, 2H), 6.68 (d, J=2.3 Hz, 1H), 6.46 (d, J=2.3 Hz, 1H), 3.7-3.89 (m, 9H), 2.92-3.00 (m, 2H), 1.76-1.85 (m, 5H), 1.36-1.48 (m, 2H); APCI MS m/z 423 [M+H]⁺.

### Example 14

### Preparation of N-(1-(4-(5,7-Dimethoxy-4-oxo-3,4-dihydroquinazolin-2-yl)phenyl)piperidin-4-yl)methanesulfonamide

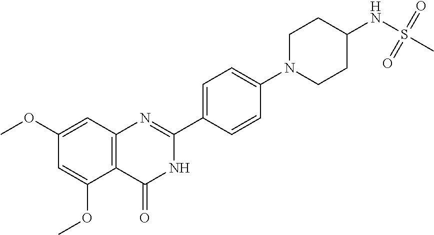

A mixture of 2-(4-(4-aminopiperidin-1-yl)phenyl)-5,7-dimethoxyquinazolin-4(3H)-one (0.105 g, 0.28 mmol), methanesulfonylchloride (0.035 g, 0.30 mmol), and Et₃N (0.057 g. 0.56 mmol) in CH₂Cl₂ (10 mL) was stirred at room temperature under nitrogen for 2 hours. The mixture was concentrated, redissolved in THF (5 mL), 2 M NaOH (5 mL) added and stirred for 20 minutes. The pH was adjusted to 8 with 1 M HCl and the mixture extracted with CH₂Cl₂ (3×150 mL). The organic phase was dried over anhydrous MgSO₄, filtered, and concentrated. The residue was purified by silica gel chromatography, eluting with 1:1 CH₂Cl₂/(92:7:1 CHCl₃/MeOH/concentrated NH₄OH) to 100% 92:7:1 CHCl₃/MeOH/concentrated NH₄OH. Further purification by reverse-phase HPLC. eluting with 10% to 90% CH₃CN in H₂O with 0.1% TFA. afforded the title compound as a yellow solid (0.075 g, 58%). ¹H NMR (300 MHz, DMSO-d₆): δ11.75 (s, 1H), 8.08 (d, J=9.0 Hz, 2H), 7.13 (d, J=7.3 Hz, 1H), 7.00 (d, J=9.0 Hz, 2H), 6.66 (d, J=2.3 Hz, 1H), 6.46 (d, J=2.3 Hz, 1H), 3.81-3.94 (m, 8H), 3.34-3.47 (m, 1H), 2.90 (m, 6H), 1.87-1.95 (m, 2H), 1.42-1.54 (m, 2H); ESI MS m/z 459 [M+H]⁺.

### Example 15

### Preparation of 3-(1-(4-(5,7-Dimethoxy-4-oxo-3,4-dihydroquinazolin-2-yl)phenyl)piperidin-4-yl)-1,1-dimethylurea

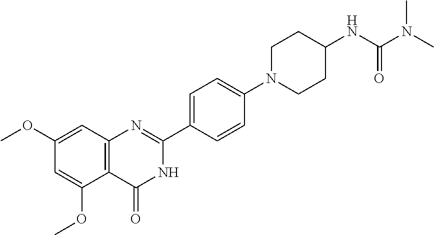

A mixture of N-(1-(4-(5,7-dimethoxy-4-oxo-3,4-dihydroquinazolin-2-yl)phenyl)piperidin-4-yl)acetamide (0.250 g, 0.59 mmol) and 2 M HCl (20 mL) was heated at reflux for 24 hours. The mixture was basified with 2 M NaOH to pH 8, extracted with CH₂Cl₂ (3×150 mL), dried over anhydrous MgSO4, filtered, and concentrated to afford 2-(4-(4-aminopiperidin-1-yl)phenyl)-5,7-dimethoxyquinazolin-4(3H)-one as a yellow solid (0.215 g, 96%).

A mixture of 2-(4-(4-aminopiperidin-1-yl)phenyl)-5,7-dimethoxyquinazolin-4(3H)-one (0.105 g, 0.28 mmol), dimethylcarbamic chloride (0.032 g, 0.30 mmol), and Et₃N (0.085 g, 0.84 mmol) in THF (10 mL) was stirred at room temperature for 18 hours. The mixture was then heated at reflux for 24 hours, then cooled to room temperature. 2 M NaOH (20 mL) was added and the mixture was stirred for 30 minutes. The reaction mixture was adjusted to pH 8, extracted with CH₂Cl₂ (3×100 mL), dried over anhydrous MgSO₄, filtered, and concentrated. The residue was dissolved in CHCl₃/MeOH and concentrated, then CH₃CN was added and concentrated to afford the title compound as a white solid (0.065 g, 51%): ¹H NMR (300 MHz, CDCl₃): δ 11.72 (s, 1H), 8.08 (d, J=9.0 Hz, 2H), 7.00 (d, J=9.0 Hz, 2H), 6.78 (d, J=2.2 Hz, 1H), 6.46 (d, J=2.2 Hz, 1H), 5.99 (d, J=7.8 Hz, 1H), 3.90-3.94 (m, 2H), 3.88 (s, 3H), 3.83 (s, 3H), 3.66-3.69 (m, 1H), 2.88-2.93 (m, 2H), 2.76 (s, 6H), 1.75-1.80 (m, 2H), 1.45-1.52 (m, 2H); ESI MS m/z 452 [M+H]⁺.

### Example 16

### Preparation of 2-(4-(4-Hexanoylpiperazin-1-yl)phenyl)-5,7-dimethoxyquinazolin-4(3H)-one

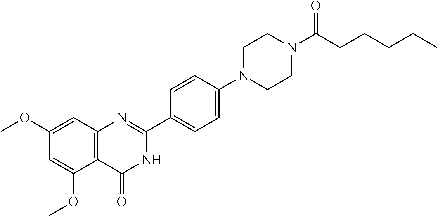 [SMILES: CCCCCC(=O)N1CCN(c2ccc(-c3nc4cc(OC)cc(OC)c4c(=O)[nH]3)cc2)CC1]

To a solution of 5,7-dimethoxy-2-(4-(piperazin-1-yl)phenyl)quinazolin-4(3H)-one (0.120 g, 0.32 mmol) in CH₂Cl₂ (10 mL) was added Et₃N (0.06 mL, 0.48 mmol) and hexanoyl chloride (0.03 mL, 0.28 mmol). The resulting solution was stirred at room temperature for 1 hour. The mixture was concentrated in vacuo. The material was purified by flash chromatography, eluting with 2% to 10% of MeOH/CH₂Cl₂, to afford the title compound (0.050 g, 38%). ¹H NMR (300 MHz, DMSO-d₆): δ 11.79 (s, 1H), 8.11 (d, J=8.7 Hz, 2H), 7.03 (d, J=8.8 Hz, 2H), 6.68 (s, 1H), 6.47 (s, 1H), 3.75-4.05 (m, 6H), 3.47-3.73 (m, 4H), 3.17-3.43 (m, 4H), 2.20-2.40 (m, 2H), 1.41-1.62 (m, 2H), 1.15-1.38 (m, 4H), 0.76-0.98 (m, 3H); APCI MS m/z 465 [M+H]⁺.

### Example 17

### Preparation of 2-(4-(4-Isobutyrylpiperazin-1-yl)phenyl)-5,7-dimethoxyquinazolin-4(3H)-one

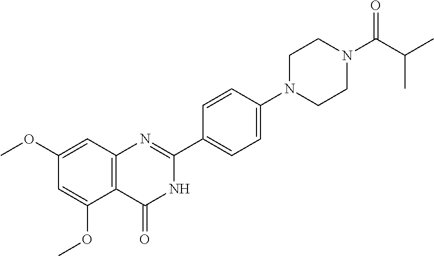 [SMILES: COc1cc(OC)c2c(=O)[nH]c(-c3ccc(N4CCN(C(=O)C(C)C)CC4)cc3)nc2c1]

To a solution of 5,7-dimethoxy-2-(4-(piperazin-1-yl)phenyl)quinazolin-4(3H)-one (0.150 g, 0.40 mmol) in CH₂Cl₂ (10 mL) was added Et₃N (0.08 mL, 0.60 mmol) and isobutyryl chloride (0.03 mL, 0.36 mmol). The resulting solution was stirred at room temperature for 1 hour. The solution was concentrated in vacuo and the residue was purified by flash chromatography on silica gel, eluting with 2% to 10% of MeOH/CH₂Cl₂. The solid was further purified by flash chromatography on silica gel, eluting with 0% to 5% of MeOH/EtOAc, to afford the title compound (0.080 g, 50%):

¹H NMR (300 MHz, DMSO-d₆): δ 11.78 (s, 1H), 8.11 (d, J=9.0 Hz, 2H), 7.03 (d, J=9.1 Hz, 2H), 6.68 (s, 1H), 6.47 (s, 1H), 3.76-3.92 (m, 6H), 3.52-3.71 (m, 4H), 3.16-3.44 (m, 4H), 2.83-3.00 (m, 1H), 1.02 (d, J=6.8 Hz, 6H); APCI MS m/z 437 [M+H]⁺.

### Example 18

### Preparation of 2-(4-(4-Benzoylpiperazin-1-yl)phenyl)-5,7-dimethoxyquinazolin-4(3H)-one

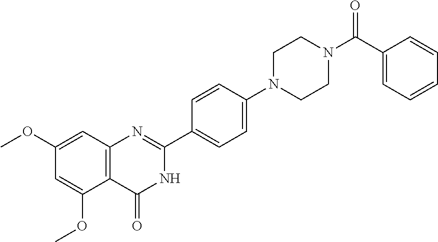 [SMILES: COc1cc(OC)c2c(=O)[nH]c(-c3ccc(N4CCN(C(=O)c5ccccc5)CC4)cc3)nc2c1]

To a solution of 5,7-dimethoxy-2-(4-(piperazin-1-yl)phenyl)quinazolin-4(3H)-one (0.150 g, 0.40 mmol) in CH₂Cl₂ (10 mL) was added Et₃N (0.08 mL, 0.60 mmol) and benzoyl chloride (0.04 mL, 0.36 mmol). The resulting solution was stirred at room temperature for 3 hours. The solution was concentrated in vacuo. The material was purified by flash chromatography on silica gel eluting with 0% to 10% of MeOH/EtOAc to afford the title compound (0.110 g, 64%). ¹H NMR (300 MHz, DMSO-d₆): δ 11.79 (s, 1H), 8.11 (d, J=8.7 Hz, 2H), 7.37-7.54 (m, 5H), 7.04 (d, J=8.9 Hz, 2H), 6.68 (s, 1H), 6.47 (s, 1H), 3.61-4.03 (m, 8H), 3.23-3.62 (m, 6H); ESI MS m/z 471 [M+H]⁺.

### Example 19

### Preparation of 2-(4-(4-(4-Fluorobenzoyl)piperazin-1-yl)phenyl)-5,7-dimethoxyquinazolin-4(3H)-one

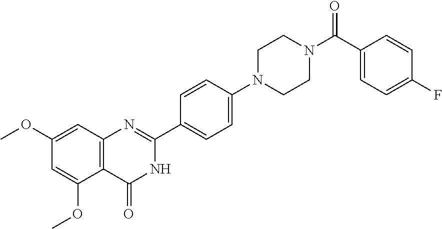 [SMILES: COc1cc(OC)c2c(=O)[nH]c(-c3ccc(N4CCN(C(=O)c5ccc(F)cc5)CC4)cc3)nc2c1]

To a solution of 5,7-dimethoxy-2-(4-(piperazin-1-yl)phenyl)quinazolin-4(3H)-one (0.150 g, 0.40 mmol) in CH₂Cl₂ (10 mL) was added Et₃N (0.08 mL, 0.60 mmol) and 4-fluorobenzoyl chloride (0.04 mL, 0.36 mmol). The resulting solution was stirred at room temperature for 3 hours. The solution was concentrated in vacuo and the residue was purified by flash chromatography on silica gel, eluting with 0% to 10% of MeOH/EtOAc, to afford the title compound (0.080 g, 45%). ¹H NMR (300 MHz, DMSO-d₆): δ 11.79 (s, 1H), 8.11 (d, J=8.8 Hz, 2H), 7.44-7.62 (m, 2H), 7.21-7.39 (m, 2H), 7.04 (d, J=9.0 Hz, 2H), 6.68 (s, 1H), 6.47 (s, 1H), 3.64-3.94 (m, 8H), 3.22-3.60 (m, 6H); APCI MS m/z 489 [M+H]⁺.

### Example 20

### Preparation of N-(1-(4-(5,7-Dimethoxy-4-oxo-3,4-dihydroquinazolin-2-yl)phenyl)piperidin-4-yl)benzamide

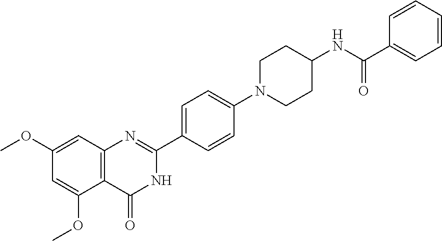 [SMILES: COc1cc(OC)c2c(=O)[nH]c(-c3ccc(N4CCC(NC(=O)c5ccccc5)CC4)cc3)nc2c1]

To a solution of ethyl 4-(4-aminopiperidin-1-yl)benzoate (3.0 g, 12.1 mmol) in CH₂Cl₂ under nitrogen was added Et₃N (2.45 g, 24.2 mmol), followed by benzoyl chloride (1.70 g, 12.1 mmol). The mixture was stirred at room temperature overnight, washed with brine (200 mL), dried over anhydrous MgSO₄, filtered, and concentrated. The resulting solids were triturated with hexanes to afford ethyl 4-(4-benzamidopiperidin-1-yl)benzoate as a yellow solid (4.2 g, 100%).

A solution of ethyl 4-(4-benzamidopiperidin-1-yl)benzoate (4.2 g, 11.9 mmol) in THF (400 mL) was cooled to 0° C. under nitrogen and treated with DIBAL-H (1.0 M in THF, 47 mL, 47 mmol). The mixture was warmed to room temperature and stirred for 1 hour. Then, the reaction mixture was quenched with Rochelle's salt (10% aqueous), concentrated to remove the THF, brine (300 mL) was added, and the organic phase was extracted with CH₂Cl₂ (3×200 mL), dried over anhydrous MgSO₄, filtered, and concentrated, to afford N-(1-(4-(hydroxymethyl)phenyl)piperidin-4-yl)benzamide as a yellow solid that was used without further purification.

To a solution of N-(1-(4-(hydroxymethyl)phenyl)piperidin-4-yl)benzamide (1.1 g, 3.5 mmol) in CH₂Cl₂ (250 mL) was added TPAP (0.123 g, 0.35 mmol) and NMO (0.623 g, 5.3 mmol). After 1 hour, the mixture was filtered through Celite, concentrated, and purified by silica gel chromatography, eluting with 30% ethyl acetate/hexanes to 100% ethyl acetate, to afford N-(1-(4-formylphenyl)piperidin-4-yl)benzamide as a white solid (0.350 g, 32%).

A mixture of N-(1-(4-formylphenyl)piperidin-4-yl)benzamide (0.350 g, 1.10 mmol), NaHSO₃ (0.180 g, 1.70 mmol) and p-TsOH (0.022 g, 0.11 mmol) and 2-amino-4,6-dimethoxybenzamide (0.223 g, 1.10 mmol) in DMA (10 mL) was heated at 150° C. overnight. The mixture was concentrated in vacuo, and the residue was dissolved in EtOAc and washed with H₂O and brine, dried (Na₂SO₄), filtered and concentrated in vacuo. The resulting solid was purified by silica gel chromatography eluting with 10% to 50% CHCl₃/MeOH/concentrated NH₄OH in CH₂Cl₂ to afford the title compound (0.050 g, 10%): ¹H NMR (300 MHz, DMSO-d₆): δ 11.75 (s, 1H), 8.26 (d, J=7.4 Hz, 1H), 8.10 (d, J=9.0 Hz, 2H), 7.83 (d, J=6.9 Hz, 2H), 7.44-7.49 (m, 3H), 7.05 (d, J=8.8 Hz, 2H), 6.68 (s, 1H), 6.46 (s, 1H), 3.93-4.17 (m, 3H), 3.88 (s, 3H), 3.83 (s, 3H), 2.91-3.08 (m, 2H), 1.82-1.93 (m, 2H), 1.52-1.72 (m, 2H); APCI MS m/z 485 [M+H]⁺.

### Example 21

### Preparation of 5,7-Dimethoxy-2-(4-(4-picolinoylpiperazin-1-yl)phenyl)quinazolin-4(3H)-one

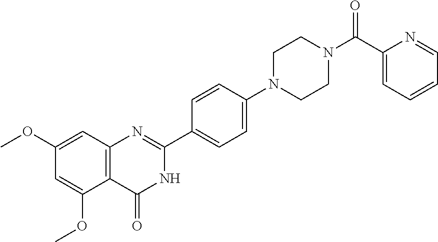 [SMILES: COc1cc(OC)c2c(=O)[nH]c(-c3ccc(N4CCN(C(=O)c5ccccn5)CC4)cc3)nc2c1]

To a solution of picolinic acid (0.066 g, 0.54 mmol) in THF (20 mL) was added HOBt (0.079 g, 0.59 mmol), EDCI (0.113 g, 0.59 mmol), Et₃N (0.08 mL, 0.59 mmol) and 5,7-dimethoxy-2-(4-(piperazin-1-yl)phenyl)quinazolin-4(3H)-one (0.200 g, 0.54 mmol). The resulting solution was stirred overnight at room temperature. The solution was concentrated in vacuo and the resulting solid was purified by flash chromatography on silica gel, eluting with 50% to 100% of 92:7:1 CHCl₃/MeOH/concentrated NH₄OH in CH₂Cl₂, to afford the title compound (0.160 g, 62%): ¹H NMR (300 MHz, DMSO-d₆): δ 11.69 (s, 1H), 8.53-8.70 (m, 1H), 8.11 (d, J=8.9 Hz, 2H), 7.86-8.04 (m, 1H), 7.64 (d, J=7.8 Hz, 1H), 7.44-7.57 (m, 1H), 7.04 (d, J=9.1 Hz, 2H), 6.69 (s, 1H), 6.47 (s, 1H), 3.74-3.97 (m, 8H), 3.53-3.68 (m, 2H), 3.41-3.53 (m, 2H), 3.23-3.39 (m, 2H). APCI MS m/z 472 [M+H]⁺.

### Example 22

### Preparation of 5,7-Dimethoxy-2-(4-(4-nicotinoylpiperazin-1-yl)phenyl)quinazolin-4(3H)-one

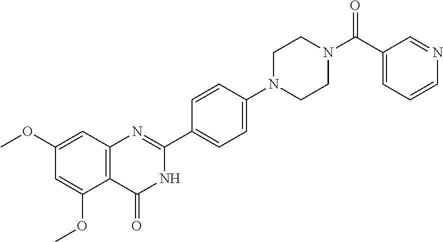 [SMILES: COc1cc(OC)c2c(=O)[nH]c(-c3ccc(N4CCN(C(=O)c5cccnc5)CC4)cc3)nc2c1]

To a solution of nicotinic acid (0.066 g, 0.54 mmol) in THF (20 mL) was added HOBt (0.079 g, 0.59 mmol), EDCI (0.113 g, 0.59 mmol), Et₃N (0.08 mL, 0.59 mmol) and 5,7-dimethoxy-2-(4-(piperazin-1-yl)phenyl)quinazolin-4(3H)-one (0.200 g, 0.54 mmol). The resulting solution was stirred overnight at room temperature. The solution was concentrated in vacuo and the resulting solid was purified by flash chromatography on silica gel, eluting with 10% to 60% of 92:7:1 CHCl₃/MeOH/concentrated NH₄OH in CH₂Cl₂, to afford the title compound (0.050 g, 19%): ¹H NMR (300 MHz, DMSO-d₆): δ 11.79 (s, 1H), 8.59-8.78 (m, 2H), 8.12 (d, J=8.8 Hz, 2H), 7.82-7.99 (m, 1H), 7.37-7.60 (m, 1H), 7.04 (d, J=9.1 Hz, 2H), 6.69 (s, 1H), 6.47 (s, 1H), 3.63-3.97 (m, 8H), 3.20-3.63 (m, 6H). APCI MS m/z 472 [M+H]⁺.

### Example 23

### Preparation of 2-(4-(4-Isonicotinoylpiperazin-1-yl)phenyl)-5,7-dimethoxyquinazolin-4(3H)-one

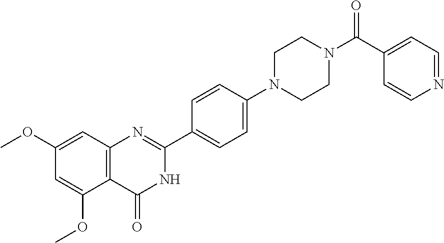 [SMILES: COc1cc(OC)c2c(=O)[nH]c(-c3ccc(N4CCN(C(=O)c5ccncc5)CC4)cc3)nc2c1]

To a solution of isonicotinic acid (0.083 g, 0.68 mmol) in THF (20 mL) was added HOBt (0.099 g, 0.74 mmol), EDCI (0.141 g, 0.74 mmol), Et₃N (0.10 mL, 0.74 mmol) and 5,7-dimethoxy-2-(4-(piperazin-1-yl)phenyl)quinazolin-4(3H)-one (0.250 g, 0.68 mmol). The resulting solution was stirred overnight at room temperature. The solution was concentrated in vacuo and the resulting material was purified by flash chromatography on silica gel, eluting with 10% to 60% of 92:7:1 CHCl₃/MeOH/concentrated NH₄OH in CH₂Cl₂, to afford the title compound (0.110 g, 34%). ¹H NMR (300 MHz, DMSO-d₆): δ 11.79 (s, 1H), 8.58-8.79 (m, 2H), 8.12 (d, J=9.0 Hz, 2H), 7.45 (d, J=6.0 Hz, 2H), 7.04 (d, J=9.0 Hz, 2H), 6.69 (s, 1H), 6.47 (s, 1H), 3.64-4.06 (m, 9H), 3.22-3.54 (m, 5H). APCI MS m/z 472 [M+H]⁺.

### Example 24

### Preparation of 5,7-Dimethoxy-2-(4-(4-(thiophene-2-carbonyl)piperazin-1-yl)phenyl)quinazolin-4(3H)-one

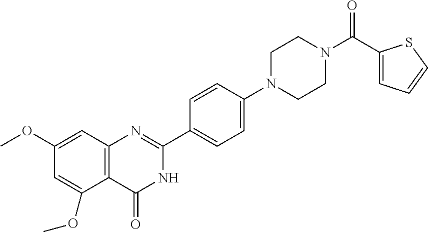 [SMILES: COc1cc(OC)c2c(=O)[nH]c(-c3ccc(N4CCN(C(=O)c5cccs5)CC4)cc3)nc2c1]

To a solution of 2-thiophenecarboxylic acid (0.087 g, 0.68 mmol) in THF (20 mL) was added HOBt (0.099 g, 0.74 mmol), EDCI (0.141 g, 0.74 mmol), Et₃N (0.10 mL, 0.74 mmol) and 5,7-dimethoxy-2-(4-(piperazin-1-yl)phenyl)quinazolin-4(3H)-one (0.250 g, 0.68 mmol). The resulting solution was stirred at room temperature for 4 hours. The solution was concentrated in vacuo. The material was purified by flash chromatography, eluting with 0% to 50% of 92:7:1 CHCl₃/MeOH/concentrated NH₄OH in CH₂Cl₂, to afford the title compound (0.100 g, 30%). ¹H NMR (300 MHz, DMSO-d₆): δ 11.78 (s, 1H), 8.12 (d, J=9.0 Hz, 2H), 7.75-7.84 (m, 1H), 7.46-7.53 (m, 1H), 7.12-7.20 (m, 1H), 7.03 (d, J=9.1 Hz, 2H), 6.69 (d, J=2.3 Hz, 1H), 6.47 (d, J=2.3 Hz, 1H), 3.88 (s, 3H), 3.83 (s, 3H), 3.74-3.92 (m, 4H), 3.37-3.49 (m, 4H). APCI MS m/z 477 [M+H]⁺.

### Example 25

### Preparation of 2-(4-(4-(5-Chloro-1-methyl-1H-pyrazole-4-carbonyl)piperazin-1-yl)phenyl)-5,7-dimethoxyquinazolin-4(3H)-one

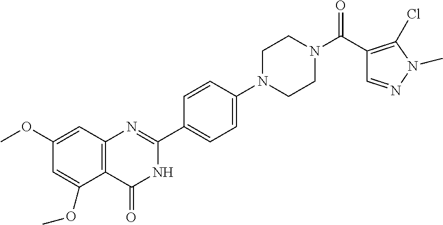

To a mixture of 5,7-dimethoxy-2-(4-(piperazin-1-yl)phenyl)quinazolin-4(3H)-one (0.150 g, 0.41 mmol) and 5-chloro-1-methyl-1H-pyrazole-4-carbonyl chloride (0.073 g, 0.41 mmol) in CH₂Cl₂ (50 mL), was added Et₃N (0.086 mL, 0.62 mmol) and the reaction stirred under nitrogen at room temperature for 1 hour. The residue was concentrated and purified by flash chromatography on silica gel, eluting with 70% CH₂Cl₂/(92:7:1 CHCl₃/MeOH/concentrated NH₄OH) to 100% (92:7:1 CHCl₃/MeOH/concentrated NH₄OH), to afford the title compound as a white solid (0.159 g, 76%). ¹H NMR (500 MHz, DMSO-d₆): δ 11.78 (s, 1H), 8.12 (d, J=9.0 Hz, 2H), 7.77 (s, 1H), 7.04 (d, J=9.1 Hz, 2H), 6.69 (d, J=2.3 Hz, 1H), 6.47 (d, J=2.3 Hz, 1H), 3.88 (s, 3H), 3.80-3.87 (m, 6H), 3.63-3.80 (m, 4H), 3.38-3.44 (m, 4H). APCI MS m/z 509 [M+H]⁺.

### Example 26

### Preparation of 5,7-Dimethoxy-2-(4-(4-(3,3,3-trifluoropropanoyl)piperazin-1-yl)phenyl)quinazolin-4(3H)-one

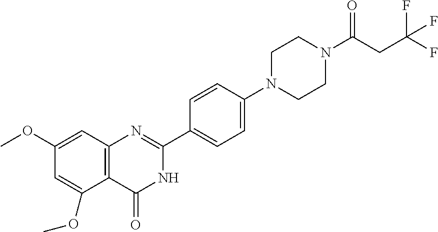 [SMILES: COc1cc(OC)c2c(=O)[nH]c(-c3ccc(N4CCN(C(=O)CC(F)(F)F)CC4)cc3)nc2c1]

To a solution of 5,7-dimethoxy-2-(4-(piperazin-1-yl)phenyl)quinazolin-4(3H)-one (0.200 g, 0.54 mmol) in THF (10 mL) was added EDCI (0.105 g, 0.54 mmol), HOBt (0.074 g, 0.54 mmol), Et₃N (0.08 mL, 0.54 mmol) and trifluoropropionic acid (0.070 g, 0.54 mmol). The reaction was stirred at room temperature for 4 hours and concentrated in vacuo. Purification by flash chromatography, eluting with 20% to 100% of 92:7:1 CHCl₃/MeOH/concentrate NH₄OH in CH₂Cl₂, afforded the title compound (0.135 g, 52%). ¹H NMR (300 MHz, DMSO-d₆): δ 11.78 (s, 1H), 8.10 (d, J=9.0 Hz, 2H), 7.03 (d, J=9.0 Hz, 2H), 6.68 (d, J=2.3 Hz, 1H), 6.47 (d, J=2.3 Hz, 1H), 3.88 (s, 3H), 3.83 (s, 3H), 3.70-3.78 (m, 2H), 3.60-3.67 (m, 4H), 3.34-3.38 (m, 4H). APCI MS m/z 477 [M+H]⁺.

### Example 27

### Preparation of 2-(4-(4-(2,5-Dichlorothiophene-3-carbonyl)piperazin-1-yl)phenyl)-5,7-dimethoxyquinazolin-4(3H)-one

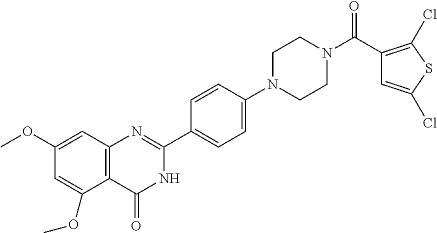 [SMILES: COc1cc(OC)c2c(=O)[nH]c(-c3ccc(N4CCN(C(=O)c5cc(Cl)sc5Cl)CC4)cc3)nc2c1]

To a mixture of 5,7-dimethoxy-2-(4-(piperazin-1-yl)phenyl)quinazolin-4(3H)-one (0.150 g, 0.41 mmol) and 2,5-dichlorothiophene-3-carbonyl chloride (0.088 g, 0.41 mmol) in CH₂Cl₂ was added Et₃N (0.086 mL, 0.62 mmol) and the mixture stirred at room temperature under nitrogen for 30 minutes. The mixture was concentrated and purified by silica gel chromatography, eluting with 70% CH₂Cl₂/(92:7:1 CHCl₃/MeOH/concentrated NH₄OH) to 100% (92:7:1 CHCl₃/MeOH/concentrated NH₄OH), to afford the title compound as a light yellow solid (0.177 g, 79%). ¹H NMR (300 MHz, DMSO-d₆): δ 11.80 (s, 1H), 8.12 (d, J=9.0 Hz, 2H), 7.27 (s, 1H), 7.05 (d, J=9.0 Hz, 2H), 6.69 (d, J=2.3 Hz, 1H), 6.48 (d, J=2.3 Hz, 1H), 3.88 (s, 3H), 3.84 (s, 3H), 3.73-3.82 (m, 2H), 3.38-3.44 (m, 6H). APCI MS m/z 545 [M+H]⁺.

### Example 28

### Preparation of 2-(4-(4-(Cyclopropanecarbonyl)piperazin-1-yl)phenyl)-5,7-dimethoxyquinazolin-4(3H)-one

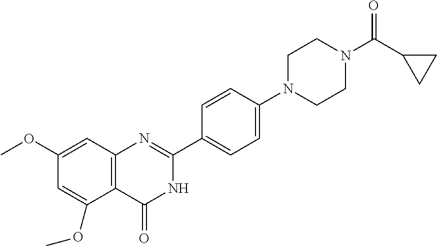 [SMILES: COc1cc(OC)c2c(=O)[nH]c(-c3ccc(N4CCN(C(=O)C5CC5)CC4)cc3)nc2c1]

To a solution of 5,7-dimethoxy-2-(4-(piperazin-1-yl)phenyl)quinazolin-4(3H)-one (0.150 g, 0.40 mmol) in CH₂Cl₂ (10 mL) was added Et₃N (0.08 mL, 0.60 mmol), and cyclopropane carbonyl chloride (0.03 mL, 0.36 mmol). The resulting solution was stirred overnight at room temperature. The solution was concentrated in vacuo and the material was purified by flash chromatography on silica gel eluting with 0% to 50% of 92:7:1 CHCl₃/MeOH/concentrated NH₄OH in CH₂Cl₂ to afford the title compound (0.100 g, 63%). ¹H NMR (300 MHz, DMSO-d₆): δ 11.78 (s, 1H), 8.12 (d, J=8.9 Hz, 2H), 7.04 (d, J=9.2 Hz, 2H), 6.63-6.74 (m, 1H), 6.39-6.52 (m, 1H), 3.73-3.95 (m, 8H), 3.51-3.73 (m, 2H), 3.21-3.49 (m, 4H), 1.93-2.10 (m, 1H), 0.56-0.83 (m, 4H). APCI MS m/z 435 [M+H]⁺.

### Example 29

### Preparation of 2-(4-(4-(4-Fluorobenzyl)piperazin-1-yl)phenyl)-5,7-dimethoxyquinazolin-4(3H)-one

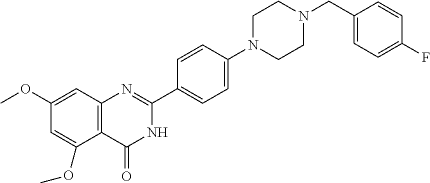 [SMILES: COc1cc(OC)c2c(=O)[nH]c(-c3ccc(N4CCN(Cc5ccc(F)cc5)CC4)cc3)nc2c1]

To a solution of 5,7-dimethoxy-2-(4-(piperazin-1-yl)phenyl)quinazolin-4(3H)-one (0.200 g, 0.55 mmol) in DMF (5 mL) was added 4-fluorobenzyl bromide (0.07 mL, 0.55 mmol) and K₂CO₃ (0.15 g, 1.10 mmol). The reaction was stirred at room temperature for 2 hours then diluted with H₂O and the solids filtered off to afford the title compound (0.17 g, 65%) as a light brown solid. ¹H NMR (300 MHz, DMSO-d₆): δ 11.76 (br s, 1H), 8.09 (d, J=8.1 Hz, 2H), 7.26-7.52 (m, 2H), 7.08-7.25 (m, 2H), 7.00 (d, J=8.0 Hz, 2H), 6.68 (s, 1H), 6.46 (s, 1H), 3.87 (s, 3H), 3.83 (s, 3H), 3.51 (s, 2H), 3.08-3.41 (m, 4H), 2.23-2.68 (m, 4H). APCI MS m/z 475 [M+H]⁺.

### Example 30

### Preparation of 2-(4-(4-Benzylpiperazin-1-yl)phenyl)-5,7-dimethoxyquinazolin-4(3H)-one

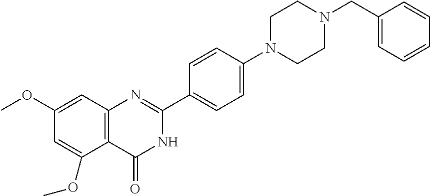 [SMILES: COc1cc(OC)c2c(=O)[nH]c(-c3ccc(N4CCN(Cc5ccccc5)CC4)cc3)nc2c1]

Following the method described for Example 29 above, the title compound was made from benzyl bromide in 45% yield. ¹H NMR (300 MHz, DMSO-d₆): δ 11.76 (s, 1H), 8.09 (d, J=8.6 Hz, 2H), 7.26-7.43 (m, 5H), 7.00 (d, J=8.8 Hz, 2H), 6.68 (s, 1H), 6.46 (s, 1H), 3.87 (s, 3H), 3.85 (s, 3H), 3.53 (s, 2H), 3.23-3.40 (m, 4H), 2.38-2.63 (m, 4H). APCI MS m/z 457 [M+H]⁺.

### Example 31

### Preparation of 2-(4-(4-(2,2,2-Trifluoroethyl)piperazin-1-yl)phenyl)quinazolin-4(3H)-one

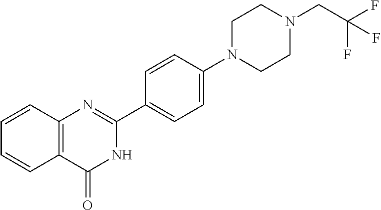 [SMILES: O=c1[nH]c(-c2ccc(N3CCN(CC(F)(F)F)CC3)cc2)nc2ccccc12]

To a mixture of 2-aminobenzamide (1.0 g, 7.35 mmol) and 4-(4-acetylpiperazin-1-yl)benzaldehyde (1.71 g, 7.35 mmol) in DMA (60 mL) was added p-TsOH (0.140 g, 0.73 mmol) and NaHSO₃ (0.841 g, 8.1 mmol). The reaction mixture was heated at 150° C. for 21 hours, concentrated to half-volume, diluted with water (300 mL), extracted with CH₂Cl₂ (2×200 mL), washed with brine (200 mL), dried over anhydrous MgSO₄, filtered, and concentrated. The residue was purified by silica gel chromatography, eluting with 100% CH₂Cl₂ to 100% (92:7:1 CHCl₃/MeOH/concentrated NH₄OH), to afford 2-(4-(4-acetylpiperazin-1-yl)phenyl)quinazolin-4(3H)-one as a yellow solid (2.27 g, 89%).

A mixture of 2-(4-(4-acetylpiperazin-1-yl)phenyl)quinazolin-4(3H)-one (2.27 g, 6.5 mmol) and 2 N HCl (100 mL) were heated at 100° C. for 4 hours. Then, the mixture was cooled to room temperature, basified to pH 8 with 2 N NaOH, extracted with CH₂Cl₂ (3×150 mL), dried over anhydrous MgSO₄, filtered, and concentrated to afford 2-(4-(piperazin-1-yl)phenyl)quinazolin-4(3H)-one as a pale yellow solid (1.8 g, 90%).

To a mixture of 2-(4-(piperazin-1-yl)phenyl)quinazolin-4(3H)-one (0.325 g, 1.06 mmol) in THF (50 mL) was added Hünig's base (0.192 g, 1.48 mmol), followed by 2,2,2-trifluoroethyl trifluoromethanesulfonate (0.295 g, 1.3 mmol). The reaction mixture was heated at reflux for 15 hours, concentrated, and purified by flash chromatography on silica gel, eluting with 100% CH₂Cl₂ to 100% ethyl acetate, to afford the title compound as an off-white solid (0.385 g, 94%). ¹H NMR (300 MHz, DMSO-d₆): δ 12.27 (br s, 1H), 8.10-8.14 (m, 3H), 7.76-7.82 (m, 1H), 7.67 (d, J=7.8 Hz, 1H), 7.42-7.47 (m, 1H), 7.05 (d, J=9.1 Hz, 2H), 3.21-3.34 (m, 6H), 2.73-2.78 (m, 4H). APCI MS m/z 389 [M+H]⁺.

### Example 32

### Preparation of 2-(4-(4-Acetyl-1,4-diazepan-1-yl)phenyl)-5,7-dimethoxyquinazolin-4(3H)-one

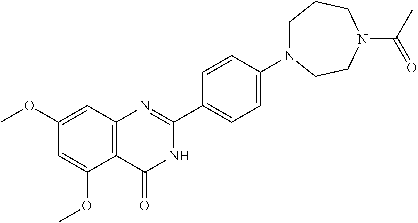 [SMILES: COc1cc(OC)c2c(=O)[nH]c(-c3ccc(N4CCCN(C(C)=O)CC4)cc3)nc2c1]

A mixture of 4-fluorobenzaldehyde (1.56 g, 12.6 mmol), 1-(1,4-diazepan-1-yl)ethanone (1.5 g, 10.5 mmol), and K₂CO₃ (1.74 g, 12.6 mmol) in DMF (10 mL) were heated at 120° C. for 20 hours. The mixture was cooled to room temperature and diluted with water. The mixture was extracted with ethyl acetate and the organic phase washed with brine, dried over anhydrous MgSO₄, filtered, and concentrated. The residue was purified by flash chromatography on silica gel, eluting with 50% ethyl acetate/hexanes to 100% ethyl acetate to 10% methanol/ethyl acetate, to afford 4-(4-acetyl-1,4-diazepan-1-yl)benzaldehyde (1.8 g, 70%).

To a mixture of 2-amino-4,6-dimethoxybenzamide (0.377 g, 1.92 mmol) and 4-(4-acetyl-1,4-diazepan-1-yl)benzaldehyde (0.520 g, 2.11 mmol) in DMA (20 mL) was added NaHSO₃ (0.240 g, 2.3 mmol) followed by p-TsOH (0.037 g, 0.192 mmol) and the reaction heated at 150° C. for 6 hours. The mixture was cooled to room temperature, diluted with CH₂Cl₂ (150 mL), washed with brine (2×150 mL), dried over anhydrous MgSO₄, filtered, and concentrated. The residue was purified by flash chromatography on silica gel, eluting with 1:1 CH₂Cl₂/92:71 CHCl₃/MeOH/concentrated NH₄OH to 100% 92:7:1 CHCl₃/MeOH/concentrated NH₄OH, to afford the title compound (0.333 g, 41%) as a yellow solid. ¹H NMR (300 MHz, CDCl₃): δ 9.12 (s, 1H), 7.88-7.91 (m, 2H), 6.78-6.82 (m, 3H), 6.42 (d, J=2.2 Hz, 1H), 3.98 (s, 3H), 3.93 (s, 3H), 3.62-3.80 (m, 6H), 3.36-3.48 (m, 2H), 1.98-2.12 (m, 5H). ESI MS m/z 421 [M−H]⁻.

### Example 33

### Preparation of 2-(4-(1,4-Diazepan-1-yl)phenyl)-5,7-dimethoxyquinazolin-4(3H)-one

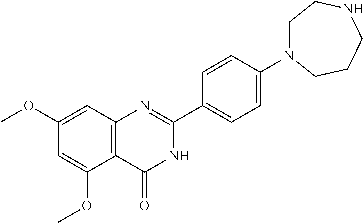 [SMILES: COc1cc(OC)c2c(=O)[nH]c(-c3ccc(N4CCCNCC4)cc3)nc2c1]

A mixture of 2-(4-(4-acetyl-1,4-diazepan-1-yl)phenyl)-5,7-dimethoxyquinazolin-4(3H)-one (0.135 g, 0.32 mmol) and 2 N HCl (10 mL) was heated at 100° C. for 4 hours. Then, the reaction mixture was cooled to room temperature, basified to pH 8, and extracted with CH₂Cl₂ (8×125 mL). The residue was purified by flash chromatography on silica gel, eluting with 1:1 CH₂Cl₂/92:71 CHCl₃/MeOH/concentrated NH₄OH to 100% 92:7:1 CHCl₃/MeOH/concentrated NH₄OH, to afford the title compound (0.040 g, 33%) as a yellow solid. ¹H NMR (300 MHz, CDCl₃): δ 8.98 (s, 1H), 7.86 (d, J=9.0 Hz, 2H), 6.76-6.79 (m, 3H), 6.40 (d, J=2.3 Hz, 1H), 3.98 (s, 3H), 3.92 (s, 3H), 3.61-3.69 (m, 5H), 3.05 (t, J=4.9 Hz, 2H), 2.83 (t, J=5.7 Hz, 2H), 1.92 (t, J=5.4 Hz, 2H). ESI MS m/z 379 [M−H]⁻.

### Example 34

### Preparation of 5,7-Dimethoxy-2-(4-(4-methyl-1,4-diazepan-1-yl)phenyl)quinazolin-4(3H)-one

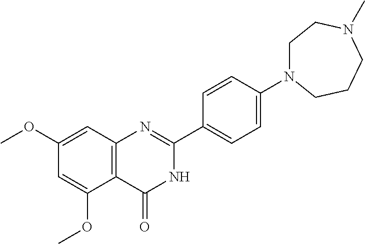

To a solution of 2-(4-(1,4-diazepan-1-yl)phenyl)-5,7-dimethoxyquinazolin-4(3H)-one (0.150 g, 0.39 mmol) in DMF (20 mL) was added CH₃I (0.067 g, 0.47 mmol) and Hünig's Base (0.138 mL, 0.79 mmol). The reaction mixture was heated at 50° C. for 1.5 hours, cooled to room temperature, diluted with ethyl acetate (150 mL), washed with brine (2×100 mL), dried over anhydrous MgSO₄, filtered, and concentrated. The residue was purified by flash chromatography on silica gel, eluting with 1:1 CH₂Cl₂/92:71 CHCl₃/MeOH/concentrated NH₄OH to 100% 92:7:1 CHCl₃/MeOH/concentrated NH₄OH, to afford the title compound (0.035 g, 23%) as a white solid. ¹H NMR (300 MHz, DMSO-d₆): δ 11.66 (s, 1H), 8.06 (d, J=9.0 Hz, 2H), 6.78 (d, J=9.0 Hz, 2H), 6.65 (d, J=2.2 Hz, 1H), 6.44 (d, J=2.2 Hz, 1H), 3.87 (s, 3H), 3.83 (s, 3H), 3.57-3.59 (m, 2H), 3.52 (t, J=6.1 Hz, 2H), 2.60-2.64 (m, 2H), 2.45-2.50 (m, 2H), 2.26 (s, 3H), 1.89-1.99 (m, 2H). ESI MS m/z 395 [M+H]⁺.

### Example 35

### Preparation of N-(1-(4-(5,7-Dimethoxy-4-oxo-3,4-dihydroquinazolin-2-yl)phenyl)piperidin-4-yl)-N-ethylacetamide

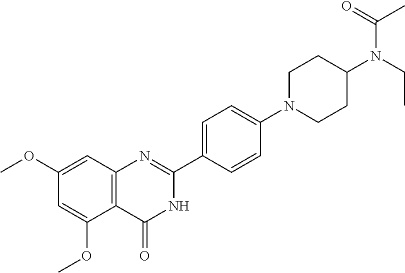 [SMILES: CCN(C(C)=O)C1CCN(c2ccc(-c3nc4cc(OC)cc(OC)c4c(=O)[nH]3)cc2)CC1]

To a solution of 4-acetamidopiperidine (2.5 g, 17.5 mmol) in DMF (10 mL) was added 4-fluorobenzaldehyde (2.2 g, 17.5 mmol) and K₂CO₃ (2.9 g, 21.2 mmol). The reaction was heated at 120° C. for 4 hours, diluted with H₂O, and extracted with EtOAc. The organics were washed sequentially with H₂O and brine, dried (Na₂SO₄), filtered, and concentrated in vacuo, to afford N-(1-(4-formylphenyl)piperidin-4-yl)acetamide (3.1 g, 92%).

A 60% suspension in oil of NaH (0.113 g, 2.8 mmol) was added to a 0° C. solution of N-(1-(4-formylphenyl)piperidin-4-yl)acetamide (0.700 g, 2.8 mmol) in DMF (10 mL) and stirred for 35 minutes. To this mixture was added Etl (0.23 mL, 2.8 mmol) and the reaction was warmed to room temperature for 2 hours, quenched with H₂O, and extracted with EtOAc. The organics were washed with brine, dried over anhydrous Na₂SO₄, filtered, and concentrated in vacuo. Purification by flash chromatography on silica gel, eluting with 0% to 5% MeOH/CH₂Cl₂, afforded N-ethyl-N-(1-(4-formylphenyl)piperidin-4-yl)acetamide (0.490 g, 64%).

A mixture of N-ethyl-N-(1-(4-formylphenyl)piperidin-4-yl)acetamide (0.385 g, 1.40 mmol), NaHSO₃ (0.162 g, 1.50 mmol), and p-TsOH (0.024 g, 0.12 mmol) were added to a solution of 2-amino-4,6-dimethoxybenzamide (0.250 g, 1.20 mmol) in DMA (10 mL). The reaction was stirred at 150° C. for 4 hours and then cooled to room temperature overnight. The mixture was diluted with H₂O and extracted with EtOAc. The organics were washed with brine, dried over anhydrous Na₂SO₄, filtered, and concentrated in vacuo. Purification by flash chromatography on silica gel, eluting with 2% to 10% MeOH/CH₂Cl₂, afforded the title compound (0.300 g, 55%) as a yellow solid. ¹H NMR (300 MHz, DMSO-d₆): mixture of rotamers δ 11.76 (s, 1H), 8.08 (d, J=8.7 Hz, 2H), 7.02 (d, J=8.7 Hz, 2H), 6.67 (d, J=2.0 Hz, 1H), 6.46 (d, J=2.0 Hz, 1H), 4.29-4.33 (m, 0.5H), 3.99-4.03 (m, 2H), 3.88 (s, 3H), 3.83 (s, 3H), 3.12-3.25 (m, 2H), 2.81-2.93 (m, 2H), 2.07 (s, 1.5H), 2.01 (s, 1.5H), 1.59-1.74 (m, 4.5H), 1.10 (t, J=6.7 Hz, 1.5H), 0.99 (t, J=6.7 Hz, 1.5H). ESI MS m/z 451 [M+H]⁺.

### Example 36

### Preparation of 2-(4-((3R,5S)-4-Acetyl-3,5-dimethylpiperazin-1-yl)phenyl)-5,7-dimethoxyquinazolin-4(3H)-one

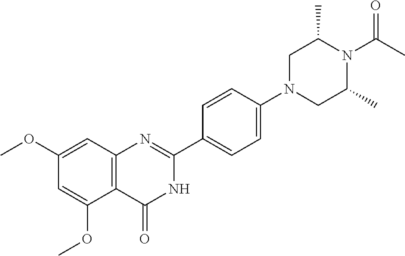 [SMILES: COc1cc(OC)c2c(=O)[nH]c(-c3ccc(N4C[C@@H](C)N(C(C)=O)[C@@H](C)C4)cc3)nc2c1]

A mixture of 4-fluorobenzaldehyde (2.0 g, 16.1 mmol), 2,6-dimethylpiperazine (2.2 g, 19.3 mmol), and K₂CO₃ (2.7 g, 19.3 mmol) in DMF (10 mL) was heated at 120° C. for 4 hours. Then, the reaction was diluted with H₂O and extracted with EtOAc. The organics were washed with brine, dried (Na₂SO₄), filtered and concentrated in vacuo. Purification by flash chromatography on silica gel eluting with 3% to 10% MeOH/CH₂Cl₂ afforded 4-(3,5-dimethylpiperazin-1-yl)benzaldehyde (2.0 g, 57%).

A solution of 4-(3,5-dimethylpiperazin-1-yl)benzaldehyde (1.0 g, 4.6 mmoL) in CH₂Cl₂ (15 mL) was cooled to 0° C. and treated with Et₃N (0.64 mL, 4.6 mmol) followed by acetyl chloride (0.33 mL, 4.6 mmol). The reaction stirred for 30 minutes, then concentrated in vacuo. Purification by flash chromatography on silica gel, eluting with 0% to 50% EtOAc/CH₂Cl₂, afforded 4-(4-acetyl-3,5-dimethylpiperazin-1-yl)benzaldehyde (1.0 g, 83%).

A mixture of 4-(4-acetyl-3,5-dimethylpiperazin-1-yl)benzaldehyde (0.580 g, 2.20 mmol), NaHSO₃ (0.260 g, 2.40 mmol), and p-TsOH (0.039 g, 0.20 mmol) was added to a solution of 2-amino-4,6-dimethoxybenzamide (0.400 g, 2.20 mmol) in DMA (15 mL). The reaction was stirred at 120° C. for 4 hours and then cooled to room temperature overnight. The mixture was diluted with H₂O and extracted with EtOAc. The organics were washed with brine, dried over anhydrous Na₂SO₄, filtered, and concentrated in vacuo. Purification by flash chromatography on silica gel, eluting with 2% to 10% MeOH/CH₂Cl₂, afforded the title compound (0.400 g, 46%) as a yellow solid. ¹H NMR (300 MHz, DMSO-d₆): δ 11.78 (br s, 1H), 8.10 (d, J=8.9 Hz, 2H), 7.05 (d, J=9.0 Hz, 2H), 6.68 (d, J=2.3 Hz, 1H), 6.46 (d, J=2.3 Hz, 1H), 4.01-4.64 (m, 2H), 3.71-3.95 (m, 8H), 2.87-3.07 (m, 2H), 2.06 (s, 3H), 1.25 (d, J=6.2 Hz, 6H). ESI MS m/z 437 [M+H]⁺.

### Example 37

### Preparation of 2-(4-((3R,5S)-3,5-Dimethylpiperazin-1-yl)phenyl)-5,7-dimethoxyquinazolin-4(3H)-one

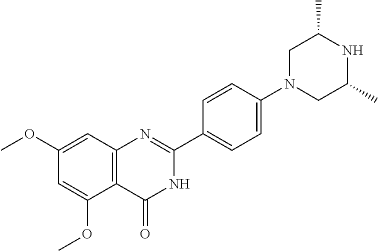

A solution of 2-(4-(4-acetyl-3,5-dimethylpiperazin-1-yl)phenyl)-5,7-dimethoxyquinazolin-4(3H)-one (0.150 g, 0.34 mmol) in 2N HCl was heated at reflux temperature for 3 days. The reaction was cooled to room temperature, basified with 1N NaOH, and extracted with CH₂Cl₂. Purification by flash chromatography on silica gel, eluting with 0% to 15% MeOH/CH₂Cl₂, followed by further purification, eluting with 30% to 100% of 92:7:1 CHCl₃/MeOH/concentrated NH₄OH, afforded the title compound (0.040 g, 30%) as a white solid. ¹H NMR (300 MHz, DMSO-d₆): δ 11.98 (br s, 1H), 8.08 (d, J=9.0 Hz, 2H), 7.00 (d, J=9.0 Hz, 2H), 6.68 (d, J=2.3 Hz, 1H), 6.46 (d, J=2.3 Hz, 1H), 3.88 (s, 3H), 3.83 (s, 3H), 3.73-3.76 (m, 2H), 2.78-2.81 (m, 2H), 2.19-2.26 (m, 2H), 1.03 (d, J=6.2 Hz, 6H). ESI MS m/z 395 [M+H]⁺.

### Example 38

### Preparation of 2-(4-(4-Acetyl-3-methylpiperazin-1-yl)phenyl)-5,7-dimethoxyquinazolin-4(3H)-one

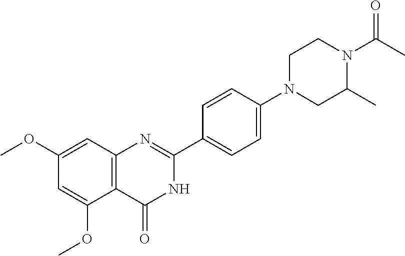 [SMILES: COc1cc(OC)c2c(=O)[nH]c(-c3ccc(N4CCN(C(C)=O)C(C)C4)cc3)nc2c1]

To a solution of 4-fluorobenzaldehyde (2.0 g, 16.1 mmol) in DMF (10 mL) was added 2-methylpiperazine (1.9 g, 19.3 mmol) and K₂CO₃ (2.7 g, 19.3 mmol). The reaction was heated at 120° C. for 6 hours, diluted with H₂O, and extracted with EtOAc. The organics were washed with brine, dried over anhydrous Na₂SO₄, filtered, and concentrated in vacuo, to afford 4-(3-methylpiperazin-1-yl)benzaldehyde (2.3 g, 69%): ¹H NMR (300 MHz, CDCl₃): δ 9.77 (s, 1H), 7.75 (d, J=9.0 Hz, 2H), 6.90 (d, J=9.0 Hz, 2H), 3.67-3.83 (m, 2H), 3.07-3.18 (m, 1H), 2.81-3.06 (m, 3H), 2.50-2.62 (m, 1H), 1.46-1.73 (br s, 1H), 1.15 (d, J=6.3 Hz, 3H). ESI MS m/z 205 [M+H]⁺.

A solution of 4-(3-methylpiperazin-1-yl)benzaldehyde (1.0 g, 4.89 mmol) in methylene chloride (15 mL) was cooled to 0° C. and treated with Et₃N (0.68 mL, 4.89 mmol), followed by acetyl chloride (0.34 mL, 4.89 mmol). The resulting solution was stirred at 0° C. for 20 minutes and then concentrated in vacuo. The material was purified by flash chromatography on silica gel, eluting with 0% to 5% of EtOAc/CH₂Cl₂, to afford 4-(4-acetyl-3-methylpiperazin-1-yl)benzaldehyde (0.88 g, 73%).

To a solution of 4-(4-acetyl-3-methylpiperazin-1-yl)benzaldehyde (0.400 g, 1.62 mmol) in DMA (15 mL) was added 2-amino-4,6-dimethoxybenzamide (0.349 g, 1.78 mmol), NaHSO₃ (0.201 g, 1.94 mmol) and p-TsOH (0.030 g, 0.16 mmol). The resulting solution was heated to 155° C. for 5 hours. The mixture was cooled to room temperature, diluted with water, extracted with CH₂Cl₂, washed with brine, dried (Na₂SO₄), filtered, and concentrated in vacuo. The material was purified by flash chromatography on silica gel, eluting with 50% to 100% of 92:7:1 CHCl₃/MeOH/concentrated NH₄OH in CH₂Cl₂, to afford the title compound (0.150 g, 21%). ¹H NMR (300 MHz, DMSO-d₆): mixture of rotamers δ 11.57 (s, 1H), 8.10 (d, J=8.9 Hz, 2H), 6.90-7.14 (m, 2H), 6.68 (s, 1H), 6.46 (s, 1H), 4.42-4.75 (m, 0.5H), 4.03-4.42 (m, 1H), 3.61-4.02 (m, 8H), 3.41-3.60 (m, 1H), 2.85-3.13 (m, 2H), 2.63-2.85 (m, 0.5H), 1.88-2.13 (m, 3H), 1.04-1.31 (m, 3H). ESI MS m/z 423 [M+H]⁺.

### Example 39

### Preparation of N-(1-(4-(5,7-Dimethoxy-4-oxo-3,4-dihydroquinazolin-2-yl)phenyl)pyrrolidin-3-yl)acetamide

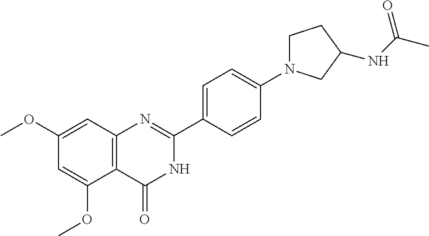 [SMILES: COc1cc(OC)c2c(=O)[nH]c(-c3ccc(N4CCC(NC(C)=O)C4)cc3)nc2c1]

A solution of 2-(4-(3-aminopyrrolidin-1-yl)phenyl)-5,7-dimethoxyquinazolin-4(3H)-one (0.150 g, 0.41 mmol) in CH₂Cl₂ (10 mL) was treated with Et₃N (0.114 mL, 0.82 mmol), cooled to 0° C., and acetyl chloride (0.029 mL, 0.41 mmol) was added. The mixture was stirred for 2 hours at room temperature, concentrated, and purified by flash chromatography on silica gel, eluting with 1:1 CH₂Cl₂/92:71 CHCl₃/MeOH/concentrated NH₄OH to 100% 92:7:1 CHCl₃/MeOH/concentrated NH₄OH. The mixture was further purified by flash chromatography on silica gel, eluting with 9:1 methylene chloride/methanol, to afford the title compound (0.130 g, 78%) as a yellow solid. ¹H NMR (300 MHz, DMSO-d₆): δ 11.67 (s, 1H), 8.18 (d, J=6.8 Hz, 1H), 8.14 (d, J=6.8 Hz, 2H), 6.66 (d, J=2.3 Hz, 1H), 6.60 (d, J=9.0 Hz, 2H), 6.44 (d, J=2.3 Hz, 1H), 4.36-4.39 (m, 1H), 3.88 (s, 3H), 3.83 (s, 3H), 3.13-3.59 (m, 5H), 2.15-2.22 (m, 1H), 1.90-1.94 (m, 1H), 1.82 (s, 3H). ESI MS m/z 409 [M+H]⁺.

### Example 40

### Preparation of N-(1-(4-(5,7-dimethoxy-4-oxo-3,4-dihydroquinazolin-2-yl)phenyl)piperidin-4-yl)-N-isopropylacetamide

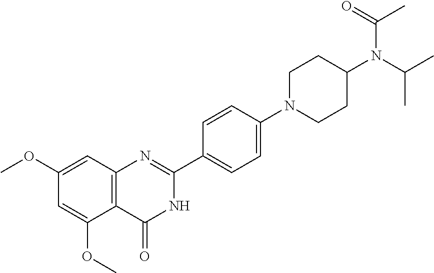 [SMILES: COc1cc(OC)c2c(=O)[nH]c(-c3ccc(N4CCC(N(C(C)=O)C(C)C)CC4)cc3)nc2c1]

To the solution of tert-butyl 4-oxopiperidine-1-carboxylate (5.0 g, 25.09 mmol) in methanol (35 mL) was added isopropylamine (1.07 mL, 12.54 mmol), acetic acid (0.94 mL, 16.30 mmol) and sodium cyanoborohydride (1.0 g, 16.30 mmol). The resulting solution was stirred at room temperature for 1 hour, then quenched with water. The solution was concentrated in vacuo and redissolved in ethyl ether. The organics were extracted with 0.1 N HCl. The aqueous extracts were basified with 1 N NaOH (pH >8) and extracted with ethyl ether. The organic extracts were dried over anhydrous Na₂SO₄, filtered, and concentrated in vacuo, to afford tert-butyl 4-(isopropylamino)piperidine-1-carboxylate (1.2 g, 41%) as a clear liquid.

To a 0° C. solution of tert-butyl 4-(isopropylamino)piperidine-1-carboxylate (1.2 g, 5.19 mmol) in CH₂Cl₂ (18 mL) was added Et₃N (1.44 mL, 10.38 mmol) followed by acetyl chloride (0.55 mL, 7.78 mmol). The resulting solution was stirred for 2.5 hours, then concentrated in vacuo. The material was purified by flash chromatography on silica gel, eluting with 0% to 5% of EtOAc/CH₂Cl₂, to afford tert-butyl 4-(N-isopropylacetamido)piperidine-1-carboxylate (0.88 g, 59%).

A solution of tert-butyl 4-(N-isopropylacetamido)piperidine-1-carboxylate (0.880 g, 3.09 mmol) in hydrogen chloride (4.0 M solution in 1,4-dioxane, 10 mL) was stirred at room temperature overnight. The resulting solution was concentrated in vacuo, basified with aqueous saturated NaHCO₃, and extracted with EtOAc. The organics were dried (Na₂SO₄), filtered, and concentrated in vacuo. The material was purified by flash chromatography on silica gel, eluting with 50% to 100% of 92:7:1 CHCl₃/MeOH/concentrated NH₄OH in CH₂Cl₂. The residue was further purified by flash chromatography on silica gel, eluting with 100% of 92:7:1 CHCl₃/MeOH/concentrated NH₄OH, to afford N-Isopropyl-N-(piperidin-4-yl)acetamide hydrogen chloride (0.260 g, 45%) as a clear liquid.

To a solution of N-isopropyl-N-(piperidin-4-yl)acetamide hydrogen chloride (0.260 g, 1.41 mmol) in DMF (5 mL) was added 4-fluorobenzaldehyde (0.18 mL, 1.69 mmol) and K₂CO₃ (0.233 g, 1.69 mmol). The resulting solution was heated to 120° C. overnight, and cooled. The cooled solution was diluted with water and extracted with CH₂Cl₂. The organics were washed with brine, dried over anhydrous Na₂SO₄, filtered, and concentrated in vacuo. The material was purified by flash chromatography on silica gel, eluting with 0% to 5% MeOH/CH₂Cl₂, to afford N-(1-(4-formylphenyl)piperidin-4-yl)-N-isopropylacetamide (0.290 g, 71%).

To a solution of N-(1-(4-formylphenyl)piperidin-4-yl)-N-isopropylacetamide (0.300 g, 1.04 mmol) in DMA (10 mL) was added 2-amino-4,6-dimethoxybenzamide (0.204 g, 1.04 mmol), NaHSO₃ (0.129 g, 1.24 mmol) and p-TsOH (0.019 g, 0.10 mmol). The resulting solution was heated to 155° C. overnight and then cooled to room temperature. The solution was diluted with water, extracted with CH₂Cl₂, washed with brine, dried over anhydrous Na₂SO₄, filtered, and concentrated in vacuo. The material was purified by flash chromatography on silica gel eluting, with 30% to 100% of 92:7:1 CHCl₃/MeOH/concentrated NH₄OH in CH₂Cl₂, to afford the title compound (0.100 g, 20%). ¹H NMR (300 MHz, DMSO-d₆): mixture of rotamers δ 11.66 (s, 1H), 8.07 (d, J=8.3 Hz, 2H), 6.89-7.15 (m, 2H), 6.67 (s, 1H), 6.46 (s, 1H), 3.90-4.11 (m, 3H), 3.88 (s, 3H), 3.83 (s, 3H), 2.80-3.02 (m, 2H), 2.39-2.66 (m, 1H), 1.92-2.06 (m, 3H), 1.63-1.82 (m, 2H), 1.32-1.47 (m, 1H), 1.21-1.32 (m, 3H), 1.08-1.21 (m, 4H). ESI MS m/z 463 [M−H]⁻.

### Example 41

### Preparation of 5-Chloro-2-(4-(4-isopropylpiperazin-1-yl)phenyl)quinazolin-4(3H)-one

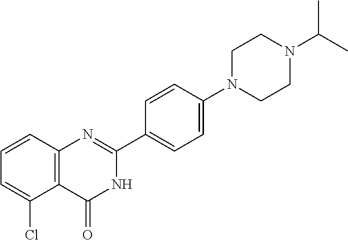 [SMILES: CC(C)N1CCN(c2ccc(-c3nc4cccc(Cl)c4c(=O)[nH]3)cc2)CC1]

A solution of 2-amino-6-chlorobenzamide (0.314 g, 1.85 mmol) and 4-(4-isopropylpiperazin-1-yl)benzaldehyde (0.430 g, 1.85 mmol) in DMA (25 mL) were treated with p-TsOH (0.035 g, 0.185 mmol) and NaHSO₃ (0.212 g, 2.04 mmol), and the mixture was heated at 140° C. for 18 hours. Then, the mixture was cooled, diluted with CH₂Cl₂ (200 mL), and washed with saturated NaHCO₃ (100 mL). The organic phase was dried over anhydrous MgSO₄, filtered, concentrated, and purified by silica gel chromatography, eluting with 1:1 CH₂Cl₂/92:7:1 CHCl₃/MeOH/concentrated NH₄OH to 100% 92:7:1 CHCl₃/MeOH/concentrated NH₄OH to 100% 6:3:1 CHCl₃/MeOH/concentrated NH₄OH. The resulting solids were rechromatographed with 9:1 CH₂Cl₂/MeOH to afford the title compound as a white solid. ¹H NMR (300 MHz, DMSO-d₆): δ 12.24 (br s, 1H), 8.11 (d, J=8.8 Hz, 2H), 7.66-7.71 (m, 1H), 7.59 (d, J=7.9 Hz, 1H), 7.42 (d, J=7.4 Hz, 1H), 7.03 (d, J=8.6 Hz, 2H), 3.28-3.34 (m, 4H), 2.64-2.73 (m, 1H), 2.55-2.59 (m, 4H), 1.01 (d, J=6.4 Hz, 6H). ESI MS m/z 383 [M+H]⁺.

### Example 42

### Preparation of 2-(4-((3R,5S)-4-Isopropyl-3,5-dimethylpiperazin-1-yl)phenyl)-5,7-dimethoxyquinazolin-4(3H)-one

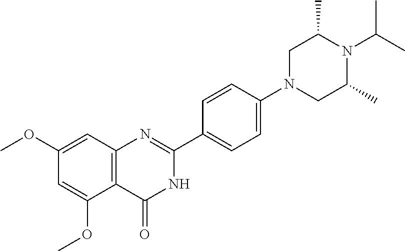 [SMILES: COc1cc(OC)c2c(=O)[nH]c(-c3ccc(N4C[C@@H](C)N(C(C)C)[C@@H](C)C4)cc3)nc2c1]

To a mixture of 4-(3,5-dimethylpiperazin-1-yl)benzaldehyde (1.0 g, 4.6 mmol) and K₂CO₃ (1.3 g, 9.2 mmol) in CH₃CN (10 mL) was added 2-iodopropane (2.3 mL, 22.9 mmol) and the reaction was stirred at reflux temperature overnight. Additional 2-iodopropane (2.3 mL, 22.9 mmol) and K₂CO₃ (1.3 g, 9.2 mmol) were added and the reaction was continued to reflux overnight. The mixture was concentrated in vacuo and purified by flash chromatography on silica gel, eluting with 1% to 10% MeOH/CH₂Cl₂, to afford 4-(4-isopropyl-3,5-dimethylpiperazin-1-yl)benzaldehyde (0.550 g, 46%).

A mixture of 4-(4-isopropyl-3,5-dimethylpiperazin-1-yl)benzaldehyde (0.400 g, 1.50 mmol), NaHSO₃ (0.195 g, 1.80 mmol), and p-TsOH (0.030 g, 0.15 mmol) was added to a solution of 2-amino-4,6-dimethoxybenzamide (0.400 g, 2.40 mmol) in DMA (10 mL). The reaction was stirred at 140° C. for 4 hours, then at room temperature overnight. The mixture was diluted with H₂O and extracted with CH₂Cl₂. The organics were washed with brine, dried (Na₂SO₄), filtered, and concentrated in vacuo. Purification by flash chromatography on silica gel, eluting with 1% to 10% MeOH/CH₂Cl₂, followed by reverse-phase chromatography, eluting with 10% to 90% CH₃CN in H₂O, afforded the title compound (0.114 g, 17%). ¹H NMR (300 MHz, DMSO-d₆): δ 11.68 (s, 1H), 8.09 (d, J=8.9 Hz, 2H), 6.78 (d, J=9.0 Hz, 2H), 6.66 (s, 1H), 6.44 (s, 1H), 3.87 (s, 3H), 3.83 (s, 3H), 3.41-3.44 (m, 2H), 3.11-3.23 (m, 5H), 1.00-1.03 (m, 12H). ESI MS m/z 437 [M+H]⁺.

### Example 43

### Preparation of 5,7-Dimethoxy-2-(4-(piperidin-4-yl)phenyl)quinazolin-4(3H)-one

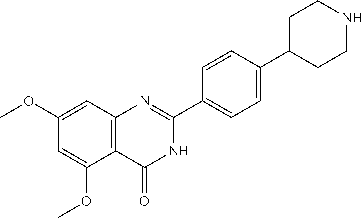 [SMILES: COc1cc(OC)c2c(=O)[nH]c(-c3ccc(C4CCNCC4)cc3)nc2c1]

To a solution of tert-butyl 4-(4-(5,7-dimethoxy-4-oxo-3,4-dihydroquinazolin-2-yl)phenyl)piperidine-1-carboxylate (0.210 g, 0.45 mmol) in 1,4-dioxane (2 mL) was added 4M HCl in 1,4-dioxane (1 mL). The resulting solution was stirred at room temperature for 5 hours. Then, the mixture was concentrated in vacuo and the resulting material was purified by flash chromatography on silica gel, eluting with 0% to 10% of MeOH/CH₂Cl₂. The residue was further purified by flash chromatography on silica gel, eluting with 100% of 92:7:1 CHCl₃/MeOH/concentrated NH₄OH followed by 100% of 6:3:1 CHCl₃/MeOH/concentrated NH₄OH, to afford the title compound (0.030 g, 18%). ¹H NMR (300 MHz, DMSO-d₆): δ 8.11 (d, J=8.3 Hz, 2H), 7.37 (d, J=8.2 Hz, 2H), 6.73 (s, 1H), 6.53 (s, 1H), 3.89 (s, 3H), 3.85 (s, 3H), 2.92-3.20 (m, 2H), 2.56-2.81 (m, 3H), 2.35-2.57 (m, 2H), 1.67-1.88 (m, 2H), 1.38-1.67 (m, 2H). ESI MS m/z 366 [M+H]⁺.

### Example 44

### Preparation of 5,7-Dimethoxy-2-(4-(3-(methylamino)pyrrolidin-1-yl)phenyl)quinazolin-4(3H)-one

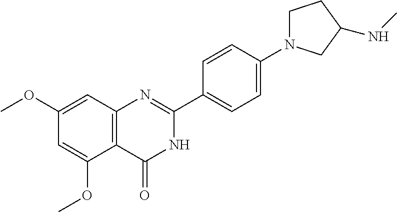

A mixture of N-(1-(4-(5,7-dimethoxy-4-oxo-3,4-dihydroquinazolin-2-yl)phenyl)pyrrolidin-3-yl)-N-methylacetamide (0.500 g, 1.18 mmol) and 2 N HCl (80 mL) was heated at 100° C. for 4 hours, cooled, basified to pH 9, extracted with CH₂Cl₂ (2×200 mL), dried (MgSO₄), filtered, and concentrated. The residue was purified by flash chromatography on silica gel, eluting with 1:1 CH₂Cl₂/92:7:1 CHCl₃/MeOH/concentrated NH₄OH to 100% 92:7:1 CHCl₃/MeOH/concentrated NH₄OH to 6:3:1 CHCl₃/MeOH/concentrated NH₄OH, to afford the title compound (0.210 g, 47%) as a pale yellow solid. ¹H NMR (300 MHz, DMSO-d₆): δ 11.65 (br s, 1H), 8.08 (d, J=8.7 Hz, 2H), 6.65 (s 1H), 6.55 (d, J=7.8 Hz, 2H), 6.43 (s, 1H), 3.88 (s, 3H), 3.83 (s, 3H), 3.46-3.49 (m, 1H), 3.38-3.42 (m, 1H), 3.26-3.28 (m, 2H), 3.07-3.10 (m, 1H), 2.31 (s, 3H), 2.08-2.11 (m, 1H), 1.81-1.84 (m, 1H). ESI MS m/z 381 [M+H]⁺.

### Example 45

### Preparation of Tert-butyl 4-(4-(5,7-dimethoxy-4-oxo-3,4-dihydroquinazolin-2-yl)phenyl)piperidine-1-carboxylate

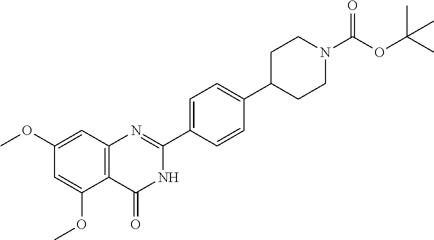 [SMILES: COc1cc(OC)c2c(=O)[nH]c(-c3ccc(C4CCN(C(=O)OC(C)(C)C)CC4)cc3)nc2c1]

A solution of 2-(4-bromophenyl)-5,7-dimethoxyquinazolin-4(3H)-one (1.1 g, 3.23 mmol), K₂CO₃ (1.3 g, 9.69 mmol), PdCl₂(dppf) (0.261 g, 0.32 mmol) and tert-butyl 4-(4,4,5,5-tetramethyl-1,3,2-dioxaborolan-2-yl)-5,6-dihydropyridine-1(2H)-carboxylate (1.0 g, 3.23 mmol) in DMF (50 mL) was heated to 110° C. overnight. The resulting solution was concentrated in vacuo and the material was purified twice by flash chromatography on silica gel, eluting with 0% to 5% of MeOH/CH₂Cl₂. The residue was further purified by flash chromatography on silica gel, eluting with 10% to 50% of EtOAc/CH₂Cl₂, to afford tert-butyl 4-(4-(5,7-dimethoxy-4-oxo-3,4-dihydroquinazolin-2-yl)phenyl)-5,6-dihydropyridine-1(2H)-carboxylate (0.030 g, 49%) as a light yellow solid.

A solution of tert-butyl 4-(4-(5,7-dimethoxy-4-oxo-3,4-dihydroquinazolin-2-yl)phenyl)-5,6-dihydropyridine-1(2H)-carboxylate (0.160 g, 0.34 mmol) in EtOH (10 mL) and HOAc (5 mL) was purged with nitrogen, and 10% Pd/C (0.016 g) was added. The mixture was stirred under 1 atmosphere of hydrogen overnight. Then, the solution was filtered through Celite, with MeOH washings, and the filtrate was concentrated in vacuo. The material was purified by flash chromatography on silica gel, eluting with 30% to 70% of 92:7:1 CHCl₃/MeOH/concentrated NH₄OH in CH₂Cl₂, to afford the title compound (0.160 g, 100%). ¹H NMR (300 MHz, DMSO-d₆): δ 11.91 (s, 1H), 8.11 (d, J=8.3 Hz, 2H), 7.40 (d, J=8.5 Hz, 2H), 6.73 (s, 1H), 6.53 (s, 1H), 4.00-4.22 (m, 2H), 3.89 (s, 3H), 3.85 (s, 3H), 2.65-2.97 (m, 3H), 1.68-1.88 (m, 2H), 1.48-1.68 (m, 2H), 1.42 (s, 9H). ESI MS m/z 466 [M+H]⁺.

### Example 46

### Preparation of N-(1-(4-(5,7-dimethoxy-4-oxo-3,4-dihydroquinazolin-2-yl)phenyl)pyrrolidin-3-yl)-N-methylacetamide

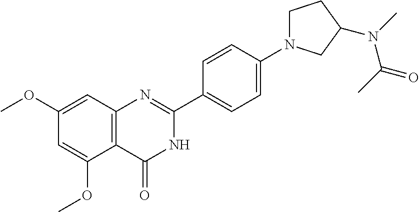 [SMILES: COc1cc(OC)c2c(=O)[nH]c(-c3ccc(N4CCC(N(C)C(C)=O)C4)cc3)nc2c1]

A solution of 4-fluorobenzaldehyde (2.01 g, 16.2 mmol) and N-methyl-N-(pyrrolidin-3-yl)acetamide (1.92 g, 13.5 mmol) in DMF (20 mL) was treated with K₂CO₃ (2.24 g, 16.2 mmol). The mixture was heated at 120° C. under nitrogen for 18 hours, cooled to room temperature, diluted with ethyl acetate (150 mL), washed with brine, dried (Na₂SO₄), filtered, and concentrated. The residue was purified by flash chromatography on silica gel, eluting with 100% ethyl acetate to 10% methanol/ethyl acetate, to afford N-(1-(4-formylphenyl)pyrrolidin-3-yl)-N-methylacetamide.

A solution of 2-amino-4,6-dimethoxybenzamide (0.797 g, 4.07 mmol) and N-(1-(4-formylphenyl)pyrrolidin-3-yl)-N-methylacetamide (1.0 g, 4.07 mmol) in DMA (75 mL) was treated with NaHSO₃ (0.466 g, 4.5 mmol) and p-TsOH (0.078 g, 0.41 mmol). The mixture was heated at 150° C. for 15 hours, cooled to room temperature, diluted with CH₂Cl₂ (200 mL), and washed with saturated NaHCO₃ (100 mL) and water (200 mL). The organic phase was dried over anhydrous MgSO₄, filtered, and concentrated. The residue was purified by flash chromatography on silica gel, eluting with 1:1 CH₂Cl₂/92:71 CHCl₃/MeOH/concentrated NH₄OH to 100% 92:7:1 CHCl₃/MeOH/concentrated NH₄OH, to afford the title compound (1.5 g, 88%) as a light brown solid. ¹H NMR (300 MHz, DMSO-d₆): δ 11.68 (s, 1H), 8.10 (d, J=8.8 Hz, 2H), 6.55-6.67 (m, 3H), 6.44 (d, J=2.2 Hz, 1H), 4.67-5.22 (m, 1H), 3.88 (s, 3H), 3.83 (s, 3H), 3.43-3.60 (m, 2H), 3.22-3.26 (m, 2H), 2.76-2.89 (m, 3H), 1.91-2.27 (m, 5H). ESI MS m/z 423 [M+H]⁺.

### Example 47

### Preparation of 2-(4-(4-(Isopropylamino)piperidin-1-yl)phenyl)-5,7-dimethoxyquinazolin-4(3H)-one

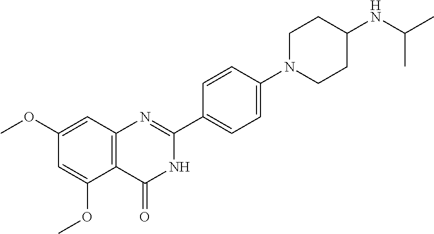 [SMILES: COc1cc(OC)c2c(=O)[nH]c(-c3ccc(N4CCC(NC(C)C)CC4)cc3)nc2c1]

A solution of N-(1-(4-(5,7-dimethoxy-4-oxo-3,4-dihydroquinazolin-2-yl)phenyl)piperidin-4-yl)-N-isopropylacetamide (0.130 g, 0.27 mmol) in 2 N HCl (8 mL) was heated to reflux and stirred overnight. The resulting solution was cooled to room temperature, basified with 2 N NaOH (pH 14), and extracted with CH₂Cl₂. The solution was concentrated in vacuo and the residue was purified by flash chromatography on silica gel, eluting with 30% to 100% of 92:7:1 CHCl₃/MeOH/concentrated NH₄OH in CH₂Cl₂, to afford the title compound (0.060 g, 52%). ¹H NMR (300 MHz, DMSO-d₆): δ 8.07 (d, J=9.0 Hz, 2H), 6.99 (d, J=9.1 Hz, 2H), 6.67 (s, 1H), 6.46 (s, 1H), 3.75-3.95 (m, 8H), 2.81-2.99 (m, 3H), 2.69-2.79 (m, 1H), 1.79-1.92 (m, 2H), 1.14-1.37 (m, 3H), 0.97 (d, J=6.1 Hz, 6H). ESI MS m/z 423 [M+H]⁺.

### Example 48

### Preparation of 5,7-Dimethoxy-2-(4-(3-methylpiperazin-1-yl)phenyl)quinazolin-4(3H)-one

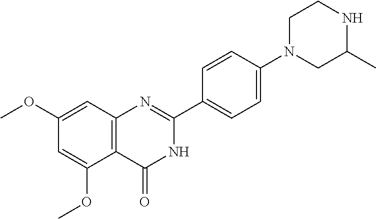

A solution of 2-(4-(4-acetyl-3-methylpiperazin-1-yl)phenyl)-5,7-dimethoxyquinazolin-4(3H)-one (0.340 g, 0.80 mmol) in 2 N HCl (5 mL) was heated to reflux and stirred for 3 days. Then, the resulting solution was cooled to room temperature, basified with 2 N NaOH, extracted with CH₂Cl₂, and concentrated in vacuo. The material was purified by flash chromatography on silica gel, eluting with 50% to 100% of 92:7:1 CHCl₃/MeOH/concentrate NH₄OH in CH₂Cl₂, to afford the title compound (0.03 g, 9%). ¹H NMR (300 MHz, DMSO-d₆): δ 10.76 (s, 1H), 8.08 (d, J=8.9 Hz, 2H), 6.99 (d, J=9.1 Hz, 2H), 6.67 (s, 1H), 6.46 (s, 1H), 3.88 (s, 3H), 3.83 (s, 3H), 3.62-3.79 (m, 2H), 2.90-3.04 (m, 1H), 2.57-2.85 (m, 4H), 2.20-2.39 (m, 1H), 1.03 (d, J=6.3 Hz, 3H). ESI MS m/z 381 [M+H]⁺.

### Example 49

### Preparation of N-Benzyl-N-(1-(5-(5,7-dimethoxy-4-oxo-3,4-dihydroquinazolin-2-yl)pyridin-2-yl)piperidin-4-yl)acetamide

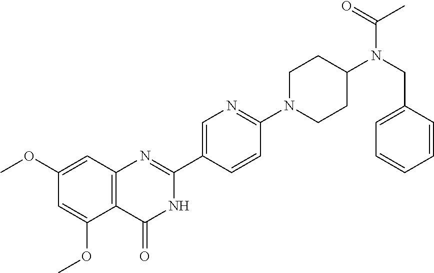 [SMILES: COc1cc(OC)c2c(=O)[nH]c(-c3ccc(N4CCC(N(Cc5ccccc5)C(C)=O)CC4)nc3)nc2c1]

To a solution of tert-butyl 4-oxopiperidine-1-carboxylate (10.0 g, 50.2 mmol) and benzylamine (2.7 mL, 25.1 mmol) in MeOH (30 mL) was added HOAc (1.9 mL, 32.6 mmol), followed by NaCNBH₃ (2.0 g, 32.6 mmol) and the reaction was stirred at room temperature overnight. The resulting mixture was quenched with H₂O (5 mL) and concentrated in vacuo. The residue was diluted with 0.1 N HCl and washed with Et₂O. The aqueous layer was then basified with 2 N NaOH and extracted with Et₂O. The organics were washed with brine, dried over anhydrous Na₂SO₄, filtered, and concentrated in vacuo, to afford tert-butyl 4-(benzylamino)piperidine-1-carboxylate (8.1 g, 56%).

To a solution of tert-butyl 4-(benzylamino)piperidine-1-carboxylate (8.1 g, 28.0 mmol) and Et₃N (7.8 mL, 56.0 mmol) in CH₂Cl₂ (100 mL) was added acetyl chloride (2.4 mL, 33.5 mmol) and the reaction was stirred at room temperature overnight, then concentrated in vacuo. Purification by flash chromatography on silica gel, eluting with 30% to 60% EtOAc/CH₂Cl₂, afforded tert-butyl 4-(N-benzylacetamido)piperidine-1-carboxylate (9.3 g, 99%).

A solution of tert-butyl 4-(N-benzylacetamido)piperidine-1-carboxylate (9.3 g, 28.0 mmol) in dioxane (20 mL) and 4 M HCl/dioxane (14.0 mL, 56.0 mmol) was stirred at room temperature overnight and then concentrated in vacuo. The residue was basified with 2 N NaOH and extracted with EtOAc. The organics were washed with brine, dried (Na₂SO₄), filtered, and concentrated in vacuo, to afford N-benzyl-N-(piperidin-4-yl)acetamide (4.4 g, 67%).

To a solution of N-benzyl-N-(piperidin-4-yl)acetamide (1.5 g, 6.3 mmol) and 2-(6-chloropyridin-3-yl)-5,7-dimethoxyquinazolin-4(3H)-one (1.0 g, 3.2 mmol) in DMF (15 mL) was added K₂CO₃ (0.875 g, 6.3 mmol) and the reaction was heated at reflux temperature overnight. The resulting mixture was concentrated in vacuo and purified by flash chromatography on silica gel, eluting with 1% to 10% MeOH/CH₂Cl₂, to afford the title compound (0.500 g, 30%) as a white solid. ¹H NMR (300 MHz, DMSO-d₆): δ 11.84 (s, 1H), 8.86 (s, 1H), 8.22 (d, J=9.2 Hz, 1H), 7.33-7.37 (m, 1H), 7.14-7.27 (m, 4H), 6.88-6.96 (m, 1H), 6.66 (d, J=1.5 Hz, 1H), 6.46 (d, J=1.5 Hz, 1H), 4.44-4.58 (m, 4.5H), 4.10-4.20 (m, 0.5H), 3.87 (s, 3H), 3.83 (s, 3H), 2.86-2.98 (m, 2H), 2.25 (s, 1.5H), 1.95 (s, 1.5H), 1.45-1.77 (m, 4H). ESI/APCI MS m/z 514 [M+H]⁺.

### Example 50

### Preparation of 2-(6-(4-(Benzylamino)piperidin-1-yl)pyridin-3-yl)-5,7-dimethoxyquinazolin-4(3H)-one

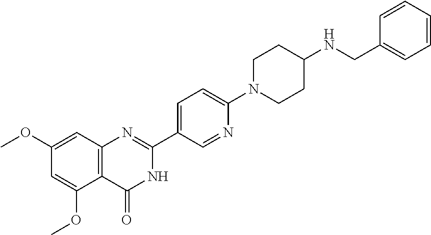 [SMILES: COc1cc(OC)c2c(=O)[nH]c(-c3ccc(N4CCC(NCc5ccccc5)CC4)nc3)nc2c1]

A solution of N-benzyl-N-(1-(5-(5,7-dimethoxy-4-oxo-3,4-dihydroquinazolin-2-yl)pyridin-2-yl)piperidin-4-yl)acetamide (0.200 g, 0.39 mmol) in 2 N HCl (15 mL) was refluxed for 3 days. The resulting mixture was basified with 2 N NaOH and extracted with CH₂Cl₂. The organics were washed with brine, dried over anhydrous Na₂SO₄, filtered, and concentrated in vacuo. Purification by flash chromatography on silica gel, eluting with 10% to 100% of 92:7:1 CHCl₃/MeOH/concentrated NH₄OH in CH₂Cl₂, afforded the title compound (0.110 g, 60%) as a white solid. ¹H NMR (300 MHz, DMSO-d₆): δ 11.11 (br s, 1H), 8.89 (d, J=2.3 Hz, 1H), 8.22-8.26 (m, 1H), 7.28-7.37 (m, 4H), 7.18-7.23 (m, 1H), 6.91 (d, J=7.2 Hz, 1H), 6.67 (d, J=2.2 Hz, 1H), 6.46 (d, J=2.2 Hz, 1H), 4.27-4.31 (m, 2H), 3.88 (s, 3H), 3.83 (s, 3H), 3.76 (s, 2H), 3.00-3.11 (m, 2H), 2.62-2.69 (m, 1H), 1.88-1.91 (m, 2H), 1.25-1.31 (m, 2H). ESI MS m/z 472 [M+H]⁺.

### Example 51

### Preparation of 4-(4-(5,7-Dimethoxy-4-oxo-3,4-dihydroquinazolin-2-yl)phenyl)piperazine-1-carbaldehyde

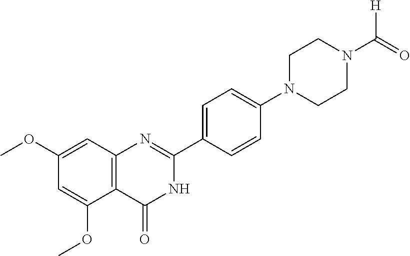 [SMILES: COc1cc(OC)c2c(=O)[nH]c(-c3ccc(N4CCN(C=O)CC4)cc3)nc2c1]

A mixture of methyl formate (75 mL) and 5,7-dimethoxy-2-(4-(piperazin-1-yl)phenyl)quinazolin-4(3H)-one (0.300 g, 0.82 mmol) was heated at reflux for 48 hours. The mixture was concentrated, and purified by silica gel chromatography, eluting with 1:1 CH₂Cl₂/92:7:1 CHCl₃/MeOH/concentrated NH₄OH, to afford the title compound (0.320 g, 99%) as a white solid. ¹H NMR (300 MHz, DMSO-d₆): δ 11.79 (br s, 1H), 8.10-8.19 (m, 3H), 7.06 (d, J=9.1 Hz, 2H), 6.69 (d, J=2.3 Hz, 1H), 6.48 (d, J=2.3 Hz, 1H), 3.88 (s, 3H), 3.84 (s, 3H), 3.46-3.59 (m, 4H), 3.32-3.38 (m, 4H). APCI MS m/z 393 [M−H]⁻.

### Example 52

### Preparation of 5,7-Dimethoxy-2-(4-(4-oxopiperidin-1-yl)phenyl)pyrido[2,3-d]pyrimidin-4(3H)-one

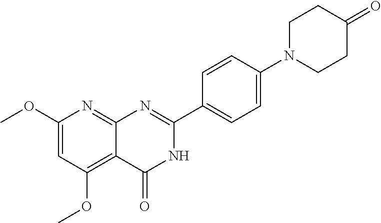 [SMILES: COc1cc(OC)c2c(=O)[nH]c(-c3ccc(N4CCC(=O)CC4)cc3)nc2n1]

To a solution of 2-[4-(4-hydroxy-piperazin-1-yl)-phenyl]-5,7-dimethoxy-3H-pyrido[2,3-d]pyrimidin-4-one (160 mg, 0.418 mmol) in DMSO (4.0 mL), 1,2-benziodexol-3(1H)-one-1-hydroxy-1-oxide (IBX) (178 mg, 0.635 mmol) was added and the reaction mixture was kept at 50° C. for 16 hours. Water was added and the precipitated solid was filtered to give crude product, which was purified by column chromatography (silica gel 230-400 mesh; eluting with 3% methanol in dichloromethane) to obtain the title compound as a yellow solid. Yield: 0.70 g (44.0%). MP >350° C. ¹H NMR (400 MHz, CDCl₃): δ 12.15 (s, 1H), 8.18 (d, J=9.2 Hz, 2H), 7.02 (d, J=9.2 Hz, 2H), 6.33 (s, 1H), 3.95 (s, 3H), 3.90 (s, 3H), 3.77 (t, J=6.4 Hz, 4H), 2.45 (t, J=6.4 Hz, 4H).

### Example 53

### Preparation of 2-(2-(Hydroxymethyl)-1H-indol-5-yl)-5,7-dimethoxyquinazolin-4(3H)-one

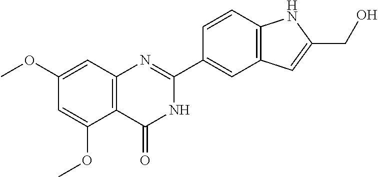 [SMILES: COc1cc(OC)c2c(=O)[nH]c(-c3ccc4[nH]c(CO)cc4c3)nc2c1]

To a solution of N-(4-formyl-phenyl)-acetamide (1.25 g, 7.67 mmol) in trifluoroacetic acid (70 mL) was slowly added thallium(III)trifluoroacetate (5.00 g, 9.20 mmol). The reaction mixture was stirred at room temperature for 30 minutes. Then, a solution of sodium iodide (1.19 g, 7.95 mmol) in water (10 mL) was added slowly. The color changed to dark purple and a lot of solid was formed. Stirring continued at room temperature for 16 hours. Solvent was evaporated to half of the volume, and water (50 mL) was added. The pH was adjusted to approximately 13 with 4 N NaOH solution. The mixture was extracted with ethyl acetate (2×100 mL). The organic phase was dried over anhydrous Na₂SO₄ and concentrated on a rotary evaporator. The solid obtained was washed with ethyl acetate (2×5 mL), ether (2×10 mL), and dried under vacuum to give N-(4-formyl-2-iodo-phenyl)-acetamide as an off-white solid. Yield: 0.760 g (34%).

To a degassed solution of N-(4-formyl-2-iodo-phenyl)-acetamide (0.760 g, 2.63 mmol) in anhydrous DMF (20 mL) were added bis(triphenylphosphine)palladium(II) dichloride (90 mg, 0.13 mmol), copper (I) iodide (0.03 g, 0.13 mmol), 1,1,3,3-tetramethyl guanidine (1.51 g, 13.1 mmol), and propargyl alcohol (0.210 g, 3.68 mmol). The reaction mixture was stirred at room temperature for 2 hours and then at 80° C. for 24 hours under nitrogen. Solvent was evaporated under reduced pressure. Water (100 mL) was added and the mixture was extracted with ethyl acetate (200 mL). The organic phase was backwashed with water (2×100 mL), brine (100 mL), and dried over anhydrous Na₂SO₄. Solvent was evaporated and crude compound was purified by the Simpliflash system (60% ethyl acetate in hexanes as eluent) to give 2-hydroxymethyl-1H-indole-5-carbaldehyde as a pale yellow solid. Yield: 0.10 g (22%).

To a solution of 2-hydroxymethyl-1H-indole-5-carbaldehyde (90 mg, 0.51 mmol) and 2-amino-4,6-dimethoxy-benzamide (0.15 g, 0.77 mmol) in N,N-dimethylacetamide (5 mL) were added sodium hydrogen sulfite (58.5 wt %) (0.14 g, 0.77 mmol) and p-toluenesulfonic acid (20 mg, 0.10 mmol). The reaction mixture was stirred at 120° C. for 16 hours under nitrogen, cooled to room temperature, and concentrated under reduced pressure. Water (20 mL) was added. The separated solid was filtered, washed with water (20 mL) and ether (20 mL), and dried under vacuum. Crude compound was purified by column chromatography (silica gel 230-400 mesh; 0-5% methanol in CH₂Cl₂ as eluent), to give the title compound as an off-white solid. Yield: 0.06 g (33%). MP 264-265° C. ¹H NMR (400 MHz, DMSO-d₆): δ 11.85 (br s, 1H), 11.36 (s, 1H), 8.39 (s, 1H), 7.93 (dd, J=8.6 and 1.6 Hz, 1H), 7.44 (d, J=9.0 Hz, 1H), 6.73 (d, J=2.3 Hz, 1H), 6.49 (d, J=2.4 Hz, 1H), 6.41 (s, 1H). 5.34 (t, J=5.8 Hz, 1H), 4.63 (d, J=5.5 Hz, 2H), 3.90 (s, 3H), 3.85 (s, 3H).

### Example 54

### Preparation of 2-(2-(2-Hydroxyethyl)-1H-indol-5-yl)-5,7-dimethoxyquinazolin-4(3H)-one

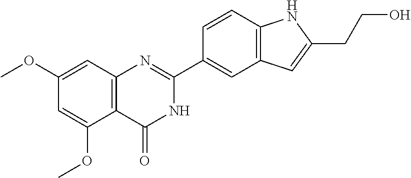 [SMILES: COc1cc(OC)c2c(=O)[nH]c(-c3ccc4[nH]c(CCO)cc4c3)nc2c1]

To a stirred solution of 4-amino-3-iodo-benzoic acid methyl ester (11.1 g, 40.0 mmol) in pyridine (80 mL) was added acetyl chloride (3.30 g, 42.0 mmol) at 0° C. under nitrogen. Stirring continued at 0° C. for 30 minutes. The ice-bath was removed, and stirring continued at room temperature for 16 hours. Pyridine was evaporated under reduced pressure. The residue was taken in ethyl acetate (300 mL). The organic phase was washed with 2 N aqueous HCl (200 mL), water (200 mL), brine (200 mL), and then dried over anhydrous Na₂SO₄. Removal of solvent gave 4-acetylamino-3-iodo-benzoic acid methyl ester as a white solid. Yield: 12.71 g (99%).

Lithium aluminium hydride (2.43 g, 64.1 mmol) was taken in a dry, three-necked, round bottom flask. Anhydrous THF (80 mL) was added and cooled to −10° C. A solution of 4-acetylamino-3-iodo-benzoic acid methyl ester (10.2 g, 32.0 mmol) in anhydrous THF (60 mL) was added dropwise at −10° C. over a period of 45 minutes under nitrogen. Stirring was continued at −10° C. for 1 hour. The reaction mixture was quenched with saturated sodium sulfate aqueous solution. The reaction mixture was then filtered, and the filtrate was concentrated. The solid was washed with methanol. The combined organic phases were dried over anhydrous Na₂SO₄. The solvent was evaporated. The crude compound was purified by the Simpliflash system (5% methanol in CH₂Cl₂ as eluent), to give N-(4-hydroxymethyl-2-iodo-phenyl)-acetamide as a white solid. Yield: 6.36 g (68%).

To a solution of IBX (0.93 g, 3.3 mmol) in dimethylsulfoxide (3.5 mL) was added N-(4-hydroxymethyl-2-iodo-phenyl)-acetamide (0.87 g, 3.0 mmol) and the reaction mixture was stirred at room temperature for 1 hour. Water (50 mL) was added and the solid was separated by filtration, and washed with ethyl acetate (20 mL). The filtrate was collected and extracted with ethyl acetate (200 mL). The organic phase was washed with brine (100 mL) and dried over anhydrous Na₂SO₄. Removal of solvent gave N-(4-formyl-2-iodo-phenyl)-acetamide as a light brown solid. Yield: 0.82 g (95%).

To a degassed solution of N-(4-formyl-2-iodo-phenyl)-acetamide (0.810 g, 2.82 mmol) in DMF (25 mL) and triethylamine (5 mL) were added PdCl₂(PPh₃)₂ (0.10 g, 0.14 mmol) and copper (I) iodide (0.16 g, 0.85 mmol). A degassed solution of but-3-yn-1-ol (0.27 g, 0.29 mmol) in DMF (8 mL) and triethylamine (2 mL) was added at 80° C. over a period of 1 hour under nitrogen. After the addition, the reaction mixture was stirred at 80° C. for 4 hours, cooled to room temperature, and concentrated under reduced pressure. The residue was diluted with water (100 mL) and extracted with ethyl acetate (200 mL). The organic phase was washed with brine (100 mL) and dried over anhydrous Na₂SO₄. Removal of solvent gave N-[4-formyl-2-(4-hydroxy-but-1-ynyl)-phenyl]-acetamide as a brown solid. Crude yield: 0.85 g (100%). The crude material was used in next step without further purification.

To a solution of N-[4-formyl-2-(4-hydroxy-but-1-ynyl)-phenyl]-acetamide (0.85 g, approximately 2.80 mmol) in THF (20 mL) was added a THF solution of TBAF (6.0 mL, 6.0 mmol) and the reaction mixture was stirred at reflux for 36 hours under nitrogen and cooled to room temperature. Solvent was evaporated and the residue was taken in ethyl acetate (200 mL). The organic phase was washed with water (2×100 mL), brine (100 mL) and dried over anhydrous Na₂SO₄. Solvent was evaporated; crude compound was purified by simpliflash system (50% ethyl acetate in hexanes as eluent) to give 2-(2-hydroxy-ethyl)-1H-indole-5-carbaldehyde as yellow solid. Yield: 0.31 g (58% for two steps).

To a solution of 2-(2-hydroxy-ethyl)-1H-indole-5-carbaldehyde (0.300 g, 1.58 mmol) and 2-amino-4,6-dimethoxy-benzamide (0.370 g, 1.90 mmol) in N,N-dimethylacetamide (5 mL) were added sodium hydrogen sulfite (58.5 wt %) (0.350 g, 1.90 mmol) and p-toluenesulfonic acid monohydrate (60 mg, 0.32 mmol). The reaction mixture was stirred at 120° C. for 16 hours under nitrogen and cooled to room temperature. The solvent was evaporated under reduced pressure. Water (20 mL) was added and the solid was separated by filtration, washed with water (30 mL) and dried under vacuum. Crude compound was purified by the Simpliflash system (5:20:75 methanol/ethyl acetate/CH₂Cl₂ as eluent) to give the title compound as an off-white solid. Yield: 0.22 g (38%). MP 237-238° C. ¹H NMR (400 MHz, DMSO-d₆): δ 11.83 (br s, 1H), 11.20 (s, 1H), 8.34 (s, 1H), 7.90 (d, J=8.2 Hz, 1H), 7.37 (d, J=8.6 Hz, 1H), 6.73 (d, J=1.9 Hz, 1H), 6.48 (d, J=1.9 Hz, 1H), 6.30 (s, 1H), 4.81 (t, J=5.1 Hz, 1H), 3.89 (s, 3H), 3.84 (s, 3H), 3.75 (q, J=6.63 Hz, 2H), 2.89 (t, J=7.0 Hz, 2H).

### Example 55

### Preparation of 5,7-Dimethoxy-2-(2-(pyrrolidin-1-ylmethyl)-1H-indol-5-yl)quinazolin-4(3H)-one

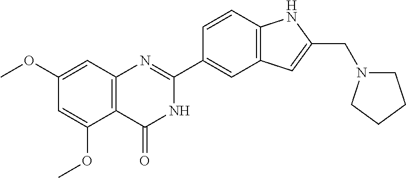 [SMILES: COc1cc(OC)c2c(=O)[nH]c(-c3ccc4[nH]c(CN5CCCC5)cc4c3)nc2c1]

To a mixture of 5-bromo-1H-indole-2-carboxylic acid (1.0 g, 4.2 mmol), 1-ethyl-3-(3′-dimethylaminopropyl)carbodiimide hydrochloride (EDCI) (1.1 g, 5.9 mmol), 1-hydroxybenzotriazole hydrate (HOBt) (0.62 g, 4.6 mmol) in THF (20 mL) was added 4-methylmorpholine (NMM) (0.65 mL, 5.9 mmol). After 10 minutes, pyrrolidine (0.73 mL, 8.8 mmol) was added. The mixture was stirred at room temperature under nitrogen for 17 hours. The solvent was removed under reduced pressure. Water was added, stirred for 0.5 hours. The solid was filtered, washed with water, and dried in air to afford (5-bromo-1H-indol-2-yl)-pyrrolidin-1-yl-methanone as a pale yellow solid. Yield: 1.2 g (95%).

To a suspension of (5-bromo-1H-indol-2-yl)-pyrrolidin-1-yl-methanone (0.53 g, 1.8 mmol) in THF (50 mL) at 0° C. was slowly added lithium aluminum hydride (0.20 g, 5.4 mmol). The mixture was stirred under nitrogen at 0° C. for a while and the cooling bath was allowed to warm to room temperature. The mixture was then stirred at room temperature for 17 hours. The reaction was quenched by careful, successive, dropwise addition of water (0.2 mL), 15% NaOH aqueous solution (0.2 mL), and water (0.6 mL). The solid was filtered and washed with MeOH and CH₂Cl₂. The filtrate was concentrated to dryness, and dried under vacuum, to give 5-bromo-2-pyrrolidin-1-ylmethyl-1H-indole as a white solid. Yield: 0.45 g (90%).

To a suspension of potassium hydride (30 wt % dispersion in mineral oil) (96 mg, 0.72 mmol) in ether (20 mL) at 0° C. was added 5-bromo-2-pyrrolidin-1-ylmethyl-1H-indole (0.20 g, 0.72 mmol). After stirring for 30 minutes, the reaction mixture was cooled to −78° C., and t-BuLi solution (1.7 M in pentane; 0.93 mL, 1.58 mmol) was added. The mixture was stirred at −78° C. for 15 minutes, then at −20° C. for approximately 3 min, and then it was cooled down to −78° C. again. DMF was added. The mixture was stirred under nitrogen at −78° C. for a while and the cooling bath was allowed to warm to room temperature. Saturated NaHCO₃ aqueous solution (approximately 5 mL) was added. The mixture was extracted with dichloromethane. The organic solution was dried over Na₂SO₄, and concentrated to dryness to afford a mixture of the desired product and starting material, at about a 1:1 ratio, from the NMR spectrum. The crude product (approximately 0.2 g) was used for next reaction without any further purification.

A mixture of 2-amino-4,6-dimethoxy-benzamide (0.20 g, 1.0 mmol), crude 2-pyrrolidin-1-ylmethyl-1H-indole-5-carbaldehyde (0.23 g, 1.0 mmol), p-toluenesulfonic acid monohydrate (0.38 g, 2.0 mmol), and sodium bisulfite (0.42 g, 4.0 mmol) in N,N-dimethylacetamide (5 mL) was stirred at 115° C. under N₂ for 17 hours and cooled to room temperature. The mixture was diluted with saturated Na₂CO₃ aqueous solution and concentrated to dryness under reduced pressure. The residue was purified by column chromatography on silica gel, eluting with CH₂Cl₂:7.0 M NH₃ in MeOH (95:5), to afford the title compound as a yellow solid. Yield: 87 mg (22%). MP 168-169.5° C. (decomposition). ¹H NMR (400 MHz, CDCl₃): δ 9.05 (s, 1H), 8.22 (s, 1H), 7.85 (d, 1H), 7.43 (d, 1H), 6.84 (s, 1H), 6.45 (s, 1H), 6.43 (s, 1H), 3.96 (s, 3H), 3.92 (s, 3H), 3.81 (s, 2H), 2.57 (m, 4H), 1.81 (m, 4H).

### Example 56

### Preparation of 2-(3-(Hydroxymethyl)-1H-indazol-5-yl)-5,7-dimethoxyquinazolin-4(3H)-one

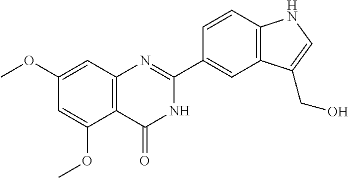 [SMILES: COc1cc(OC)c2c(=O)[nH]c(-c3ccc4[nH]cc(CO)c4c3)nc2c1]

To a solution of sodium nitrite (20.0 g, 290.0 mmol) in THF (1000 mL) and water (50 mL) was added 1H-indole-5-carboxylic acid methyl ester (5.00 g, 28.5 mmol). The mixture was cooled to 0° C. and aqueous 6 N HCl (70 mL) was added dropwise at 0° C. After stirring for 3 days at room temperature, solvent was evaporated, and extracted with ethyl acetate (3×200 mL). The combined organic phase was washed brine (200 mL) and dried over anhydrous Na₂SO₄. The solvent was evaporated. The residue was purified by the Simpliflash system (20-30% ethyl acetate in hexanes as eluent), to give 3-formyl-1H-indazole-5-carboxylic acid methyl ester as a yellow solid. Yield: 1.47 g, (25%).

To a solution of 3-formyl-1H-indazole-5-carboxylic acid methyl ester (0.37 g, 1.80 mmol) in anhydrous methanol (15 mL) was added sodium borohydride (68 mg, 1.80 mmol) in small portions at 0° C. After the addition, the reaction mixture was stirred at 0° C. for 30 minutes. Solvent was evaporated; water (100 mL) was added and the mixture was extracted with ethyl acetate (150 mL). The organic phase was washed with brine (100 mL) and dried over anhydrous Na₂SO₄. Solvent was evaporated to give 3-hydroxymethyl-1H-indazole-5-carboxylic acid methyl ester as a yellow solid. Yield: 0.32 g (87%).

To a solution of 3-hydroxymethyl-1H-indazole-5-carboxylic acid methyl ester (0.32 g, 1.55 mmol) in a mixture of anhydrous dichloromethane and THF (2:1, 60 mL) was added pyridinium p-toluene sulfonate (0.08 g, 0.31 mmol) and then 3,4-dihydro-2H-pyran (0.19 g, 2.32 mmol) was added. The reaction mixture was stirred at room temperature for 16 hours under nitrogen. Solvent was evaporated; water (100 mL) was added, and the mixture was extracted with ethyl acetate (100 mL). The organic phase was washed with brine (100 mL) and dried over anhydrous Na₂SO₄. Removal of solvent gave 3-(tetrahydro-pyran-2-yloxymethyl)-1H-indazole-5-carboxylic acid methyl ester as a yellow gummy material. Yield: 0.55 g (crude). This product was used in next step without further purification.

3-(Tetrahydro-pyran-2-yloxymethyl)-1H-indazole-5-carboxylic acid methyl ester (0.53 g crude, approximately 1.55 mmol) was taken in anhydrous THF (20 mL) and cooled to −10° C. A solution of lithium aluminium hydride (1.0 M solution in THF, 0.12 g, 3.10 mmol) was added drop-wise at −10° C. over a period of 15 minutes under nitrogen. Stirring continued at −10° C. for 1 hour and the reaction was then allowed to warm to room temperature and stirring continued at room temperature for 16 hours. The reaction mixture was carefully quenched with saturated aq. saturated ammonium chloride solution (100 mL). Then, reaction mixture was diluted with ethyl acetate (100 mL). The organic phase was separated, washed with brine (50 mL) and dried over anhydrous Na₂SO₄. Solvent was evaporated to give [3-(tetrahydro-pyran-2-yloxymethyl)-1H-indazol-5-yl]-methanol as a yellow gummy material. Yield: 0.40 g (crude). This product was used in the next step without further purification.

To a solution of [3-(tetrahydro-pyran-2-yloxymethyl)-1H-indazol-5-yl]-methanol (0.40 g, 1.50 mmol) in DMSO (3 mL), IBX (0.42 g, 1.50 mmol) was added and the reaction mixture was stirred at room temperature for 3 hours under nitrogen. Water (50 mL) was added; the separated solid was filtered, and the solid was washed with ethyl acetate (100 mL). The filtrate was collected and the organic phase was separated, washed with brine (100 mL), and dried over anhydrous Na₂SO₄. Removal of solvent gave 3-(tetrahydro-pyran-2-yloxymethyl)-1H-indazole-5-carbaldehyde as an off-white solid. Yield: 0.33 g (84%).

To a solution of 3-(tetrahydro-pyran-2-yloxymethyl)-1H-indazole-5-carbaldehyde (0.32 g, 1.23 mmol) and 2-amino-4,6-dimethoxy-benzamide (0.24 g, 1.23 mmol) in N,N-dimethylacetamide (10 mL) were added NaHSO₃ (58.5 wt %, 0.27 g, 1.48 mmol) and p-toluenesulfonic acid monohydrate (0.05 g, 0.25 mmol); the reaction mixture was heated at 120° C. for 16 hours, then cooled to room temperature. Solvent was removed under reduced pressure. The residue was diluted with water (100 mL). The separated solid was filtered and washed with water and dried under vacuum. The residue was purified by the Simpliflash system (0-5% methanol in CH₂Cl₂ as eluent) to give the title compound as an off-white solid. Yield: 30 mg (7%). MP 264-266° C. ¹H NMR (400 MHz, CD₃OD): δ 8.60 (s, 1H), 8.10 (d, J=8.98 Hz, 1H), 7.65 (d, J=8.98 Hz, 1H), 6.85 (d, J=1.95 Hz, 1H), 6.55 (d, J=1.95 Hz, 1H), 5.05 (s, 2H), 3.96 (s, 6H).

### Example 57

### Preparation of 5,7-Dimethoxy-2-(2-(2-(pyrrolidin-1-yl)ethyl)-1H-indol-5-yl)quinazolin-4(3H)-one

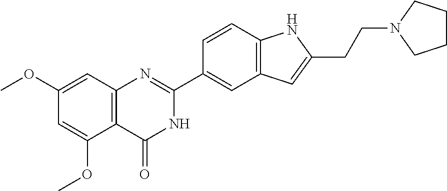 [SMILES: COc1cc(OC)c2c(=O)[nH]c(-c3ccc4[nH]c(CCN5CCCC5)cc4c3)nc2c1]

To a stirred solution of 4-amino-3-iodo-benzoic acid methyl ester (11.1 g, 40.0 mmol) in anhydrous pyridine (80 mL) was added acetyl chloride (3.30 g, 42.0 mmol) at 0° C. under nitrogen. Stirring was continued at 0° C. for 30 minutes. The ice-bath was removed, and stirring continued at room temperature for 16 hours. Pyridine was evaporated under reduced pressure. The residue was taken in ethyl acetate (300 mL). The organic phase washed with 2 N aqueous HCl (200 mL), water (200 mL), brine (200 mL), and was dried over anhydrous Na₂SO₄. Removal of solvent gave 4-acetylamino-3-iodo-benzoic acid methyl ester as a white solid. Yield: 12.7 g (99%).

To but-3-yn-1-ol (40.0 g, 570.0 mmol) and 3,4-dihydro-2H-pyran (48.0 g, 570.0 mmol) in anhydrous dichloromethane (350 mL) was added pyridium p-toluenesulfonate (0.45 g, 1.80 mmol). The mixture was stirred at room temperature for 16 hours. Solvent was evaporated, and the residue was purified by vacuum distillation to give 2-but-3-ynyloxy-tetrahydro-pyran as a light yellow liquid. Yield: 60.0 g (68%).

To a degassed solution of 4-acetylamino-3-iodo-benzoic acid methyl ester (41.4 g, 130 mmol) in DMF (200 mL) and triethylamine (40 mL) were added PdCl₂(PPh₃)₂ (3.99 g, 5.68 mmol) and copper (I) iodide (7.43 g, 39.0 mmol). A degassed solution of 2-but-3-ynyloxy-tetrahydro-pyran (30.1 g, 195 mmol) in DMF (100 mL) and triethylamine (20 mL) was added at 80° C. over a period of 1 hour under nitrogen. After the addition, the reaction mixture was stirred at 80° C. for 2 hours and then cooled to room temperature. Solvent was evaporated under reduced pressure. Ethyl acetate (200 mL) was added. The solid was filtered, and washed with ethyl acetate. The ethyl acetate solution was washed with brine, and dried over anhydrous Na₂SO₄. The organic phase was concentrated to dryness, to afford 66.8 g crude 4-acetylamino-3-[4-(tetrahydro-pyran-2-yloxy)-but-1-ynyl]-benzoic acid methyl ester. This was used in next step without further purification.

A solution of crude 4-acetylamino-3-[4-(tetrahydro-pyran-2-yloxy)-but-1-ynyl]-benzoic acid methyl ester (33.4 g, approximately 65 mmol) in anhydrous THF (300 mL) was mixed with a 1.0 M solution of tetrabutylammonium fluoride in THF (110 mL, 110 mmol); the reaction mixture was stirred at 90° C. for 4 hours under nitrogen, and then cooled to room temperature. Solvent was evaporated and the residue was taken in ethyl acetate (300 mL). The organic phase was washed with water (300 mL), brine (200 mL), and dried over anhydrous Na₂SO₄. The solvent was evaporated and the crude compound was purified by column chromatography on silica gel, eluting with hexanes and ethyl acetate (3:1), to give 2-[2-(tetrahydro-pyran-2-yloxy)-ethyl]-1H-indole-5-carboxylic acid methyl ester. Yield: 14.9 g (76%).

Lithium aluminum hydride (3.38 g, 89.0 mmol) in anhydrous THF (100 mL) was cooled to −30° C. 2-[2-(Tetrahydro-pyran-2-yloxy)-ethyl]-1H-indole-5-carboxylic acid methyl ester (13.5 g, 44.5 mmol) in anhydrous THF (100 mL) was added dropwise. The reaction mixture was stirred at −20° C. for 1 hour and then at room temperature for 4 hours. The reaction mixture was cooled to 0° C. and water (6 mL) was added slowly. Ammonium chloride solution (200 mL) was added and extracted with ethyl acetate (2×200 mL). The organic phase was washed with water (100 mL), then brine (100 mL), and dried over anhydrous sodium sulfate. The solvent was evaporated to give {2-[2-(tetrahydro-pyran-2-yloxy)-ethyl]-1H-indol-5-yl}-methanol as a white solid. Yield: 11.50 g (94%).

{2-[2-(Tetrahydro-pyran-2-yloxy)-ethyl]-1H-indol-5-yl}-methanol (11.5 g 41.7 mmol) in anhydrous DMSO (45 mL) was added IBX (12.3 g, 43.8 mmol) and the reaction was stirred at room temperature for 2 hours. The reaction mixture was poured into water (300 mL) and extracted with ethyl acetate (300 mL), the organic phase was washed with water, then brine, and was purified by column chromatography on silica gel, eluting with dichloromethane, to give 2-[2-(tetrahydro-pyran-2-yloxy)-ethyl]-1H-indole-5-carbaldehyde as a white solid. Yield: 8.50 g (75%).

To a solution of 2-amino-4,6-dimethoxy-benzamide (6.10 g, 31.1 mmol) and 2-[2-(tetrahydro-pyran-2-yloxy)-ethyl]-1H-indole-5-carbaldehyde (8.50 g, 31.1 mmol) in N,N-dimethylacetamide (45 mL) was added NaHSO₃ (58.5 wt %, 6.08 g, 34.2 mmol) and p-TSA (0.60 g, 3.11 mmol). The reaction mixture was heated at 115° C. for 16 hours and then cooled to room temperature. N,N-dimethylacetamide was removed under reduced pressure, the residue was diluted with water (50 mL) and the solid was collected and mixed with dichloromethane (100 mL), ether (100 mL), and then filtered to give a mixture of 5,7-dimethoxy-2-{2-[2-(tetrahydro-pyran-2-yloxy)-ethyl]-1H-indol-5-yl}-3H-quinazolin-4-one and 2-[2-(2-hydroxy-ethyl)-1H-indol-5-yl]-5,7-dimethoxy-3H-quinazolin-4-one as a white solid, which was used in next step without further purification. Yield: 7.50 g (crude).

A mixture of 5,7-dimethoxy-2-{2-[2-(tetrahydro-pyran-2-yloxy)-ethyl]-1H-indol-5-yl}-3H-quinazolin-4-one and 2-[2-(2-hydroxy-ethyl)-1H-indol-5-yl]-5,7-dimethoxy-3H-quinazolin-4-one (7.50 g, 16.6 mmol) was dissolved in anhydrous methanol (60 mL). 1.0 M HCl in ether (42 mL) was added and the reaction was stirred at room temperature for 2 hours. The solid was filtered and the mother liquor was evaporated to dryness and the residue was combined with the solid. Sodium bicarbonate solution (200 mL) was added and stirred for 1 hours. The separated solid was filtered and washed with cold water and dried under vacuum to give 2-[2-(2-hydroxy-ethyl)-1H-indol-5-yl]-5,7-dimethoxy-3H-quinazolin-4-one as a white solid. Yield: 6.2 g (55%; 3 steps).

To a solution of 2-[2-(2-hydroxy-ethyl)-1H-indol-5-yl]-5,7-dimethoxy-3H-quinazolin-4-one (6.20 g, 16.9 mmol) in anhydrous DMF (25 mL) was added carbon tetrabromide (6.47 g, 19.5 mmol) and triphenylphosphine (5.11 g, 19.5 mmol). The reaction mixture was stirred at 40° C. for 16 hours. DMF was removed under vacuum and water (150 mL) was added. The separated solid was filtered and mixed with ether (150 mL) and heated for 10 minutes. The solid was filtered and dried under vacuum to give 2-[2-(2-bromo-ethyl)-1H-indol-5-yl]-5,7-dimethoxy-3H-quinazolin-4-one as a white solid. Yield: 6.1 g (84%).

To a solution of 2-[2-(2-bromo-ethyl)-1H-indol-5-yl]-5,7-dimethoxy-3H-quinazolin-4-one (6.10 g, 14.2 mmol) in anhydrous DMF (45 mL) was added pyrrolidine (6.07 g, 85.4 mmol) and the reaction mixture was stirred at 45° C. for 15 hours. DMF was removed under reduced pressure, the residue was taken in water (150 mL), and stirred for 30 minutes. Separated solid was filtered, washed with water, and dried under vacuum. Crude compound was purified by column chromatography (silica gel 230-400 mesh, eluting with 5% 7.0 M ammonia in methanol solution in dichloromethane) to give the title compound as a white solid. Yield: 3.4 g (57%). MP 215-217° C. ¹H NMR (400 MHz, DMSO-d₆): δ 11.79 (s, 1H), 11.21 (s, 1H), 8.31 (s, 1H), 7.88 (dd, J=8.8 and 1.6 Hz, 1H), 7.35 (d, J=8.8 Hz, 1H), 6.71 (d, J=2.4 Hz, 1H), 6.46 (d, J=2.4 Hz, 1H), 6.28 (s, 1H), 3.87 (s, 3H), 3.83 (s, 3H), 2.89 (t, J=8.0 Hz, 2H), 2.74 (t, J=8.0 Hz, 2H), 2.48 (m, 4H), 1.67 (m, 4H).

### Example 58

### Preparation of 2-(2-((Dimethylamino)methyl)-1H-indol-5-yl)-5,7-dimethoxyquinazolin-4(3H)-one

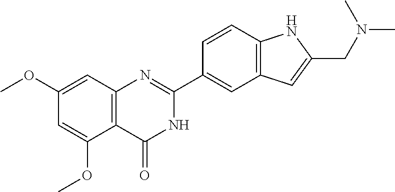

To a solution of 5-bromo-1H-indole-2-carboxylic acid (2.40 g, 10.0 mmol) in THF (100 mL) were added EDCI (2.11 g, 30.0 mmol), HOBt (1.49 g, 11.0 mmol). The reaction mixture was stirred at room temperature for 10 minutes. Then, a solution of N,N-dimethyl amine (2.0 M solution in THF, 15 mL, 30.0 mmol) was added. The mixture was stirred for 16 hours at room temperature. Solvent was evaporated, the residue was taken in ethyl acetate (200 mL), and water (200 mL) was added. The organic phase was separated; the aqueous phase was extracted with ethyl acetate (200 mL). The combined organic phase was washed with water (100 mL), then brine (100 mL), and dried over anhydrous sodium sulfate. Solvent was evaporated and dried under vacuum to give 5-bromo-1H-indole-2-carboxylic acid dimethylamide as an off-white solid. Yield: 2.56 g (96%).

5-Bromo-1H-indole-2-carboxylic acid dimethylamide (1.34 g, 5.00 mmol) was taken in anhydrous THF (50 mL) (suspension), and cooled to −20° C. A solution of lithium aluminium hydride (1.0 M solution in THF, 10.0 mL, 10.0 mmol) was added dropwise at −20° C. over a period of 15 minutes under nitrogen, and allowed to warm to 10° C.; stirring was continued at 10° C. for 1 hour. The reaction mixture was carefully quenched with aq. saturated ammonium chloride solution (10 mL). The reaction mixture was diluted with ethyl acetate (150 mL). The organic phase was separated, washed with water (100 mL), then brine (100 mL), and dried over anhydrous Na₂SO₄. Solvent was evaporated, to give (5-bromo-1H-indole-2-ylmethyl)-dimethyl amine as an off-white solid. Yield: 1.27 g (crude).

To a cold (0° C.) solution of potassium hydride (suspension in mineral oil, 0.79 g, 5.90 mmol) in anhydrous THF (60 mL) was added a solution of (5-bromo-1H-indole-2-ylmethyl)-dimethyl amine (1.24 g, 4.90 mmol) in anhydrous THF (20 mL) was added dropwise at 0° C. over a period of 15 minutes under nitrogen. Stirring was continued for 30 minutes at 0° C., then cooled to −10° C. n-Butyl lithium (1.6 M solution in hexanes, 7.4 mL, 11.7 mmol) was added rapidly. Stirring was continued at −10° C. for 1 h. Then, anhydrous DMF (5.0 mL) was added, and the mixture was allowed to warm to room temperature over 2 h. The reaction mixture was carefully quenched with 1N aq. HCl (10 mL). The reaction mixture was diluted with ethyl acetate (150 mL). The organic phase was separated, washed with water (100 mL), then brine (100 mL), and dried over anhydrous Na₂SO₄. The solvent was evaporated to give 2-dimethylaminomethyl-1H-indole-5-carbaldehyde as an orange-colored gummy material. Yield: 0.91 g (crude). This product was used in next step without further purification.

To a solution of 2-dimethylaminomethyl-1H-indole-5-carbaldehyde (0.88 g crude, 4.35 mmol) and 2-amino-4,6-dimethoxy-benzamide (0.85 g, 4.35 mmol) in N,N-dimethylacetamide (15 mL) were added sodium hydrogen sulfite (58.5 wt %, 0.95 g, 5.22 mmol) and p-toluenesulfonic acid (0.99 g, 5.22 mmol). The reaction mixture was stirred at 120° C. for 5 hours under nitrogen, then cooled to room temperature, and concentrated under reduced pressure. 30% aqueous sodium carbonate (50 mL) was then added. The separated solid was filtered, washed with water (50 mL), and dried under vacuum. Crude compound was purified by the Simpliflash system (0-5% methanol in CH₂Cl₂ and 7 N ammonia in methanol 5% in CH₂Cl₂ as eluent) to give the title compound as an off-white solid. Yield: 0.83 g (50%). MP 187-188° C. ¹H NMR (400 MHz, DMSO-d₆): δ 11.82 (s, 1H), 11.34 (s, 1H), 8.38 (s, 1H), 7.93 (d, J=8.59 Hz, 1H), 7.40 (d, J=8.59 Hz, 1H), 6.73 (s, 1H), 6.49 (s, 1H), 6.40 (s, 1H), 3.90 (s, 3H), 3.85 (s, 3H), 3.57 (s, 2H), 2.21 (s, 6H).

### Example 59

### Preparation of N-(4-(5,7-Dimethoxy-4-oxo-3,4-dihydroquinazolin-2-yl)phenyl)methanesulfonamide

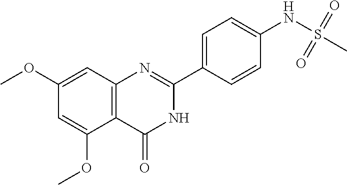

A mixture of 4-bromobenzaldehyde (0.250 g, 1.40 mmol), methanesulfonamide (0.154 g, 1.62 mmol), copper iodide (0.0510 g, 0.270 mmol), N,N-dimethylglycine (0.0280 g, 0.270 mmol), and potassium phosphate tribasic (0.716 g, 3.38 mmol) in DMF (5.00 mL) was stirred at reflux for 16 hours. The mixture was diluted with EtOAc (50 mL), washed with water (50 mL), and then saturated aqueous LiCl (5 mL). The combined aqueous layers were then back-extracted with EtOAc (50 mL). The organic layers were combined, washed with brine (50 mL), dried over Na₂SO₄, filtered, and the solvent was removed under reduced pressure, to provide N-(4-formylphenyl)methanesulfonamide (0.161 g, 58%) as a yellow oil.

A mixture of N-(4-formylphenyl)methanesulfonamide (0.161 g, 0.0800 mmol), 2-amino-4,6-dimethoxybenzamide (0.159 g, 0.0800 mmol), NaHSO₃ (94%, 0.00460 g, 0.0240 mmol), and p-TsOH.H₂O (0.0125 g, 0.120 mmol) in DMA (1.00 mL) was heated at 155° C. for 16 hours. The mixture was diluted with EtOAc (50 mL), washed with water (2×50 mL), then brine (50 mL), dried over Na₂SO₄, filtered, and the solvent was removed under vacuum. The residue was purified over silica gel (12 g, CH₂Cl₂/MeOH) and the product was freeze-dried from MeCN/H₂O to provide the title compound (0.0341 g, 11%) as a pale yellow solid. ¹H NMR (300 MHz, DMSO-d₆): δ 11.94 (s, 1H), 10.21 (s, 1H), 8.16 (d, J=8.76 Hz, 2H), 7.30 (d, J=8.76 Hz, 2H), 6.72 (d, J=2.25 Hz, 1H), 6.52 (d, J=2.25 Hz, 1H), 3.89 (s, 3H), 3.85 (s, 3H), 3.09 (s, 3H). MS (ESI) m/z 376 [C₁₇H₁₇N₃O₆S+H]⁺.

### Example 60

### Preparation of 5,7-Dimethoxy-2-(4-(pyridin-4-ylamino)phenyl)quinazolin-4(3H)-one

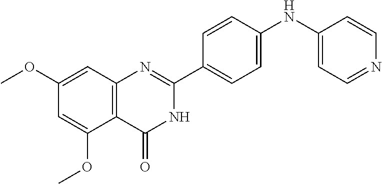 [SMILES: COc1cc(OC)c2c(=O)[nH]c(-c3ccc(Nc4ccncc4)cc3)nc2c1]

A mixture of compound 2-(4-bromophenyl)-5,7-dimethoxyquinazolin-4(3H)-one) (0.200 g, 0.554 mmol), 4-aminopyridine (0.0573 g, 0.609 mmol), Pd₂(dba)₃ (0.0025 g, 0.0028 mmol), Xantphos (0.0018 g, 0.0031 mmol), and Cs₂CO₃ (0.253 g, 0.776 mmol) in 1,4-dioxane (2.22 mL) under nitrogen was heated at 105° C. for 2 days. The mixture was cooled to room temperature, diluted with EtOAc (200 mL), washed with water (3×75 mL), then brine (75 mL), dried over anhydrous Na₂SO₄, filtered, and the solvent was removed under vacuum. The resulting residue was purified over silica gel (12 g, EtOAc/CHCl₃/MeOH/NH₄OH), to provide the title compound as a white solid. ¹H NMR (300 MHz, DMSO-d₆): δ 11.90 (s, 1H), 9.19 (s, 1H), 8.29 (d, J=6.29 Hz, 2H), 8.17 (d, J=8.75 Hz, 2H), 7.30 (d, J=8.75 Hz, 2H), 7.05 (d, J=6.29 Hz, 2H), 6.72 (d, J=2.26 Hz, 1H), 6.51 (d, J=2.26 Hz, 1H), 3.89 (s, 3H), 3.85 (s, 3H). MS (ESI) m/z 375 [C₂₁H₁₈N₄O₃+H]⁺.

### Example 61

### Preparation of 5,7-Dimethoxy-2-(4-(p-tolylamino)phenyl)quinazolin-4(3H)-one

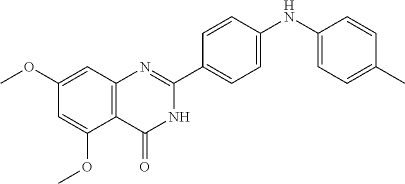

To a mixture of Pd(OAc)₂ (0.0112 g, 0.0166 mmol) and (S)-(−)-BINAP (0.0155 g, 0.0249 mmol) was added a degassed solution of toluene/t-BuOH (5:1, 3.00 mL) and the mixture was heated at 100° C. for 1 minute. In a second flask, 2-(4-bromophenyl)-5,7-dimethoxyquinazolin-4(3H)-one) (0.300 g, 0.831 mmol) and degassed toluene/t-BuOH (5:1, 4.00 mL) was heated at 100° C. for 1 minute, t-BuOK (0.130 g, 1.17 mmol) was added, and the mixture heated until most of the solids dissolved. This mixture was then cooled, additional t-BuOK (0.130 g, 1.17 mmol) was added, followed by p-toluidine (0.107 g, 0.997 mmol), the Pd catalyst/ligand mixture, and additional toluene/t-BuOH (5:1, 3.00 mL). The reaction was heated at 105° C. for 3 days, then cooled to room temperature, diluted with water (100 mL), and extracted with EtOAc (2×100 mL). The combined organic layers were washed with brine (50 mL), dried over Na₂SO₄, filtered, and the solvent was removed under vacuum. The resulting residue was purified over silica gel (4 g, CH₂Cl₂/MeOH) and the product was freeze-dried from MeCN/H₂O to provide the title compound (0.0212 g, 6%) as a yellow solid. ¹H NMR (300 MHz, DMSO-d₆): δ 11.71 (s, 1H), 8.54 (s, 1H), 8.06 (d, J=8.82 Hz, 2H), 7.18-6.99 (m, 6H), 6.67 (d, J=2.21 Hz, 1H), 6.47 (d, J=2.21 Hz, 1H), 3.88 (s, 3H), 3.84 (s, 3H), 2.27 (s, 3H). MS (ESI) m/z 388 [C₂₃H₂₁N₃O₃+H]⁺.

### Example 62

### Preparation of 5,7-Dimethoxy-2-(4-(pyridin-3-ylamino)phenyl)quinazolin-4(3H)-one

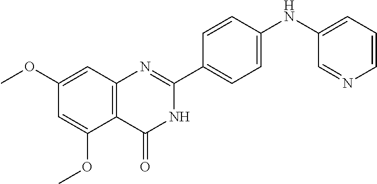 [SMILES: COc1cc(OC)c2c(=O)[nH]c(-c3ccc(Nc4cccnc4)cc3)nc2c1]

A mixture of 2-(4-bromophenyl)-5,7-dimethoxyquinazolin-4(3H)-one (0.200 g, 0.55 mmol), 3-aminopyridine (0.057 g, 0.61 mmol), Cs₂CO₃ (0.253 g, 0.776 mmol), Xantphos (0.002 g, 0.003 mmol), and Pd₂(dba)₃ (0.003 g, 0.003 mmol) in dioxane (2 mL) were combined in a microwave tube under nitrogen and irradiated at 300 W, 105° C. for 30 minutes. Then, DMF (1 mL) was added and the flask was irradiated for 1 hour at 300 W, 105° C. Then, the mixture was concentrated and purified by silica gel chromatography, eluting with 92:7:1 CHCl₃/MeOH/concentrated NH₄OH. The residue was further purified by reverse-phase HPLC, eluting with 10% to 90% CH₃CN in H₂O with 0.1% TFA, to afford the title compound (0.105 g, 51%) as a white solid. ¹H NMR (300 MHz, DMSO-d₆): δ 11.83 (s, 1H), 8.82 (s, 1H), 8.44 (d, J=2.4 Hz, 1H), 8.11-8.16 (m, 3H), 7.59-7.62 (m, 1H), 7.31-7.35 (m, 1H), 7.13 (d, J=8.7 Hz, 2H), 6.68 (d, J=1.8 Hz, 1H), 6.46 (d, J=1.8 Hz, 1H), 3.88 (s, 3H), 3.83 (s, 3H). APCI MS m/z 375 [M+H]⁺.

### Example 63

### Preparation of 4-(4-(5,7-Dimethoxy-4-oxo-3,4-dihydroquinazolin-2-yl)phenoxy)-N,N-dimethylpiperidine-1-carboxamide

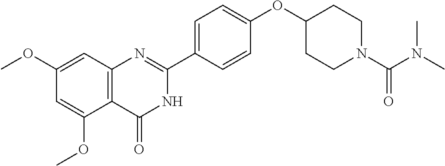

To a solution of 4-hydroxypiperidine (5.0 g, 49 mmol) in THF (70 mL) was added triethylamine (14.4 mL, 103 mmol) and dimethylcarbamyl chloride (9.0 mL, 98 mmol) slowly. The mixture was stirred at room temperature for 1.5 hours. The white precipitate was filtered off, washed with THF. The THF solution was concentrated to dryness then purified with column chromatography (SiO₂, MeOH/CH₂Cl₂=1:19) to afford 4-hydroxypiperidine-1-carboxylic acid dimethylamide as colorless oil. Yield: 7.8 g (94%).

4-Hydroxypiperidine-1-carboxylic acid dimethylamide (1.45 g, 8.40 mmol), 4-hydroxbenzenaldehyde (1.02 g, 8.40 mmol) and triphenylphosphine (3.31 g, 12.6 mmol) were stirred in THF (6 mL). Diisopropylazodicarboxylate (2.51 mL, 12.6 mmol) was added dropwise to the reaction mixture at room temperature over the course of 5 minutes. The mixture was stirred at room temperature for 21 hours, concentrated, and purified by column chromatography (SiO₂, hexanes/ethyl acetate=1:1 to neat ethyl acetate), to afford 4-(4-formylphenoxy)-piperidine-1-carboxylic acid dimethylamide a white solid. Yield: 0.7 g (30%).

To a 100 mL round-bottom flask was added 2-amino-4,6-dimethoxybenzamide (196 mg, 1.00 mmol), 4-(4-formylphenoxy)-piperidine-1-carboxylic acid dimethylamide (300 mg, 1.10 mmol), p-toluenesulfonic acid monohydrate (21 mg, 0.10 mmol), sodium hydrogensulfite (216 mg, 1.20 mmol) and dimethylacetamide (5 mL). The mixture was stirred at 115° C. under N₂ for 17 hours and cooled to room temperature. Water (20 mL) was added and stirred for 0.5 hours. The precipitate was filtered off, washed with water, and air dried. The crude product was purified by column chromatography (SiO₂, neat ethyl acetate, then ethyl acetate/methanol=19:1, then CH₂Cl₂/methanol=19:1) to afford the title compound as a white solid. Yield: 110 mg (24%). MP 248-249° C. ¹H NMR (400 MHz, DMSO-d₆): δ 11.91 (s, 1H), 8.15 (d, J=8.8 Hz, 2H), 7.10 (d, J=8.8 Hz, 2H), 6.72 (s, 1H), 6.51 (s, 1H), 4.71-4.69 (m, 1H), 3.89 (s, 3H), 3.85 (s, 3H), 3.44-3.39 (m, 2H), 3.06-2.99 (m, 2H), 2.74 (s, 6H), 2.00-1.96 (m, 2H), 1.64-1.59 (m, 2H).

### Example 64

### Preparation of 2-(4-(1-Acetylpiperidin-4-yloxy)phenyl)-5,7-dimethoxyquinazolin-4(3H)-one

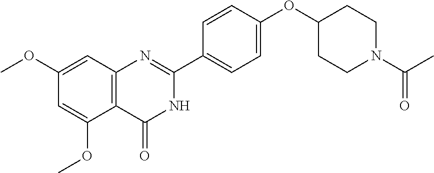 [SMILES: COc1cc(OC)c2c(=O)[nH]c(-c3ccc(OC4CCN(C(C)=O)CC4)cc3)nc2c1]

To a solution of 4-hydroxypiperidine (5.00 g, 49.4 mmol) in anhydrous THF (30 mL) and triethylamine (10 mL, 75 mmol) was added acetyl chloride (3.52 mL, 49.4 mmol). After the addition, the mixture was stirred for another 2 hours at room temperature. The solid formed was filtered and the mother liquid was concentrated to yield 5.0 g of crude product, which was purified by column chromatography on silica gel (230-400 mesh), using 5% methanol in dichloromethane as eluent, to give 1-(4-hydroxy-piperidin-1-yl)-ethanone. Yield: 2.40 g (34%).

To a solution of 1-(4-hydroxy-piperidin-1-yl)ethanone (1.00 g, 6.90 mmol), 4-hydroxybenzaldehyde (0.854 g, 6.90 mmol) and triphenylphosphine (1.83 g, 6.90 mmol) in THF (10 mL) was added dropwise diisopropyl azodicarboxylate (DIAD) (1.41 g, 6.90 mmol). The reaction mixture was stirred at room temperature for 16 hours, THF was evaporated, and the residue was purified by column chromatography, using dichloromethane:ethyl acetate:methanol (1:2:0.05) as eluent, to give 4-(1-acetyl-piperidin-4-yloxy)-benzaldehyde. Yield: 0.40 g (23%).

To a solution of 2-amino-4,6-dimethoxy-benzamide (0.20 g, 1.0 mmol) and 4-(1-acetyl-piperidin-4-yloxy)-benzaldehyde (0.25 g, 1.0 mmol) in N,N-dimethyl acetamide (5 mL), NaHSO₃ (0.20 g, 1.1 mmol) and p-TSA (20 mg, 0.10 mmol) were added and the reaction mixture was heated at 115° C. for 16 hours. The reaction mixture was cooled to room temperature. N,N-dimethylacetamide was removed under reduced pressure. The residue was diluted with water and the solid was collected; the crude product was purified by column chromatography on silica gel (230-400 mesh), using 5% methanol in CH₂Cl₂ as eluent, to give the title compound. Yield: 0.2 g (47%). MP 275-277° C. ¹H NMR (400 Hz, CDCl₃): δ 11.94 (s, 1H), 8.16 (d, 2H), 7.10 (d, 2H), 6.70 (d, 1H), 6.50 (d, 1H), 4.76 (m, 1H), 3.88 (s, 3H), 3.82 (s, 3H), 3.70 (m, 1H), 3.30 (m, 2H), 3.20 (m, 1H), 2.04 (s, 3H), 1.95 (m, 2H), 1.64 (m, 1H), 1.52 (m, 1H).

### Example 65

### Preparation of 2-(4-(2-(Isoindolin-2-yl)ethoxy)-3,5-dimethylphenyl)-5,7-dimethoxyquinazolin-4(3H)-one

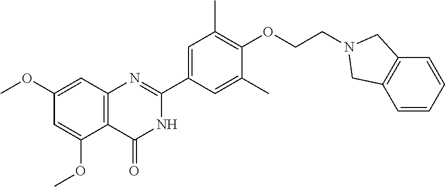 [SMILES: COc1cc(OC)c2c(=O)[nH]c(-c3cc(C)c(OCCN4Cc5ccccc5C4)c(C)c3)nc2c1]

To a suspension of 2-[4-(2-bromoethoxy)-3,5-dimethylphenyl]-5,7-dimethoxy-3H-quinazolin-4-one (0.50 g, 1.15 mmol) in anhydrous DMF (9 mL) was added isoindoline (0.41 mL, 3.46 mmol) and the reaction mixture was stirred at room temperature for 16 hours under nitrogen. The solvent was removed under reduced pressure and the residue was triturated with water (50 mL). The separated solid was filtered, washed with water and ether, and dried under vacuum to give the title compound as a white solid. Yield: 0.45 g (83%). MP 202-202.5° C. ¹H NMR (400 MHz, CDCl₃): δ 10.09 (br s, 1H), 7.77 (s, 2H), 7.22 (br s, 4H), 6.83 (d, J=2.4 Hz, 1H), 6.46 (d, J=2.4 Hz, 1H), 4.11 (s, 4H), 4.03 (t, J=6.0 Hz, 2H), 3.96 (s, 3H), 3.93 (s, 3H), 3.22 (t, J=6.0 Hz, 2H), 2.42 (s, 6H).

### Example 66

### Preparation of 2-(3,5-Dimethyl-4-(2-(pyrrolidin-1-yl)ethoxy)phenyl)-5-methoxyquinazolin-4(3H)-one

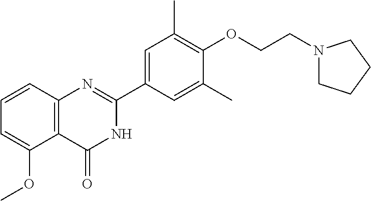 [SMILES: COc1cccc2nc(-c3cc(C)c(OCCN4CCCC4)c(C)c3)[nH]c(=O)c12]

To a stirred solution of 2-amino-6-methoxy-benzoicacid (3.00 g, 17.9 mmol) in THF (90 mL), EDCI (7.89 g, 41.1 mmol) and HOBt (7.95 g, 51.9 mmol) were added and stirred at room temperature for 30 minutes then N-methylmorpholine (6.15 g, 60.0 mmol) and aqueous 50% v/v NH₄OH (12 mL, 171.4 mmol) was added. The mixture was stirred for 16 hours at room temperature. The solvent was removed under reduced pressure and the residue was extracted with ethylacetate (4×100 mL), the combined organic phase was washed with water and brine, and dried over anhydrous sodium sulfate; the solvent was evaporated to give 2-amino-6-methoxy-benzamide as an off-white solid. Yield: 1.90 g, (65%).

To a solution of 2-amino-6-methoxy-benzamide (1.00 g, 6.01 mmol) and 4-(2-hydroxy-ethoxy)-3,5-dimethyl-benzaldehyde (1.28 g, 6.59 mmol) in N,N-dimethylacetamide (15 mL) were added NaHSO₃ (58.5 wt %, 0.68 g, 6.50 mmol) and p-TSA (0.23 g, 1.20 mmol) and the reaction mixture was heated at 115° C. for 20 hours, and cooled to room temperature. N,N-dimethylacetamide was removed under reduced pressure. The residue was diluted with water (50 mL), stirred for 30 minutes, and then filtered. The solid was suspended in dichloromethane (30 mL), stirred for 1 h, filtered, and dried under vacuum to give 2-[4-(2-hydroxy-ethoxy)-3,5-dimethyl-phenyl]-5-methoxy-3H-quinazolin-4-one as an off-white solid. Yield: 1.1 g (55%).

To a solution of 2-[4-(2-hydroxy-ethoxy)-3,5-dimethyl-phenyl]-5-methoxy3H-quinazolin-4-one (1.10 g, 3.20 mmol) in anhydrous N,N-dimethylformamide (16 mL) were added triphenylphosphine (0.92 g, 3.50 mmol) and carbontetrabromide (1.17 g, 3.50 mmol). The reaction mixture was stirred at room temperature for 16 hours. DMF was removed under reduced pressure. The residue was purified by column chromatography (silica gel 230-400 mesh; 3% methanol in dichloromethane as eluent) to give 2-[4(2-bromo-ethoxy)-3,5-dimethyl-phenyl]-5-methoxy3H-quinazolin-4-one as an off-white solid. Yield: 0.60 g (46%).

To a solution of 2-[4(2-bromo-ethoxy)-3,5-dimethyl-phenyl]-5-methoxy3H-quinazolin-4-one (0.50 g, 1.20 mmol) in N,N-dimethylformamide (10 mL) was added pyrrolidine (0.53 g, 7.40 mmol) and the reaction mixture was stirred at room temperature for 15 hours. DMF was removed under reduced pressure, the residue was purified by column chromatography (silica gel 230-400 mesh; 5% methanol in dichloromethane as eluent) to give the title compound as a white solid. Yield: 0.25 g (52%). MP 157-158° C. ¹H NMR (400 MHz, DMSO-d₆): δ 11.95 (s, 1H), 7.89 (s, 2H), 7.70 (t, J=8.19 Hz, 1H), 7.24 (d, J=7.8 Hz, 1H), 6.99 (d, J=8.1 Hz, 1H), 3.91-3.89 (m, 2H), 3.87 (s, 3H), 2.82 (t, J=6.2 Hz 2H), 2.53-2.50 (m, 4H), 2.30 (s, 6H), 1.69 (m, 4H). MS (ES⁺) m/z: 394.61 (M+1).

### Example 67

### Preparation of 5,7-Dichloro-2-(3,5-dimethyl-4-(2-(pyrrolidin-1-yl)ethoxy)phenyl)quinazolin-4(3H)-one

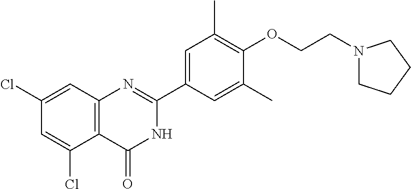 [SMILES: Cc1cc(-c2nc3cc(Cl)cc(Cl)c3c(=O)[nH]2)cc(C)c1OCCN1CCCC1]

To a solution of 2-amino-4,6-dichloro-benzoic acid (4.12 g, 20.0 mmol) in THF (120 mL) were added EDCI (4.22 g, 22.0 mmol), HOBt (2.70 g, 20.0 mmol) and N-methylmorpholine (2.22 g, 22.0 mmol). The reaction mixture was stirred at room temperature for 20 minutes, then 50% (v/v) aqueous NH₄OH solution (2.8 mL, 40.0 mmol) was added. The mixture was stirred for 20 hours at room temperature. The solvent was evaporated, the residue was taken in ethyl acetate (200 mL), and water (200 mL) was added. The organic phase was separated; the aqueous phase was extracted with ethyl acetate (200 mL). The combined organic phase was washed with water (100 mL), then brine (100 mL), and dried over anhydrous sodium sulfate. The solvent was evaporated and dried under vacuum to give 2-amino-4,6-dichloro-benzamide as an off-white solid. Yield: 3.83 g (93%).

To a solution of 2-amino-4,6-dichloro-benzamide (1.54 g, 7.50 mmol) and 4-(2-hydroxy-ethoxy)-3,5-dimethyl-benzaldehyde (1.46 g, 7.50 mmol) in N,N-dimethylacetamide (15 mL) were added sodium hydrogen sulfite (58.5 wt %, 1.51 g, 8.25 mmol) and p-toluenesulfonicacid monohydrate (0.28 g, 1.50 mmol). The reaction mixture was stirred at 120° C. for 16 hours under nitrogen, and then cooled to room temperature. Solvent was evaporated under reduced pressure. Water (100 mL) was added. The separated solid was filtered, washed with water (50 mL), and dried under vacuum. Crude compound was further washed with ether and dried under vacuum to give 5,7-dichloro-2-[4-(2-hydroxy-ethoxy)-3,5-dimethylphenyl]-3H-quinazolin-4-one as a white solid. Yield: 2.42 g (85%).

To a solution of 5,7-dichloro-2-[4-(2-hydroxy-ethoxy)-3,5-dimethylphenyl]-3H-quinazolin-4-one (1.14 g, 3.00 mmol) in anhydrous DMF (15 mL) was added carbon tetrabromide (1.10 g, 3.30 mmol). Then, triphenylphosphine (0.86 g, 3.30 mmol) was added in small portions. The reaction mixture was stirred at room temperature for 16 hours under nitrogen. Solvent was evaporated under reduced pressure. The residue was washed with ethyl acetate (50 mL) and dried under vacuum to give 2-[4-(2-bromo-ethoxy)-3,5-dimethylphenyl]-5,7-dichloro-3H-quinazolin-4-one as a white solid. Yield: 0.46 g (35%).

To a solution of 2-[4-(2-bromo-ethoxy)-3,5-dimethylphenyl]-5,7-dichloro-3H-quinazolin-4-one (0.44 g, 1.00 mmol) in anhydrous DMF (10 mL) was added pyrrolidine (0.28 g, 4.00 mmol). The reaction mixture was stirred at room temperature for 6 hours under nitrogen. Solvent was evaporated under reduced pressure. Water (50 mL) was added. The separated solid was filtered, washed with water (20 mL), and dried under vacuum. The crude compound was purified by the Simpliflash system (0-5% methanol in CH₂Cl₂ and 5% methanol (containing 7.0 M ammonia) in CH₂Cl₂ as eluent) to give the title compound as a white solid. Yield: 0.31 g (72%). MP 209-210° C. ¹H NMR (400 MHz, DMSO-d₆): δ 12.39 (br s, 1H), 7.90 (s, 2H), 7.71 (d, J=1.95 Hz, 1H), 7.60 (d, J=1.95 Hz, 1H), 3.91 (t, J=5.85 Hz, 2H), 2.83 (t, J=6.05 Hz, 2H), 2.55 (m, 4H), 2.31 (s, 6H), 2.01 (m, 4H). MS (ES+) m/z 432.54 (100%), 434.49 (90%).

### Example 68

### Preparation of 2-(4-(2-(4-Acetylpiperazin-1-yl)ethoxy)-3,5-dimethylphenyl)-5,7-dimethoxyquinazolin-4(3H)-one

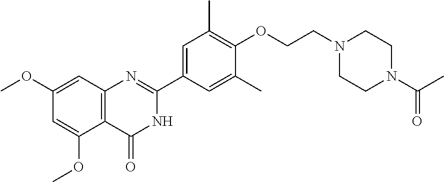 [SMILES: COc1cc(OC)c2c(=O)[nH]c(-c3cc(C)c(OCCN4CCN(C(C)=O)CC4)c(C)c3)nc2c1]

To a suspension of 2-[4-(2-bromoethoxy)-3,5-dimethylphenyl]-5,7-dimethoxy-3H-quinazolin-4-one (0.35 g, 0.81 mmol) in anhydrous DMF (9 mL) was added 1-acetylpyperazine (0.31 g, 2.42 mmol) and the reaction mixture was stirred at room temperature under nitrogen for 32 hours. Solvent was removed under reduced pressure and water (50 mL) was added. The separated solid was filtered, washed with water and ether, and dried under vacuum, to give the title compound as a white solid. Yield: 0.28 g (72%). MP 213-214° C. ¹H NMR (400 MHz, CDCl₃): δ 9.87 (br s, 1H), 7.74 (s, 2H), 6.83 (d, J=2.4 Hz, 1H), 6.46 (d, J=2.4 Hz, 1H), 3.97 (s, 3H), 3.95 (t, J=6.0 Hz, 2H), 3.93 (s, 3H), 3.69 (t, J=5.0 Hz, 2H), 3.53 (t, J=5.0 Hz, 2H), 2.84 (t, J=5.6 Hz, 2H), 2.62 (t, J=5.0 Hz, 2H), 2.57 (t, J=5.0 Hz, 2H), 2.39 (s, 6H), 2.11 (s, 3H). MS (ES⁻) m/z 479.65 (100%, M−1).

### Example 69

### Preparation of 2-(4-(2-(1H-Imidazol-1-yl)ethoxy)-3,5-dimethylphenyl)-5,7-dimethoxyquinazolin-4(3H)-one

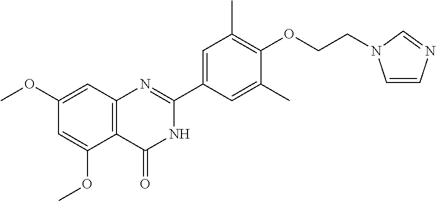 [SMILES: COc1cc(OC)c2c(=O)[nH]c(-c3cc(C)c(OCCn4ccnc4)c(C)c3)nc2c1]

To a solution of 2-[4-(2-bromoethoxy)-3,5-dimethylphenyl]-5,7-dimethoxy-3H-quinazolin-4-one (0.12 g, 0.27 mmol) in acetone (5 mL) was added imidazole (0.18 g, 2.70 mmol) and Cs₂CO₃ (0.26 g, 0.80 mmol). The reaction mixture was stirred at room temperature for 16 hours. Solvent was removed under reduced pressure, and the residue was purified by column chromatography (silica gel 230-400 mesh; 3% methanol in dichloromethane as eluent) to give the title compound as a white solid. Yield: 0.04 g (35%). MP 218-219° C. ¹H NMR (400 MHz, DMSO-d₆): δ 11.80 (br s, 1H), 7.83 (s, 2H), 7.72 (s, 1H), 7.29 (s, 1H), 6.92 (s, 1H), 6.70 (d, J=2.4 Hz, 1H), 6.49 (d, J=2.4 Hz, 1H), 4.36 (t, J=4.8 Hz, 2H), 4.02 (t, J=4.8 Hz, 2H), 3.86 (s, 3H), 3.81 (s, 3H), 2.06 (s, 6H). MS (ES) m/z: 419.57 (M−1).

### Example 70

### Preparation of 2-(3,5-Dimethyl-4-(2-(pyrrolidin-1-yl)ethoxy)phenyl)-7-methoxyquinazolin-4(3H)-one

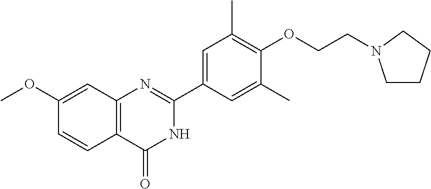 [SMILES: COc1ccc2c(=O)[nH]c(-c3cc(C)c(OCCN4CCCC4)c(C)c3)nc2c1]

To a stirred solution of 2-amino-4-methoxy-benzoic acid (3.00 g, 17.9 mmol) in THF (90 mL), EDCI (7.89 g, 41.1 mmol) and HOBt (7.95 g, 51.9 mmol) were added and stirred at room temperature for 30 minutes. Then, N-methylmorpholine (6.15 g, 60.0 mmol) and aqueous 50% (v/v) NH₄OH (12 mL, 171.4 mmol) were added. The mixture was stirred for 16 hours at room temperature. The solvent was removed under reduced pressure and the residue was extracted with ethyl acetate (4×100 mL). The combined organic phase was washed with water, then brine, and dried over anhydrous sodium sulfate. Solvent was evaporated to give 2-amino-4-methoxy-benzamide as an off-white solid. Yield: 1.80 g, (60%).

To a solution of 2-amino-4-methoxy-benzamide (1.00 g, 6.01 mmol) and 4-(2-hydroxy-ethoxy)-3,5-dimethyl-benzaldehyde (1.28 g, 6.59 mmol) in N,N-dimethylacetamide (15 mL) were added NaHSO₃ (58.5 wt %, 0.68 g, 6.50 mmol) and p-TSA (0.23 g, 1.20 mmol) and the reaction mixture was stirred at 115° C. for 16 hours, and cooled to room temperature. Solvent was removed under reduced pressure. The residue was diluted with water (50 mL), stirred for 30 minutes, and then filtered. The solid was suspended in dichloromethane (30 mL), stirred for 1 hour, filtered, and dried under vacuum, to give 2-[4-(2-hydroxy-ethoxy)-3,5-dimethyl-phenyl]-7-methoxy-3H-quinazolin-4-one as an off-white solid. Yield: 1.20 g (58%).

To a solution of 2-[4-(2-hydroxy-ethoxy)-3,5-dimethyl-phenyl]-7-methoxy-3H-quinazolin-4-one (1.20 g, 3.52 mmol) in anhydrous DMF (15 mL) were added triphenylphosphine (1.00 g, 3.80 mmol) and carbontetrabromide (1.27 g, 3.80 mmol). The reaction mixture was stirred at room temperature for 16 hours. DMF was removed under reduced pressure. The residue was purified by column chromatography (silica gel 230-400 mesh; 3% methanol in dichloromethane as eluent) to give 2-[4(2-bromo-ethoxy)-3,5-dimethyl-phenyl]-7-methoxy3H-quinazolin-4-one as an off-white solid. Yield: 0.37 g (26%).

To a solution of 2-[4-(2-bromo-ethoxy)-3,5-dimethyl-phenyl]-7-methoxy-3H-quinazolin-4-one (0.30 g, 0.74 mmol) in DMF (5 mL) was added pyrrolidine (0.31 g, 4.36 mmol) and the reaction mixture was stirred at room temperature for 15 hours. DMF was removed under reduced pressure, and the residue was purified by column chromatography (silica gel 230-400 mesh; 5% methanol in dichloromethane as eluent) to give the title compound as a white solid. Yield: 0.13 g (44%). MP 218-220° C. ¹H NMR (400 MHz, DMSO-d₆): δ 12.13 (br s, 1H), 8.03 (d, J=8.98 Hz, 1H), 7.90 (s, 2H), 7.16 (d, J=2.3 Hz, 1H), 7.07 (dd, J=8.9 and 2.7 Hz, 1H), 3.92-3.89 (m, 5H), 2.83 (t, J=5.8 Hz, 2H), 2.54-2.50 (m, 4H), 2.31 (s, 6H), 1.73 (m, 4H). MS (ES⁺) m/z: 394.62 (M+1).

### Example 71

### Preparation of 2-(3,5-Dimethyl-4-(2-(4-methylpiperazin-1-yl)ethoxy)phenyl)-5,7-dimethoxyquinazolin-4(3H)-one

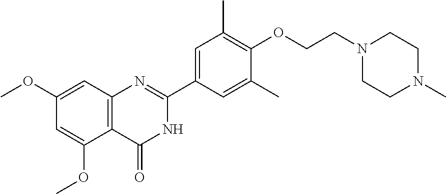

To a solution of 2-[4-(2-bromo-ethoxy)-3,5-dimethyl-phenyl]-5,7-dimethoxy-3H-quinazolin-4-one (0.17 g, 0.39 mmol) in N,N-dimethylformamide (0.5 mL) was added N-methylpiperazine (0.44 mL, 3.92 mmol) and the reaction mixture was stirred at room temperature for 15 hours. N,N-dimethylformamide was removed under reduced pressure. The residue was purified by column chromatography (silica gel 230-400 mesh; 5% methanol in dichloromethane as eluent) to give the title compound as a white solid. Yield: 60 mg (33.8%). MP 180-182° C. ¹H NMR (400 MHz, DMSO-d₆): δ 11.76 (s, 1H), 7.89 (s, 2H), 6.73 (d, J=2.4 Hz, 1H), 6.51 (d, J=2.4 Hz, 1H), 3.88 (m, 5H), 3.84 (s, 3H), 2.68 (t, J=5.6 Hz, 2H), 2.50 (br s, 4H), 2.32 (br s, 4H), 2.30 (s, 6H), 2.15 (s, 3H). MS (ES⁺) m/z: 453.21 (M+1).

### Example 72

### Preparation of 2-(3,5-Dimethyl-4-(2-(piperidin-1-yl)ethoxy)phenyl)-5,7-dimethoxyquinazolin-4(3H)-one

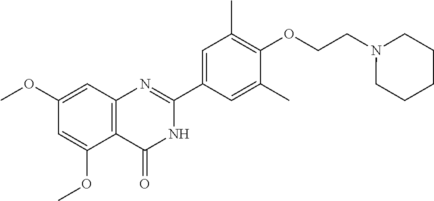 [SMILES: COc1cc(OC)c2c(=O)[nH]c(-c3cc(C)c(OCCN4CCCCC4)c(C)c3)nc2c1]

To a solution of 2-[4-(2-bromo-ethoxy)-3,5-dimethyl-phenyl]-5,7-dimethoxy-3H-quinazolin-4-one (0.34 g, 0.78 mmol) in DMF (10 mL) was added piperidine (0.27 g, 3.14 mmol). The reaction mixture was stirred at room temperature for 16 hours. Then, water was added and the product was extracted with ethyl acetate (2×200 mL). The combined organic layer was washed with water, then brine, and dried over anhydrous Na₂SO₄. Solvent was evaporated to give the title compound as a white solid. Yield: 0.33 g (96%). ¹H NMR (400 MHz, DMSO-d₆): δ 11.80 (br s, 1H), 7.87 (s, 2H), 6.72 (d, J=2.4 Hz, 1H), 6.49 (d, J=2.0 Hz, 1H), 3.86 (m, 6H), 3.82 (s, 2H), 2.63 (t, J=5.6 Hz, 2H), 2.42 (m, 4H), 2.28 (s, 6H), 1.50 (m, 4H), 1.37 (m, 2H). MS (ES) m/z 438.63 (M+1).

### Example 73

### Preparation of 5,7-Dimethoxy-2-(3-methyl-4-(2-(pyrrolidin-1-yl)ethoxy)phenyl)quinazolin-4(3H)-one

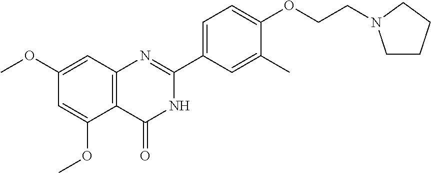 [SMILES: COc1cc(OC)c2c(=O)[nH]c(-c3ccc(OCCN4CCCC4)c(C)c3)nc2c1]

To a solution of 4-hydroxy-3-methylbenzaldehyde (1.10 g, 8.08 mmol) in anhydrous DMF (12 mL) was added K₂CO₃ (2.23 g, 16.16 mmol) and ethylene carbonate (1.42 g, 16.16 mmol) at room temperature. The resulting reddish-orange suspension was stirred at 110° C. for 6 hours under nitrogen. DMF was removed and the residue was diluted with water (50 mL) and dichloromethane (50 mL). The organic phase was separated, and the aqueous phase was extracted with dichloromethane (2×20 mL). The combined organic phase was washed with brine and dried over anhydrous magnesium sulfate. The solvent was removed under reduced pressure to obtain 4-(2-hydroxy-ethoxy)-3-methylbenzaldehyde as a brown oil. Yield: 1.46 g (100%).

To a solution of 4-(2-hydroxy-ethoxy)-3-methylbenzaldehyde (1.46 g, 8.08 mmol) and 2-amino-4,6-dimethoxybenzamide (1.58 g, 8.08 mmol) in N,N-dimethylacetamide (20 mL) were added NaHSO₃ (58.5 wt %, 2.20 g, 12.12 mmol) and p-toluenesulfonic acid monohydrate (0.38 g, 2.02 mmol). The reaction mixture was stirred at 110° C. for 16 hours, then cooled to room temperature. N,N-dimethylacetamide was removed under reduced pressure. The residue was triturated with water (50 mL). The resulting slurry was filtered and solid was washed with water, ether, and hexanes to obtain 2-[4-(2-hydroxy-ethoxy)-3-methyl-phenyl]-5,7-dimethoxy-3H-quinazolin-4-one as a beige solid. Yield: 2.75 g (95%).

Tetrabromomethane (3.26 g, 9.82 mmol) was added to a solution of triphenylphosphine (2.58 g, 9.82 mmol) in anhydrous DMF (20 mL) at 0° C. A solution of 2-[4-(2-hydroxy-ethoxy)-3-methyl-phenyl]-5,7-dimethoxy-3H-quinazolin-4-one (1.75 g, 4.91 mmol) in DMF (7 mL) was then added dropwise and stirred the reaction mixture at room temperature for 16 hours. The solvent was removed under reduced pressure and the residue was diluted with water (50 mL) and extracted with dichloromethane (4×25 mL). The combined organic phase was washed with brine and dried over anhydrous magnesium sulfate. The solvent was removed and the solid was triturated with ether. The resulting slurry was filtered and washed with ether several times (to remove the triphenylphosphine oxide) and finally with a solution of dichloromethane-ether (1:1) to obtain 2-[4-(2-bromo-ethoxy)-3-methyl-phenyl]-5,7-dimethoxy-3H-quinazolin-4-one as an off-white solid. Yield: 0.70 g (34%).

To a suspension of 2-[4-(2-bromo-ethoxy)-3-methyl-phenyl]-5,7-dimethoxy-3H-quinazolin-4-one (0.70 g, 1.67 mmol) in anhydrous DMF (9 mL) was added pyrrolidine (0.55 mL, 6.68 mmol) and the reaction mixture was stirred at room temperature under nitrogen for 20 hours. Solvent was removed under reduced pressure and the residue was purified by column chromatography (silica gel 230-400 mesh; 9% methanol in dichloromethane as eluent) to give the title compound as an off-white solid. Yield: 0.62 g (90.6%). MP 230-231° C. ¹H NMR (400 MHz, CDCl₃): δ 9.96 (br s, 1H), 7.91-7.89 (m, 2H), 6.93 (d, J=7.6 Hz, 1H), 6.82 (d, J=2.4 Hz, 1H), 6.44 (d, J=2.4 Hz, 1H), 4.21 (t, J=6.0 Hz, 2H), 3.98 (s, 3H), 3.93 (s, 3H), 2.98 (t, J=6.0 Hz, 2H), 2.69 (br s, 4H), 2.32 (s, 3H), 1.84-1.81 (m, 4H). MS (ES⁻) m/z 408.13 (M−1, 100%), MS (ES⁺) m/z 410.14 (M+1, 75%).

### Example 74

### Preparation of 3-(2-(4-(5,7-Dimethoxy-4-oxo-3,4-dihydroquinazolin-2-yl)-2,6-dimethylphenoxy)ethyl)-1-isopropylimidazolidine-2,4-dione

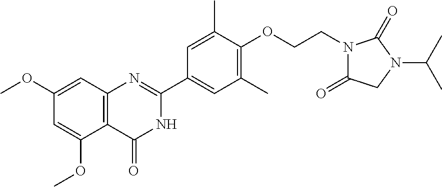 [SMILES: COc1cc(OC)c2c(=O)[nH]c(-c3cc(C)c(OCCN4C(=O)CN(C(C)C)C4=O)c(C)c3)nc2c1]

To a mixture of urea (5.00 g, 83.0 mmol) in anhydrous toluene (13 mL) was added chloroacetyl chloride (6.6 mL, 83.0 mmol) and the reaction mixture was heated to reflux for 2 hours. The reaction mixture was cooled to room temperature and toluene was removed by filtration. The resulting solid was further washed with toluene (10 mL) and mixed with water (100 mL). The solid was filtrated and washed with cold water (50 mL) and dried to give (2-chloroacetyl)-urea as a white solid. Yield: 10.3 g (91%).

(2-Chloroacetyl)-urea (0.68 g, 5.00 mmol) and isopropylamine (0.86 mL, 10.0 mmol) in DMF (10 mL) was stirred for 6 h at room temperature and then heated to 135° C. for 4 hours. DMF was removed under vacuum and the residue was purified by column chromatography (silica gel 230-400 mesh; eluting with hexane: dichloromethane: ethyl acetate 2.5:1.0:0.5) to give 1-isopropyl-imidazolidine-2,4-dione as a white solid. Yield: 0.20 g (28%).

To a solution of 1-isopropyl-imidazolidine-2,4-dione (0.10 g, 0.70 mmol) in N,N-dimethylformamide (5 mL) was added sodium hydride (60% in mineral oil, 31 mg, 0.77 mmol) and the reaction mixture was stirred for 10 minutes. Then, 2-[4-(2-bromo-ethoxy)-3,5-dimethyl-phenyl]-5,7-dimethoxy-3H-quinazolin-4-one (0.32 g, 0.73 mmol) was added. The reaction mixture was stirred at 55° C. for 16 hours, then poured into water (100 mL). The solid was filtered and dried. The crude compound was purified by column chromatography (silica gel 230-400 mesh; eluting with 2:1 ethyl acetate and dichloromethane) to give the title compound as a white solid. Yield: 0.09 g (26.0%). MP 219-221° C. ¹H NMR (400 MHz, DMSO): δ 9.64 (s, 1H), 7.69 (s, 2H), 6.82 (d, J=2.4 Hz, 1H), 6.45 (d, J=2.4 Hz, 1H), 4.42 (m, 1H), 4.02 (m, 2H), 3.98 (m, 2H), 3.96 (s, 3H), 3.92 (s, 3H), 3.85 (s, 2H), 2.32 (s, 6H) 1.22 (d, J=6.4 Hz, 6H). MS (ES⁺) m/z: 495.16 (M+1).

### Example 75

### Preparation of 2-(3,5-Dimethyl-4-(3-(pyrrolidin-1-yl)propoxy)phenyl)-5,7-dimethoxyquinazolin-4(3H)-one

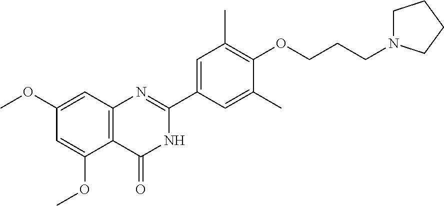 [SMILES: COc1cc(OC)c2c(=O)[nH]c(-c3cc(C)c(OCCCN4CCCC4)c(C)c3)nc2c1]

To a solution of 4-hydroxy-3,5-dimethyl benzaldehyde (5.0 g, 33.29 mmol) in DMF (30 mL) were added 3-bromo propan-1-ol (5.56 g, 39.95 mmol) and Cs₂CO₃ (16.24 g, 50.0 mmol). Then, the reaction mixture was stirred at room temperature for 48 hours. Then, water was added and the products were extracted with ethyl acetate (2×250 mL). The combined organic phase was washed with water (100 mL), then brine (100 mL), and dried over anhydrous Na₂SO₄. Removal of solvent gave 4-(3-hydroxypropoxy)-3,5-dimethyl benzaldehyde as a colorless liquid. Yield: 5.38 g (77%).

To a solution of 2-amino-4,6-dimethoxy-benzamide (1.3 g, 6.63 mmol) and 4-(3-hydroxypropoxy)-3,5-dimethyl benzaldehyde (1.38 g, 6.63 mmol) in N,N-dimethyl acetamide (10 mL), NaHSO₃ (1.30 g, 7.3 mmol), and p-TSA (252 mg, 1.32 mmol) were added and the reaction mixture was heated at 115° C. for 26 hours, then cooled to room temperature. The solvent was removed under reduced pressure. Then, water (100 mL) was added and stirred for 1 hour at room temperature. The separated solids were filtered and dried. The solids were again washed with diethyl ether to give crude product 2-[4-(3-hydroxy-propoxy)-3,5-dimethyl-phenyl]-5,7-dimethoxy-3H-quinazolin-4-one as an off-white solid. Yield: 1.69 g (66%).

To a solution of 2-[4-(3-hydroxy-propoxy)-3,5-dimethyl-phenyl]-5,7-dimethoxy-3H-quinazolin-4-one (1.39 g, 3.62 mmol) in DMF (15 mL) were added PPh₃ (1.04 g, 3.98 mmol) and CBr₄ (1.32 g, 3.98 mmol). The reaction mixture was stirred at room temperature for 16 hours. Then, solvent was removed under reduced pressure. The residue was triturated with ether and ethyl acetate. The solids were dried and purified by the Simpliflash system, using 2% methanol in CH₂Cl₂, to give 2-[4-(3-bromo-propoxy)-3,5-dimethyl-phenyl]-5,7-dimethoxy-3H-quinazolin-4-one as a white solid. Yield: 940 mg (58%).

To a solution of 2-[4-(3-bromo-propoxy)-3,5-dimethyl-phenyl]-5,7-dimethoxy-3H-quinazolin-4-one (340 mg, 0.76 mmol) in DMF (10 mL) was added pyrrolidine (433 mg, 6.08 mmol). Then, the reaction mixture was stirred at room temperature for 16 hours. Then, water was added and the solids were filtered. The solids were washed with water and dried to give the title compound as a white solid. Yield: 307 mg (92%). ¹H NMR (400 MHz, DMSO-d₆): δ 11.80 (s, 1H), 7.87 (s, 2H), 6.71 (d, J=2.0 Hz, 1H), 6.49 (d, J=2.0 Hz, 1H), 3.86 (s, 3H), 3.82 (m, 5H), 2.59 (t, J=6.8 Hz, 2H), 2.42 (m, 4H), 2.26 (s, 6H), 1.89 (m, 2H), 1.67 (m, 4H). MS (ES) m/z: 438.16 (M+1).

### Example 76

### Preparation of 5,7-Dimethoxy-2-(4-(2-(pyrrolidin-1-yl)ethoxy)phenyl)quinazolin-4(3H)-one

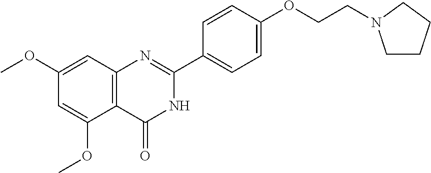 [SMILES: COc1cc(OC)c2c(=O)[nH]c(-c3ccc(OCCN4CCCC4)cc3)nc2c1]

Carbon tetrabromide (0.26 g, 0.77 mmol) was added to a solution of triphenylphosphine (0.24 g, 0.92 mmol) in anhydrous DMF (5 mL) at 0° C. A solution of 2-[4-(2-hydroxy-ethoxy)-phenyl]-5,7-dimethoxy-3H-quinazolin-4-one (0.21 g, 0.61 mmol) in DMF (2 mL) was then added dropwise and stirred at room temperature for 16 hours. Solvent was removed under reduced pressure and the residue was diluted with water (10 mL) and extracted with dichloromethane (4×10 mL). The combined organic phase was washed with brine and dried over anhydrous magnesium sulfate. Solvent was removed and the residual solid was triturated with ether. The resulting slurry was filtered and washed with ether several times (to remove the triphenylphosphine oxide) and finally with a solution of dichloromethane-ether (1:4) to obtain 2-[4-(2-bromo-ethoxy)-phenyl]-5,7-dimethoxy-3H-quinazolin-4-one as an off-white solid. Yield: 0.25 g (quantitative).

To a suspension of 2-[4-(2-bromo-ethoxy)-phenyl]-5,7-dimethoxy-3H-quinazolin-4-one (0.25 g, 0.61 mmol) in anhydrous DMF (10 mL) was added pyrrolidine (0.20 mL, 2.45 mmol) and the reaction mixture was stirred at room temperature under nitrogen for about 20 hours. Solvent was removed under reduced pressure and the residual solid was triturated with water. The resulting slurry was filtered and washed with ether and hexanes. The crude product was purified by column chromatography (silica gel 230-400 mesh; 10% methanol in dichloromethane as eluent) to give the title compound as a white solid. Yield: 0.11 g (44%). MP 226-227° C. ¹H NMR (400 MHz, CDCl₃): δ 10.08 (br s, 1H), 8.07 (d, J=8.4 Hz, 2H), 7.06 (d, J=8.8 Hz, 2H), 6.81 (d, J=1.95 Hz, 1H), 6.45 (d, J=1.95 Hz, 1H), 4.21 (t, J=5.6 Hz, 2H), 3.99 (s, 3H), 3.93 (s, 3H), 2.97 (t, J=5.6 Hz, 2H), 2.68 (br s, 4H), 1.84 (br s, 4H). MS (ES⁺): m/z 198.65 (100%), 396.10 (M+1, 70%).

### Example 77

### Preparation of 2-(3,5-Dimethyl-4-(3-(pyrrolidin-1-yl)propyl)phenyl)-5,7-dimethoxyquinazolin-4(3H)-one

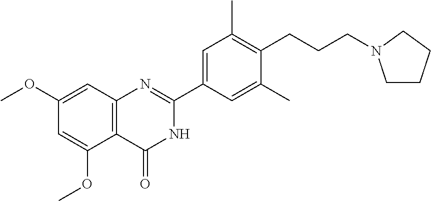 [SMILES: COc1cc(OC)c2c(=O)[nH]c(-c3cc(C)c(CCCN4CCCC4)c(C)c3)nc2c1]

To a solution of 2-amino-4,6-dimethoxy-benzamide (0.80 g, 4.00 mmol) and 4-(3-hydroxy-propyl)-3,5-dimethyl-benzaldehyde (0.98 g, 5.1 mmol) in N,N-dimethylacetamide (15 mL) were added NaHSO₃ (58.5 wt %, 0.80 g, 4.40 mmol) and p-TSA (0.155 g, 0.81 mmol) and the reaction mixture was heated at 115° C. for 16 hours, then cooled to room temperature. N,N-dimethylacetamide was removed under reduced pressure. The residue was diluted with water (50 mL), stirred for 30 minutes, and then filtered and washed with water. The crude compound was purified by column chromatography (silica gel 230-400 mesh; 5% methanol in dichloromethane as eluent) to give 2-[4-(3-hydroxy-propyl)-3,5-dimethyl-phenyl]-5,7-dimethoxy-3H-quinazolin-4-one as an off-white solid. Yield: 1.10 g (73%).

To a solution of 2-[4-(3-hydroxy-propyl)-3,5-dimethyl-phenyl]-5,7-dimethoxy-3H-quinazolin-4-one (1.00 g, 2.70 mmol) in anhydrous N,N-dimethylformamide (15 mL) were added triphenylphosphine (0.78 g, 3.00 mmol) and carbon tetrabromide (1.00 g, 3.00 mmol). The reaction mixture was stirred at room temperature for 16 hours. DMF was removed under reduced pressure. The residue was purified by column chromatography (silica gel 230-400 mesh; 3% methanol in dichloromethane as eluent) to give 2-[4-(3-bromo-propyl)-3,5-dimethyl-phenyl]-5,7-dimethoxy-3H-quinazolin-4-one as an off-white solid. Yield: 0.60 g (51%).

To a solution of 2-[4-(3-bromo-propyl)-3,5-dimethyl-phenyl]-5,7-dimethoxy-3H-quinazolin-4-one (0.40 g, 0.92 mmol) in N,N-dimethylformamide (10 mL) was added pyrrolidine (0.39 g, 5.52 mmol) and the reaction mixture was stirred at room temperature for 16 hours. DMF was removed under reduced pressure, the residue was purified by column chromatography (silica gel 230-400 mesh; 5% methanol ammonia in dichloromethane as eluent) to give the title compound as a white solid. Yield: 0.27 g (69%). MP 194-196° C. ¹H NMR (400 MHz, DMSO-d₆): δ 11.79 (br s, 1H), 7.81 (s, 2H), 6.72 (d, J=2.3 Hz, 1H), 6.50 (d, J=2.3 Hz, 1H), 4.00 (s, 3H), 3.87 (s, 3H), 2.67-2.63 (m, 2H), 2.49-2.46 (m, 6H), 2.33 (s, 6H), 1.70-1.67 (m, 4H), 1.59-1.53 (m, 2H). MS (ES⁺) m/z: 422.17 (M+1).

### Example 78

### Preparation of 2-(3,5-Dimethyl-4-(4-(pyrrolidin-1-yl)butoxy)phenyl)-5,7-dimethoxyquinazolin-4(3H)-one

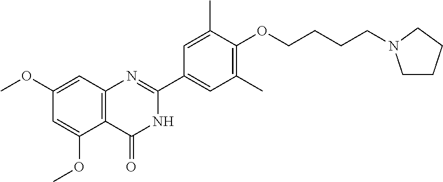 [SMILES: COc1cc(OC)c2c(=O)[nH]c(-c3cc(C)c(OCCCCN4CCCC4)c(C)c3)nc2c1]

To a solution of 4-hydroxy-3,5-dimethyl benzaldehyde (5.00 g, 33.3 mmol) in DMF (30 mL) were added 4-bromo-butan-1-ol (6.11 g, 39.9 mmol) and Cs₂CO₃ (16.2 g, 50.0 mmol). The reaction mixture was stirred at room temperature for 48 hours, then water (100 mL) was added, and the products were extracted with ethyl acetate (2×200 mL). The combined organic phase was washed with water (100 mL), then brine (100 mL), and dried over anhydrous Na₂SO₄. Solvent was removed and the crude product was purified by the Simpliflash system, using 40% ethyl acetate in hexane as eluent, to give 4-(4-hydroxybutoxy)-3,5-dimethyl benzaldehyde as a colorless liquid. Yield: 0.66 g (7%).

To a solution of 2-amino-4,6-dimethoxy-benzamide (497 mg, 2.53 mmol) and 4-(4-hydroxybutoxy)-3,5-dimethyl benzaldehyde (660 mg, 2.53 mmol) in N,N-dimethyl acetamide (10 mL), NaHSO₃ (58.5 wt %, 496 mg, 2.79 mmol) and p-TSA (96 mg, 0.50 mmol) were added and the reaction mixture was heated at 115° C. for 16 hours and then cooled to room temperature. The solvent was removed under reduced pressure. Water (100 mL) was added and stirred for 1 hour at room temperature. The solid separated was filtered and dried. The solid was further washed with diethyl ether to give product 2-[4-(4-hydroxy-butoxy)-3,5-dimethyl-phenyl]-5,7-dimethoxy-3H-quinazolin-4-one as a white solid. Yield: 1.69 g (82%).

To a solution of 2-[4-(4-hydroxy-butoxy)-3,5-dimethyl-phenyl]-5,7-dimethoxy-3H-quinazolin-4-one (675 mg, 1.69 mmol) in DMF (10 mL) were added PPh₃ (489 mg, 1.86 mmol) and CBr₄ (619 mg, 1.86 mmol). The reaction mixture was stirred at room temperature for 16 hours. Solvent was removed under reduced pressure. The residue was triturated with ether and ethyl acetate. The solid was dried and then purified by the Simpliflash system using 5% methanol in CH₂Cl₂ as the eluent to give 2-[4-(4-bromo-butoxy)-3,5-dimethyl-phenyl]-5,7-dimethoxy-3H-quinazolin-4-one as a white solid. Yield: 494 mg (63%).

To a solution of 2-[4-(4-bromo-butoxy)-3,5-dimethyl-phenyl]-5,7-dimethoxy-3H-quinazolin-4-one (494 mg, 1.07 mmol) in DMF (10 mL) was added pyrrolidine (609 mg, 8.57 mmol). The reaction mixture was stirred at room temperature for 16 hours. Water (100 mL) was added and the product was extracted with ethyl acetate (2×200 mL). The combined organic phase was washed with water, then brine, and dried over anhydrous Na₂SO₄. Solvent was evaporated to give the title compound as a white solid. Yield: 278 mg (57%). MP 180-181° C. ¹H NMR (400 MHz, CDCl₃): δ 7.68 (s, 2H), 6.83 (d, J=2.4 Hz, 1H), 6.46 (d, J=2.4 Hz, 1H), 3.97 (s, 3H), 3.92 (s, 3H), 3.83 (t, J=6.4 Hz, 2H), 2.56 (m, 6H), 2.36 (s, 6H), 1.88 (m, 2H), 1.79 (m, 6H). MS (ES) m/z: 452.21 (M+1).

### Example 79

### Preparation of 3-(2-(4-(5,7-Dimethoxy-4-oxo-3,4-dihydroquinazolin-2-yl)-2,6-dimethylphenoxy)ethyl)-5-phenylimidazolidine-2,4-dione

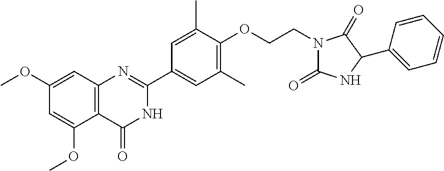 [SMILES: COc1cc(OC)c2c(=O)[nH]c(-c3cc(C)c(OCCN4C(=O)NC(c5ccccc5)C4=O)c(C)c3)nc2c1]

To a suspension of 2-[4-(2-hydroxy-ethoxy)-3,5-dimethyl-phenyl]-5,7-dimethoxy-3H-quinazolin-4-one (0.50 g, 1.35 mmol) in THF (20 mL), were added 5-phenyl-imidazolidine-2,4-dione (0.24 g, 1.35 mmol) and triphenyl phosphine (0.35 g, 1.35 mmol), then diethyl azodicarboxylate (0.43 mL, 2.70 mmol) was added and the reaction mixture was stirred at room temperature for 16 hours. Solvent was evaporated in vacuo and the residue was washed with dichloromethane and ether. The residue was dissolved in acetic acid and purified by preparative HPLC. The compound was further washed with dichloromethane and ether (1:1, 20 mL) to obtain the title compound as a white solid. Yield: 0.07 g (10%). MP 219.6-221.4° C. ¹H NMR (400 MHz, DMSO-d₆): δ 8.81 (s, 1H), 7.86 (s, 2H), 7.37 (m, 5H), 6.71 (s, 1H), 6.48 (s, 1H), 3.94 (m, 4H), 3.86 (s, 3H), 3.82 (s, 3H), 2.18 (s, 6H). MS (ES) m/z: 529.29 (M⁺+1).

### Example 80

### Preparation of 3-(4-(5,7-Dimethoxy-4-oxo-3,4-dihydroquinazolin-2-yl)benzyl)imidazolidine-2,4-dione

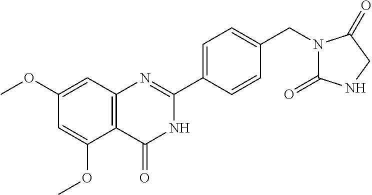 [SMILES: COc1cc(OC)c2c(=O)[nH]c(-c3ccc(CN4C(=O)CNC4=O)cc3)nc2c1]

Hydantoin (0.80 g, 8.00 mmol) was dissolved in DMF (10 mL) and cooled to 0° C. Sodium hydride (60% in mineral oil, 88 mg, 2.20 mmol) was added. The mixture was stirred at room temperature for 3 hours. 4-(Bromomethyl)benzaldehyde (0.40 g, 2.00 mmol) was added. The mixture was stirred at room temperature for 2.5 days. Saturated aqueous NH₄Cl (1 mL) was added. The mixture was concentrated to dryness. Water (10 mL) was added, extracted with dichloromethane, and the organic phase was dried over anhydrous Na₂SO₄. Solvent was removed and the crude compound was purified by column chromatography (silica gel 230-400 mesh; 5% methanol in CH₂Cl₂ as eluent) to give 4-(2,5-dioxo-imidazolidin-1-ylmethyl)-benzaldehyde as a white solid. Yield: 0.28 g (64%).

To a solution of 2-amino-4,6-dimethoxy-benzamide (0.19 g, 0.98 mmol) in N,N-dimethylacetamide (4 mL) were added 4-(2,5-dioxo-imidazolidin-1-ylmethyl)-benzaldehyde (0.19 g, 0.89 mmol), sodium hydrogen sulfite (58.5 wt %, 0.24 g, 1.30 mmol) and p-toluenesulfonic acid monohydrate (34 mg, 0.18 mmol) and the reaction mixture was stirred at 115° C. for 17 hours under nitrogen, then cooled to room temperature. The precipitate was filtered, washed with methanol, water, then methanol, and dried in air. The solid was suspended in hot DMSO (approximately 3 mL); saturated aqueous NaHCO₃ (approximately 3 mL) and water were added. The solid was filtered, washed with water, then methanol, and air dried to give the title compound as a light yellow solid. Yield: 0.16 g (46%). MP 317-318° C. ¹H NMR (400 MHz, DMSO-d₆): δ 12.05 (s, 1H), 8.17 (s, 1H), 8.12 (d, J=8.4 Hz, 2H), 7.40 (d, J=8.4 Hz, 2H), 6.74 (d, J=2.0 Hz, 1H), 6.54 (d, J=2.0 Hz, 1H), 4.61 (s, 2H), 4.02 (s, 2H), 3.89 (s, 3H), 3.85 (s, 3H). MS (ES⁺) m/z: 395.09 (M+1).

### Example 81

### Preparation of 2-(3,5-Dimethyl-4-(2-(pyrrolidin-1-yl)ethoxy)phenyl)-5,7-dimethoxypyrido[2,3-d]pyrimidin-4(3H)-one

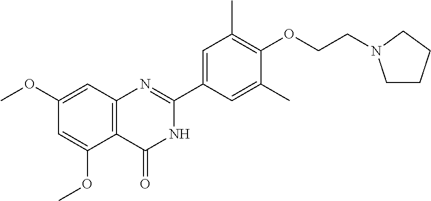 [SMILES: COc1cc(OC)c2c(=O)[nH]c(-c3cc(C)c(OCCN4CCCC4)c(C)c3)nc2c1]

To a solution of 2-amino-4,6-dimethoxy-nicotinamide (0.60 g, 3.00 mmol) and 4-(2-hydroxy-ethoxy)-3,5-dimethyl-benzaldehyde (0.59 g, 3.00 mmol) in N,N-dimethylacetamide (8 mL) was added NaHSO₃ (58.5 wt %, 0.59 g, 3.30 mmol) and p-TSA (0.22 g, 1.20 mmol). The reaction mixture was heated to 145-148° C. for 16 hours, then cooled to room temperature. N,N-dimethylacetamide was removed under reduced pressure, the residue was diluted with sodium bicarbonate solution (50 mL), and the solid separated was filtered and dried under vacuum. The crude compound was purified by column chromatography (silica gel 230-400 mesh; 5% methanol in dichloromethane as eluent) to give 2-[4-(2-hydroxy-ethoxy)-3,5-dimethyl-phenyl]-5,7-dimethoxy-3H-pyrido[2,3-d]pyrimidin-4-one as a white solid. Yield: 0.50 g (49%).

To a solution of 2-[4-(2-hydroxy-ethoxy)-3,5-dimethyl-phenyl]-5,7-dimethoxy-3H-pyrido[2,3-d]pyrimidin-4-one (0.50 g, 1.34 mmol) in anhydrous DMF (6 mL) was added carbon tetrabromide (0.53 g, 1.61 mmol) and triphenylphosphine (0.42 g, 1.61 mmol). The reaction mixture was stirred at 25° C. for 16 hours. DMF was removed under vacuum and dichloromethane (200 mL) was added. The organic phase was washed with water (100 mL), then brine (100 mL), and dried over anhydrous sodium sulfate. Solvent was removed and the residue was washed with ether (100 mL) to give 2-[4-(2-bromo-ethoxy)-3,5-dimethyl-phenyl]-5,7-dimethoxy-3H-pyrido[2,3-d]pyrimidin-4-one as a white solid. Yield: 0.23 g (40%).

A solution of 2-[4-(2-bromo-ethoxy)-3,5-dimethyl-phenyl]-5,7-dimethoxy-3H-pyrido[2,3-d]pyrimidin-4-one (0.20 g, 0.46 mmol) in pyrrolidine (2 mL) was stirred at room temperature for 3 hours. The excess pyrrolidine was removed under reduced pressure, and the residue was purified by column chromatography (silica gel 230-400 mesh; eluting with 2% 2.0 M ammonia in methanol solution and dichloromethane) to give the title compound as a white solid. Yield: 0.17 g (87%). MP 228-230° C. ¹H NMR (400 MHz, CDCl₃): δ 10.06 (s, 1H), 7.83 (s, 2H), 6.22 (s, 1H), 4.12 (s, 3H), 4.00 (s, 3H), 3.95 (t, J=6.0 Hz, 2H), 2.93 (t, J=6.0 Hz, 2H), 2.64 (m, 4H), 2.37 (s, 6H), 1.80 (m, 4H). MS (ES⁺) m/z: 425.19 (M+1).

### Example 82

### Preparation of 2-(3,5-Dimethyl-4-(2-(pyrrolidin-1-yl)ethoxy)phenyl)-7-fluoro-5-(pyrrolidin-1-yl)quinazolin-4(3H)-one

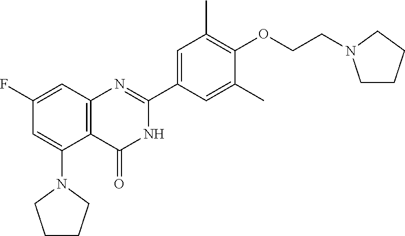 [SMILES: Cc1cc(-c2nc3cc(F)cc(N4CCCC4)c3c(=O)[nH]2)cc(C)c1OCCN1CCCC1]

A mixture of 2-amino-4,6-difluoro-benzamide (0.96 g, 5.60 mmol), 4-(2-hydroxy-ethoxy)-3,5-dimethyl-benzaldehyde (1.09 g, 5.60 mmol), NaHSO₃ (58.5 wt %, 1.00 g, 5.60 mmol) and p-toluenesulfonic acid monohydrate (1.44 g, 7.06 mmol) in N,N-dimethylacetamide (25 mL) was stirred at 120° C. for 16 hours, then cooled to room temperature. Solvent was removed under reduced pressure. The residue was diluted with water (100 mL). The solid separated was filtered and washed with water and dried under vacuum to give 5,7-difluoro-2-[4-(2-hydroxy-ethoxy)-3,5-dimethyl-phenyl]-3H-quinazolin-4-one as a white solid. Yield: 1.55 g (79%).

A mixture of 5,7-difluoro-2-[4-(2-hydroxy-ethoxy)-3,5-dimethyl-phenyl]-3H-quinazolin-4-one (1.54 g, 4.44 mmol), PPh₃ (1.52 g, 5.78 mmol), and CBr₄ (1.92 g, 5.78 mmol) in anhydrous DMF (30 mL) was stirred at room temperature for 36 hours. DMF was evaporated under vacuum, water (100 mL) was added, and stirred for 30 minutes. The solid separated was filtered, washed with water, then ether, and dried under vacuum to give 2-[4-(2-bromo-ethoxy)-3,5-dimethyl-phenyl]-5,7-difluoro-3H-quinazolin-4-one as pale yellow solid. Yield: 1.38 g (crude). This product was used in the next step without further purification.

A solution of 2-[4-(2-bromo-ethoxy)-3,5-dimethyl-phenyl]-5,7-difluoro-3H-quinazolin-4-one (1.38 g, crude) and pyrrolidine (10 mL) was stirred at room temperature for 16 hours. Excess pyrrolidine was evaporated, the residue was purified by column chromatography (silica gel 230-400 mesh; 30-50% ethyl acetate in hexanes as eluent). The compound was further purified by preparative HPLC to give the title compound as a white solid. Yield: 260 mg (13% for two steps). MP 206.6-206.8° C. ¹H NMR (400 MHz, DMSO-d₆): δ 11.85 (s, 1H), 6.63 (d, J=8 Hz, 1H), 6.51 (d, J=12 Hz, 1H), 3.90 (t, J=4 Hz, 2H), 2.83 (t, J=4 Hz, 2H), 2.50 (s, 6H), 2.30 (s, 4H), 1.89 (s, 4H), 1.70 (s, 4H).

### Example 83

### Preparation of 5-Chloro-2-(3,5-dimethyl-4-(2-(pyrrolidin-1-yl)ethoxy)phenyl)quinazolin-4(3H)-one

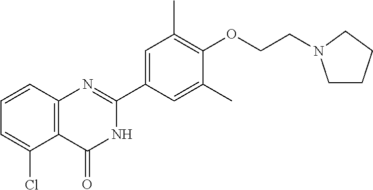 [SMILES: Cc1cc(-c2nc3cccc(Cl)c3c(=O)[nH]2)cc(C)c1OCCN1CCCC1]

To a solution of 2-amino-6-chlorobenzoic acid (2.00 g, 11.65 mmol) in anhydrous THF (20 mL) were added 4-methylmorpholine (1.40 mL, 12.82 mmol), HOBT (1.73 g, 12.82 mmol), and EDCI (2.45 g, 12.82 mmol); the reaction mixture was stirred at room temperature for 30 minutes. 50% (v/v) Ammonium hydroxide solution (10 mL, 132.0 mmol) was added and the mixture was stirred at room temperature for 23 hours. Solvent was evaporated to about 20 mL, poured into aqueous NaHCO₃ solution (200 mL) and extracted with ethyl acetate (7×100 mL). The organic phase was washed with water (3×100 mL), dried (Na₂SO₄), filtered, and evaporated, to give 2-amino-6-chlorobenzamide as a white solid. Yield: 1.65 g (83%).

4-(2-Hydroxyethoxy)-3,5-dimethylbenzaldehyde (0.70 g, 3.58 mmol), 2-amino-6-chlorobenzamide (0.60 g, 3.51 mmol), sodium bisulfite (0.71 g, 3.86 mmol) and p-toluenesulfonic acid monohydrate (0.133 g, 0.699 mmol) in anhydrous N,N-dimethyl acetamide (14 mL) were heated at 120° C. under nitrogen for 23 hours. The solvent was evaporated and the white solid was triturated with water (50 mL), filtered, and washed with water (20 mL). The solid was dried in vacuo and triturated with Et₂O (20 mL), filtered, and dried to give 5-chloro-2-(4-(2-hydroxyethoxy)-3,5-dimethylphenyl)quinazolin-4(3H)-one as a white solid. Yield: 0.77 g, (64%).

To a solution of 5-chloro-2-(4-(2-hydroxyethoxy)-3,5-dimethylphenyl)quinazolin-4(3H)-one (0.40 g, 1.16 mmol) in anhydrous DMF (10 mL) was added carbon tetrabromide (0.42 g, 1.27 mmol) and triphenylphoshine (0.33 g, 1.27 mmol). The reaction mixture was stirred at room temperature for 27 hours. Solvent was evaporated to dryness in vacuo and the residue triturated with Et₂O (15 mL)/EtOAc (15 mL) to give 2-(4-(2-bromoethoxy)-3,5-dimethylphenyl)-5-chloroquinazolin-4(3H)-one (0.42 g). It was used without further purification. The ¹H NMR indicated a purity of about 45%.

To a solution of 2-(4-(2-bromoethoxy)-3,5-dimethylphenyl)-5-chloroquinazolin-4(3H)-one (0.40 g, crude) in anhydrous DMF (10 mL) was added pyrrolidine (0.36 mL, 4.35 mmol) and the reaction mixture was stirred at room temperature, under nitrogen, for 25 hours. Solvent was evaporated to dryness in vacuo. The residue was triturated with water (50 mL), filtered, and the brown solid washed with Et₂O (20 mL). The crude material was purified by column chromatography (silica gel 230-400 mesh; 6% methanol in dichloromethane as the eluent) and then by reverse-phase HPLC (0.1% aqueous trifluoroacetic acid/acetonitrile as the eluent), to give a white solid. The solid was dissolved in CH₂Cl₂ (20 mL)/MeOH (4.5 mL), washed with 1 M Na₂CO₃ (4.5 mL) and the organic phase separated. The aqueous phase was extracted with CH₂Cl₂ (4×20 mL). The combined organic phase was washed with water (10 mL), dried (Na₂SO₄), filtered, and evaporated to give the title compound as a white solid. Yield: 0.091 g (21%, for two steps). MP 179-180° C. ¹H NMR (400 MHz, DMSO-d₆): δ 12.30 (br s, 1H), 7.89 (s, 2H), 7.77-7.66 (m, 1H), 7.66-7.60 (m, 1H), 7.47 (d, J=7.42 Hz, 1H), 3.89 (t, J=5.85 Hz, 2H), 2.80 (t, J=5.85 Hz, 2H), 2.53 (br s, 4H), 2.30 (s, 6H), 1.68 (br s, 4H). MS (ES+) m/z: 398.11 (100%), 400.13, 401.07.

### Example 84

### Preparation of 2-(4-(2-(Azepan-1-yl)ethoxy)-3,5-dimethylphenyl)-5,7-dimethoxyquinazolin-4(3H)-one

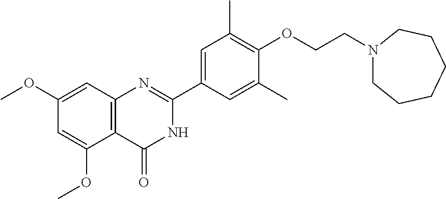 [SMILES: COc1cc(OC)c2c(=O)[nH]c(-c3cc(C)c(OCCN4CCCCCC4)c(C)c3)nc2c1]

To a suspension of 2-[4-(2-bromo-ethoxy)-3,5-dimethyl-phenyl]-5,7-dimethoxy-3H-quinazolin-4-one (0.22 g, 0.50 mmol) in DMF (2 mL) was added hexamethyleneimine (azepane) (0.22 mL, 2.0 mmol) and the reaction mixture was stirred at room temperature for 17 hours. Saturated aqueous NaHCO₃ solution (2 mL) was added and stirred for 2 hours. Water (10 mL) was added and stirred for another 0.5 hours. The solid was filtered, washed with water, and dried under vacuum to give the title compound as a white solid. Yield: 0.22 g (95%). MP 198-199° C. ¹H NMR (400 MHz, CD₃OD): δ 7.70 (s, 2H), 6.79 (s, 1H), 6.55 (s, 1H), 3.97 (t, J=6.0 Hz, 2H), 3.92 (s, 3H), 3.91 (s, 3H), 2.98 (t, J=6.0 Hz, 2H), 2.82 (t, J=5.2 Hz, 4H), 2.37 (s, 6H), 1.72 (m, 4H), 1.66 (m, 4H). MS (ES⁺) m/z: 452.27 (M+1). Analysis calculated for C₂₆H₃₃N₃O₄ (451.56), %: C, 69.16; H, 7.37; N, 9.31. Found, %: C, 68.94; H, 6.90; N, 9.30.

### Example 85

### Preparation of 2-(3,5-Dimethyl-4-(2-(pyrrolidin-1-yl)ethoxy)phenyl)-5,7-difluoroquinazolin-4(3H)-one

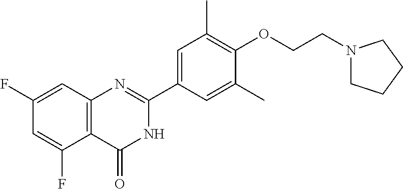 [SMILES: Cc1cc(-c2nc3cc(F)cc(F)c3c(=O)[nH]2)cc(C)c1OCCN1CCCC1]

To a solution of 2-amino-4,6-difluoro-benzamide (0.80 g, 4.60 mmol) and 3,5-dimethyl-4-(2-pyrrolidin-1-yl-ethoxy)-benzaldehyde (1.14 g, 4.60 mmol) in N,N-dimethylacetamide (60 mL) were added sodium hydrogen sulfite (58.5 wt %, 1.25 g, 6.9 mmol) and p-toluenesulfonic acid monohydrate (3.50 g, 18.4 mmol). The reaction mixture was stirred at 145° C. for 16 hours under nitrogen atmosphere, then cooled to room temperature. Solvent was evaporated under reduced pressure. Water (50 mL) was added, followed by saturated aqueous sodium bicarbonate solution (15 mL). The mixture was extracted with CH₂Cl₂ (2×100 mL) and washed with water. The organic phase was evaporated and the residue was washed with hexane/ether (90:10, 100 mL). The solid was filtered and dried under vacuum to give the title compound as a brown solid. Yield: 1.48 g (80%). MP 234-235° C. ¹H NMR (400 MHz, DMSO-d₆): δ 12.36 (s, 1H), 7.90 (s, 1H), 7.32 (m, 2H), 3.91 (t, J=4 Hz, 2H), 2.83 (t, J=4 Hz, 2H), 2.55 (s, 4H), 2.31 (s, 6H), 1.70 (s, 4H).

### Example 86

### Preparation of 2-(4-(2-(Azetidin-1-yl)ethoxy)-3,5-dimethylphenyl)-5,7-dimethoxyquinazolin-4(3H)-one

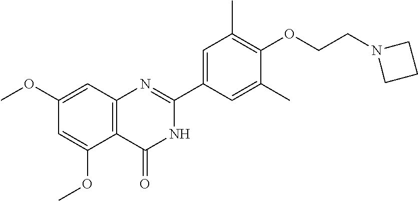 [SMILES: COc1cc(OC)c2c(=O)[nH]c(-c3cc(C)c(OCCN4CCC4)c(C)c3)nc2c1]

To a suspension of 2-[4-(2-bromoethoxy)-3,5-dimethyl-phenyl]-5,7-dimethoxy-3H-quinazolin-4-one (216 mg, 0.50 mmol) in DMF (5 mL) was added azetidine (154 mg, 2.70 mmol). The reaction mixture was stirred at room temperature for 2 days. The solid was collected by filtration, washed with methanol, ethyl acetate, and water, and dried under vacuum to give the title compound as a white solid. Yield: 58 mg (28%). MP 204-205° C. ¹H NMR (400 MHz, DMSO-d₆): δ 7.85 (s, 2H), 6.71 (d, J=2.4 Hz, 1H), 6.49 (d, J=2.4 Hz, 1H), 3.86 (s, 3H), 3.81 (s, 1H), 3.70 (t, J=6.0 Hz, 2H), 3.18 (t, J=6.8 Hz, 4H), 2.70 (t, J=6.0 Hz, 2H), 2.26 (s, 6H), 1.97 (m, 2H). MS (ES) m/z: 410.20 (M+1) (100%).

### Example 87

### Preparation of N-(1-(2-(4-(5,7-Dimethoxy-4-oxo-3,4-dihydroquinazolin-2-yl)-2,6-dimethylphenoxy)ethyl)azetidin-3-yl)acetamide

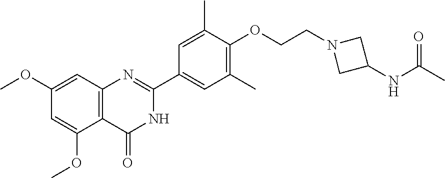 [SMILES: COc1cc(OC)c2c(=O)[nH]c(-c3cc(C)c(OCCN4CC(NC(C)=O)C4)c(C)c3)nc2c1]

To a solution of N-(1-benzhydryl-azetidin-3-yl)acetamide (1.00 g, 3.57 mmol) in ethanol (20 mL) were added palladium hydroxide on carbon (20 wt %, 0.20 g) and concentrated HCl (0.6 mL). The reaction mixture was hydrogenated at 50 psi at 40° C. for 2 hours. Then, the solid was filtered and washed with methanol (50 mL). The filtrate was collected; the solvent was evaporated to give N-azetidin-3-yl-acetamide as a green gummy material. Yield: 0.40 g (crude). This product was used in next step without further purification.

To a suspension of N-azetidin-3-yl-acetamide (0.30 g crude, 1.99 mmol) and 2-[4-(2-bromo-ethoxy)-3,5-dimethyl-phenyl]-5,7-dimethoxy-3H-quinazolin-4-one (0.43 g, 1.00 mmol) in anhydrous DMF (10 mL) was added triethylamine (3 mL). The reaction mixture was stirred at room temperature for 3 days under nitrogen. Solvent was evaporated under reduced pressure, water (50 mL) was added, and the precipitated solid was filtered. The aqueous phase was extracted with ethyl acetate (2×100 mL). The organic phase was dried over anhydrous Na₂SO₄. Solvent was evaporated, and crude compound was purified by the Simpliflash system (0-5% 7 N ammonia in methanol and CH₂Cl₂ as eluent) to give the title compound as a white solid. Yield: 0.30 g (63%). MP 111.8-113.6° C. ¹H NMR (400 MHz, CDCl₃): δ9.60 (br s, 1H), 7.69 (s, 2H), 6.82 (d, J=2.34 Hz, 1H), 6.46 (d, J=2.34 Hz, 1H), 6.10 (d, J=7.81 Hz, 1H), 4.71-4.44 (m, 1H), 3.97 (s, 3H), 3.93 (s, 3H), 3.85-3.67 (m, 4H), 3.26-3.13 (m, 2H), 2.90 (t, J=5.46 Hz, 2H), 2.36 (s, 6H), 2.01 (s, 3H). MS (ES⁺) m/z: 467.20 (M+1).

### Example 88

### Preparation of 2-(3,5-Dimethyl-4-(2-(pyrrolidin-1-yl)ethoxy)phenyl)-5,7-diisopropoxyquinazolin-4(3H)-one

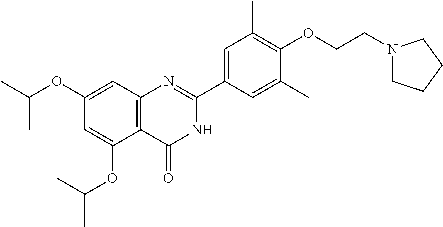 [SMILES: Cc1cc(-c2nc3cc(OC(C)C)cc(OC(C)C)c3c(=O)[nH]2)cc(C)c1OCCN1CCCC1]

To a solution of 2-[4-(2-hydroxy-ethoxy)-3,5-dimethyl-phenyl]-5,7-diisopropoxy-3H-quinazolin-4-one (0.73 g, 1.70 mmol) in DMF (10 mL) were added PPh₃ (0.49 g, 1.87 mmol) and CBr₄ (0.62 g, 1.87 mmol). The reaction mixture was stirred at room temperature for 16 hours. Then, solvent was removed under reduced pressure. The residue was triturated with ether and ethyl acetate. The solid was dried and purified by the Simpliflash system (2% methanol in CH₂Cl₂ as eluent) to give 2-[4-(2-bromo-ethoxy)-3,5-dimethyl-phenyl]-5,7-diisopropoxy-3H-quinazolin-4-one as a white solid.

Yield: 0.39 g (47%).

To a solution of 2-[4-(2-bromo-ethoxy)-3,5-dimethyl-phenyl]-5,7-diisopropoxy-3H-quinazolin-4-one (0.39 g, 0.79 mmol) in DMF (10 mL) was added pyrrolidine (0.45 g, 6.37 mmol). The reaction mixture was stirred at room temperature for 4 hours. Then, water was added and product was extracted with ethyl acetate (2×200 mL). The combined organic phase was washed with water, then brine, and dried over anhydrous Na₂SO₄. Solvent was evaporated to give the title compound as a white solid. Yield: 0.32 g (83%). MP 65-68° C. ¹H NMR (400 MHz, CDCl₃): δ 9.05 (br s, 1H), 7.63 (s, 2H), 6.78 (s, 1H), 6.42 (s, 1H), 4.70 (m, 1H), 4.63 (m, 1H), 3.94 (m, 2H), 2.94 (m, 2H), 2.64 (br s, 4H), 2.38 (s, 6H), 1.84 (m, 4H), 1.46 (m, 3H), 1.42 (m, 3H). MS (ES) m/z: 480.29 (M+1).

### Example 89

### Preparation of 2-(3,5-Dimethyl-4-(2-(pyrrolidin-1-yl)ethoxy)phenyl)-5,7-dimethylquinazolin-4(3H)-one

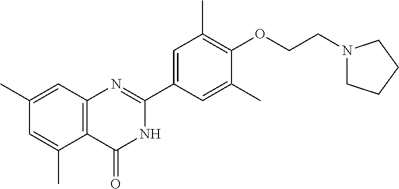 [SMILES: Cc1cc(C)c2c(=O)[nH]c(-c3cc(C)c(OCCN4CCCC4)c(C)c3)nc2c1]

Chloral hydrate (15.29 g, 92.42 mmol) was taken in water (335 mL). Sodium sulfate (78.14 g, 550.13 mmol) was added at room temperature. Then, a suspension of hydroxylamine hydrochloride (18.35 g, 264.06 mmol), 3,5-dimethylaniline (10.0 g, 82.52 mmol) and concentrated hydrochloric acid (36.5%, 10 mL) was added. The mixture was heated at 45° C. for 1.5 hours, then 75° C. for 1 hour. The reaction mixture was cooled to room temperature. The precipitated brown solid was filtered and washed with cold water (50 mL) and hexane (50 mL). The crude compound was dried under vacuum to give N-(3,5-dimethyl-phenyl)-2-hydroxyimino-acetamide as a brown solid. Yield: 13.7 g (86%). The crude compound was used in the next step without further purification.

N-(3,5-Dimethyl-phenyl)-2-hydroxyimino-acetamide (13.7 g, 71.3 mmol) was added to concentrated sulfuric acid (70 mL) in a 250 mL flask. The reaction mixture was then heated at 80° C. for 30 minutes, then cooled to room temperature, and poured into ice-water (200 mL). The precipitated solid was filtered and washed with water (100 mL) and dried under vacuum to give 4,6-dimethyl-1H-indole-2,3-dione as an orange solid. Yield: 5.53 g (44%).

To a heated (70° C. bath temperature) deep red solution of 4,6-dimethyl-1H-indole-2,3-dione (1.00 g, 5.71 mmol) in 33% aqueous sodium hydroxide (35 mL) was added 35% hydrogen peroxide (3.33 g, 34.3 mmol) over a period of 5 minutes. The reaction mixture was heated for another 15 min, then cooled to room temperature, and ice was added. The pH was adjusted to approximately 8 with concentrated HCl at 0° C. and acidified further to pH approximately 6 with glacial acetic acid. The solid precipitated was filtered, washed well with cold water, and dried under vacuum at 40° C. overnight to obtain 2-amino-4,6-dimethyl-benzoic acid as a pale brown solid. Yield: 0.35 g (37%).

To a solution of 2-amino-4,6-dimethyl-benzoic acid (0.35 g, 2.08 mmol) in anhydrous THF (10 mL) was added EDCI (0.80 g, 4.17 mmol), HOBt (0.80 g, 5.22 mmol) and N-methyl-morpholine (0.7 mL, 6.24 mmol). The reaction mixture was stirred at room temperature for 30 minutes, then ammonium hydroxide (50% v/v, 2.5 mL) was added. The mixture was stirred at room temperature for 17 hours. The solvent was removed under reduced pressure. Water (50 mL) was added, and the mixture was extracted with dichloromethane (2×100 mL). The combined organic phase was washed with water, and dried over anhydrous Na₂SO₄. Removal of the solvent gave the crude product. The crude product was purified by column chromatography (silica gel 230-400 mesh; 3% methanol in dichloromethane as eluent) to give 2-amino-4,6-dimethyl-benzamide. Yield: 0.20 g (59%).

To a solution of 2-amino-4,6-dimethyl-benzamide (0.20 g, 1.22 mmol) and 3,5-dimethyl-4-(2-pyrrolidin-1-yl-ethoxy)-benzaldehyde (0.36 g, 1.46 mmol) in N,N-dimethylacetamide (10 mL) was added NaHSO₃ (58.5 wt %, 0.55 g, 3.05 mmol) and p-TSA (0.46 g, 2.44 mmol). The reaction mixture was heated to 110° C. for 2 hours, then cooled to room temperature. N,N-dimethylacetamide was removed under reduced pressure, the residue was diluted with sodium bicarbonate solution (50 mL), and the solid separated was filtered and dried under vacuum. The crude compound was purified by column chromatography (silica gel 230-400 mesh; 6% methanol in dichloromethane as eluent) to give the title compound as a white solid. Yield: 0.145 g (30%). MP 181-182° C. ¹H NMR (400 MHz, DMSO-d₆): δ 10.62 (s, 1H), 7.75 (s, 2H), 7.44 (s, 1H), 7.03 (s, 1H), 3.95 (t, J=6.0 Hz, 2H), 2.94 (t, J=6.0 Hz, 2H), 2.85 (s, 3H), 2.65 (s, 4H), 2.44 (s, 3H), 2.39 (s, 6H), 1.84 (s, 4H). MS (ES⁺) m/z: 392.13 (M+1).

### Example 90

### Preparation of 2-(2-(4-(6,8-Dimethoxy-1-oxo-1,2-dihydroisoquinolin-3-yl)-2,6-dimethylphenoxy)ethyl)isoindoline-1,3-dione

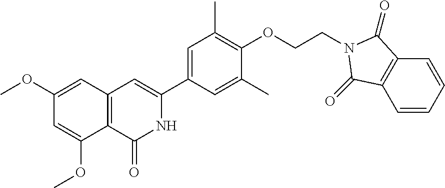 [SMILES: COc1cc(OC)c2c(=O)[nH]c(-c3cc(C)c(OCCN4C(=O)c5ccccc5C4=O)c(C)c3)cc2c1]

To a suspension of 3-[4-(2-hydroxy-ethoxy)-3,5-dimethyl-phenyl]-6,8-dimethoxy-2H-isoquinolin-1-one (0.80 g, 2.16 mmol), isoindole-1,3-dione (0.35 g, 2.38 mmol), and triphenylphosphine (0.85 g, 3.25 mmol) in THF (30 mL), was added diethyl azodicarboxylate (0.56 g, 3.25 mmol) and the reaction mixture was stirred at room temperature for 16 hours. The solvent was evaporated in vacuo and the residue was washed with ether to give the title compound as an off-white solid. Yield: 1.11 g (crude). ¹H NMR (400 MHz, CDCl₃): δ 8.34 (s, 1H), 7.89 (m, 2H), 7.77 (m, 2H), 7.21 (s, 2H), 6.49 (br s, 2H), 6.44 (s, 1H), 4.16 (m, 2H), 4.08 (m, 2H), 3.97 (s, 3H), 3.89 (s, 3H), 2.25 (s, 6H). MS (ES) m/z: 499.06 (M+1) (100%).

### Example 91

### Preparation of 2-(3,5-Dimethyl-4-(2-(pyrrolidin-1-yl)ethoxy)phenyl)-5,7-diisopropoxypyrido[2,3-d]pyrimidin-4(3H)-one

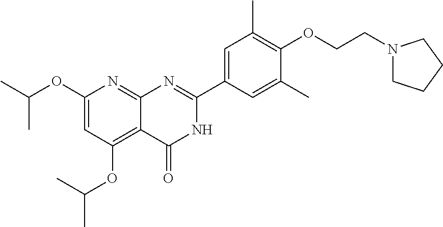 [SMILES: Cc1cc(-c2nc3nc(OC(C)C)cc(OC(C)C)c3c(=O)[nH]2)cc(C)c1OCCN1CCCC1]

To a suspension of 2-amino-4-hydroxy-6-oxo-1,6-dihydro-pyridine-3-carboxylic acid methyl ester (7.0 g, 38.04 mmol), 2-iodopropane (14.22 g, 83.69 mmol), and K₂CO₃ (11.56 g, 83.69 mmol) in DMF (200 mL), was heated at 60° C. for 48 hours, then cooled to the room temperature and filtered. Water (400 mL) was added to the filtrate and the product was extracted with ethyl acetate (3×200 mL). The combined organic layer was washed with water, then brine, dried over Na₂SO₄, and evaporated to give crude product. The crude product was purified by Simpliflash, using 10% ethyl acetate in hexane, to give 2-amino-4,6-diisopropoxy-nicotinic acid methyl ester as a colorless oil. Yield: 1.30 g (13%). ¹H NMR (400 MHz, DMSO-d₆): δ 6.91 (s, 2H), 5.57 (s, 1H), 5.19 (m, 1H), 4.59 (m, 1H), 3.66 (s, 3H), 1.23 (d, J=2.0 Hz, 6H), 1.21 (d, J=1.2 Hz, 6H).

To the solution of 2-amino-4,6-diisopropoxy-nicotinic acid methyl ester (1.6 g, 5.97 mmol) in methanol (9.0 mL) and water (1.0 mL), was added lithium hydroxide (750 mg, 17.91 mmol). The reaction mixture was heated to 50° C. for 8 hours. The solvent was removed; the residue was diluted with water and neutralized with 2 N HCl. The product was extracted with ethyl acetate (3×100 mL). The combined organic layer was washed with water, then brine, dried over Na₂SO₄, and evaporated, to give crude 2-amino-4,6-diisopropoxy-nicotinic acid as a light yellow solid. Yield: 1.48 g (98%, crude).

To a solution of 2-amino-4,6-diisopropoxy-nicotinic acid (1.48 g, 5.83 mmol) in THF (30 mL) were added EDCI (1.34 g, 6.99 mmol), HOBt (0.94 g, 6.99 mmol), NMM (0.70 g, 6.99 mmol) and liquid NH₃ (10 mL). Then, the reaction mixture was stirred at room temperature for 24 hours. Then, water (100 mL) was added and the products were extracted with ethyl acetate (2×200 mL). The combined organic phase was washed with water, then brine, and dried over anhydrous Na₂SO₄. Removal of solvent gave crude 2-amino-4,6-diisopropoxy-nicotinamide as a yellow oil. Yield: 450 mg (26%, crude).

To a solution of 2-amino-4,6-diisopropoxy-nicotinamide (450 mg, 1.78 mmol) and 3,5-dimethyl-4-(2-pyrrolidin-1-yl-ethoxy)-benzaldehyde (440 mg, 1.78 mmol) in N,N-dimethyl acetamide (10 mL) were added NaHSO₃ (790 mg, 4.44 mmol) and p-TSA (845 mg, 4.44 mmol). The reaction mixture was heated at 120° C. for 16 hours, then cooled to room temperature. The solvent was removed under reduced pressure. Then, water (100 mL) was added and stirred for 30 min at room temperature. The separated solids were filtered and dried to give crude product, which was purified by the Simpliflash system, using 2% methanol in dichloromethane, to give a yellow oil, which dissolved in ether. 2N HCl in ether was added, and the separated solids were filtered and dried to give the hydrochloride salt of the title compound as a yellow solid. Yield: 59 mg (6%). ¹H NMR (400 MHz, DMSO-d₆): δ 10.7 (br s, 1H), 7.88 (s, 2H), 6.31 (s, 1H), 5.41 (m, 1H), 4.80 (m, 1H), 4.14 (t, J=4.8 Hz, 2H), 3.61 (m, 2H), 3.16 (m, 4H), 2.34 (s, 6H), 2.03 (m, 2H), 1.91 (m, 2H), 1.32 (s, 6H), 1.30 (s, 6H). MS (ES) m/z: 481.18 (M+1).

### Example 92

### Preparation of (S)-2-(3,5-Dimethyl-4-((5-oxopyrrolidin-2-yl)methoxy)phenyl)-5,7-dimethoxyquinazolin-4(3H)-one

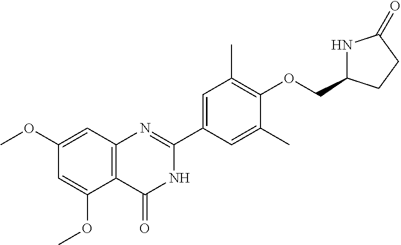 [SMILES: COc1cc(OC)c2c(=O)[nH]c(-c3cc(C)c(OC[C@@H]4CCC(=O)N4)c(C)c3)nc2c1]

To a solution of (S)-5-(hydroxymethyl)pyrrolidin-2-one (3.85 g, 33.5 mmol) in acetonitrile (60 mL) under nitrogen was added PPh₃ (9.16 g, 34.8 mmol). The mixture was cooled to 0° C. and CBr₄ (11.55 g, 34.8 mmol) added dropwise as a solution in acetonitrile (40 mL) over 15 minutes. Then, the reaction mixture was warmed to room temperature and stirred for 18 hours. The mixture was then concentrated and heptane (100 mL) and water (100 mL) added. After stirring for 1 hour, the solids were filtered and washed with 1:1 heptane/water (100 mL). The filtrate layers were separated and the aqueous layer extracted with Et₂O (2×100 mL) and CHCl₃ (2×100 mL). The combined organic phase was dried over anhydrous Na₂SO₄, filtered, concentrated, and purified by silica gel chromatography, eluting with 100% CHCl₃ to 10% MeOH/CHCl₃, to afford (S)-5-(bromomethyl)pyrrolidin-2-one as a white solid (3.15 g, 53%).

To a solution of 4-hydroxy-3,5-dimethylbenzaldehyde (2.65 g, 17.7 mmol) in DMF (100 mL) was added K₂CO₃ (3.66 g, 26.6 mmol). The mixture was stirred at room temperature under nitrogen for 30 minutes. Then, a solution of (S)-5-(bromomethyl)pyrrolidin-2-one (3.15 g, 17.7 mmol) in DMF (100 mL) was added, and the mixture heated at reflux for 16 hours. The mixture was then concentrated, ethyl acetate (250 mL) added, and the organic phase washed sequentially with water (2×150 mL), and brine (200 mL), dried (Na₂SO₄), filtered, and concentrated. The residue was purified by silica gel chromatography, eluting with 100% ethyl acetate to 10% MeOH/ethyl acetate, followed by a second chromatography, eluting with 1:1 CH₂Cl₂/92:71 CHCl₃/MeOH/concentrated NH₄OH to 100% 92:7:1 CHCl₃/MeOH/concentrated NH₄OH, to afford (S)-3,5-dimethyl-4-((5-oxopyrrolidin-2-yl)methoxy)benzaldehyde as a white solid (0.200 g, 5%).

A mixture of (S)-3,5-dimethyl-4-((5-oxopyrrolidin-2-yl)methoxy)benzaldehyde (0.200 g, 0.81 mmol), 2-amino-4,6-dimethoxybenzamide (0.159 g, 0.81 mmol), NaHSO₃ (0.093 g, 0.89 mmol), and p-TsOH (0.015 g, 0.08 mmol) in DMA (10 mL) was heated at 150° C. for 48 hours. The reaction mixture was cooled to room temperature, diluted with ethyl acetate (200 mL), washed with water (2×200 mL), dried over anhydrous Na₂SO₄, filtered, and concentrated. The residue was purified by flash chromatography on silica gel, eluting with 1:1 CH₂Cl₂/92:7:1 CHCl₃/MeOH/concentrated NH₄OH to 100% 92:7:1 CHCl₃/MeOH/concentrated NH₄OH to 6:3:1 CHCl₃/MeOH/concentrated NH₄OH, to afford the title compound as an off-white solid (0.108 g, 31%). ¹H NMR (300 MHz, DMSO-d₆): δ 11.85 (s, 1H), 7.79-7.91 (m, 3H), 6.74 (d, J=2.2 Hz, 1H), 6.52 (d, J=2.2 Hz, 1H), 3.88-3.94 (m, 4H), 3.84 (s, 3H), 3.63-3.75 (m, 2H), 2.30 (s, 6H), 2.09-2.27 (m, 3H), 1.91-2.00 (m, 1H). APCI MS m/z 424 [M+H]⁺.

### Example 93

### Preparation of 2-(4-((4-Isopropylpiperazin-1-yl)methyl)phenyl)-5,7-dimethoxyquinazolin-4(3H)-one

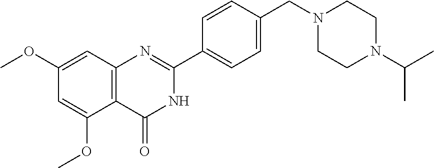 [SMILES: COc1cc(OC)c2c(=O)[nH]c(-c3ccc(CN4CCN(C(C)C)CC4)cc3)nc2c1]

To a mixture of 4-(bromoethyl) benzaldehyde (0.200 g, 1.0 mmol) and K₂CO₃ (0.277 g, 2.0 mmol) in DMF (5 mL) was added N-isopropylpiperazine (0.129 g, 1.0 mmol) and the reaction was stirred at room temperature for 5 hours, then concentrated in vacuo. The resulting mixture was diluted with H₂O and extracted with EtOAc. The organics were washed with brine, dried over anhydrous Na₂SO₄, filtered, and concentrated in vacuo to afford 4-((4-Isopropylpiperazin-1-yl)methyl)benzaldehyde (0.240 g, 97%).

A mixture of 4-((4-isopropylpiperazin-1-yl)methyl)benzaldehyde (0.240 g, 0.97 mmol), NaHSO₃ (0.155 g, 1.50 mmol), and p-TsOH (0.019 g, 0.10 mmol) was added to a solution of 2-amino-4,6-dimethoxybenzamide (0.190 g, 0.97 mmol) in DMA (7 mL). The reaction was stirred at 130° C. overnight. Then, the mixture was diluted with H₂O and extracted with CH₂Cl₂. The organics were washed with brine, dried over anhydrous Na₂SO₄, filtered, and concentrated in vacuo. Purification by flash chromatography on silica gel, eluting with 2% to 10% MeOH/CH₂Cl₂, afforded the title compound (0.122 g, 30%) as a light yellow solid. ¹H NMR (300 MHz, DMSO-d₆): δ 12.02 (s, 1H), 8.12 (d, J=8.0 Hz, 2H), 7.43 (d, J=8.0 Hz, 2H), 6.74 (s, 1H), 6.53 (s, 1H), 3.89 (s, 3H), 3.85 (s, 3H), 3.51 (s, 2H), 2.54-2.71 (m, 1H), 2.27-2.44 (m, 8H), 0.95 (d, J=6.4 Hz, 6H). ESI MS m/z 423 [M+H]⁺.

### Example 94

### Preparation of N-(1-(4-(5,7-Dimethoxy-4-oxo-3,4-dihydroquinazolin-2-yl)benzyl)piperidin-4-yl)-N-isopropylacetamide

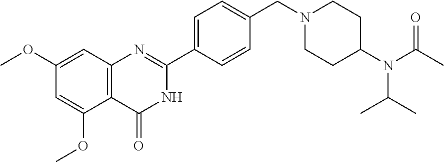 [SMILES: COc1cc(OC)c2c(=O)[nH]c(-c3ccc(CN4CCC(N(C(C)=O)C(C)C)CC4)cc3)nc2c1]

To a mixture of 4-(bromoethyl) benzaldehyde (0.840 g, 4.2 mmol) and K₂CO₃ (1.75 g, 12.6 mmol) in DMF (15 mL) was added N-isopropyl-N-(piperidin-4-yl)acetamide (0.92 g, 4.2 mmol) and the reaction was stirred at room temperature 5 hours, then concentrated in vacuo. The resulting mixture was diluted with H₂O and extracted with EtOAc. The organics were washed with brine, dried over anhydrous Na₂SO₄, filtered, and concentrated in vacuo. Purification by flash chromatography on silica gel, eluting with 1% to 10% MeOH/CH₂Cl₂, afforded N-(1-(4-formylbenzyl)piperidin-4-yl)-N-isopropylacetamide (0.770 g, 61%).

A mixture of N-(1-(4-formylbenzyl)piperidin-4-yl)-N-isopropylacetamide (0.770 g, 2.5 mmol), NaHSO₃ (0.350 g, 3.3 mmol), and p-TsOH (0.100 g, 0.51 mmol) was added to a solution of 2-amino-4,6-dimethoxybenzamide (0.500 g, 2.5 mmol) in DMA (20 mL). The reaction was stirred at 130° C. for 5 hours and concentrated in vacuo. The residue was diluted with H₂O and saturated NaHCO₃, then extracted with CH₂Cl₂. The organics were washed with brine, dried over anhydrous Na₂SO₄, filtered, and concentrated in vacuo. Purification by flash chromatography on silica gel, eluting with 1% to 10% MeOH/CH₂Cl₂, afforded the title compound (0.670 g, 56%) as a light yellow solid. ¹H NMR (300 MHz, DMSO-d₆): δ 12.02 (s, 1H), 8.13 (d, J=8.1 Hz, 2H), 7.43 (d, J=8.0 Hz, 2H), 6.74 (d, J=1.9 Hz, 1H), 6.54 (d, J=2.0 Hz, 1H), 3.85-3.95 (m, 7H), 3.43-3.71 (m, 3H), 2.55-3.00 (m, 3H), 1.97-2.09 (m, 5H), 1.70-1.77 (m, 1H), 1.58-1.61 (m, 1H), 1.25-1.30 (m, 4H), 1.11-1.13 (m, 3H). ESI MS m/z 479 [M+H]⁺.

### Example 95

### Preparation of 2-(4-((4-(Isopropylamino)piperidin-1-yl)methyl)phenyl)-5,7-dimethoxyquinazolin-4(3H)-one

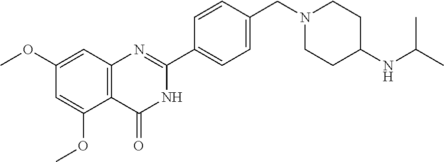 [SMILES: COc1cc(OC)c2c(=O)[nH]c(-c3ccc(CN4CCC(NC(C)C)CC4)cc3)nc2c1]

A solution of 2-(4-((4-isopropylpiperazin-1-yl)methyl)phenyl)-5,7-dimethoxyquinazolin-4(3H)-one (0.470 g, 0.98 mmol) in 2N HCl (20 mL) was refluxed for 3 days. The resulting mixture was basified with 2N NaOH and extracted with CH₂Cl₂. The organics were washed with brine, dried over anhydrous Na₂SO₄, filtered and concentrated in vacuo. Purification by flash chromatography on silica gel, eluting with 30% to 100% of 92:7:1 CHCl₃/MeOH/concentrated NH₄OH in CH₂Cl₂, afforded the title compound (0.090 g, 21%) as a light yellow solid. ¹H NMR (300 MHz, DMSO-d₆): δ 8.12 (d, J=8.3 Hz, 2H), 7.42 (d, J=8.3 Hz, 2H), 6.73 (d, J=2.3 Hz, 1H), 6.53 (d, J=2.3 Hz, 1H), 3.89 (s, 3H), 3.85 (s, 3H), 3.50 (s, 2H), 2.86-2.92 (m, 1H), 2.73-2.77 (m, 2H), 1.85-2.01 (m, 2H), 1.72-1.77 (m, 2H), 1.09-1.38 (m, 4H), 0.94 (d, J=6.2 Hz, 6H). ESI/APCI MS m/z 437 [M+H]⁺.

### Example 96

### Preparation of 2-(4-((1H-Tetrazol-5-yl)methyl)phenyl)-5,7-dimethoxyquinazolin-4(3H)-one

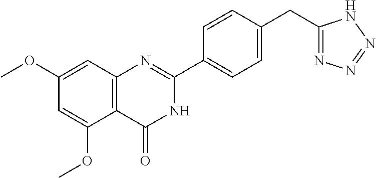 [SMILES: COc1cc(OC)c2c(=O)[nH]c(-c3ccc(Cc4nnn[nH]4)cc3)nc2c1]

To a solution of 4-cyanomethyl benzoic acid methyl ester (2.63 g, 15 mmol) in anhydrous toluene (100 mL) was added sodium azide (1.95 g, 30 mmol) and triethylamine hydrochloride (4.13 g, 30 mmol). The reaction mixture was stirred at 100° C. for 24 hours under nitrogen. The reaction mixture was cooled to room temperature, then extracted with water (2×100 mL). The aqueous layer was acidified with concentrated HCl to pH approximately 4. The white solid was filtered off, washed with water, and dried under vacuum at 40° C. overnight, to give methyl-4-(1H-tetrazol-5-ylmethyl) benzoate (2.88 g, 88%) as an off-white solid.

Lithium aluminium hydride (0.142 g, 3.75 mmol) was taken in a dry, three-necked flask, fitted with a reflux condenser. Anhydrous ether (10 mL) was added. A solution of methyl-4-(1H-tetrazol-5-ylmethyl) benzoate (0.654 g, 3.0 mmol) in anhydrous THF (5 mL) was added dropwise. After the addition was complete, the mixture was heated to reflux for 2 hours. Then, the reaction mixture was cooled to 0° C. and quenched by cautious addition of water (10 mL) and 15% sodium hydroxide solution (10 mL). The reaction mixture was stirred for 30 minutes and then allowed to warm to room temperature. The aqueous phase was acidified to pH 4 and left for 2 days. A white precipitate was formed and filtered off, washed with water, and dried under vacuum, to give [4-(1H-tetrazol-5-ylmethyl)-phenyl]-methanol as a white solid. Yield: 0.290 g (51%).

IBX (0.437 g, 1.562 mmol) was dissolved in anhydrous DMSO (5 mL) and [4-(1H-tetrazol-5-ylmethyl)-phenyl]-methanol (0.270 g, 1.562 mmol) was added. The reaction mixture was stirred at room temperature under nitrogen for 4 hours. Water (20 mL) was added. The white precipitate was filtered off, washed with water, and dried under vacuum. The crude compound was mixed with methanol (20 mL) and stirred for 30 minutes, before being filtered. The filtrate was concentrated to give 4-(1H-tetrazol-5-ylmethyl)-benzaldehyde as a white solid. Yield: 0.267 g (99%). To a solution of 2-amino-4,6-dimethoxybenzamide (0.157 g, 0.8 mmol) in N,N-dimethyl acetamide (5 mL) were added 4-(1H-tetrazol-5-ylmethyl)-benzaldehyde (0.260 g, 1.4 mmol), sodium hydrogen sulfite (58.5%, 0.159 g, 0.88 mmol) and p-toluenesulfonic acid (19 mg, 0.08 mmol). The reaction mixture was stirred at 150° C. for 3 h, then cooled to room temperature. Water (40 mL) was then added. A yellow precipitate was formed and filtered off, washed with water, and small amount of methanol. It was triturated with 10% methanol in ether to give 0.107 g of solid, which was further purified by preparative HPLC, to give the title compound (0.082 g, 28%) as a white solid. MS (ES) m/z: 365.1 (M+1). MP 295-296° C.

### Example 97

### Preparation of 1-(2-(4-(5,7-Dimethoxy-4-oxo-3,4-dihydroquinazolin-2-yl)-2,6-dimethylphenoxy)ethyl)pyrrolidine-2,5-dione

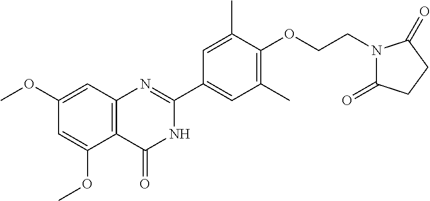 [SMILES: COc1cc(OC)c2c(=O)[nH]c(-c3cc(C)c(OCCN4C(=O)CCC4=O)c(C)c3)nc2c1]

To a solution of 2-[4-(2-hydroxy-ethoxy)-3,5-dimethyl-phenyl]-5,7-dimethoxy-3H-quinazolin-4-one (0.50 g, 1.35 mmol) in anhydrous THF (20 mL) were added triphenyl phosphine (0.53 g, 2.02 mmol), pyrrolidine-2,5-dione (0.20 g, 2.02 mmol), and N,N-diisopropylethyl amine (0.44 g, 3.38 mmol). To this stirred solution was added diethylazodicarboxylate (0.35 g, 2.02 mmol). The reaction mixture was stirred at room temperature for 8 hours under nitrogen. Ethyl acetate (400 mL) was added. The organic phase was separated, washed with water (100 mL), then brine (100 mL), and dried over anhydrous Na₂SO₄. The solvent was removed under reduced pressure. The crude material was purified by the Simpliflash system (4:96 methanol:CH₂Cl₂ as eluent) to give the title compound as a white solid. Yield: 0.3 g. (49%). ¹H NMR (400 MHz, CDCl₃): δ 9.30 (br s, 1H), 7.66 (s, 2H), 6.82 (d, J=2.4 Hz, 1H), 6.46 (d, J=1.6 Hz, 1H), 3.99 (s, 3H), 3.97 (s, 3H), 3.92 (s, 4H), 2.78 (s, 4H), 2.31 (s, 6H). MS (ES) m/z: 452.51 (M+1) (100%).

### Example 98

### Preparation of 7-(2-(Benzyloxy)ethoxy)-5-methoxy-2-(pyridin-4-yl)quinazolin-4(3H)-one

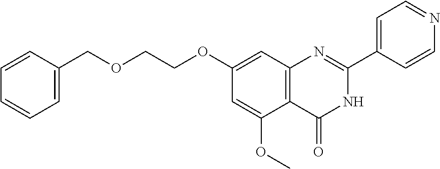 [SMILES: COc1cc(OCCOCc2ccccc2)cc2nc(-c3ccncc3)[nH]c(=O)c12]

To a stirred solution of 2-amino-4,6-difluoro-benzamide (0.50 g, 2.9 mmol) and pyridine-4-carbaldehyde (0.35 g, 3.2 mmol) in N,N-dimethylacetamide (10 mL) were added sodium hydrogen sulfite (0.63 g, 3.5 mmol) and p-toluenesulfonic acid (0.06 g, 0.3 mmol); the reaction mixture was stirred at 115° C. for 16 hours. The solvent was evaporated in vacuo, water was added, and the precipitated solid was filtered off to obtain 5,7-difluoro-2-pyridin-4-yl-3H-quinazolin-4-one as a yellow solid, which was used in the next step without further purification. Yield: 0.4 g (53%).

To a suspension of 5,7-difluoro-2-pyridin-4-yl-3H-quinazolin-4-one (0.20 g, 0.80 mmol) in DMF (3 mL) was added sodium methoxide in methanol (0.43 g, 8.0 mmol) and the reaction mixture was stirred at room temperature for 16 hours. Water was added, the mixture was acidified with acetic acid to pH approximately 4-5, and the precipitated solid was filtered off to obtain 7-fluoro-5-methoxy-2-pyridin-4-yl-3H-quinazolin-4-one as a yellowish solid. Yield: 0.20 g (83%).

To a solution of 2-benzyloxy-ethanol (2 mL) in dimethyl sulfoxide (3 mL) was added sodium hydride (0.30 g, 7.4 mmol) in portions, and the reaction mixture was stirred at room temperature for 45 minutes. To this mixture was added 7-fluoro-5-methoxy-2-pyridin-4-yl-3H-quinazolin-4-one (0.20 g, 0.74 mmol) and the reaction mixture was heated at 80° C. for 16 hours. Water was added, the mixture was acidified with acetic acid to pH approximately 4-5, and the precipitated solid was filtered off, to obtain a crude product, which was purified by preparative HPLC to obtain the title compound as a light yellow solid. Yield: 0.12 g (40%). MP 228.2-229.9° C. ¹H NMR (400 MHz, DMSO-d₆): δ 12.29 (s, 1H), 8.77 (d, 2H), 8.08 (d, 2H), 7.36 (m, 5H), 6.82 (s, 1H), 6.62 (s, 1H), 4.58 (s, 2H), 4.32 (t, 2H), 3.87 (s, 3H), 3.83 (t, 2H). MS (ES⁺) m/z: 404.51 (M+1).

### Example 99

### Preparation of 2-(2,6-Dimethylpyridin-4-yl)-5,7-dimethoxyquinazolin-4(3H)-one

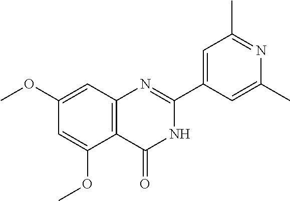

A solution of 2,6-lutidine N-oxide (24.0 g, 0.20 mol) in anhydrous dichloromethane (400 mL) was added to trimethyloxonium tetrafluoroborate (29.6 g, 0.20 mol) at room temperature under nitrogen atmosphere and the reaction mixture was stirred at room temperature for 3 hours. The mixture was concentrated in vacuo to give the crude product, 1-methoxy-2,6-dimethyl-pyridinium tetrafluoroborate.

The crude product was dissolved in MeOH (300 mL) and heated to reflux under nitrogen. Then, a solution of ammonium persulfate (14.2 g, 0.06 mol) in water (57 mL) was added. The mixture was stirred under reflux for 16 hours; TLC showed completion of the reaction. Half of the solvent was removed in vacuo, then quenched with 10% aqueous NaOH solution to pH 7, and evaporated to dryness in vacuo. The residue was dissolved in methanol and filtered, the filtrate was concentrated in vacuo, and the crude compound was purified by column chromatography (silica gel 230-400 mesh; 5-15% methanol in CH₂Cl₂ as eluent) to give 4-hydroxymethyl-2,6-dimethylpyridine as a white solid. Yield: 11.0 g (40.0%).

4-Hydroxymethyl-2,6-dimethylpyridine (1.00 g, 7.28 mmol) was dissolved in ethanol (20 mL), and activated MnO₂ (2.24 g, 21.8 mmol) was added; the reaction mixture was refluxed for 17 hours. The mixture was cooled and concentrated, purified by column chromatography (silica gel 230-400 mesh; 20% ethyl acetate in hexanes as eluent) to give 2,6-dimethyl-4-pyridinecarboxaldehyde as a yellow oil. Yield: 0.14 g (14%).

To a solution of 2,6-dimethylpyridine-4-carbaldehyde (0.14 g, 1.00 mmol) in N,N-dimethyl acetamide (10 mL) were added 2-amino-4,6-dimethoxybenzamide (0.20 g, 1.00 mmol), sodium hydrogen sulfite (0.21 g, 2.00 mmol), and p-toluenesulfonic acid (0.28 g, 1.50 mmol). The reaction mixture was stirred at 110° C. for 16 hours under nitrogen. After cooling to room temperature, solvent was evaporated under reduced pressure. The residue was dissolved in ethyl acetate, washed with saturated NaHCO₃ solution (30 mL), water (30 mL), and brine (30 mL), and dried over anhydrous sodium sulfate. Solvent was evaporated, and the residue was purified by column chromatography (silica gel 230-400 mesh; 2-5% methanol in dichloromethane as eluent) to give the title compound as a yellow solid. Yield: 0.030 g (10%). MP 291-292° C. ¹H NMR (400 MHz, CDCl₃): δ 9.86 (br s, 1H), 7.60 (s, 2H), 6.87 (d, J=2.2 Hz, 1H), 6.53 (d, J=2.2 Hz, 1H), 3.99 (s, 3H), 3.95 (s, 3H), 2.66 (s, 6H). MS (ES) m/z: 312.50 (M+1) (100%).

### Example 100

### Preparation of 2-(2,6-Dimethylpyridin-4-yl)-5-methoxy-7-(2-methoxyethoxy)quinazolin-4(3H)-one

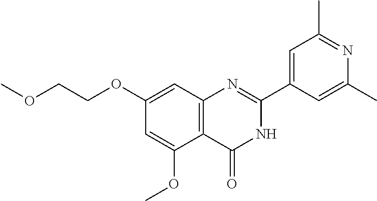

To a suspension of 2,6-dimethyl-pyridin-4-yl)-methanol (1.00 g, 7.30 mmol) in acetonitrile (20 mL), 1,2-benziodexol-3(1H)-one-1-hydroxy-1-oxide (IBX) (2.00 g, 7.30 mmol) was added and the reaction mixture was refluxed for 6 hours. The solid was filtered off and washed with acetonitrile. The filtrate was evaporated in vacuo to give 2,6-dimethyl-pyridine-4-carbaldehyde as a brown liquid. Yield: 0.81 g (82%).

To a stirred solution of 2-amino-4,6-difluoro-benzamide (1.03 g, 6.00 mmol) and 2,6-dimethyl-pyridine-4-carbaldehyde (0.81 g, 6.00 mmol) in N,N-dimethyl acetamide (15 mL), sodium hydrogen sulfite (58.5 wt %, 1.31 g, 7.20 mmol), and p-toluenesulfonic acid monohydrate (0.11 g, 0.60 mmol) were added and the reaction mixture was stirred at 115° C. for 16 hours. The solvent was evaporated in vacuo, water was added, and the precipitated solid was filtered, to give 2-(2,6-dimethyl-pyridin-4-yl)-5,7-difluoro-3H-quinazolin-4-one as a yellow solid, which was used in the next step without further purification. Yield: 0.72 g (42%).

To a suspension of 2-(2,6-dimethyl-pyridin-4-yl)-5,7-difluoro-3H-quinazolin-4-one (0.72 g, 2.51 mmol) in DMF (10 mL), a solution of sodium methoxide in methanol (25 wt %, 1.36 g, 25.1 mmol) was added and the reaction mixture was stirred at room temperature for 16 h. Water was added, the mixture was acidified to pH approximately 4-5 with acetic acid, and the precipitated solid was filtered and dried under vacuum to give 2-(2,6-dimethyl-pyridin-4-yl)-7-fluoro-5-methoxy-3H-quinazolin-4-one as a light yellow solid. Yield: 0.28 g (37%).

To a solution of 2-methoxyethanol (3 mL) in dimethyl sulfoxide (8 mL), sodium hydride (60% suspension in mineral oil, 0.40 g, 9.40 mmol) was added in portions and the reaction mixture was stirred at room temperature for 1 hour. To this reaction mixture was added 2-(2,6-dimethyl-pyridin-4-yl)-7-fluoro-5-methoxy-3H-quinazolin-4-one (0.28 g, 0.94 mmol) and the reaction mixture was stirred at 90° C. for 16 hours. Water was added, acidified to pH approximatley 4-5 with acetic acid, and the precipitated solid was filtered to give crude product, which was purified by preparative HPLC, to obtain the title compound as a white solid. Yield: 0.12 g (36%). MP 228.8-230.4° C. MS (ES) m/z: 356.05 (M⁺+1). ¹H NMR (400 MHz, CDCl₃): δ 10.45 (s, 1H), 7.65 (s, 2H), 6.85 (d, J=1.6 Hz, 1H), 6.61 (d, J=1.6 Hz, 1H), 4.27 (t, J=4.8 Hz, 2H), 3.97 (s, 3H), 3.82 (t, J=4.8 Hz, 2H), 3.49 (s, 3H), 2.66 (s, 6H).

### Example 101

### Preparation of 2-(2,6-Dimethylpyridin-4-yl)-5,7-bis(2-methoxyethoxy)quinazolin-4(3H)-one

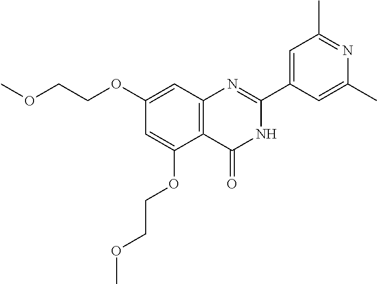

To a suspension of 2,6-dimethyl-pyridin-4-yl)-methanol (1.00 g, 7.30 mmol) in acetonitrile (20 mL), 1,2-benziodexol-3(1H)-one-1-hydroxy-1-oxide (IBX) (2.00 g, 7.30 mmol) was added and the reaction mixture was refluxed for 6 hours. The solid was filtered off and washed with acetonitrile. The filtrate was evaporated in vacuo, to give 2,6-dimethyl-pyridine-4-carbaldehyde as a brown liquid. Yield: 0.81 g (82%).

To a stirred solution of 2-amino-4,6-difluoro-benzamide (1.03 g, 6.00 mmol) and 2,6-dimethyl-pyridine-4-carbaldehyde (0.81 g, 6.00 mmol) in N,N-dimethyl acetamide (15 mL), sodium hydrogen sulfite (58.5 wt %, 1.31 g, 7.20 mmol) and p-toluenesulfonic acid monohydrate (0.11 g, 0.60 mmol) were added and the reaction mixture was stirred at 115° C. for 16 hours. The solvent was evaporated in vacuo, water was added, and the precipitated solid was filtered to give 2-(2,6-dimethyl-pyridin-4-yl)-5,7-difluoro-3H-quinazolin-4-one as a yellow solid, which was used in the next step without further purification. Yield: 0.72 g (42%).

To a suspension of 2-(2,6-dimethyl-pyridin-4-yl)-5,7-difluoro-3H-quinazolin-4-one (0.72 g, 2.51 mmol) in DMF (10 mL), a solution of sodium methoxide in methanol (25 wt %, 1.36 g, 25.1 mmol) was added and the reaction mixture was stirred at room temperature for 16 hours. Water was added, the mixture was acidified to pH approximately 4-5 with acetic acid, and the precipitated solid was filtered and dried under vacuum, to give 2-(2,6-dimethyl-pyridin-4-yl)-7-fluoro-5-methoxy-3H-quinazolin-4-one as a light yellow solid. Yield: 0.28 g (37%).

To a solution of 2-methoxyethanol (3 mL) in dimethyl sulfoxide (8 mL), sodium hydride (60% suspension in mineral oil, 0.40 g, 9.40 mmol) was added in portions and the reaction mixture was stirred at room temperature for 1 hour. To this reaction mixture was added 2-(2,6-dimethyl-pyridin-4-yl)-7-fluoro-5-methoxy-3H-quinazolin-4-one (0.28 g, 0.94 mmol); the reaction mixture was stirred at 90° C. for 16 hours. Water was added, the mixture was acidified to pH approximatley 4-5 with acetic acid, and the precipitated solid was filtered, to give crude product, which was purified by preparative HPLC to obtain the title compound. Yield: 0.03 g (8%). MP 149.8-151.4° C. MS (ES) m/z: 400.13 (M⁺+1). ¹H NMR (400 MHz, CDCl₃): δ 7.54 (s, 2H), 6.85 (s, 1H), 6.61 (s, 1H), 4.24 (m, 4H), 3.87 (t, J=5.2 Hz, 2H), 3.81 (t, J=5.2 Hz, 2H), 3.49 (br s, 6H), 2.65 (s, 6H).

### Example 102

### Preparation of 2-(2,6-Dimethylpyridin-4-yl)-7-methoxy-5-(2-(pyrrolidin-1-yl)ethoxy)quinazolin-4(3H)-one

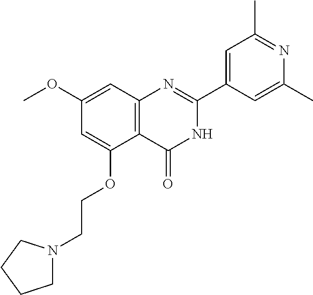 [SMILES: COc1cc(OCCN2CCCC2)c2c(=O)[nH]c(-c3cc(C)nc(C)c3)nc2c1]

To a solution of 2,6-dimethyl-pyridine-4-carbaldehyde (0.99 g, 7.32 mmol) and 2-amino-4,6-difluorobenzamide (1.26 g, 7.32 mmol) in N,N-dimethyl acetamide (20 mL) were added sodium hydrogen sulfite (58.5 wt %, 1.59 g, 8.78 mmol) and p-toluenesulfonic acid (0.21 g, 1.09 mmol). The reaction mixture was stirred at 115° C. for 16 hours under nitrogen. After cooling to room temperature, the solvent was evaporated under reduced pressure. Water (50 mL) was added, the precipitated solid was filtered, washed with water, and dried under vacuum, to give 2-(2,6-dimethyl-pyridin-4-yl)-5,7-difluoro-3H-quinazolin-4-one as a yellow solid. Yield: 0.63 g (30%).

To a solution of 2-pyrrolidin-1-yl-ethanol (5.09 g, 44.2 mmol) in DMF (10 mL) was added sodium hydride (60% suspension in mineral oil, 0.88 g, 22.1 mmol) in small portions and the reaction mixture was stirred at room temperature for 30 minutes. To this mixture was added 2-(2,6-dimethyl-pyridin-4-yl)-5,7-difluoro-3H-quinazolin-4-one 0.63 g, 2.21 mmol) and the reaction mixture was stirred at room temperature for 16 hours. Water (20 mL) was added, and the mixture was neutralized, to pH approximately 6 with acetic acid. Solvent was evaporated, and the residue was dissolved in ethyl acetate, washed with water, and dried over anhydrous sodium sulfate, and concentrated in vacuo. The crude compound was purified by the Simpliflash system (0-4% methanol in CH₂Cl₂ as eluent) to give 2-(2,6-dimethyl-pyridin-4-yl)-7-fluoro-5-(2-pyrrolidin-1-yl-ethoxy)-3H-quinazolin-4-one as a yellow solid. Yield: 0.61 g (72%).

To a solution of 2-(2,6-dimethyl-pyridin-4-yl)-7-fluoro-5-(2-pyrrolidin-1-yl-ethoxy)-3H-quinazolin-4-one (0.30 g, 0.80 mmol) in anhydrous DMF (5 mL) was added a solution of sodium methoxide in methanol (25 wt %, 0.43 g, 8.00 mmol) and the reaction mixture was stirred at 70° C. for 16 h. After cooling to room temperature, water (10 mL) was added, and the mixture was neutralized to pH approximatley 6 with acetic acid. The solvent was evaporated, and the residue was purified by the Simpliflash system (2% methanol in CH₂Cl₂ and then 4% 7.0 M ammonia in methanol and CH₂Cl₂ as eluent) to give the title compound as a yellow solid. Yield: 0.100 g (32%). MP 190-191° C. ¹H NMR (400 MHz, CDCl₃): δ 7.59 (s, 2H), 6.86 (d, J=1.95 Hz, 1H), 6.53 (d, J=1.95 Hz, 1H), 4.25 (t, J=6.05 Hz, 2H), 3.93 (s, 3H), 3.03 (t, J=6.24 Hz, 2H), 2.69 (br s, 4H), 2.64 (s, 6H), 1.93-1.70 (m, 4H). MS (ES⁺) m/z: 395.22 (M+1) and 298.12 (100%).

### Example 103

### Preparation of 2-(2,6-Dimethylpyridin-4-yl)-5-methoxy-7-(2-phenoxyethoxy)quinazolin-4(3H)-one

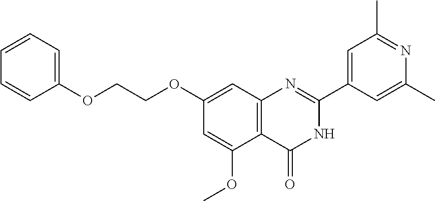 [SMILES: COc1cc(OCCOc2ccccc2)cc2nc(-c3cc(C)nc(C)c3)[nH]c(=O)c12]

To a solution of 2-phenoxy-ethanol (0.90 g, 6.50 mmol) in DMSO (5 mL) was added sodium hydride (60% in mineral oil, 0.16 g, 4.00 mmol) in small portions. The reaction mixture was stirred at room temperature under nitrogen for 1 hour. 2-(2,6-Dimethyl-pyridin-4-yl)-7-fluoro-5-methoxy-3H-quinazolin-4-one (0.20 g, 0.67 mmol) was added and stirring continued at 90° C. for 17 hours. The reaction was then cooled to room temperature, water (100 mL) was added, and was extracted with ethyl acetate (200 mL). The organic phase was washed with brine and dried over anhydrous Na₂SO₄. Solvent was removed and the crude compound was purified by column chromatography (silica gel 230-400 mesh; 5% methanol in CH₂Cl₂ as eluent) to give the title compound as a white solid. Yield: 70 mg (25%). MP 223-224° C. ¹H NMR (400 MHz, CDCl₃): δ 11.35 (s, 1H), 7.75 (s, 2H), 7.32 (t, J=8.0 Hz, 2H), 7.02-6.97 (m, 3H), 6.91 (d, J=2.0 Hz, 1H), 6.60 (d, J=1.6 Hz, 1H), 4.49-4.47 (m, 2H), 4.41-4.39 (m, 2H), 3.97 (s, 3H), 2.67 (s, 6H). MS (ES⁺) m/z: 418.08 (M+1).

### Example 104

### Preparation of 2-(2,6-Dimethylpyridin-4-yl)-7-methoxy-5-(2-phenoxyethoxy)quinazolin-4(3H)-one

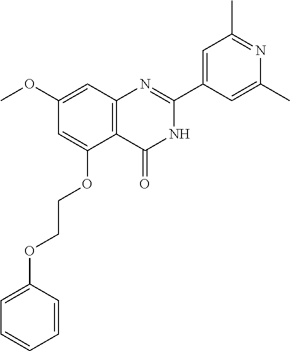

A solution of 2,6-lutidine N-oxide (41.6 g, 0.337 mol, 1.0 equiv.) in dry DCM (650 mL) was added to a flask containing trimethyloxonium tetrafluoroborate (50.0 g, 0.337 mol, 1.0 equiv.) at room temperature. under a nitrogen atmosphere. The mixture was stirred at room temperature for 3.0 hours, then concentrated in vacuo to give 78 g of crude 4-hydroxymethyl-2,6-dimethylpyridine. The crude product was dissolved in methanol (500 mL) and the solution was heated to reflux under a nitrogen atmosphere, then a solution of ammonium persulfate (24.6 g, 0.101 mol) in water (100 mL) was added dropwise. The mixture was stirred at reflux for 16 hours; TLC indicated complete reaction. Half of the solvents were removed in vacuo, then quenched with 10% NaOH solution to pH approximately 7, and evaporated to dryness. The residue was dissolved in methanol and filtered, the filtrate was concentrated in vacuum, and purified by column chromatography (eluting with methanol: DCM=5-15%) to give the title compound as a white solid. Yield: 24.7 g (52%).

4-Hydroxymethyl-2,6-dimethylpyridine (24.7 g, 180 mmol, 1.0 equiv.) was dissolved in DMSO (200 mL), and IBX (53.0 g, 189 mmol, 1.05 equiv.) was added in portions, the mixture was stirred at room temperature for 2 hours; TLC indicated complete reaction. The mixture was filtered, washed with water and ether. The filtrate was extracted with ether (4×150 mL); the combined extracts were washed with water and brine, dried over anhydrous sodium sulfate, and concentrated to give the crude product, which was purified by column chromatography (20% ether in hexanes as eluent) to give 2,6-dimethyl-4-pyridinecarboxaldehyde as a yellow oil. Yield: 20.0 g (82%).

To a solution of 2,6-dimethyl-pyridine-4-carbaldehyde (5.0 g, 36.5 mmol) and 2-amino-4,6-difluorobenzamide (6.28 g, 36.5 mmol) in N,N-dimethyl acetamide (80 mL) were added sodium hydrogen sulfite (7.95 g, 43.8 mmol) and p-toluenesulfonic acid (0.7 g, 3.65 mmol). The reaction mixture was stirred at 115° C. for 16 hours under nitrogen. The reaction mixture was cooled to room temperature, diluted with water, the precipitate was collected by filtration, washed with sat. NaHCO₃ and brine, and dried in vacuo to give 2-(2,6-dimethylpyridin-4-yl)-5,7-difluoro-3H-quinazolin-4-one as a white solid. Yield: 2.82 g (26.8%).

To a solution of 2-phenoxyethanol (4.81 g, 34.8 mmol) in DMF (20 mL) was added sodium hydride (60% suspension in mineral oil, 0.70 g, 17.4 mmol) in portions and the reaction mixture was stirred at room temperature for 1 hour. To this mixture was added 2-(2,6-dimethylpyridin-4-yl)-5,7-difluoro-3H-quinazolin-4-one (0.50 g, 1.74 mmol) and the reaction mixture was stirred at room temperature for 16 hours. Water (1 mL) was added, neutralized to pH approximatley 6-7 with acetic acid, concentrated, dissolved in ethyl acetate, washed with water, dried over anhydrous sodium sulfate, and concentrated in vacuo. The residue was purified by column chromatography (eluted with 50% ethyl acetate in hexanes, then 5% methanol in DCM) to give 2-(2,6-dimethylpyridin-4-yl)-7-fluoro-5-(2-phenoxyethoxy)-3H-quinazolin-4-one as a light yellow solid. Yield: 0.59 g (83%).

To a suspension of 2-(2,6-dimethylpyridin-4-yl)-7-fluoro-5-(2-phenoxyethoxy)-3H-quinazolin-4-one (0.59 g, 1.45 mmol) in DMF (10 mL) was added a solution of sodium methoxide in methanol (25 wt %, 3.15 g, 14.5 mmol) and the reaction mixture was stirred at approximatley 70-80° C. for 48 hours, then cooled to room temperature. Water (1 mL) was added, the mixture was neutralized to pH approximatley 6-7 with acetic acid, concentrated, dissolved in DCM, washed with water and brine, dried over anhydrous sodium sulfate, concentrated in vacuo, and the residue was passed through a column (eluted with 2% methanol in DCM), to give 0.12 g of the desired product. The crude product was washed with acetonitrile, then solubilized in dioxane, and precipitated by adding water to afford the title compound as a white solid. Yield: 70 mg (11%). ¹H NMR (400 MHz, DMSO-d₆): δ 12.08 (br s, 1H), 7.77 (s, 2H), 7.31 (t, J=7.81 Hz, 2H), 7.04 (d, J=8.20 Hz, 2H), 6.96 (t, J=7.42 Hz, 1H), 6.83 (d, J=1.56 Hz, 1H), 6.69 (s, 1H), 4.40-4.53 (m, 2H), 3.90 (s, 3H), 3.33 (s, 6H). MS (ES⁺) m/z: 418.14 (M+1)⁺; MP 172.3-173.2° C.

### Example 105

### Preparation of 2-(2,6-Dimethylpyridin-4-yl)-7-methoxy-5-(2-methoxyethoxy)quinazolin-4(3H)-one

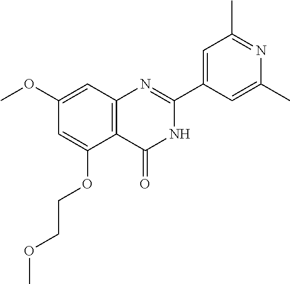

To a solution of 2-methoxyethanol (2.65 g, 34.8 mmol) in DMF (38 mL) was added sodium hydride (60% suspension in mineral oil, 0.70 g, 17.4 mmol) in portions and the reaction mixture was stirred at room temperature for 0.5 hours. To this mixture was added 2-(2,6-dimethylpyridin-4-yl)-5,7-difluoro-3H-quinazolin-4-one (0.50 g, 1.74 mmol) and the reaction mixture was stirred at room temperature for 16 hours. Water (1.5 mL) was added, the mixture was neutralized to pH approximatley 6-7 with acetic acid, concentrated, dissolved in ethyl acetate (200 mL), washed with water and brine, dried over anhydrous sodium sulfate, and concentrated in vacuo. The residue was washed with hexanes to give 2-(2,6-dimethylpyridin-4-yl)-7-fluoro-5-(2-methoxyethoxy)-3H-quinazolin-4-one) as a pale solid. Yield: 0.52 g (87%).

To a suspension of 2-(2,6-dimethylpyridin-4-yl)-7-fluoro-5-(2-methoxyethoxy)-3H-quinazolin-4-one (0.42 g, 1.22 mmol) in DMF (10 mL) was added a solution of sodium methoxide in methanol (25 wt %, 2.8 g, 12.8 mmol) and the reaction mixture was stirred at 70° C. for 16 hours, then cooled to room temperature. Water (1 mL) was added, the mixture was neutralized to pH approximately 6 with acetic acid, diluted with water (50 mL), and extracted with ethyl acetate. The combined extracts were washed with water and brine, dried over anhydrous sodium sulfate, and concentrated in vacuo, to give 0.30 g of crude compound. Further purification by crystallization in acetone:Et₂O (1:3) gave the title compound as a white solid. Yield: 91 mg (15%). ¹H NMR (400 MHz, CDCl₃): δ 10.08 (br s, 1H), 7.60 (br s, 2H), 6.87 (d, J=1.95 Hz, 2H), 6.55 (d, J=1.95 Hz, 2H), 4.25 (t, J=4.88 Hz, 2H), 3.93 (s, 3H), 3.83 (d, J=4.29 Hz, 2H), 3.44 (s, 3H), 2.64 (s, 6H). MS (ES⁺) m/z: 356.11 (M+1)⁺

### Example 106

### Preparation of 2-(2,6-Dimethylpyridin-4-yl)-5-methoxy-7-(2-(pyrrolidin-1-yl)ethoxy)quinazolin-4(3H)-one

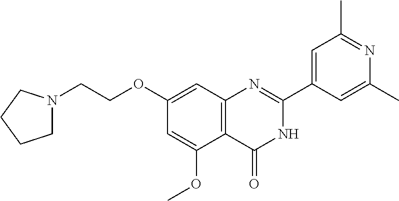 [SMILES: COc1cc(OCCN2CCCC2)cc2nc(-c3cc(C)nc(C)c3)[nH]c(=O)c12]

To a suspension of 2,6-dimethyl-pyridin-4-yl)-methanol (6.00 g, 0.043 mol) in acetonitrile (150 mL), 1,2-benziodexol-3(1H)-one-1-hydroxy-1-oxide (IBX) (14.8 g, 0.0503 mol) was added and the reaction mixture was refluxed for 2 hours. The solid was filtered off and washed with acetonitrile. The filtrate was evaporated in vacuo to give 2,6-dimethyl-pyridine-4-carbaldehyde as a brown liquid. Yield: 4.30 g (72.7%).

To a stirred solution of 2-amino-4,6-difluoro-benzamide (4.00 g, 0.0237 mol) and 2,6-dimethyl-pyridine-4-carbaldehyde (3.20 g, 0.0237 mol) in N,N-dimethyl acetamide (15 mL), sodium hydrogen sulfite (58.5 wt %, 5.05 g, 0.0284 mol) and p-toluenesulfonic acid monohydrate (0.90 g, 4.74 mmol) were added and the reaction mixture was stirred at 130° C. for 16 hours. The solvent was evaporated in vacuo, water was added, and the precipitated solid was filtered to give 2-(2,6-dimethyl-pyridin-4-yl)-5,7-difluoro-3H-quinazolin-4-one as a yellow solid, which was used in the next step without further purifications. Yield: 3.70 g (42%).

To a suspension of 2-(2,6-dimethyl-pyridin-4-yl)-5,7-difluoro-3H-quinazolin-4-one (2.70 g, 9.4 mmol) in DMF (15 mL), a solution of sodium methoxide in methanol (25 wt %, 6.0 g, 28.2 mmol) was added and the reaction mixture was stirred at room temperature for 16 hours. Water was added, the mixture was acidified to pH approximatley 4-5 with acetic acid, and the precipitated solid was filtered and dried under vacuum to give crude 2-(2,6-dimethyl-pyridin-4-yl)-7-fluoro-5-methoxy-3H-quinazolin-4-one (2.40 g), which was further purified by column chromatography (silica gel 230-400 mesh; eluting with 2% methanol solution in dichloromethane) to yield pure compound as a light yellow solid. Yield: 0.35 g (12.4%).

To a solution of 2-pyrrolidin-1-yl-ethanol (1.15 g, 10 mmol) in dimethyl sulfoxide (4 mL), sodium hydride (60% suspension in mineral oil, 0.20 g, 5.0 mmol) was added in portions and the reaction mixture was stirred at room temperature for 20 minutes. To this reaction mixture was added 2-(2,6-dimethyl-pyridin-4-yl)-7-fluoro-5-methoxy-3H-quinazolin-4-one (0.30 g, 1.0 mmol) and the reaction mixture was stirred at 75° C. for 16 hours. The reaction mixture was loaded onto a column and purified by column chromatography (silica gel 230-400 mesh; eluting with 5% 7.0 M ammonia in methanol solution in dichloromethane), to obtain the title compound as a white solid. Yield: 0.163 g (41.3%). MP 227-229° C. MS (ES) m/z: 395.15 (M⁺+1). ¹H NMR (400 MHz, CDCl₃): δ 7.78 (s, 2H), 6.87 (d, J=2.4 Hz, 1H), 6.58 (d, J=2.4 Hz, 1H), 4.25 (t, J=6.0 Hz, 2H), 3.95 (s, 3H), 2.97 (t, J=6.0 Hz, 2H), 2.66 (s, 6H), 2.63 (m, 4H), 1.83 (m, 4H).

### Example 107

### Preparation of 2-(2,6-Dimethylpyridin-4-yl)-7-(2-isopropoxyethoxy)-5-methoxyquinazolin-4(3H)-one

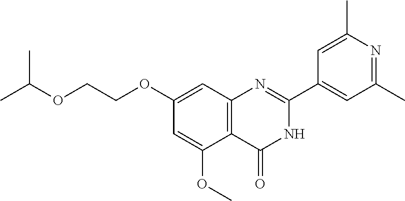 [SMILES: COc1cc(OCCOC(C)C)cc2nc(-c3cc(C)nc(C)c3)[nH]c(=O)c12]

To a suspension of 2-(2,6-dimethyl-pyridin-4-yl)-5,7-difluoro-3H-quinazolin-4-one (0.97 g, 3.38 mmol) in anhydrous DMF (10 mL) was added a solution of sodium methoxide in methanol (25 wt %, 1.09 g, 20.3 mmol). The reaction mixture became clear. The reaction mixture was stirred at room temperature for 16 hours. Water (100 mL) was added, neutralized to pH approximatley 6 with aqueous 2N HCl. The separated solid was filtered, washed with water (50 mL), and dried under vacuum to give an off-white solid. Yield: 0.94 g (93%).

To a suspension of sodium hydride (60% suspension in mineral oil, 0.24 g, 6.00 mmol) in anhydrous DMSO (10 mL) was added 2-isopropoxy-ethanol at room temperature under nitrogen. The mixture was stirred for 20 minutes at room temperature, then 2-(2,6-dimethyl-pyridin-4-yl)-7-fluoro-5-methoxy-3H-quinazolin-4-one (0.30 g, 1.00 mmol) was added and the reaction mixture was stirred at 80° C. for 16 hours, then cooled to room temperature. Water (50 mL) was added, and the mixture was extracted with a mixture of ethyl acetate and THF (4:1, 200 mL). The organic phase was washed with brine and dried over anhydrous sodium sulfate. Solvent was evaporated, and the crude compound was purified by the Simpliflash system (3:15:82 methanol, ethyl acetate and dichloromethane as eluent) to give the title compound as a white solid. Yield: 127 mg (33%). MP 188-189° C. ¹H NMR (400 MHz, CDCl₃): δ 11.14 (br s, 1H), 7.72 (s, 2H), 6.86 (d, J=2.34 Hz, 1H), 6.59 (d, J=2.34 Hz, 1H), 4.35-4.15 (m, 2H), 3.97 (s, 3H), 3.89-3.79 (m, 2H), 3.78-3.64 (m, 1H), 2.66 (s, 6H), 1.23 (d, J=5.85 Hz, 6H). MS (ES⁺) m/z: 384.20 (100%).

### Example 108

### Preparation of 2-(2,6-dimethylpyridin-4-yl)-5,7-bis(2-isopropoxyethoxy)quinazolin-4(3H)-one

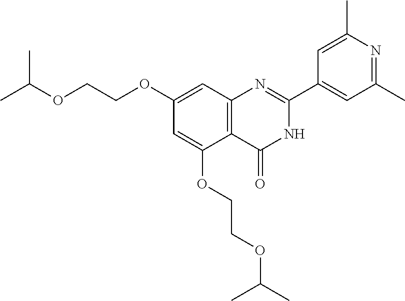 [SMILES: Cc1cc(-c2nc3cc(OCCOC(C)C)cc(OCCOC(C)C)c3c(=O)[nH]2)cc(C)n1]

The title compound was isolated using the process described for Example 113 as a white solid. Yield: 124 mg (27%). MP 124-125° C. ¹H NMR (400 MHz, CDCl₃): δ 10.04 (br s, 1H), 7.60 (s, 2H), 6.85 (d, J=2.34 Hz, 1H), 6.63 (d, J=2.34 Hz, 1H), 4.23 (t, J=4.88 Hz, 4H), 3.85 (dt, J=10.54 and 5.27 Hz, 4H), 3.80-3.64 (m, 2H), 2.64 (s, 6H), 1.23 (d, J=6.24 Hz, 6H), 1.17 (d, J=6.24 Hz, 6H). MS (ES⁺) m/z: 456.17 (100%).

### Example 109

### Preparation of 7-(2-(Benzyloxy)ethoxy)-2-(2,6-dimethylpyridin-4-yl)-5-methoxyquinazolin-4(3H)-one

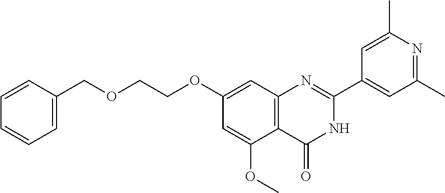 [SMILES: COc1cc(OCCOCc2ccccc2)cc2nc(-c3cc(C)nc(C)c3)[nH]c(=O)c12]

To a suspension of 2,6-dimethyl-pyridin-4-yl)-methanol (6.00 g, 0.043 mol) in acetonitrile (150 mL), 1,2-benziodexol-3(1H)-one-1-hydroxy-1-oxide (IBX) (14.8 g, 0.0503 mol) was added and the reaction mixture was refluxed for 2 hours. The solid was filtered off and washed with acetonitrile. The filtrate was evaporated in vacuo to give 2,6-dimethyl-pyridine-4-carbaldehyde as a brown liquid. Yield: 4.30 g (72.7%).

To a stirred solution of 2-amino-4,6-difluoro-benzamide (4.00 g, 0.0237 mol) and 2,6-dimethyl-pyridine-4-carbaldehyde (3.20 g, 0.0237 mol) in N,N-dimethyl acetamide (15 mL), sodium hydrogen sulfite (58.5 wt %, 5.05 g, 0.0284 mol), and p-toluene sulfonic acid monohydrate (0.90 g, 4.74 mmol) were added and the reaction mixture was stirred at 130° C. for 16 hours. The solvent was evaporated in vacuo, water was added, and the precipitated solid was filtered to give 2-(2,6-dimethyl-pyridin-4-yl)-5,7-difluoro-3H-quinazolin-4-one as a yellow solid, which was used in the next step without further purification. Yield: 3.70 g (54.3%).

To a suspension of 2-(2,6-dimethyl-pyridin-4-yl)-5,7-difluoro-3H-quinazolin-4-one (2.70 g, 9.4 mmol) in DMF (15 mL), a solution of sodium methoxide in methanol (25 wt %, 6.0 g, 28.2 mmol) was added and the reaction mixture was stirred at room temperature for 16 h. Water was added, acidified to pH approximately 4-5 with acetic acid and the precipitated solid was filtered and dried under vacuum to give crude 2-(2,6-dimethyl-pyridin-4-yl)-7-fluoro-5-methoxy-3H-quinazolin-4-one (2.40 g), which was further purified by column chromatography (silica gel 230-400 mesh; eluting with 2% methanol solution in dichloromethane) to yield pure compound as a light yellow solid. Yield: 0.35 g (12.4%).

To a solution of 2-benzyloxy-ethanol (1.15 g, 10.0 mmol) in dimethyl sulfoxide (4 mL), sodium hydride (60% suspension in mineral oil, 0.20 g, 5.0 mmol) was added in portions and the reaction mixture was stirred at room temperature for 20 minutes. To this reaction mixture was added 2-(2,6-dimethyl-pyridin-4-yl)-7-fluoro-5-methoxy-3H-quinazolin-4-one (0.30 g, 1.0 mmol) and the reaction mixture was stirred at 85° C. for 24 hours. Water was added, and the mixture was acidified to pH approximately 4-5 with acetic acid and the precipitated solid was filtered to give crude product, which was purified by column chromatography (silica gel 230-400 mesh; eluting with hexane and ethyl acetate 10:1) to obtain the title compound as a white solid. Yield: 0.140 g (32.4%). MP 178-180° C. MS (ES) m/z: 432.18 (M⁺+1). ¹H NMR (400 MHz, CDCl₃): δ 10.90 (s, 1H), 7.69 (s, 2H), 7.29-7.40 (m, 5H), 6.85 (d, J=2.0 Hz, 1H), 6.59 (d, J=2.0 Hz, 1H), 4.66 (s, 2H), 4.29 (m, 2H), 3.97 (s, 3H), 3.89 (m, 2H), 2.66 (s, 6H).

### Example 110

### Preparation of 5-methoxy-7-(2-methoxyethoxy)-2-(2-methylpyridin-4-yl)quinazolin-4(3H)-one

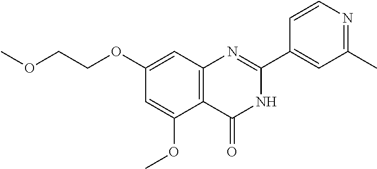

To a solution of 2-amino-4,6-difluoro-benzamide (0.71 g, 4.10 mmol) and 2-methyl-pyridine-4-carbaldehyde (0.50 g, 4.10 mmol) in N,N-dimethylacetamide (10 mL) were added NaHSO₃ (58.5 wt %, 1.00 g, 5.70 mmol) and p-TSA (0.16. g, 0.08 mmol). The reaction mixture was heated at 115° C. for 30 hours, then cooled to room temperature. The solvent was removed under reduced pressure. The crude compound was purified by column chromatography (silica gel 230-400 mesh; 5% methanol in dichloromethane) to afford 5,7-difluoro-2-(2-methyl-pyridin-4-yl)-3H-quinazolin-4-one as a light yellow solid. Yield: 0.30 g (26%).

To a suspension of 5,7-difluoro-2-(2-methyl-pyridin-4-yl)-3H-quinazolin-4-one (0.30 g, 1.09 mmol) in anhydrous DMF (8 mL) was added a solution of sodium methoxide in methanol (25 wt %, 0.59 g, 10.9 mmol) and the reaction mixture was stirred at room temperature for 3 hours. Water was added, the mixture was acidified to pH approximately 5 with acetic acid, and the precipitated solid was filtered and dried under vacuum to give 7-fluoro-5-methoxy-2-(2-methyl-pyridin-4-yl)-3H-quinazolin-4-one as a light yellow solid. Yield: 0.24 g (76%).

To a solution of 2-methoxy-ethanol (0.64 g, 8.40 mmol) in anhydrous DMSO (4 mL) was added sodium hydride (60% suspension in mineral oil, 0.12 g, 5.00 mmol) in small portions and the reaction mixture was stirred at room temperature for 30 minutes. To this mixture was added a solution of 7-fluoro-5-methoxy-2-(2-methyl-pyridin-4-yl)-3H-quinazolin-4-one (0.24 g, 0.84 mmol) in anhydrous DMSO (12 mL). The reaction mixture was stirred at 80° C. for 3 hours, then cooled to room temperature, and diluted with ether (500 mL). The solid was filtered and washed with ether. The crude compound was purified by column chromatography (silica gel 230-400 mesh; 4% methanol in dichloromethane). The compound was further purified by preparative HPLC to give the title compound as a white solid. Yield: 60 mg (21%). MP 260-262° C. ¹H NMR (400 MHz, DMSO-d₆): δ 8.62 (d, J=5.07 Hz, 1H), 7.98 (s, 1H), 7.88 (d, J=5.07 Hz, 1H), 6.80 (d, J=2.34 Hz, 1H), 6.61 (d, J=2.34 Hz, 1H), 4.25 (t, J=4.68 Hz, 2H), 3.86 (s, 3H), 3.71 (t, J=3.90 Hz, 2H), 3.33 (s, 3H), 2.57 (s, 3H). MS (ES) m/z: 342.07 (M+1) (100%).

### Example 111

### Preparation of 2-(2,6-Dimethylpyridin-4-yl)-7-(2-methoxyethoxy)-5-(2-(pyrrolidin-1-yl)ethoxy)quinazolin-4(3H)-one

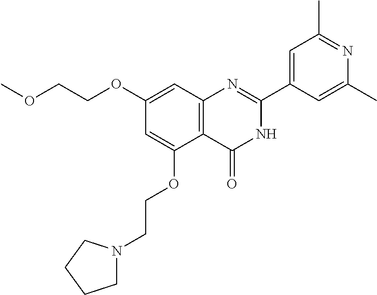

To a solution of 2-pyrrolidin-1-yl-ethanol (5.09 g, 44.2 mmol) in DMF (10 mL) was added sodium hydride (60% suspension in mineral oil, 0.88 g, 22.1 mmol) in small portions and the reaction mixture was stirred at room temperature for 30 minutes. To this mixture was added 2-(2,6-dimethyl-pyridin-4-yl)-5,7-difluoro-3H-quinazolin-4-one (0.63 g, 2.21 mmol) and the reaction mixture was stirred at room temperature for 16 hours. Water (20 mL) was added, and the mixture was neutralized to pH approximately 6 with acetic acid. Solvent was evaporated, the residue was dissolved in ethyl acetate, washed with water, dried over anhydrous sodium sulfate, and concentrated in vacuo. Crude compound was purified by the Simpliflash system (0-4% methanol in CH₂Cl₂ as eluent) to afford 2-(2,6-dimethyl-pyridin-4-yl)-7-fluoro-5-(2-pyrrolidin-1-yl-ethoxy)-3H-quinazolin-4-one as a yellow solid. Yield: 0.61 g (72%).

To a solution of 2-methoxy-ethanol (1.35 g, 17.8 mmol) in DMF (10 mL) was added sodium hydride (60% suspension in mineral oil, 0.36 g, 8.89 mmol) in small portions and the reaction mixture was stirred at room temperature for 30 minutes. To this mixture was added 2-(2,6-dimethyl-pyridin-4-yl)-7-fluoro-5-(2-pyrrolidin-1-yl-ethoxy)-3H-quinazolin-4-one (0.34 g, 0.89 mmol) and the reaction mixture was stirred at 70-80° C. for 16 h, then cooled to room temperature. Water (10 mL) was added, and the mixture was neutralized to pH approximatley 6 with acetic acid. Solvent was evaporated; the residue was purified by the Simpliflash system (2-5% 7.0M ammonia in methanol and CH₂Cl₂ as eluent). The compound was further purified by preparative HPLC to give the title compound as a yellow solid. Yield: 72 mg (18%). MP 60.4-62.3° C. ¹H NMR (400 MHz, CDCl₃): δ 10.23 (br s, 1H), 8.50 (br s, 1H), 7.60 (s, 2H), 6.76 (br s, 1H), 6.43 (br s, 1H), 4.35 (m., 2H), 4.21 (m, 2H), 3.79 (s, 3H), 3.47-3.38 (m, 6H), 2.64 (s, 6H), 1.99 (m, 4H). MS (ES) m/z: 437.09 (M−1) (100%).

### Example 112

### Preparation of 2-(3-(2-Hydroxyethoxy)phenyl)-5,7-dimethoxyquinazolin-4(3H)-one

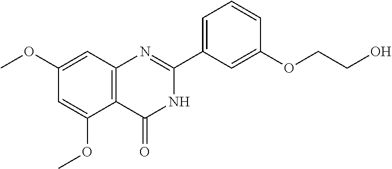 [SMILES: COc1cc(OC)c2c(=O)[nH]c(-c3cccc(OCCO)c3)nc2c1]

To a suspension of sodium hydride (0.426 g, 10.7 mmol) in DMF (30 mL) at room temperature was added 3-hydroxybenzaldehyde (1.00 g, 8.20 mmol). The resulting suspension was stirred at room temperature for 1 hour and (2-bromo-ethoxy)-tert-butyl-dimethyl-silane (4.4 mL, 20.5 mmol), was then added. The resulting mixture was stirred at 60° C. under nitrogen for 14 hours, cooled to room temperature, diluted with water (100 mL), extracted with ethyl acetate (250 mL), and concentrated. The crude product was purified by column chromatography (SiO₂, hexane/ethyl acetate=4:1) to afford 3-[2-(tert-butyl-dimethyl-silanyloxy)-ethoxy]-benzaldehyde. It was re-dissolved in THF (50 mL), mixed with 1 N tetra-n-butylammonium fluoride in THF (15 mL), and stirred at room temperature for 8 h. The reaction mixture was then concentrated and the residue was purified by column chromatography (SiO₂, hexane/ethyl acetate=4:1) to afford 3-(2-hydroxy-ethoxy)-benzaldehyde as a colorless oil.

Yield: 0.68 g (50% for two steps).

A mixture of 2-amino-4,6-dimethoxy-benzamide (195 mg, 1.00 mmol), 3-(2-hydroxy-ethoxy)-benzaldehyde (166 mg, 1.00 mmol), p-toluenesulfonic acid monohydrate (38 mg, 0.20 mmol), and sodium bisulfite (264 mg, 1.50 mmol) in N,N-dimethylacetamide (10 mL) was stirred at 130° C. under nitrogen for 14 hours, cooled to room temperature, and diluted with 0.2 N potassium carbonate aqueous solution (50 mL). It was extracted with ethyl acetate (250 mL), dried over sodium sulfate, and concentrated. The solid residue was re-dissolved in dichloromethane (5 mL), and precipitated with ethyl acetate (15 mL) and hexanes (50 mL). It was filtered and washed with hexanes to afford the title compound as a yellow solid. Yield: 70 mg (20%). MP 244.8-246.0° C. ¹H NMR (400 MHz, CDCl₃): δ 7.64 (d, 1H), 7.60 (d, 1H), 7.45 (t, 1H), 7.12 (dd, 1H), 6.84 (d, 1H), 6.48 (d, 1H), 4.21 (t, 2H), 4.03 (t, 2H), 3.99 (s, 3H), 3.94 (s, 3H). MS (ES⁺) m/z: 343.55 (M+1).

### Example 113

### Preparation of 2-(3-(2-Hydroxyethoxy)-5-methylphenyl)-5,7-dimethoxyquinazolin-4(3H)-one

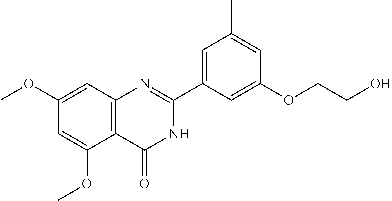 [SMILES: COc1cc(OC)c2c(=O)[nH]c(-c3cc(C)cc(OCCO)c3)nc2c1]

To a solution of 3,5-dimethyl-phenol (3.000 g, 24.55 mmol) in N,N-dimethylformamide (120 mL) under nitrogen were added potassium carbonate (16.96 g, 122.7 mmol) and (2-bromoethoxy)-tert-butyldimethylsilane (7.90 mL, 36.8 mmol). The resulting slurry was heated at reflux for 20 hours; then, the solvent was removed under high vacuum. The residue was dissolved in ethyl acetate and the solution was backwashed with 0.2 N aqueous sodium hydroxide, water, and then brine, dried over sodium sulfate, and concentrated. The crude material (5.69 g) was purified by column chromatography (silica gel 230-400 mesh; methylene chloride as eluent) to give tert-butyl-[2-(3,5-dimethyl-phenoxy)-ethoxy]-dimethylsilane as light yellow oil. Yield: 3.72 g (47%).

To a solution of tert-butyl-[2-(3,5-dimethyl-phenoxy)-ethoxy]-dimethylsilane (2.22 g, 7.91 mmol) in carbon tetrachloride (50 mL) under nitrogen was added N-bromosuccinimide (1.57 g, 8.70 mmol) and benzoyl peroxide (0.38 g, 1.58 mmol). The resulting mixture was heated at reflux for 3 hours with simultaneous illumination by a sun lamp. The precipitate was filtered off and the filtrate was concentrated under reduced pressure. The crude material (3.99 g) was purified by column chromatography (silica gel 230-400 mesh; 1/0 to 4/1 hexanes/EtOAc as eluent) to give [2-(3-bromomethyl-5-methyl-phenoxy)-ethoxy]-tert-butyl-dimethyl-silane as a light yellow oil. Yield: 2.17 g (75%).

To a solution of [2-(3-bromomethyl-5-methyl-phenoxy)-ethoxy]-tert-butyl-dimethyl-silane (2.17 g, 6.04 mmol) under nitrogen in 2-nitopropane (2.0 mL, 20 mmol) was added sodium ethoxide (0.620 g, 9.06 mmol). The resulting mixture was heated at 90° C. for 15 hours, and was then diluted with ethyl acetate and quenched with saturated aqueous ammonium chloride. The aqueous layer was extracted with ethyl acetate and the combined organic layers were backwashed with water and brine, dried over sodium sulfate, and concentrated. The crude material (1.81 g) was purified by column chromatography (silica gel 230-400 mesh; 1/0 to 4/1 hexanes/EtOAc as eluent) to give 3-[2-(tert-butyl-dimethyl-silanyloxy)-ethoxy]-5-methyl-benzaldehyde as a yellow oil. Yield: 0.97 g (55%).

To a solution of 2-amino-4,6-dimethoxy-benzamide (0.350 g, 1.78 mmol) in N,N-dimethylacetamide (20 mL) under nitrogen was added 3-[2-(tert-butyl-dimethyl-silanyloxy)-ethoxy]-5-methyl-benzaldehyde (0.520 g, 1.78 mmol) followed by sodium hydrogensulfite (0.270 g, 2.67 mmol), and p-toluenesulfonic acid (0.033 g, 0.18 mmol). The resulting mixture was heated at 120° C. for 24 hours, then the solvent was concentrated to 5 mL under reduced pressure, and water was added to obtain a precipitate, which was filtered off and washed with Et₂O and methylene chloride. The resulting solid was dissolved in hot CH₂Cl₂/MeOH, and then precipitated by adding Et₂O, and purified by preparative thin-layer chromatography (DC-Fertigplatten SIL G-100 UV, 9/1 methylene chloride/MeOH as eluent) to give the title compound as a yellow solid. Yield: 81 mg (13%). MP 106.9-109.1° C. ¹H NMR (400 MHz, CDCl₃): δ 7.86 (s, 1H), 7.41 (d, 2H), 6.82 (s, 1H), 6.57 (s, 1H), 4.15-4.13 (m, 2H), 3.94-3.90 (m, 8H), 2.43 (s, 3H). MS (ES⁺) m/z: 357.53 (M+1).

### Example 114

### Preparation of 5,7-Dimethoxy-2-(3-methoxy-5-(2-(pyrrolidin-1-yl)ethoxy)phenyl)quinazolin-4(3H)-one

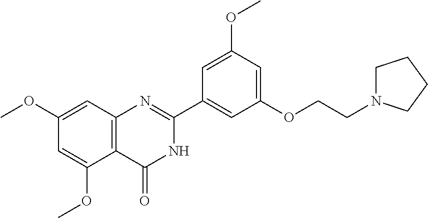 [SMILES: COc1cc(OCCN2CCCC2)cc(-c2nc3cc(OC)cc(OC)c3c(=O)[nH]2)c1]

To a 1.0-L three-neck flask was added sodium ethanethiolate (80%, 28.5 g, 271.0 mmol) and anhydrous DMF (225 mL). The mixture was heated to 145° C. for 1.5 hours. Then, 3,5-dimethoxy-benzaldehyde (15.0 g, 90.0 mmol) in anhydrous DMF (350 mL) was added over a period of 8 minutes. The reaction was kept at 145° C. for another 1 hour, then cooled to room temperature. Saturated sodium chloride solution (2.5 L) and formaline (37%, 240 mL) together with acetic acid (500 mL) was added. The resulting solution was thoroughly extracted with ethyl acetate, the organic phase was dried with sodium sulfate, and the solvent was removed under vacuum. The crude compound was purified by column chromatography (silica gel 230-400 mesh; eluting with dichloromethane and ethyl acetate 7:1) to give 3-hydroxy-5-methoxy-benzaldehyde as a white solid. Yield: 12.0 g (88%).

3-Hydroxy-5-methoxy-benzaldehyde (12.0 g, 78.9 mmol) and [1,3]dioxolan-2-one (13.9 g, 157.0 mmol) in anhydrous DMF (50 mL) was added potassium carbonate (21.6 g, 157.0 mmol). The mixture was then heated to 110° C. for 16 hours. The reaction mixture was cooled to room temperature. Solid potassium carbonate was filtered and washed with ethyl acetate. The organic phase was collected and solvent was removed. The residue was purified by column chromatography (silica gel 230-400 mesh; eluting with dichloromethane and ethyl acetate 7:1), to give 3-(2-hydroxy-ethoxy)-5-methoxy-benzaldehyde as a brown liquid. Yield: 10.0 g (65%).

To a solution of 2-amino-4,6-dimethoxy-benzamide (7.50 g, 38.2 mmol) and 3-(2-hydroxy-ethoxy)-5-methoxy-benzaldehyde (7.50 g, 38.2 mmol) in N,N-dimethylacetamide (30 mL) was added NaHSO₃ (58.5 wt %, 4.37 g, 42.0 mmol) and p-TSA (0.72 g, 3.8 mmol). The reaction mixture was heated to 115-120° C. for 16 hours, and then cooled to room temperature. N,N-dimethylacetamide was removed under reduced pressure, the residue was diluted with water (50 mL), and the solid was filtered, collected, and mixed with ether (50 mL), then filtered and dried under vacuum, to give 2-[3-(2-hydroxy-ethoxy)-5-methoxy-phenyl]-5,7-dimethoxy-3H-quinazolin-4-one as a white solid. Yield: 10 g (70%).

To a solution of 2-[3-(2-hydroxy-ethoxy)-5-methoxy-phenyl]-5,7-dimethoxy-3H-quinazolin-4-one (8.00 g, 21.5 mmol) in anhydrous DMF (30 mL) was added carbon tetrabromide (9.80 g, 29.5 mmol) and triphenylphosphine (7.78 g, 29.5 mmol). The reaction mixture was stirred at 40° C. for 7 hours. DMF was removed under vacuum and dichloromethane (200 mL) was added. The organic phase was washed with water (150 mL), brine (100 mL), and dried over anhydrous sodium sulfate. Solvent was removed and the residue was washed three times with a mixture of ether and dichloromethane (20:1, 200 mL) to give 2-[3-(2-bromo-ethoxy)-5-methoxy-phenyl]-5,7-dimethoxy-3H-quinazolin-4-one (5) as a white solid. Yield: 8.9 g (95%).

To a solution of 2-[3-(2-bromo-ethoxy)-5-methoxy-phenyl]-5,7-dimethoxy-3H-quinazolin-4-one (7.10 g, 16.0 mmol) in THF (20 mL) was added pyrrolidine (11.38 g, 160.0 mmol) and the reaction mixture was stirred at room temperature for 15 hours. THF was removed under reduced pressure, the residue was purified by column chromatography (silica gel 230-400 mesh; eluting with 5% 2.0 M ammonia in methanol solution in dichloromethane) to give the title compound as a white solid. Yield: 3.2 g (47%). MP 159-160° C. ¹H NMR (400 MHz, CDCl₃): δ 10.66 (s, 1H), 7.25 (m, 2H), 6.84 (d, J=2.0 Hz, 1H), 6.67 (t, J=2.4 Hz, 1H), 6.45 (d, J=2.0 Hz, 1H), 4.21 (t, J=6.0 Hz, 2H), 3.95 (s, 3H), 3.93 (s, 3H), 3.89 (s, 3H), 2.93 (t, J=6.0 Hz, 2H), 2.64 (m, 4H), 1.80 (m, 4H). MS (ES⁺) m/z: 426.20 (M+1).

### Example 115

### Preparation of N-(2-(3-(5,7-dimethoxy-4-oxo-3,4-dihydroquinazolin-2-yl)-5-methoxyphenoxy)ethyl)acetamide

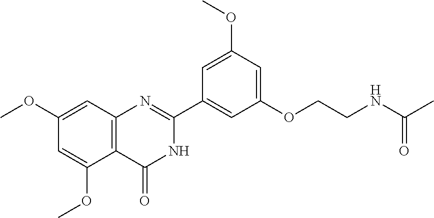 [SMILES: COc1cc(OCCNC(C)=O)cc(-c2nc3cc(OC)cc(OC)c3c(=O)[nH]2)c1]

To a 1.0-L three-neck flask was added sodium ethanethiolate (80%, 28.5 g, 271.0 mmol) and anhydrous DMF (225 mL). The mixture was heated to 145° C. for 1.5 hours; then, a solution of 3,5-dimethoxy-benzaldehyde (15.0 g, 90.0 mmol) in anhydrous DMF (350 mL) was added over a period of 8 minutes. The reaction was kept at 145° C. for 1 hour, then cooled to room temperature. Saturated sodium chloride solution (2.5 L) and formaline (37%, 240 mL), together with acetic acid (500 mL), was added. The resulting solution was thoroughly extracted with ethyl acetate, and the organic phase was dried over anhydrous sodium sulfate. Solvent was removed under vacuum, and the crude compound was purified by column chromatography (silica gel 230-400 mesh; eluting with 7:1 dichloromethane and ethyl acetate) to give 3-hydroxy-5-methoxy-benzaldehyde as a white solid. Yield: 12.0 g (88%).

To a solution of 3-hydroxy-5-methoxy-benzaldehyde (12.0 g, 78.9 mmol) in anhydrous DMF (50 mL) was added [1,3]dioxolan-2-one (13.9 g, 157.0 mmol) and potassium carbonate (21.6 g, 157.0 mmol). The reaction mixture was then heated to 110° C. for 16 hours, then cooled to room temperature. Solid potassium carbonate was filtered and washed with ethyl acetate. The organic phase was collected and solvent was removed. The residue was purified by column chromatography (silica gel 230-400 mesh; eluting with 7:1 dichloromethane and ethyl acetate) to give 3-(2-hydroxy-ethoxy)-5-methoxy-benzaldehyde as a brown liquid. Yield: 10.0 g (65%).

To a solution of 2-amino-4,6-dimethoxy-benzamide (7.50 g, 38.2 mmol) and 3-(2-hydroxy-ethoxy)-5-methoxy-benzaldehyde (7.50 g, 38.2 mmol) in N,N-dimethylacetamide (30 mL) were added NaHSO₃ (58.5 wt %, 4.37 g, 42.0 mmol) and p-TSA (0.72 g, 3.8 mmol). The reaction mixture was heated to 115-120° C. for 16 hours, and then cooled to room temperature. N,N-dimethylacetamide was removed under reduced pressure, the residue was diluted with water (50 mL), and the solid was filtered, collected and mixed with ether (50 mL), filtered, and dried under vacuum, to give 2-[3-(2-hydroxy-ethoxy)-5-methoxy-phenyl]-5,7-dimethoxy-3H-quinazolin-4-one as a white solid. Yield: 10 g (70%).

To a solution of 2-[3-(2-hydroxy-ethoxy)-5-methoxy-phenyl]-5,7-dimethoxy-3H-quinazolin-4-one (8.00 g, 21.5 mmol) in anhydrous DMF (30 mL) was added carbon tetrabromide (9.80 g, 29.5 mmol) and triphenylphosphine (7.78 g, 29.5 mmol). The reaction mixture was stirred at 40° C. for 7 hours. DMF was removed under vacuum and dichloromethane (200 mL) was added. The organic phase was washed with water (150 mL), then brine (100 mL), and dried over anhydrous sodium sulfate. Solvent was removed and the residue was washed three times with a mixture of ether and dichloromethane (20:1, 200 mL) to give 2-[3-(2-bromo-ethoxy)-5-methoxy-phenyl]-5,7-dimethoxy-3H-quinazolin-4-one as a white solid. Yield: 8.9 g (95%).

To a solution of 2-[3-(2-bromo-ethoxy)-5-methoxy-phenyl]-5,7-dimethoxy-3H-quinazolin-4-one (0.37 g, 0.84 mmol) in DMF (10 mL) was added sodium azide (0.14 g, 2.11 mmol) and the reaction mixture was stirred at 70° C. for 7 hours. DMF was removed under reduced pressure and dichloromethane (100 mL) was added. The organic phase was washed with water (50 mL), then brine (50 mL), and dried over anhydrous sodium sulfate. Solvent was removed and the residue was purified by column chromatography (silica gel 230-400 mesh; 30-40% ethyl acetate in dichloromethane as eluent) to give a white solid. Yield: 0.23 g (69%).

2-[3-(2-Azido-ethoxy)-5-methoxy-phenyl]-5,7-dimethoxy-3H-quinazolin-4-one (90 mg, 0.22 mmol) was taken in thioacetic acid (2 mL) and the reaction mixture was stirred at room temperature for 2 hours. Thioacetic acid was removed under reduced pressure, and the residue was purified by column chromatography (silica gel 230-400 mesh; 3.5% methanol in dichloromethane as eluent) to give the title compound as a white solid. Yield: 45 mg (49%). MP 264-265° C. ¹H NMR (400 MHz, DMSO-d₆): δ 12.05 (s, 1H), 8.13 (t, J=5.86 Hz, 1H), 7.39 (d, J=1.56 Hz, 2H), 6.76 (d, J=2.34 Hz, 1H), 6.69 (t, J=2.15 Hz, 1H), 6.55 (d, J=2.34 Hz, 1H), 4.07 (t, J=5.67 Hz, 2H), 3.90 (s, 3H), 3.85 (s, 3H), 3.83 (s, 3H), 3.43 (q, J=5.47 Hz, 2H), 1.84 (s, 3H). MS (ES⁺) m/z: 414.11 (M+1).

### Example 116

### Preparation of 5,7-Dimethoxy-2-(3-methoxyphenyl)quinazolin-4(3H)-one

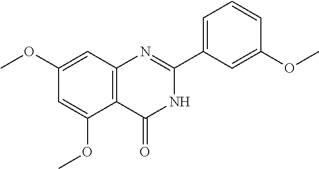

A mixture of 2-amino-4,6-dimethoxybenzamide (0.0600 g, 0.306 mmol), 3-methoxybenzaldehyde (0.306 mmol), NaHSO₃ (94%, 0.0474 g, 0.428 mmol), and p-TsOH.H₂O (0.0175 g, 0.0918 mmol) in DMA (3.06 mL) was heated at 140° C. for 20 hours. The mixture was diluted with EtOAc (300 mL), washed with water (3×75 mL), then brine (75 mL), dried over sodium sulfate, filtered, and concentrated under vacuum. The residue was purified on silica gel (40 g, CH₂Cl₂/MeOH) and the product was freeze-dried from MeCN/H₂O to provide the title compound (69%) as an off-white solid. ¹H NMR (300 MHz, DMSO-d₆): δ 12.04 (s, 1H), 7.82-7.70 (m, 2H), 7.43 (t, J=7.98 Hz, 1H), 7.13 (dd, J=8.19, 2.46 Hz, 1H), 6.76 (d, J=2.19 Hz, 1H), 6.55 (d, J=2.19 Hz, 1H), 3.92-3.82 (m, 9H); MS (APCI) m/z 313 [C₁₇H₁₆N₂O₄+H]⁺.

### Example 117

### Inhibition of Tetra-Acetylated Histone H4 Binding Individual BET Bromodomains

Proteins were cloned and overexpressed with a N-terminal 6×His tag, then purified by nickel affinity followed by size exclusion chromatography. Briefly, E. coli BL21(DE3) cells were transformed with a recombinant expression vector encoding N-terminally Nickel affinity tagged bromodomains from Brd2, Brd3, Brd4. Cell cultures were incubated at 37° C. with shaking to the appropriate density and induced overnight with IPTG. The supernatant of lysed cells was loaded onto Ni-IDA column for purification. Eluted protein was pooled, concentrated and further purified by size exclusion chromatography. Fractions representing monomeric protein were pooled, concentrated, aliquoted, and frozen at −80° C. for use in subsequent experiments.

Binding of tetra-acetylated histone H4 and BET bromodomains was confirmed by a Time Resolved Fluorescence Resonance Energy Transfer (TR-FRET) method. N-terminally His-tagged bromodomains (200 nM) and biotinylated tetra-acetylated histone H4 peptide (25-50 nM, Millipore) were incubated in the presence of Europium Cryptate-labeled streptavidin (Cisbio Cat. #610SAKLB) and XL665-labeled monoclonal anti-His antibody (Cisbio Cat. #61 HISXLB) in a white 96 well microtiter plate (Greiner). For inhibition assays, serially diluted test compound was added to these reactions in a 0.2% final concentration of DMSO. Final buffer concentrations were 30 mM HEPES pH 7.4, 30 mM NaCl, 0.3 mM CHAPS, 20 mM phosphate pH 7.0, 320 mM KF, 0.08% BSA). After 2 hours incubation at room temperature, the fluorescence by FRET was measured at 665 and 620 nm by a SynergyH4 plate reader (Biotek). Illustrative results with the first bromodomain of Brd4 Results are shown in Table 2. The binding inhibitory activity was shown by a decrease in 665 nm fluorescence relative to 620 nm. IC₅₀ values were determined from a dose response curve. Compounds with an IC₅₀ value less than 50 uM were deemed to be active.

TABLE 2
Inhibition of Binding of Tetra-acetylated Histone H4
and Brd4 bromodomain 1 as Measured by FRET

| | FRET |
|---|---|
| | activity |
| Compound | (<50 uM) |
| 2-(4-(4-(2-hydroxyethyl)piperazin-1-yl)phenyl)-5,7- | Active |
| dimethoxyquinazolin-4(3H)-one (Example 1) | |
| 2-(4-(4-butylpiperazin-1-yl)phenyl)-5,7- | Active |
| dimethoxyquinazolin-4(3H)-one (Example 2) | |
| 2-(4-(1-acetylpiperidin-4-yl)phenyl)-5,7- | Active |
| dimethoxyquinazolin-4(3H)-one (Example 3) | |
| 2-(4-(3-(cyclopropylmethylamino)pyrrolidin-1-yl)phenyl)- | Active |
| 5,7-dimethoxyquinazolin-4(3H)-one (Example 4) | |
| 2-(4-(2-(1-acetylazetidin-3-yl)ethoxy)-3,5-dimethylphenyl)- | Active |
| 5,7-dimethoxyquinazolin-4(3H)-one (Example 5) | |
| 2-(2,6-dimethylpyridin-4-yl)-5-(2-isopropoxyethoxy)-7- | Active |
| methoxyquinazolin-4(3H)-one (Example 6) | |
| 2-(4-((3R,5S)-4-Acetyl-3,5-dimethylpiperazin-1-yl)phenyl)- | Active |
| 5,7-dimethoxypyrido[2,3-d]pyrimidin-4(3H)-one (Example | |
| 7) | |
| 2-(4-(4-Hydroxypiperidin-1-yl)phenyl)-5,7- | Active |
| dimethoxypyrido[2,3-d]pyrimidin-4(3H)-one (Example 8) | |
| 2-(4-((3R,5S)-4-Acetyl-3,5-dimethylpiperazin-1-yl)phenyl)- | Active |
| 5-methoxy-7-(2-methoxyethoxy)quinazolin-4(3H)-one | |
| (Example 9) | |
| 2-(4-(4-Isopropylpiperazin-1-yl)phenyl)-5,7- | Active |
| dimethoxyquinazolin-4(3H)-one (Example 10) | |
| 2-(4-(4-Acetylpiperazin-1-yl)phenyl)-5,7- | Active |
| dimethoxyquinazolin-4(3H)-one (Example 11) | |
| 5,7-Dimethoxy-2-(4-(piperazin-1-yl)phenyl)quinazolin- | Active |
| 4(3H)-one (Example 12) | |
| N-(1-(4-(5,7-Dimethoxy-4-oxo-3,4-dihydroquinazolin-2- | Active |
| yl)phenyl)piperidin-4-yl)acetamide (Example 13) | |
| N-(1-(4-(5,7-Dimethoxy-4-oxo-3,4-dihydroquinazolin-2- | Active |
| yl)phenyl)piperidin-4-yl)methanesulfonamide (Example | |
| 14) | |
| 3-(1-(4-(5,7-Dimethoxy-4-oxo-3,4-dihydroquinazolin-2- | Active |
| yl)phenyl)piperidin-4-yl)-1,1-dimethylurea (Example 15) | |
| 2-(4-(4-Hexanoylpiperazin-1-yl)phenyl)-5,7- | Inactive |
| dimethoxyquinazolin-4(3H)-one (Example 16) | |
| 2-(4-(4-Isobutyrylpiperazin-1-yl)phenyl)-5,7- | Active |
| dimethoxyquinazolin-4(3H)-one (Example 17) | |
| 2-(4-(4-Benzoylpiperazin-1-yl)phenyl)-5,7- | Inactive |
| dimethoxyquinazolin-4(3H)-one (Example 18) | |
| 2-(4-(4-(4-Fluorobenzoyl)piperazin-1-yl)phenyl)-5,7- | Inactive |
| dimethoxyquinazolin-4(3H)-one (Example 19) | |
| N-(1-(4-(5,7-Dimethoxy-4-oxo-3,4-dihydroquinazolin-2- | Inactive |
| yl)phenyl)piperidin-4-yl)benzamide (Example 20) | |
| 5,7-Dimethoxy-2-(4-(4-picolinoylpiperazin-1- | Active |
| yl)phenyl)quinazolin-4(3H)-one (Example 21) | |
| 5,7-Dimethoxy-2-(4-(4-nicotinoylpiperazin-1- | Active |
| yl)phenyl)quinazolin-4(3H)-one (Example 22) | |
| 2-(4-(4-Isonicotinoylpiperazin-1-yl)phenyl)-5,7- | Active |
| dimethoxyquinazolin-4(3H)-one (Example 23) | |
| 5,7-Dimethoxy-2-(4-(4-(thiophene-2-carbonyl)piperazin-1- | Active |
| yl)phenyl)quinazolin-4(3H)-one (Example 24) | |
| 2-(4-(4-(5-Chloro-1-methyl-1H-pyrazole-4- | Inactive |
| carbonyl)piperazin-1-yl)phenyl)-5,7-dimethoxyquinazolin- | |
| 4(3H)-one (Example 25) | |
| 5,7-Dimethoxy-2-(4-(4-(3,3,3-trifluoropropanoyl)piperazin- | Active |
| 1-yl)phenyl)quinazolin-4(3H)-one (Example 26) | |
| 2-(4-(4-(2,5-Dichlorothiophene-3-carbonyl)piperazin-1- | Active |
| yl)phenyl)-5,7-dimethoxyquinazolin-4(3H)-one (Example | |
| 27) | |
| 2-(4-(4-(Cyclopropanecarbonyl)piperazin-1-yl)phenyl)-5,7- | Active |
| dimethoxyquinazolin-4(3H)-one (Example 28) | |
| 2-(4-(4-(4-Fluorobenzyl)piperazin-1-yl)phenyl)-5,7- | Inactive |
| dimethoxyquinazolin-4(3H)-one (Example 29) | |
| 2-(4-(4-Benzylpiperazin-1-yl)phenyl)-5,7- | Inactive |
| dimethoxyquinazolin-4(3H)-one (Example 30) | |
| 2-(4-(4-(2,2,2-Trifluoroethyl)piperazin-1- | Inactive |
| yl)phenyl)quinazolin-4(3H)-one (Example 31) | |
| 2-(4-(4-Acetyl-1,4-diazepan-1-yl)phenyl)-5,7- | Active |
| dimethoxyquinazolin-4(3H)-one (Example 32) | |
| 2-(4-(1,4-Diazepan-1-yl)phenyl)-5,7-dimethoxyquinazolin- | Active |
| 4(3H)-one (Example 33) | |
| 5,7-Dimethoxy-2-(4-(4-methyl-1,4-diazepan-1- | Active |
| yl)phenyl)quinazolin-4(3H)-one (Example 34) | |
| N-(1-(4-(5,7-Dimethoxy-4-oxo-3,4-dihydroquinazolin-2- | Active |
| yl)phenyl)piperidin-4-yl)-N-ethylacetamide (Example 35) | |
| 2-(4-((3R,5S)-4-Acetyl-3,5-dimethylpiperazin-1-yl)phenyl)- | Active |
| 5,7-dimethoxyquinazolin-4(3H)-one (Example 36) | |
| 2-(4-((3R,5S)-3,5-Dimethylpiperazin-1-yl)phenyl)-5,7- | Active |
| dimethoxyquinazolin-4(3H)-one (Example 37) | |
| 2-(4-(4-Acetyl-3-methylpiperazin-1-yl)phenyl)-5,7- | Active |
| dimethoxyquinazolin-4(3H)-one (Example 38) | |
| N-(1-(4-(5,7-Dimethoxy-4-oxo-3,4-dihydroquinazolin-2- | Active |
| yl)phenyl)pyrrolidin-3-yl)acetamide (Example 39) | |
| N-(1-(4-(5,7-dimethoxy-4-oxo-3,4-dihydroquinazolin-2- | Inactive |
| yl)phenyl)piperidin-4-yl)-N-isopropylacetamide (Example | |
| 40) | |
| 5-Chloro-2-(4-(4-isopropylpiperazin-1- | Inactive |
| yl)phenyl)quinazolin-4(3H)-one (Example 41) | |
| 2-(4-((3R,5S)-4-Isopropyl-3,5-dimethylpiperazin-1- | Active |
| yl)phenyl)-5,7-dimethoxyquinazolin-4(3H)-one (Example | |
| 42) | |
| 5,7-Dimethoxy-2-(4-(piperidin-4-yl)phenyl)quinazolin- | Active |
| 4(3H)-one (Example 43) | |
| 5,7-Dimethoxy-2-(4-(3-(methylamino)pyrrolidin-1- | Active |
| yl)phenyl)quinazolin-4(3H)-one (Example 44) | |
| N-(1-(4-(5,7-dimethoxy-4-oxo-3,4-dihydroquinazolin-2- | Active |
| yl)phenyl)pyrrolidin-3-yl)-N-methylacetamide (Example | |
| 46) | |
| 2-(4-(4-(Isopropylamino)piperidin-1-yl)phenyl)-5,7- | Active |
| dimethoxyquinazolin-4(3H)-one (Example 47) | |
| 5,7-Dimethoxy-2-(4-(3-methylpiperazin-1- | Active |
| yl)phenyl)quinazolin-4(3H)-one (Example 48) | |
| N-Benzyl-N-(1-(5-(5,7-dimethoxy-4-oxo-3,4- | Active |
| dihydroquinazolin-2-yl)pyridin-2-yl)piperidin-4- | |
| yl)acetamide (Example 49) | |
| 2-(6-(4-(Benzylamino)piperidin-1-yl)pyridin-3-yl)-5,7- | Active |
| dimethoxyquinazolin-4(3H)-one (Example 50) | |
| 4-(4-(5,7-Dimethoxy-4-oxo-3,4-dihydroquinazolin-2- | Active |
| yl)phenyl)piperazine-1-carbaldehyde (Example 51) | |
| 5,7-Dimethoxy-2-(4-(4-oxopiperidin-1-yl)phenyl)pyrido[2,3- | Inactive |
| d]pyrimidin-4(3H)-on (Example 52) | |
| 2-(2-(Hydroxymethyl)-1H-indol-5-yl)-5,7- | Active |
| dimethoxyquinazolin-4(3H)-one (Example 53) | |
| 2-(2-(2-Hydroxyethyl)-1H-indol-5-yl)-5,7- | Active |
| dimethoxyquinazolin-4(3H)-one (Example 54) | |
| 5,7-Dimethoxy-2-(2-(pyrrolidin-1-ylmethyl)-1H-indol-5- | Active |
| yl)quinazolin-4(3H)-one (Example 55) | |
| 2-(3-(Hydroxymethyl)-1H-indazol-5-yl)-5,7- | Active |
| dimethoxyquinazolin-4(3H)-one (Example 56) | |
| 5,7-Dimethoxy-2-(2-(2-(pyrrolidin-1-yl)ethyl)-1H-indol-5- | Active |
| yl)quinazolin-4(3H)-one (Example 57) | |
| 2-(2-((Dimethylamino)methyl)-1H-indol-5-yl)-5,7- | Active |
| dimethoxyquinazolin-4(3H)-one (Example 58) | |
| N-(4-(5,7-Dimethoxy-4-oxo-3,4-dihydroquinazolin-2- | Active |
| yl)phenyl)methanesulfonamide (Example 59) | |
| 5,7-Dimethoxy-2-(4-(pyridin-4-ylamino)phenyl)quinazolin- | Active |
| 4(3H)-one (Example 60) | |
| 5,7-Dimethoxy-2-(4-(p-tolylamino)phenyl)quinazolin- | Inactive |
| 4(3H)-one (Example 61) | |
| 5,7-Dimethoxy-2-(4-(pyridin-3-ylamino)phenyl)quinazolin- | Active |
| 4(3H)-one (Example 62) | |
| 4-(4-(5,7-Dimethoxy-4-oxo-3,4-dihydroquinazolin-2- | Active |
| yl)phenoxy)-N,N-dimethylpiperidine-1-carboxamide | |
| (Example 63) | |
| 2-(4-(1-Acetylpiperidin-4-yloxy)phenyl)-5,7- | Active |
| dimethoxyquinazolin-4(3H)-one (Example 64) | |
| 2-(4-(2-(Isoindolin-2-yl)ethoxy)-3,5-dimethylphenyl)-5,7- | Active |
| dimethoxyquinazolin-4(3H)-one (Example 65) | |
| 2-(3,5-Dimethyl-4-(2-(pyrrolidin-1-yl)ethoxy)phenyl)-5- | Active |
| methoxyquinazolin-4(3H)-one (Example 66) | |
| 5,7-Dichloro-2-(3,5-dimethyl-4-(2-(pyrrolidin-1- | Inactive |
| yl)ethoxy)phenyl)quinazolin-4(3H)-one (Example 67) | |
| 2-(4-(2-(4-Acetylpiperazin-1-yl)ethoxy)-3,5- | Active |
| dimethylphenyl)-5,7-dimethoxyquinazolin-4(3H)-one | |
| (Example 68) | |
| 2-(4-(2-(1H-Imidazol-1-yl)ethoxy)-3,5-dimethylphenyl)-5,7- | Active |
| dimethoxyquinazolin-4(3H)-one (Example 69) | |
| 2-(3,5-Dimethyl-4-(2-(pyrrolidin-1-yl)ethoxy)phenyl)-7- | Inactive |
| methoxyquinazolin-4(3H)-one (Example 70) | |
| 2-(3,5-Dimethyl-4-(2-(4-methylpiperazin-1- | Active |
| yl)ethoxy)phenyl)-5,7-dimethoxyquinazolin-4(3H)-one | |
| (Example 71) | |
| 2-(3,5-Dimethyl-4-(2-(piperidin-1-yl)ethoxy)phenyl)-5,7- | Active |
| dimethoxyquinazolin-4(3H)-one (Example 72) | |
| 5,7-Dimethoxy-2-(3-methyl-4-(2-(pyrrolidin-1- | Active |
| yl)ethoxy)phenyl)quinazolin-4(3H)-one (Example 73) | |
| 3-(2-(4-(5,7-Dimethoxy-4-oxo-3,4-dihydroquinazolin-2-yl)- | Active |
| 2,6-dimethylphenoxy)ethyl)-1-isopropylimidazolidine-2,4- | |
| dione (Example 74) | |
| 2-(3,5-Dimethyl-4-(3-(pyrrolidin-1-yl)propoxy)phenyl)-5,7- | Active |
| dimethoxyquinazolin-4(3H)-one (Example 75) | |
| 5,7-Dimethoxy-2-(4-(2-(pyrrolidin-1- | Active |
| yl)ethoxy)phenyl)quinazolin-4(3H)-one (Example 76) | |
| 2-(3,5-Dimethyl-4-(3-(pyrrolidin-1-yl)propyl)phenyl)-5,7- | Active |
| dimethoxyquinazolin-4(3H)-one (Example 77) | |
| 2-(3,5-Dimethyl-4-(4-(pyrrolidin-1-yl)butoxy)phenyl)-5,7- | Active |
| dimethoxyquinazolin-4(3H)-one (Example 78) | |
| 3-(2-(4-(5,7-Dimethoxy-4-oxo-3,4-dihydroquinazolin-2-yl)- | Active |
| 2,6-dimethylphenoxy)ethyl)-5-phenylimidazolidine-2,4- | |
| dione (Example 79) | |
| 3-(4-(5,7-Dimethoxy-4-oxo-3,4-dihydroquinazolin-2- | Inactive |
| yl)benzyl)imidazolidine-2,4-dione (Example 80) | |
| 2-(3,5-Dimethyl-4-(2-(pyrrolidin-1-yl)ethoxy)phenyl)-5,7- | Active |
| dimethoxypyrido[2,3-d]pyrimidin-4(3H)-one (Example 81) | |
| 2-(3,5-Dimethyl-4-(2-(pyrrolidin-1-yl)ethoxy)phenyl)-7- | Inactive |
| fluoro-5-(pyrrolidin-1-yl)quinazolin-4(3H)-one (Example | |
| 82) | |
| 5-Chloro-2-(3,5-dimethyl-4-(2-(pyrrolidin-1- | Inactive |
| yl)ethoxy)phenyl)quinazolin-4(3H)-one (Example 83) | |
| 2-(4-(2-(Azepan-1-yl)ethoxy)-3,5-dimethylphenyl)-5,7- | Active |
| dimethoxyquinazolin-4(3H)-one (Example 84) | |
| 2-(3,5-Dimethyl-4-(2-(pyrrolidin-1-yl)ethoxy)phenyl)-5,7- | Inactive |
| difluoroquinazolin-4(3H)-one (Example 85) | |
| 2-(4-(2-(Azetidin-1-yl)ethoxy)-3,5-dimethylphenyl)-5,7- | Active |
| dimethoxyquinazolin-4(3H)-one (Example 86) | |
| N-(1-(2-(4-(5,7-Dimethoxy-4-oxo-3,4-dihydroquinazolin-2- | |
| yl)-2,6-dimethylphenoxy)ethyl)azetidin-3-yl)acetamide | Active |
| (Example 87) | |
| 2-(3,5-Dimethyl-4-(2-(pyrrolidin-1-yl)ethoxy)phenyl)-5,7- | Inactive |
| diisopropoxyquinazolin-4(3H)-one (Example 88) | |
| 2-(3,5-Dimethyl-4-(2-(pyrrolidin-1-yl)ethoxy)phenyl)-5,7- | Active |
| dimethylquinazolin-4(3H)-one (Example 89) | |
| 2-(2-(4-(6,8-Dimethoxy-1-oxo-1,2-dihydroisoquinolin-3-yl)- | Inactive |
| 2,6-dimethylphenoxy)ethyl)isoindoline-1,3-dione | |
| (Example 90) | |
| 2-(3,5-Dimethyl-4-(2-(pyrrolidin-1-yl)ethoxy)phenyl)-5,7- | Inactive |
| diisopropoxypyrido[2,3-d]pyrimidin-4(3H)-one (Example | |
| 91) | |
| (S)-2-(3,5-Dimethyl-4-((5-oxopyrrolidin-2- | Active |
| yl)methoxy)phenyl)-5,7-dimethoxyquinazolin-4(3H)-one | |
| (Example 92) | |
| 2-(4-((4-Isopropylpiperazin-1-yl)methyl)phenyl)-5,7- | Active |
| dimethoxyquinazolin-4(3H)-one (Example 93) | |
| N-(1-(4-(5,7-Dimethoxy-4-oxo-3,4-dihydroquinazolin-2- | Active |
| yl)benzyl)piperidin-4-yl)-N-isopropylacetamide (Example | |
| 94) | |
| 2-(4-((4-(Isopropylamino)piperidin-1-yl)methyl)phenyl)-5,7- | Active |
| dimethoxyquinazolin-4(3H)-one (Example 95) | |
| 2-(4-((1H-Tetrazol-5-yl)methyl)phenyl)-5,7- | Active |
| dimethoxyquinazolin-4(3H)-one (Example 96) | |
| 1-(2-(4-(5,7-Dimethoxy-4-oxo-3,4-dihydroquinazolin-2-yl)- | Active |
| 2,6-dimethylphenoxy)ethyl)pyrrolidine-2,5-dione (Example | |
| 97) | |
| 7-(2-(Benzyloxy)ethoxy)-5-methoxy-2-(pyridin-4- | Inactive |
| yl)quinazolin-4(3H)-one (Example 98) | |
| 2-(2,6-Dimethylpyridin-4-yl)-5,7-dimethoxyquinazolin- | Active |
| 4(3H)-one (Example 99) | |
| 2-(2,6-Dimethylpyridin-4-yl)-5-methoxy-7-(2- | Active |
| methoxyethoxy)quinazolin-4(3H)-one (Example 100) | |
| 2-(2,6-Dimethylpyridin-4-yl)-5,7-bis(2- | Active |
| methoxyethoxy)quinazolin-4(3H)-one (Example 101) | |
| 2-(2,6-Dimethylpyridin-4-yl)-7-methoxy-5-(2-(pyrrolidin-1- | Active |
| yl)ethoxy)quinazolin-4(3H)-one (Example 102) | |
| 2-(2,6-Dimethylpyridin-4-yl)-5-methoxy-7-(2- | Active |
| phenoxyethoxy)quinazolin-4(3H)-one (Example 103) | |
| 2-(2,6-Dimethylpyridin-4-yl)-7-methoxy-5-(2- | Active |
| phenoxyethoxy)quinazolin-4(3H)-one (Example 104) | |
| 2-(2,6-Dimethylpyridin-4-yl)-7-methoxy-5-(2- | Active |
| methoxyethoxy)quinazolin-4(3H)-one (Example 105) | |
| 2-(2,6-Dimethylpyridin-4-yl)-5-methoxy-7-(2-(pyrrolidin-1- | Active |
| yl)ethoxy)quinazolin-4(3H)-one (Example 106) | |
| 2-(2,6-dimethylpyridin-4-yl)-5,7-bis(2- | Active |
| isopropoxyethoxy)quinazolin-4(3H)-one (Example 108) | |
| 7-(2-(Benzyloxy)ethoxy)-2-(2,6-dimethylpyridin-4-yl)-5- | Active |
| methoxyquinazolin-4(3H)-one (Example 109) | |
| 5-methoxy-7-(2-methoxyethoxy)-2-(2-methylpyridin-4- | Active |
| yl)quinazolin-4(3H)-one (Example 110) | |
| 2-(2,6-Dimethylpyridin-4-yl)-7-(2-methoxyethoxy)-5-(2- | Active |
| (pyrrolidin-1-yl)ethoxy)quinazolin-4(3H)-one (Example | |
| 111) | |
| 2-(3-(2-Hydroxyethoxy)phenyl)-5,7-dimethoxyquinazolin- | Active |
| 4(3H)-one (Example 112) | |
| 2-(3-(2-Hydroxyethoxy)-5-methylphenyl)-5,7- | Active |
| dimethoxyquinazolin-4(3H)-one (Example 113) | |
| 5,7-Dimethoxy-2-(3-methoxy-5-(2-(pyrrolidin-1- | Active |
| yl)ethoxy)phenyl)quinazolin-4(3H)-one (Example 114) | |
| N-(2-(3-(5,7-dimethoxy-4-oxo-3,4-dihydroquinazolin-2-yl)- | Inactive |
| 5-methoxyphenoxy)ethyl)acetamide (Example 115) | |
| 5,7-Dimethoxy-2-(3-methoxyphenyl)quinazolin-4(3H)-one | Inactive |
| (Example 116) | |

### Example 118

### Inhibition of c-myc Expression in Cancer Cell Lines

MV4-11 cells (2.5×10⁴ cells) were plated in 96 well U-bottom plates with test compound or DMSO (0.1%), and incubated for 3 hours at 37° C. Cells were then harvested by centrifugation, lysed, and mRNA was isolated using the mRNA catcher plus kit (Invitrogen). Reverse transcription of the mRNA and duplex amplification of the c-myc and cyclophilin cDNAs was performed using the RNA Ultrasense kit (Invitrogen) and a ViiA7 real-time PCR machine (Applied Biosystems). IC₅₀ values were determined from a dose response curve. Compounds with an IC₅₀ value less than 30 uM were deemed to be active.

TABLE 3
Inhibition of c-myc Activity in Human AML MV4-11 cells

| | c-myc |
|---|---|
| | activity |
| Compound | (<30 uM) |
| 2-(4-(4-Isopropylpiperazin-1-yl)phenyl)-5,7- | Active |
| dimethoxyquinazolin-4(3H)-one (Example 10) | |
| 5,7-Dimethoxy-2-(3-methyl-4-(2-(pyrrolidin-1- | Active |
| yl)ethoxy)phenyl)quinazolin-4(3H)-one (Example 73) | |
| 5,7-Dimethoxy-2-(3-methoxy-5-(2-(pyrrolidin-1- | Active |
| yl)ethoxy)phenyl)quinazolin-4(3H)-one (Example 114) | |
| N-(2-(3-(5,7-dimethoxy-4-oxo-3,4-dihydroquinazolin-2-yl)-5- | Inactive |
| methoxyphenoxy)ethyl)acetamide (Example 115) | |

### Example 119

### Inhibition of Cell Proliferation in Human AML MV-4-11 Cells

MV4-11 cells: 96-well plates were seeded with 5×10⁴ cells per well of exponentially growing human AML MV-4-11 (CRL-9591) cells and immediately treated with two-fold dilutions of test compounds, ranging from 30 μM to 0.2 μM. Triplicate wells were used for each concentration, as well as a media only and three DMSO control wells. The cells and compounds were incubated at 37° C., 5% CO₂ for 72 hours before adding 20 μL of the CellTiter Aqueous One Solution (Promega) to each well and incubated at 37° C., 5% CO₂ for an additional 3-4 hours. The absorbance was taken at 490 nm in a spectrophotometer and the percentage of proliferation relative to DMSO-treated cells was calculated after correction from the blank well. IC₅₀ were calculated using the GraphPad Prism software. Compounds with an IC₅₀ value less than 30 μM were deemed to be active.

TABLE 4
Inhibition of Cell Proliferation in Human AML MV-4-11 cells

| | cell |
|---|---|
| | proliferation |
| | activity |
| Compound | (<30 uM) |
| 2-(4-(4-Isopropylpiperazin-1-yl)phenyl)-5,7- | Inactive |
| dimethoxyquinazolin-4(3H)-one (Example 10) | |
| 5,7-Dimethoxy-2-(3-methoxy-5-(2-(pyrrolidin-1- | Active |
| yl)ethoxy)phenyl)quinazolin-4(3H)-one (Example 114) | |
| N-(2-(3-(5,7-dimethoxy-4-oxo-3,4-dihydroquinazolin-2-yl)- | Active |
| 5-methoxyphenoxy)ethyl)acetamide (Example 115) | |

### Example 120

### Lipopolysaccharide (LPS) Stimulated Whole Blood Assay for Measuring TNFa and IL-6 Levels

Activation of monocytic cells by agonists of toll-like receptors such as bacterial lipopolysaccharide (LPS) results in production of key inflammatory mediators including IL-6 and TNFa. Such pathways are widely considered to be central to the pathophysiology of a range of auto-immune and inflammatory disorders. Compounds to be tested are diluted to give a range of appropriate concentrations and 1 μl of the dilution stocks are added to wells of a 96 plate. Following addition of whole blood (130 μL) the plates are incubated at 37 degrees (5% CO₂) for 30 min before the addition of 10 μl of 2.8 μg/mL lipopolysaccharides (LPS), diluted in complete RPMI 1640 (final concentration=200 ng/mL), to give a total volume of 140 μL per well. After further incubation for 24 hours at 37 degrees, 140 μL of PBS are added to each well. The plates are sealed, shaken for 10 minutes and then centrifuged (2500 rpm×10 min). 100 μL of the supernatant are removed and IL-6 and TNFa levels assayed by immunoassay (typically by MesoScale Discovery technology) either immediately or following storage at −20 degrees. BET inhibitors tested in this assay will inhibit the production of the key inflammatory mediator IL-6 and/or TNFa.

### Example 121

### In Vivo Mouse Endotoxemia Model Assay

High doses of Endotoxin (bacterial lipopolysaccharide) are administered to animals produce a profound shock syndrome including a strong inflammatory response, dysregulation of cardiovascular function, organ failure and ultimately mortality. This pattern of response is very similar to human sepsis and septic shock, where the body's response to a significant bacterial infection can be similarly life threatening. To test the compounds for use in the invention groups of Balb/c male mice are given a lethal dose of 15 mg/kg LPS by intraperitoneal injection. Ninety minutes later, animals are dosed intravenously with vehicle (20% cyclodextrin 1% ethanol in apyrogen water) or test compound (10 mg/kg). The survival of animals is evaluated at 4 days. BET inhibitors tested in the mouse endotoxemia model assay will result in a significant animal survival effect following intravenous administration.

### Example 122

### Growth Suppressive Activity Test Against Cancer Cells

Using RPMI 1640 medium (manufactured by SIGMA) supplemented with 10% fetal bovine serum, human promyelocytic leukemia-derived cell line HL-60, human acute lymphoblastic leukemia-derived cell line MOLT4, human Burkitt's lymphoma-derived cell line Daudi, and human multiple myeloma-derived cell line RPMI-8226 are each cultured at 37° C., 5% CO₂. In addition, using ISKOV medium (manufactured by SIGMA) supplemented with 10% fetal bovine serum, human chronic myeloid leukemia-derived cell line MV4-11 is cultured at 37° C., 5% CO₂. Moreover, using DMEM/F-12 medium (manufactured by SIGMA) supplemented with 10% fetal bovine serum, human lung cancer cell-derived cell line EBC-1, human hepatocellular cancer-derived cell line Kim-1, human colorectal cancer-derived cell line HCT-116, human prostate cancer-derived cell line PC-3, human ovarian cancer-derived cell line A2780, and human osteosarcoma-derived cell line Saos2 are each cultured at 37° C., 5% CO₂. These cells are plated on a 96 well plate, and cultured for 1 day. To each culture test compound diluted with the medium to a final concentration of 0.0003⁻¹⁰ μm (final DMSO concentration, 0.4%) is added. After culture for 3 more days, WST-8 (0.16 mg/mL) is added to the culture medium and the cells are cultured for 2 hr. The absorbance at 650 nm is subtracted from the absorbance at 450 nm. The growth suppressive activity is shown by a decrease rate of the absorbance of the group receiving test compound to that of the control group, and GI₅₀ value is determined from a dose-reaction curve plotting a decrease rate of the absorbance obtained by changing the compound compound concentrations.

This assay demonstrates that a compound that inhibits binding between acetylated histone, more specifically acetylated histone H4, and a bromodomain-containing protein, more specifically human-derived BET family protein BRD2, BRD3 or BRD4 can be used as an antitumor agent.

### Example 123

### HIV Tat-Mediated Transactivation Inhibition Assay

This assay evaluates inhibition of Tat-mediated transactivation by BET inhibitors that block the PCAF bromodomain interaction with HIV-1 Tat-AcK50. The effect is assessed by a microinjection study as described previously by Dorr et al. (EMBO J. 21; 2715-2723, 2002). In this microinjection assay, HeLa-Tat cells are grown on Cellocate coverslips and microinjected at room temperature with an automated injection system (Carl Zeiss). Samples are prepared as a 20 μl injection mix containing the LTR-luciferase (100 ng/ml) and CMV-GFP (50 ng/ml) constructs together with 5 mg/ml a chemical compound or pre-immune IgGs. Live cells are examined on a Zeiss Axiovert microscope to determine the number of GFP-positive cells. Four hours after injection, cells are washed in cold phosphate buffer and processed for luciferase assays (Promega). BET inhibitors tested in this assay will inhibit Tat-mediated transactivation by the PCAF BRD inhibitor.

What is claimed is:

1. A method for inhibiting BET proteins comprising administering a therapeutically effective amount of at least one compound of Formula I: 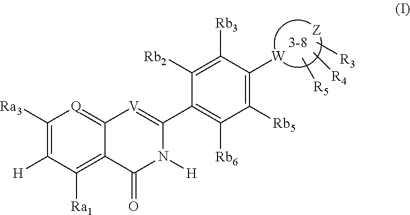
or stereoisomer, tautomer, pharmaceutically acceptable salt, or hydrate thereof, wherein:
Q and V are independently selected from CH and nitrogen;
Ra₁ and Ra₃ are independently selected from hydrogen, C₁-C₆ alkyl, C₁-C₆ alkoxy, halogen, amino, amide, hydroxyl, heterocycle, and C₃-C₆ cycloalkyl;
Rb₂ and Rb₆ are independently selected from hydrogen;
Rb₃ and Rb₅ are independently selected from hydrogen, halogen, C₁-C₆ alkyl, C₁-C₆ alkoxy, C₃-C₆ cycloalkyl, hydroxyl, and amino;
or wherein Rb₂ and Rb₃ and/or Rb₅ and Rb₆ are optionally connected to form a cycloalkyl or a heterocycle; 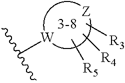
represents a 3-8 membered ring system wherein:
W is selected from carbon and nitrogen;
Z is selected from CR₆R₇, NR₈, oxygen, sulfur, —S(O)—, and —SO₂—;
said ring system being optionally fused to another ring selected from cycloalkyl, heterocycle, and phenyl, and wherein said ring system is optionally selected from rings having the structures: 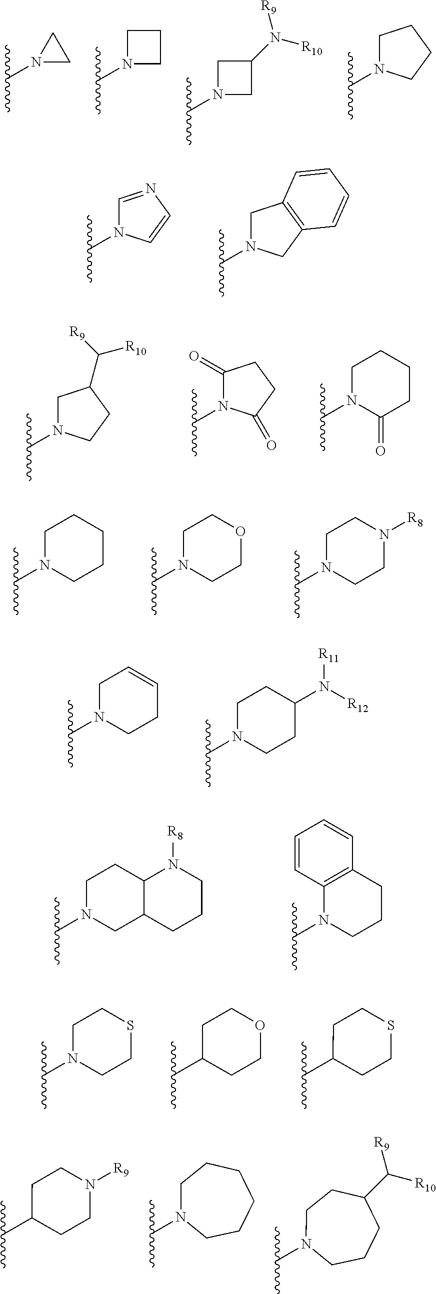 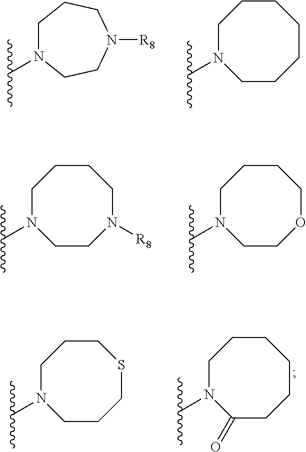
R₃, R₄, and R₅ are independently selected from hydrogen, C₁-C₆ alkyl, C₁-C₆ alkenyl, C₁-C₆ alkynyl, C₁-C₆ alkoxy, C₃-C₆ cycloalkyl, aryl, aryloxy, hydroxyl, amino, amide, oxo, —CN, and sulfonamide;
R₆ and R₇ are independently selected from hydrogen, C₁-C₆ alkyl, C₁-C₆ alkenyl, C₁-C₆ alkynyl, C₃-C₆ cycloalkyl, aryl, halogen, hydroxyl, —CN, amino, and amido; and
R₈ is selected from hydrogen, C₁-C₆ alkyl, C₁-C₆ alkenyl, C₁-C₆ alkynyl, acyl, and C₃-C₆ cycloalkyl; and
R₉, R₁₀, R₁₁, and R₁₂ are independently selected from hydrogen, C₁-C₆ alkyl, C₁-C₆ alkenyl, C₁-C₆ alkynyl, C₃-C₆ cycloalkyl, aryl, heterocycle, hydroxyl, sulfonyl, and acyl;
provided that:
if Q is CH, then at least one of Ra₁ and Ra₃ is not hydrogen;
if Z is NAc, then Ra₁ and Ra₃ are not hydrogen, and Ra₁ is not —OCH₂CH₂OMe; and
if Ra₁ and Ra₃ are both OMe, then R₈ is not —C(O)CH₂OH.

2. The method according to claim 1, wherein: 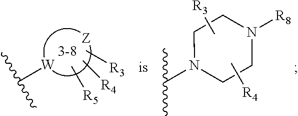
Ra₁ is selected from methyl, ethyl, methoxy, ethoxy, and propoxy; and
R₃ and R₄ are independently selected from hydrogen, C₁-C₆ alkyl, C₁-C₆ alkenyl, C₁-C₆ alkynyl, C₁-C₆ alkoxy, C₃-C₆ cycloalkyl, aryloxy, aryl, hydroxyl, amino, amide, oxo, —CN, and sulfonamide; and
R₈ is selected from hydrogen, C₁-C₆ alkyl, C₁-C₆ alkenyl, acyl, and C₁-C₆ alkynyl.

3. The method according to claim 1, wherein: 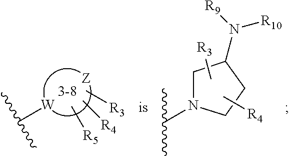
Ra₁ is selected from methyl, ethyl, methoxy, ethoxy, and propoxy; and
R₃ and R₄ are independently selected from hydrogen, C₁-C₆ alkyl, C₁-C₆ alkenyl, C₁-C₆ alkynyl, C₁-C₆ alkoxy, C₃-C₆ cycloalkyl, aryloxy, aryl, hydroxyl, amino, amide, oxo, —CN, and sulfonamide; and
R₉ and R₁₀ are independently selected from hydrogen, C₁-C₆ alkyl, C₁-C₆ alkenyl, C₁-C₆ alkynyl, C₃-C₆ cycloalkyl, aryl, heterocycle, sulfonyl, carbamate, carboxamide, and acyl.

4. The method according to claim 1, wherein: 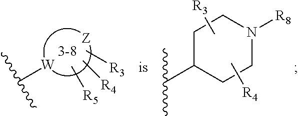
Ra₁ is selected from methyl, ethyl, methoxy, ethoxy, and propoxy;
R₃ and R₄ are independently selected from hydrogen, C₁-C₆ alkyl, C₁-C₆ alkenyl, C₁-C₆ alkynyl, C₁-C₆ alkoxy, C₃-C₆ cycloalkyl, aryloxy, aryl, hydroxyl, amino, amido, oxo, —CN, and sulfonamide; and
R₈ is selected from hydrogen, C₁-C₆ alkyl, C₁-C₆ alkenyl, C₁-C₆ alkynyl, acyl, and C₃-C₆ cycloalkyl.

5. The method according to claim 1, wherein:
Ra₁ is selected from methyl, ethyl, methoxy, ethoxy, and propoxy
Ra₃ is selected from C₁-C₆ alkoxy, hydrogen, and halogen;
Rb₂, Rb₃, Rb₅, and Rb₆ are each hydrogen; 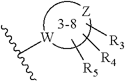
is selected from 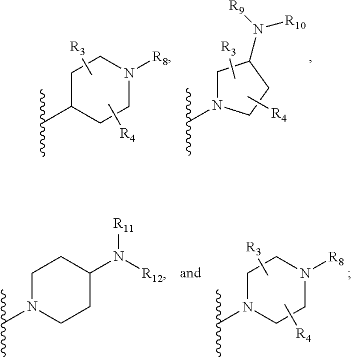
R₃ and R₄ are independently selected from hydrogen and C₁-C₆ alkyl;
R₈ is selected from C₁-C₆ alkyl and hydrogen; and
R₉, R₁₀, R₁₁, and R₁₂ are independently selected from C₁-C₆ alkyl, hydrogen, acyl, and sulfonyl.

6. The method according to claim 1, wherein:
Ra₁ is selected from methyl, ethyl, methoxy, ethoxy, and propoxy;
Ra₃ is selected from methoxy, hydrogen, and halogen;
Rb₃ and Rb₅ are each hydrogen; 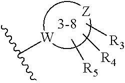
is selected from 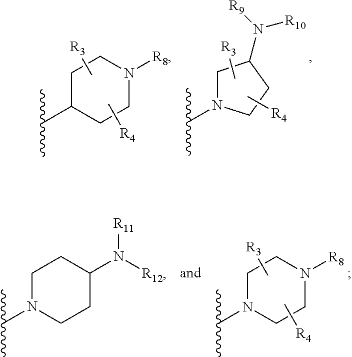
R₃ and R₄ are independently selected from hydrogen and methyl;
R₈ is selected from hydrogen, hydroxyethyl, butyl, acetyl, isopropyl, 4-hexanoyl, 4-isobutyryl, benzoyl, 4-fluorobenzoyl, 4-picolinoyl, 4-nicotinoyl, 4-isonicotinoyl, thiophene-2-carbonyl, 5-chloro-1-methyl-1H-pyrazole-4-carbonyl, 3,3,3-trifluoropropanoyl, 2,5-dichlorothiopene-3-carbonyl, cyclopropanecarbonyl, 4-fluorobenzyl, benzyl, 2,2,2-trifluoroethyl, tertbutoxycarbonyl, and formyl;
R₉ and R₁₀ are independently selected from hydrogen, methyl, cyclopropylmethyl, and acetyl; and
R₁₁ and R₁₂ are independently selected from hydrogen, acetyl, methanesulfonyl, dimethylaminocarbonyl, benzoyl, benzyl, ethyl, and isopropyl.

7. The method according to claim 1, wherein the compound of Formula I is selected from:
5,7-dimethoxy-2-(4-morpholinophenyl)quinazolin-4(3H)-one;
2-(4-((3R,5S)-4-acetyl-3,5-dimethylpiperazin-1-yl)phenyl)-5,7-dimethoxypyrido[2,3-d]pyrimidin-4(3H)-one;
2-(4-(4-hydroxypiperidin-1-yl)phenyl)-5,7-dimethoxypyrido[2,3-d]pyrimidin-4(3H)-one;
2-(4-((3R,5S)-4-acetyl-3,5-dimethylpiperazin-1-yl)phenyl)-5-methoxy-7-(2-methoxyethoxy)quinazolin-4(3H)-one;
2-(4-(4-isopropylpiperazin-1-yl)phenyl)-5,7-dimethoxyquinazolin-4(3H)-one;
2-(4-(4-acetylpiperazin-1-yl)phenyl)-5,7-dimethoxyquinazolin-4(3H)-one;
5,7-dimethoxy-2-(4-(piperazin-1-yl)phenyl)quinazolin-4(3H)-one;
N-(1-(4-(5,7-dimethoxy-4-oxo-3,4-dihydroquinazolin-2-yl)phenyl)piperidin-4-yl)acetamide;
N-(1-(4-(5,7-dimethoxy-4-oxo-3,4-dihydroquinazolin-2-yl)phenyl)piperidin-4-yl)methanesulfonamide;
3-(1-(4-(5,7-dimethoxy-4-oxo-3,4-dihydroquinazolin-2-yl)phenyl)piperidin-4-yl)-1,1-dimethylurea;
2-(4-(4-hexanoylpiperazin-1-yl)phenyl)-5,7-dimethoxyquinazolin-4(3H)-one;
2-(4-(4-isobutyrylpiperazin-1-yl)phenyl)-5,7-dimethoxyquinazolin-4(3H)-one;
2-(4-(4-benzoylpiperazin-1-yl)phenyl)-5,7-dimethoxyquinazolin-4(3H)-one;
2-(4-(4-(4-fluorobenzoyl)piperazin-1-yl)phenyl)-5,7-dimethoxyquinazolin-4(3H)-one;
N-(1-(4-(5,7-dimethoxy-4-oxo-3,4-dihydroquinazolin-2-yl)phenyl)piperidin-4-yl)benzamide;
5,7-dimethoxy-2-(4-(4-picolinoylpiperazin-1-yl)phenyl)quinazolin-4(3H)-one;
5,7-dimethoxy-2-(4-(4-nicotinoylpiperazin-1-yl)phenyl)quinazolin-4(3H)-one;
2-(4-(4-isonicotinoylpiperazin-1-yl)phenyl)-5,7-dimethoxyquinazolin-4(3H)-one;
5,7-dimethoxy-2-(4-(4-(thiophene-2-carbonyl)piperazin-1-yl)phenyl)quinazolin-4(3H)-one;
2-(4-(4-(5-chloro-1-methyl-1H-pyrazole-4-carbonyl)piperazin-1-yl)phenyl)-5,7-dimethoxyquinazolin-4(3H)-one;
5,7-dimethoxy-2-(4-(4-(3,3,3-trifluoropropanoyl)piperazin-1-yl)phenyl)quinazolin-4(3H)-one;
2-(4-(4-(2,5-dichlorothiophene-3-carbonyl)piperazin-1-yl)phenyl)-5,7-dimethoxyquinazolin-4(3H)-one;
2-(4-(4-(cyclopropanecarbonyl)piperazin-1-yl)phenyl)-5,7-dimethoxyquinazolin-4(3H)-one;
2-(4-(4-(4-fluorobenzyl)piperazin-1-yl)phenyl)-5,7-dimethoxyquinazolin-4(3H)-one;
2-(4-(4-benzylpiperazin-1-yl)phenyl)-5,7-dimethoxyquinazolin-4(3H)-one;
2-(4-(4-(2,2,2-trifluoroethyl)piperazin-1-yl)phenyl)quinazolin-4(3H)-one;
2-(4-(4-butylpiperazin-1-yl)phenyl)-5,7-dimethoxyquinazolin-4(3H)-one;
2-(4-(4-acetyl-1,4-diazepan-1-yl)phenyl)-5,7-dimethoxyquinazolin-4(3H)-one;
2-(4-(1,4-diazepan-1-yl)phenyl)-5,7-dimethoxyquinazolin-4(3H)-one;
5,7-dimethoxy-2-(4-(4-methyl-1,4-diazepan-1-yl)phenyl)quinazolin-4(3H)-one;
N-(1-(4-(5,7-dimethoxy-4-oxo-3,4-dihydroquinazolin-2-yl)phenyl)piperidin-4-yl)-N-ethylacetamide;
2-(4-((3R,5S)-4-acetyl-3,5-dimethylpiperazin-1-yl)phenyl)-5,7-dimethoxyquinazolin-4(3H)-one;
2-(4-((3R,5S)-3,5-dimethylpiperazin-1-yl)phenyl)-5,7-dimethoxyquinazolin-4(3H)-one;
2-(4-(4-acetyl-3-methylpiperazin-1-yl)phenyl)-5,7-dimethoxyquinazolin-4(3H)-one;
N-(1-(4-(5,7-dimethoxy-4-oxo-3,4-dihydroquinazolin-2-yl)phenyl)pyrrolidin-3-yl)acetamide;
2-(4-(4-(2-hydroxyethyl)piperazin-1-yl)phenyl)-5,7-dimethoxyquinazolin-4(3H)-one;
N-(1-(4-(5,7-dimethoxy-4-oxo-3,4-dihydroquinazolin-2-yl)phenyl)piperidin-4-yl)-N-isopropylacetamide;
5-chloro-2-(4-(4-isopropylpiperazin-1-yl)phenyl)quinazolin-4(3H)-one;
2-(4-((3R,5S)-4-isopropyl-3,5-dimethylpiperazin-1-yl)phenyl)-5,7-dimethoxyquinazolin-4(3H)-one;
5,7-dimethoxy-2-(4-(piperidin-4-yl)phenyl)quinazolin-4(3H)-one;
5,7-dimethoxy-2-(4-(3-(methylamino)pyrrolidin-1-yl)phenyl)quinazolin-4(3H)-one;
tert-butyl 4-(4-(5,7-dimethoxy-4-oxo-3,4-dihydroquinazolin-2-yl)phenyl)piperidine-1-carboxylate;
N-(1-(4-(5,7-dimethoxy-4-oxo-3,4-dihydroquinazolin-2-yl)phenyl)pyrrolidin-3-yl)-N-methylacetamide;
2-(4-(4-(isopropylamino)piperidin-1-yl)phenyl)-5,7-dimethoxyquinazolin-4(3H)-one;
2-(4-(1-acetylpiperidin-4-yl)phenyl)-5,7-dimethoxyquinazolin-4(3H)-one;
5,7-dimethoxy-2-(4-(3-methylpiperazin-1-yl)phenyl)quinazolin-4(3H)-one;
N-benzyl-N-(1-(5-(5,7-dimethoxy-4-oxo-3,4-dihydroquinazolin-2-yl)pyridin-2-yl)piperidin-4-yl)acetamide;
2-(6-(4-(benzylamino)piperidin-1-yl)pyridin-3-yl)-5,7-dimethoxyquinazolin-4(3H)-one;
4-(4-(5,7-dimethoxy-4-oxo-3,4-dihydroquinazolin-2-yl)phenyl)piperazine-1-carbaldehyde;
2-(4-(3-(cyclopropylmethylamino)pyrrolidin-1-yl)phenyl)-5,7-dimethoxyquinazolin-4(3H)-one;
5,7-dimethoxy-2-(4-(4-oxopiperidin-1-yl)phenyl)pyrido[2,3-d]pyrimidin-4(3H)-one;
and stereoisomers, tautomers, pharmaceutically acceptable salts, and hydrates thereof.

8. A method of inhibiting BET proteins comprising administering a therapeutically effective amount of at least one compound of Formula II: 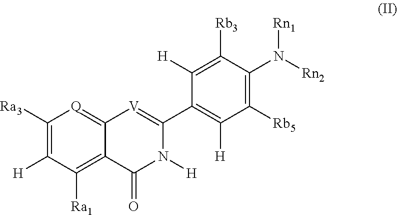
or stereoisomer, tautomer, pharmaceutically acceptable salt, or hydrate thereof, wherein:
Q and V are independently selected from CH and nitrogen;
Ra₁ and Ra₃ are independently selected from hydrogen, C₁-C₆ alkyl, C₁-C₆ alkoxy, C₃-C₆ cycloalkyl, halogen, amino, amide, hydroxyl, cycloalkyl, and heterocycle;
Rb₃ and Rb₅ are independently selected from hydrogen, halogen, C₁-C₆ alkyl, C₁-C₆ alkoxy, C₃-C₆ cycloalkyl, hydroxyl, and amino;
Rn₁ is selected from hydrogen, C₁-C₆ alkyl, and C₃-C₆ cycloalkyl;
Rn₂ is selected from C₁-C₆ alkyl, C₃-C₆ cycloalkyl, heterocycle, aryl, alkenyl, sulfonyl and acyl;
or wherein Rn₁ and/or Rn₂ are optionally connected with Rb₃ and/or Rb₅ to form a 5- or 6-membered heterocyclic ring;
provided that:
at least one of Ra₁ and Ra₃ are not hydrogen; and
Rn₁ and Rn₂ are not both methyl or ethyl.

9. The method according to claim 8, wherein:
Q is CH;
V is nitrogen;
Ra₁ and Ra₃ are each C₁-C₆ alkoxy;
Rb₃ is hydrogen;
Rn₁ is hydrogen;
Rn₂ is selected from sulfonyl, heterocycle, and aryl; and
Rb₅ is hydrogen or is connected with Rn₂ to form a heterocycle.

10. The method according to claim 8, wherein:
Q is CH;
V is nitrogen;
Ra₁ and Ra₃ are each methoxy;
Rb₃ is hydrogen;
Rn₁ is hydrogen;
Rn₂ is selected from methanesulfonyl, pyridin-4-yl, 4-methylphenyl, and pyridin-3-yl; and
Rb₅ is hydrogen or is connected with Rn₂ to form a heterocycle selected from (2-hydroxymethyl)-1H-pyrrol-5-yl, (2-hydroxyethyl)-1H-pyrrol-5-yl, 2-(pyrrolidin-1-yl-ylmethyl)-1H-pyrrol-5-yl, 3-(hydroxymethyl)-1H-pyrazol-5-yl, 2-(pyrrolidin-1-yl-ylethyl)-1H-pyrrol-5-yl, and 2-((dimethylamino)methyl)-1H-pyrrol-5-yl.

11. The method according to claim 8, wherein the compound of Formula II is selected from:
2-(4-(dimethylamino)naphthalen-1-yl)-6,7-dimethoxyquinazolin-4(3H)-one;
2-(4-(bis(2-hydroxyethyl)amino)phenyl)-5,7-dimethoxypyrido[2,3-d]pyrimidin-4(3H)-one;
2-(2-(hydroxymethyl)-1H-indol-5-yl)-5,7-dimethoxyquinazolin-4(3H)-one;
2-(2-(2-hydroxyethyl)-1H-indol-5-yl)-5,7-dimethoxyquinazolin-4(3H)-one;
5,7-dimethoxy-2-(2-(pyrrolidin-1-ylmethyl)-1H-indol-5-yl)quinazolin-4(3H)-one;
2-(3-(hydroxymethyl)-1H-indazol-5-yl)-5,7-dimethoxyquinazolin-4(3H)-one;
5,7-dimethoxy-2-(2-(2-(pyrrolidin-1-yl)ethyl)-1H-indol-5-yl)quinazolin-4(3H)-one;
2-(2-((dimethylamino)methyl)-1H-indol-5-yl)-5,7-dimethoxyquinazolin-4(3H)-one;
N-(4-(5,7-dimethoxy-4-oxo-3,4-dihydroquinazolin-2-yl)phenyl)methanesulfonamide;
5,7-dimethoxy-2-(4-(pyridin-4-ylamino)phenyl)quinazolin-4(3H)-one;
5,7-dimethoxy-2-(4-(p-tolylamino)phenyl)quinazolin-4(3H)-one;
5,7-dimethoxy-2-(4-(pyridin-3-ylamino)phenyl)quinazolin-4(3H)-one;
and stereoisomers, tautomers, pharmaceutically acceptable salts, and hydrates thereof.

12. A method of inhibiting BET proteins in a subject, comprising administering a therapeutically effective amount of at least one compound of Formula III: 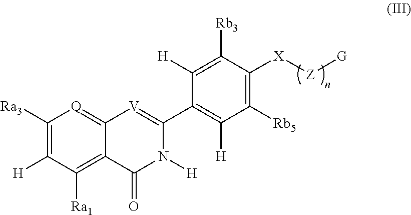
or stereoisomer, tautomer, pharmaceutically acceptable salt, or hydrate thereof, wherein:
Q is selected from CH and nitrogen;
V is selected from CH and nitrogen;
X is selected from oxygen, sulfur, SR₁, nitrogen, NR₆R₇, and CR₆R₇;
Z is selected from unsubstituted C₁-C₆ alkyl and C₁-C₆ alkyl substituted with one or more groups selected from C₁-C₃ alkyl, C₁-C₃ alkoxy, cyclopropyl, hydroxyl, amino, and halogen;
n is selected from 0, 1, 2, or 3;
G is selected from heterocycle, cycloalkyl, and aryl;
R₁ is selected from hydrogen, and C₁-C₆ alkyl;
R₆ and R₇ are independently selected from hydrogen, C₁-C₆ alkyl, C₃-C₆ cycloalkyl, heterocycle, C₁-C₆ alkoxy, and halogen;
Ra₁ and Ra₃ are independently selected from hydrogen, C₁-C₆ alkyl, C₁-C₆ alkoxy, C₃-C₆ cycloalkyl, halogen, amino, amide, hydroxyl, and heterocycle; and
Rb₃ and Rb₅ are independently selected from hydrogen, halogen, C₁-C₆ alkyl, C₃-C₆ cycloalkyl, C₁-C₆ alkoxy, hydroxyl, and amino;
provided that:
if Ra₁ and Ra₃ are OMe, and Q is CH, then 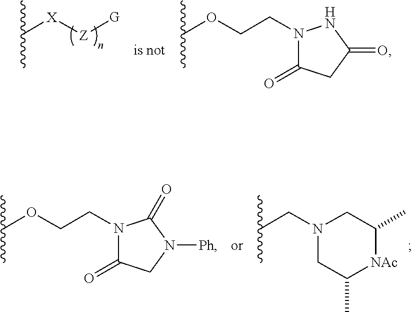
at least one of Ra₁ and Ra₃ is not hydrogen; and
if Ra₃ is chloro, then Ra₁ is not hydrogen.

13. The method according to claim 12, wherein:
Q is selected from CH and nitrogen;
V is nitrogen;
Z is selected from unsubstituted C₁-C₆ alkyl;
Ra1 is selected from methyl, ethyl, methoxy, ethoxy, and propoxy;
Ra₃ is selected from hydrogen, C₁-C₆ alkyl, C₁-C₆ alkoxy, halogen, and heterocycle;
Rb₃ and Rb₅ are independently selected from hydrogen and C₁-C₆ alkyl;
X is selected from oxygen and CH₂;
n is selected from 0, 1, 2, or 3; and
G is selected from heterocycle, cycloalkyl, and aryl.

14. The method according to claim 12, wherein:
Q is selected from CH and nitrogen;
V is nitrogen;
Ra₁ is selected from methyl, ethyl, methoxy, ethoxy, and propoxy;
Ra₃ is selected from hydrogen, methyl, chlorine, fluorine, methoxy, isopropoxy, and pyrrolidin-1-yl;
Rb₃ and Rb₅ are independently selected from hydrogen and methyl; and 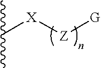
is selected from (N,N-dimethylpiperidine-1-carboxamide)-4-oxy, 1-acetylpiperidin-4-yloxy, 2-(isoindolin-2-yl)ethoxy, 2-(pyrrolidin-1-yl)ethoxy, 3-(pyrrolidin-1-yl)propoxy, 4-(pyrrolidin-1-yl)butoxy, (4-acetylpiperazin-1-yl)ethoxy, (1H-imidazol-1-yl)ethoxy, (4-methylpiperazin-1-yl)ethoxy, (piperidin-1-yl)ethoxy, (1-isopropylimidazolidine-2,4-dione)-3-ethoxy, (5-phenylimidazolidine-2,4-dione)-3-ethoxy, (imidazolidine-2,4-dione)-3-methyl, (2-azepan-1-yl)ethoxy, (2-azetidin-1-yl)ethoxy, N-(azetidin-3-yl)acetamide-1-ethoxy, (isoindoline-1,3-dione)-2-ethoxy, (5-oxopyrrolidin-2-yl)methoxy, (4-isopropylpiperazin-1-yl)methyl, N-isopropyl-N-(piperidin-4-methyl)acetamide-1-methyl, (4-(isopropylamino)piperidin-1-yl)methyl, (pyrrolidine-2,5-dione)ethoxy, and (1H-tetrazol-5-yl)methyl.

15. The method according to claim 12, wherein the compound of Formula III is selected from:
3-(3,5-dimethyl-4-(2-morpholinoethoxy)phenyl)-6,8-dimethoxyisoquinolin-1(2H)-one;
2-(3,5-dimethyl-4-(2-morpholinoethoxy)phenyl)-5,7-dimethoxyquinazolin-4(3H)-one;
3-(3,5-dimethyl-4-(2-(4-methylpiperazin-1-yl)ethoxy)phenyl)-6,8-dimethoxyisoquinolin-1(2H)-one;
2-(3,5-dimethyl-4-(2-morpholinoethoxy)phenyl)quinazolin-4(3H)-one;
7-(3,5-dimethyl-4-(2-morpholinoethoxy)phenyl)-2,4-dimethoxy-1,6-naphthyridin-5(6H)-one;
5,7-dimethoxy-2-(4-((4-methylpiperazin-1-yl)methyl)phenyl)quinazolin-4(3H)-one;
5,7-dimethoxy-2-(4-(morpholinomethyl)phenyl)quinazolin-4(3H)-one;
2-(4-((4-ethylpiperazin-1-yl)methyl)phenyl)-5,7-dimethoxyquinazolin-4(3H)-one;
2-(3,5-dimethyl-4-(2-(pyrrolidin-1-yl)ethoxy)phenyl)-5,7-dimethoxyquinazolin-4(3H)-one;
4-(4-(5,7-dimethoxy-4-oxo-3,4-dihydroquinazolin-2-yl)phenoxy)-N,N-dimethylpiperidine-1-carboxamide;
2-(4-(1-acetylpiperidin-4-yloxy)phenyl)-5,7-dimethoxyquinazolin-4(3H)-one;
2-(4-(2-(isoindolin-2-yl)ethoxy)-3,5-dimethylphenyl)-5,7-dimethoxyquinazolin-4(3H)-one;
2-(3,5-dimethyl-4-(2-(pyrrolidin-1-yl)ethoxy)phenyl)-5-methoxyquinazolin-4(3H)-one;
5,7-dichloro-2-(3,5-dimethyl-4-(2-(pyrrolidin-1-yl)ethoxy)phenyl)quinazolin-4(3H)-one;
2-(4-(2-(4-acetylpiperazin-1-yl)ethoxy)-3,5-dimethylphenyl)-5,7-dimethoxyquinazolin-4(3H)-one;
2-(4-(2-(1H-imidazol-1-yl)ethoxy)-3,5-dimethylphenyl)-5,7-dimethoxyquinazolin-4(3H)-one;
2-(3,5-dimethyl-4-(2-(pyrrolidin-1-yl)ethoxy)phenyl)-7-methoxyquinazolin-4(3H)-one;
2-(3,5-dimethyl-4-(2-(4-methylpiperazin-1-yl)ethoxy)phenyl)-5,7-dimethoxyquinazolin-4(3H)-one;
2-(3,5-dimethyl-4-(2-(piperidin-1-yl)ethoxy)phenyl)-5,7-dimethoxyquinazolin-4(3H)-one;
5,7-dimethoxy-2-(3-methyl-4-(2-(pyrrolidin-1-yl)ethoxy)phenyl)quinazolin-4(3H)-one;
3-(2-(4-(5,7-dimethoxy-4-oxo-3,4-dihydroquinazolin-2-yl)-2,6-dimethylphenoxy)ethyl)-1-isopropylimidazolidine-2,4-dione;
2-(3,5-dimethyl-4-(3-(pyrrolidin-1-yl)propoxy)phenyl)-5,7-dimethoxyquinazolin-4(3H)-one;
5,7-dimethoxy-2-(4-(2-(pyrrolidin-1-yl)ethoxy)phenyl)quinazolin-4(3H)-one;
2-(3,5-dimethyl-4-(3-(pyrrolidin-1-yl)propyl)phenyl)-5,7-dimethoxyquinazolin-4(3H)-one;
2-(3,5-dimethyl-4-(4-(pyrrolidin-1-yl)butoxy)phenyl)-5,7-dimethoxyquinazolin-4(3H)-one;
3-(2-(4-(5,7-dimethoxy-4-oxo-3,4-dihydroquinazolin-2-yl)-2,6-dimethylphenoxy)ethyl)-5-phenylimidazolidine-2,4-dione;
3-(4-(5,7-dimethoxy-4-oxo-3,4-dihydroquinazolin-2-yl)benzyl)imidazolidine-2,4-dione;
2-(3,5-dimethyl-4-(2-(pyrrolidin-1-yl)ethoxy)phenyl)-5,7-dimethoxypyrido[2,3-d]pyrimidin-4(3H)-one;
2-(3,5-dimethyl-4-(2-(pyrrolidin-1-yl)ethoxy)phenyl)-7-fluoro-5-(pyrrolidin-1-yl)quinazolin-4(3H)-one;
5-chloro-2-(3,5-dimethyl-4-(2-(pyrrolidin-1-yl)ethoxy)phenyl)quinazolin-4(3H)-one;
2-(4-(2-(azepan-1-yl)ethoxy)-3,5-dimethylphenyl)-5,7-dimethoxyquinazolin-4(3H)-one;
2-(3,5-dimethyl-4-(2-(pyrrolidin-1-yl)ethoxy)phenyl)-5,7-difluoroquinazolin-4(3H)-one;
2-(4-(2-(azetidin-1-yl)ethoxy)-3,5-dimethylphenyl)-5,7-dimethoxyquinazolin-4(3H)-one;
N-(1-(2-(4-(5,7-dimethoxy-4-oxo-3,4-dihydroquinazolin-2-yl)-2,6-dimethylphenoxy)ethyl)azetidin-3-yl)acetamide;
2-(3,5-dimethyl-4-(2-(pyrrolidin-1-yl)ethoxy)phenyl)-5,7-diisopropoxyquinazolin-4(3H)-one;
2-(3,5-dimethyl-4-(2-(pyrrolidin-1-yl)ethoxy)phenyl)-5,7-dimethylquinazolin-4(3H)-one;
2-(2-(4-(6,8-dimethoxy-1-oxo-1,2-dihydroisoquinolin-3-yl)-2,6-dimethylphenoxy)ethyl)isoindoline-1,3-dione;
2-(3,5-dimethyl-4-(2-(pyrrolidin-1-yl)ethoxy)phenyl)-5,7-diisopropoxypyrido[2,3-d]pyrimidin-4(3H)-one;
2-(2-(4-(5,7-dimethoxy-4-oxo-3,4-dihydroquinazolin-2-yl)-2,6-dimethylphenoxy)ethyl)isoindoline-1,3-dione;
(S)-2-(3,5-dimethyl-4-((5-oxopyrrolidin-2-yl)methoxy)phenyl)-5,7-dimethoxyquinazolin-4(3H)-one;
2-(4-((4-isopropylpiperazin-1-yl)methyl)phenyl)-5,7-dimethoxyquinazolin-4(3H)-one;
N-(1-(4-(5,7-dimethoxy-4-oxo-3,4-dihydroquinazolin-2-yl)benzyl)piperidin-4-yl)-N-isopropylacetamide;
2-(4-(2-(1-acetylazetidin-3-yl)ethoxy)-3,5-dimethylphenyl)-5,7-dimethoxyquinazolin-4(3H)-one;
2-(4-((4-(isopropylamino)piperidin-1-yl)methyl)phenyl)-5,7-dimethoxyquinazolin-4(3H)-one;
2-(4-((1H-tetrazol-5-yl)methyl)phenyl)-5,7-dimethoxyquinazolin-4(3H)-one;
and stereoisomers, tautomers, pharmaceutically acceptable salts, and hydrates thereof.

16. A method of inhibiting BET proteins comprising administering a therapeutically effective amount of at least one compound of Formula IV: 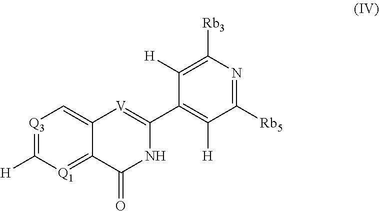
or a stereoisomer, tautomer, pharmaceutically acceptable salt, or hydrate thereof, wherein:
Q₁ is selected from nitrogen and C—Ra₁;
Q₃ is selected from nitrogen and C—Ra₃;
V is selected from CH and nitrogen;
Ra₁ and Ra₃ are independently selected from hydrogen, C₁-C₆ alkyl, C₁-C₆ alkenyl, C₁-C₆ alkynyl, C₁-C₆ alkoxy, C₃-C₆ cycloalkyl, amino, amide, and heterocycle;
or wherein Ra₁ and Ra₂ and/or Ra₂ and Ra₃ are connected to form a cycloalkyl or a heterocycle; and
Rb₃ and Rb₅ are independently selected from hydrogen, methyl, ethyl, C₃-C₆ cycloalkyl, O₁—O₃ alkoxy, and amino;
provided that:
if Ra₃ is alkoxy, then Ra₁ is not hydrogen; and
if Rb₅ is hydrogen, then Rb₃ is not —CH₂OH.

17. The method according to claim 16, wherein
V is nitrogen;
Rb₃ and Rb₅ are independently selected from C₁-C₆ alkyl and hydrogen;
Ra₃ is selected from hydrogen and C₁-C₆ alkoxy;
Ra₁ is selected from methyl, ethyl, methoxy, ethoxy, and propoxy.

18. The method according to claim 16, wherein
V is nitrogen;
Rb₃ and Rb₅ are independently selected from methyl and hydrogen;
Ra₁ is selected from methyl, ethyl, methoxy, ethoxy, and propoxy; and
Ra₃ is selected from hydrogen, benzyloxyethoxy, methoxy, methoxyethoxy, (pyrrolidin-1-yl)ethoxy, phenoxyethoxy, and isopropoxyethoxy.

19. The method according to claim 16, wherein the compound of Formula IV is selected from:
1-(2-(4-(5,7-dimethoxy-4-oxo-3,4-dihydroquinazolin-2-yl)-2,6-dimethylphenoxy)ethyl)pyrrolidine-2,5-dione;
7-(2-(benzyloxy)ethoxy)-5-methoxy-2-(pyridin-4-yl)quinazolin-4(3H)-one;
2-(2,6-dimethylpyridin-4-yl)-5,7-dimethoxyquinazolin-4(3H)-one;
2-(2,6-dimethylpyridin-4-yl)-5-methoxy-7-(2-methoxyethoxy)quinazolin-4(3H)-one;
2-(2,6-dimethylpyridin-4-yl)-5,7-bis(2-methoxyethoxy)quinazolin-4(3H)-one;
2-(2,6-dimethylpyridin-4-yl)-7-methoxy-5-(2-(pyrrolidin-1-yl)ethoxy)quinazolin-4(3H)-one;
2-(2,6-dimethylpyridin-4-yl)-5-methoxy-7-(2-phenoxyethoxy)quinazolin-4(3H)-one;
2-(2,6-dimethylpyridin-4-yl)-7-methoxy-5-(2-phenoxyethoxy)quinazolin-4(3H)-one;
2-(2,6-dimethylpyridin-4-yl)-7-methoxy-5-(2-methoxyethoxy)quinazolin-4(3H)-one;
2-(2,6-dimethylpyridin-4-yl)-5-methoxy-7-(2-(pyrrolidin-1-yl)ethoxy)quinazolin-4(3H)-one;
2-(2,6-dimethylpyridin-4-yl)-7-(2-isopropoxyethoxy)-5-methoxyquinazolin-4(3H)-one;
2-(2,6-dimethylpyridin-4-yl)-5,7-bis(2-isopropoxyethoxy)quinazolin-4(3H)-one;
7-(2-(benzyloxy)ethoxy)-2-(2,6-dimethylpyridin-4-yl)-5-methoxyquinazolin-4(3H)-one;
5-methoxy-7-(2-methoxyethoxy)-2-(2-methylpyridin-4-yl)quinazolin-4(3H)-one;
2-(2,6-dimethylpyridin-4-yl)-5-(2-isopropoxyethoxy)-7-methoxyquinazolin-4(3H)-one;
2-(2,6-dimethylpyridin-4-yl)-7-(2-methoxyethoxy)-5-(2-(pyrrolidin-1-yl)ethoxy)quinazolin-4(3H)-one;
and stereoisomers, tautomers, pharmaceutically acceptable salts, and hydrates thereof.

20. A method of inhibiting BET proteins comprising administering a therapeutically effective amount of at least one compound of Formula V: 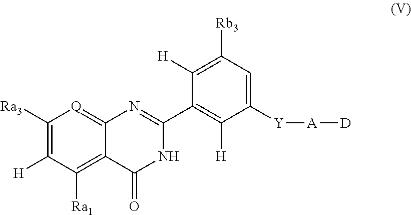
or a stereoisomer, tautomer, pharmaceutically acceptable salt, or hydrate thereof, wherein:
Q is selected from CH and nitrogen;
Y is selected from oxygen, nitrogen, sulfur, NR₆, CR₆R₇;
A is C₁-C₄ alkyl, wherein the alkyl chain may be connected to Y, D, and/or Rb₃ to form a cycloalkyl or heterocycle;
D, if present, is selected from —OR₁, —NR₁R₂;
R₁ and R₂ are independently selected from hydrogen, C₁-C₆ alkyl, C₃-C₆ cycloalkyl, sulfonamide, carboxamide, acyl, and nitrile, wherein R₁ and R₂ may be connected to form a cycloalkyl or a heterocycle;
R₆ and R₇ are independently selected from hydrogen, C₁-C₆ alkyl, C₃-C₆ cycloalkyl, C₁-C₆ alkoxy, hydroxyl, and halogen;
Ra₁ and Ra₃ are independently selected from hydrogen, C₁-C₆ alkyl, C₁-C₆ alkoxy, C₃-C₆ cycloalkyl, halogen, amino, amide, hydroxyl, and heterocycle; and
Rb₃ is selected from hydrogen, halogen, C₁-C₆ alkyl, C₃-C₆ cycloalkyl, C₁-C₆ alkoxy, hydroxyl, and amino;
provided that at least one of Ra₁ and Ra₃ is not hydrogen.

21. The method according to claim 20, wherein:
Ra₁ is selected from methyl, ethyl, methoxy, ethoxy, and propoxy;
Ra₃ is selected from hydrogen and C₁-C₆ alkoxy;
Q is CH;
Rb₃ is selected from hydrogen, C₁-C₆ alkyl, and C₁-C₆ alkoxy;
Y is selected from oxygen;
A is C₁-C₄ alkyl;
D, if present, is selected from hydroxy, heterocycle, and NR₁R₂; and
R₁ and R₂ are independently selected from hydrogen and C₁-C₆ alkyl, or alternatively, R₁ and R₂ are connected to form a cycloalkyl or a heterocycle.

22. The method according to claim 20, wherein:
Ra₁ is selected from methyl, ethyl, methoxy, ethoxy, and propoxy;
Ra₃ is selected from hydrogen and C₁-C₆ alkoxy;
Q is CH;
Rb₃ is selected from hydrogen, methyl, and methoxy;
Y is oxygen;
A is selected from methyl and ethyl;
D, if present, is selected from hydroxy, pyrrolidin-1-yl, and NR₁R₂; and
R₁ and R₂ are independently selected from hydrogen and acetyl, or alternatively, R₁ and R₂ are connected to form a cycloalkyl or a heterocycle.

23. The method according to claim 20, wherein the compound of Formula V is selected from:
2-(3,5-dimethoxyphenyl)-5,7-dimethoxyquinazolin-4(3H)-one;
2-(3-(2-hydroxyethoxy)phenyl)-5,7-dimethoxyquinazolin-4(3H)-one;
2-(3-(2-hydroxyethoxy)-5-methylphenyl)-5,7-dimethoxyquinazolin-4(3H)-one;
5,7-dimethoxy-2-(3-methoxy-5-(2-(pyrrolidin-1-yl)ethoxy)phenyl)quinazolin-4(3H)-one;
N-(2-(3-(5,7-dimethoxy-4-oxo-3,4-dihydroquinazolin-2-yl)-5-methoxyphenoxy)ethyl)acetamide;
5,7-dimethoxy-2-(3-methoxyphenyl)quinazolin-4(3H)-one;
and stereoisomers, tautomers, pharmaceutically acceptable salts, and hydrates thereof.

24. The method according to claim 1, wherein the therapeutically effective amount of the compound is administered with at least one pharmaceutically acceptable carrier in a pharmaceutically acceptable composition.

25. The method according to claim 1, wherein the compound of Formula I is administered to treat or prevent a cancer selected from cancers that exhibit c-myc overexpression, cancers that overexpress n-myc, cancers that that rely on the recruitment of p-TEFb to regulate activated oncogenes, Burkitt's lymphoma, acute myelogenous leukemia, multiple myeloma, aggressive human medulloblastoma, hematological, epithelial cancers, lung cancers, breast cancers, colon carcinomas, midline carcinomas, mesenchymal tumors, hepatic tumors, renal tumors, and neurological tumors.

26. The method of claim 25, wherein the compound of Formula I is administered in combination with another anti-cancer agent selected from the group consisting of bortezomib, thalidomide, dexamethasone, 5-azacitidine, decitabine, vorinostat, cyclophosphamide, a PI3K or mTOR inhibitor, rapamycin or a rapamycin analog, a gamma secretase inhibitor, an AMPK inducer, metformin, phenformin, an ornithine decarboxylase inhibitor, and difluoromethylornithine.
